(19) United States
(12) Patent Application Publication
(10) Pub. No.: US 2025/0270341 A1
(43) Pub. Date: Aug. 28, 2025

(54) CD98HC ANTIGEN-BINDING DOMAINS AND USES THEREFOR

(71) Applicant: ALECTOR LLC, South San Francisco, CA (US)

(72) Inventors: Eric BROWN, South San Francisco, CA (US); Alexander Gregory GULEVICH, Pinole, CA (US); Hamid SALIMI, San Mateo, CA (US); Angie Grace YEE, San Francisco, CA (US); Margaret L. TANG, Mountain View, CA (US); Tarangsri NIVITCHANYONG, Castro Valley, CA (US); Rajkumar GANESAN, Thousand Oaks, CA (US); Sarah A. JAHN, San Francisco, CA (US); Lu SHAN, Belmont, CA (US); Thunga BIENLY, Daly City, CA (US); Raymond Ka-Hang TONG, San Francisco, CA (US); Alexander Hyun-min YANG, San Jose, CA (US)

(21) Appl. No.: 19/039,230

(22) Filed: Jan. 28, 2025

Related U.S. Application Data
(63) Continuation of application No. PCT/US2023/071238, filed on Jul. 28, 2023.
(60) Provisional application No. 63/495,514, filed on Apr. 11, 2023.
(60) Provisional application No. 63/489,675, filed on Mar. 10, 2023.
(60) Provisional application No. 63/369,885, filed on Jul. 29, 2022.

(51) Int. Cl. C07K 16/28; A61K 39/00

(52) U.S. Cl. CPC C07K 16/2896; A61K 2039/505; C07K 2317/24; C07K 2317/31; C07K 2317/33; C07K 2317/34; C07K 2317/622; C07K 2317/732; C07K 2317/734; C07K 2317/77; C07K 2317/92; C07K 2319/00

(57) ABSTRACT

The present disclosure is generally directed to antigen-binding domains that specifically bind to human CD98 heavy chain (CD98hc) and their use in transport across the blood brain barrier (BBB).

### CROSS-REFERENCE TO RELATED APPLICATIONS

This application claims the priority benefit of U.S. Provisional Appl. Nos. 63/369,885, filed Jul. 29, 2022; 63/489,675, filed Mar. 10, 2023; and 63/495,514, filed Apr. 11, 2023; each of which is herein incorporated by reference in its entirety.

### REFERENCE TO SEQUENCE LISTING SUBMITTED ELECTRONICALLY

The content of the electronically submitted sequence listing (Name: 4503_021PC03_SequenceListing_ST26.xml; Size: 498,296 bytes; and Date of Creation: Jul. 27, 2023) is herein incorporated by reference in its entirety.

### FIELD OF THE PRESENT DISCLOSURE

The present disclosure relates to antigen-binding domains that specifically bind to human CD98 heavy chain (CD98hc). These antigen-binding domains can cross the blood brain barrier and can transport other agents (e.g., therapeutically active agents) associated with the antigen-binding domain across the blood brain barrier.

### BACKGROUND

Passive transfer of substances from blood to brain is restricted by the blood brain barrier (BBB). The BBB provides precise control of central nervous system (CNS) homeostasis allowing for proper neuronal function and also protecting neural tissue from toxins and pathogens. Alterations of the BBB are an important component of pathology and progression of different neurological diseases. However, the BBB poses a problem with regard to delivering therapeutics to the CNS. While recombinant proteins and antibody therapeutics have shown much success outside the CNS, such biologics do not cross the BBB efficiently. As a result, delivery of some therapeutics to the CNS has relied on injection of the therapeutic directly into the CNS. However, such injections are invasive procedures that have efficacy that is limited by the rapid export of cerebral spinal fluid (CSF) containing the therapeutic from the brain to the blood. Alternatively, a therapeutic intended for the CNS may be administered systemically at a high dose to allow for sufficient penetration of the BBB by the therapeutic. However, this approach may result in unintended effects due to the high dose in the periphery or increased manufacturing and formulation burdens to achieve the high dose. Accordingly, improved products and methods for delivering therapeutics across the BBB are needed.

### SUMMARY OF THE PRESENT DISCLOSURE

Provided herein are antigen-binding domains, fusion proteins, antibodies, and multi-specific proteins that specifically bind to human CD98 heavy chain (CD98hc), and methods of making and using the same.

In some aspects, provided herein is an antigen-binding domain that specifically binds to human CD98 heavy chain (CD98hc), wherein the antigen-binding domain comprises heavy chain variable region (VH) complementarity determining region (CDR) 1, VH CDR2, VH CDR3 and light chain variable region (VL) CDR1, CDR2, and CDR3 sequences comprising the amino acid sequences of: SEQ ID NOs: 413, 414, 112, and 176-178, respectively; SEQ ID NOs: 50-52 and 116-118, respectively; SEQ ID NOs: 53-55 and 119-121, respectively; SEQ ID NOs: 56-58 and 122-124, respectively; SEQ ID NOs: 59-61 and 125-127, respectively; SEQ ID NOs: 62-64 and 128-130, respectively; SEQ ID NOs: 65-67 and 131-133, respectively; SEQ ID NOs: 68-70 and 134-136, respectively; SEQ ID NOs: 71-73 and 137-139, respectively; SEQ ID NOs: 74-76 and 140-142, respectively; SEQ ID NOs: 77-79 and 143-145, respectively; SEQ ID NOs: 80-82 and 146-148, respectively; SEQ ID NOs: 83-85 and 149-151, respectively; SEQ ID NOs: 86-88 and 152-154, respectively; SEQ ID NOs: 89-91 and 155-157, respectively; SEQ ID NOs: 92-94 and 158-160, respectively; SEQ ID NOs: 95-97 and 161-163, respectively; SEQ ID NOs: 98-100 and 164-166, respectively; SEQ ID NOs: 101-103 and 167-169, respectively; SEQ ID NOs: 104-106 and 170-172, respectively; SEQ ID NOs: 107-109 and 173-175, respectively; SEQ ID NOs: 110⁻¹¹² and 176-178, respectively; SEQ ID NOs: 113-115 and 179-181, respectively; SEQ ID NOs: 226-228 and 273-275, respectively; SEQ ID NOs: 229-231 and 276-278, respectively; SEQ ID NOs: 232-234 and 279-281, respectively; SEQ ID NOs: 235-237 and 282-284, respectively; SEQ ID NOs: 238-240 and 285-287, respectively; SEQ ID NOs: 241-243 and 288-290, respectively; SEQ ID NOs: 244-246, 273, 274, and 291, respectively; SEQ ID NOs: 247-249, 292, 274, and 293, respectively; SEQ ID NOs: 250-252, 294, 274, and 291, respectively; SEQ ID NOs: 253-255 and 295-297, respectively; SEQ ID NOs: 256-258, 298, 274, and 299, respectively; SEQ ID NOs: 259-261 and 300-302, respectively; SEQ ID NOs: 262-264, 303, 274, and 304, respectively; SEQ ID NOs: 265-267, 305, 306, and 291, respectively; SEQ ID NOs: 268-270 and 307-309, respectively; SEQ ID NOs: 265, 271, 272, 310, 274, and 299, respectively; SEQ ID NOs: 110, 414, 112, and 176-178, respectively; SEQ ID NOs: 110, 414, 112, and 176-178, respectively; SEQ ID NOs: 413, 415, 112, and 176-178, respectively; SEQ ID NOs: 110, 415, 112, and 176-178, respectively; SEQ ID NOs: 110, 416, 112, and 176-178, respectively; SEQ ID NOs: 413, 416, 112, and 176-178, respectively; SEQ ID NOs: 413, 415, 112, 417, 418, and 178, respectively; SEQ ID NOs: 419, 422, 112, and 176-178, respectively; SEQ ID NOs: 419, 423, 112, and 176-178, respectively; SEQ ID NOs: 419, 424, 112, and 176-178, respectively; SEQ ID NOs: 419, 425, 112, and 176-178, respectively; SEQ ID NOs: 419, 426, 112, and 176-178, respectively; SEQ ID NOs: 419, 422, 112, 427, 177, and 178, respectively; SEQ ID NOs: 419, 422, 112, 428, 177, and 178, respectively; SEQ ID NOs: 419, 422, 112, 429, 177, and 178, respectively; SEQ ID NOs: 419, 422, 112, 430, 177, and 178, respectively; SEQ ID NOs: 419, 422, 112, 431, 177, and 178, respectively; SEQ ID NOs: 419, 422, 112, 432, 177, and 178, respectively; SEQ ID NOs: 419, 422, 112, 433, 177, and 178, respectively; SEQ ID NOs: 420, 422, 112, and 176-178, respectively; SEQ ID NOs: 421, 422, 112, and 176-178, respectively; or SEQ ID NOs: 421, 426, 112, 434, 177, and 178, respectively.

In some aspects, the antigen-binding domain comprises a VH and a VL, wherein the VH and VL comprise amino acid sequences at least 80%, at least 85%, at least 90%, at least 95%, or at least 99% identical to the amino acid sequences of: SEQ ID NOs: 363 and 364, respectively; SEQ ID NOs: 6 and 7, respectively; SEQ ID NOs: 8 and 9, respectively; SEQ ID NOs: 10 and 11, respectively; SEQ ID NOs: 12 and 13, respectively; SEQ ID NOs: 14 and 15, respectively; SEQ ID NOs: 16 and 17, respectively; SEQ ID NOs: 18 and 19, respectively; SEQ ID NOs: 20 and 21, respectively; SEQ ID NOs: 22 and 23, respectively; SEQ ID NOs: 24 and 25, respectively; SEQ ID NOs: 26 and 27, respectively; SEQ ID NOs: 28 and 29, respectively; SEQ ID NOs: 30 and 31, respectively; SEQ ID NOs: 32 and 33, respectively; SEQ ID NOs: 34 and 35, respectively; SEQ ID NOs: 36 and 37, respectively; SEQ ID NOs: 38 and 39, respectively; SEQ ID NOs: 40 and 41, respectively; SEQ ID NOs: 42 and 43, respectively; SEQ ID NOs: 44 and 45, respectively; SEQ ID NOs: 46 and 47, respectively; SEQ ID NOs: 48 and 49, respectively; SEQ ID NOs: 194 and 195, respectively; SEQ ID NOs: 196 and 197, respectively; SEQ ID NOs: 198 and 199, respectively; SEQ ID NOs: 200 and 201, respectively; SEQ ID NOs: 202 and 203, respectively; SEQ ID NOs: 204 and 205, respectively; SEQ ID NOs: 206 and 207, respectively; SEQ ID NOs: 208 and 209, respectively; SEQ ID NOs: 210 and 211, respectively; SEQ ID NOs: 212 and 213, respectively; SEQ ID NOs: 214 and 215, respectively; SEQ ID NOs: 216 and 217, respectively; SEQ ID NOs: 218 and 219, respectively; SEQ ID NOs: 220 and 221, respectively; SEQ ID NOs: 222 and 223, respectively; SEQ ID NOs: 224 and 225, respectively; SEQ ID NOs: 355 and 356, respectively; SEQ ID NOs: 357 and 358, respectively; SEQ ID NOs: 359 and 360, respectively; SEQ ID NOs: 361 and 362, respectively; SEQ ID NOs: 365 and 366, respectively; SEQ ID NOs: 367 and 368, respectively; SEQ ID NOs: 369 and 370, respectively; SEQ ID NOs: 371 and 372, respectively; SEQ ID NOs: 373 and 374, respectively; SEQ ID NOs: 375 and 376, respectively; SEQ ID NOs: 377 and 378, respectively; SEQ ID NOs: 379 and 380, respectively; SEQ ID NOs: 381 and 382, respectively; SEQ ID NOs: 383 and 384, respectively; SEQ ID NOs: 385 and 386, respectively; SEQ ID NOs: 387 and 388, respectively; SEQ ID NOs: 389 and 390, respectively; SEQ ID NOs: 391 and 392, respectively; SEQ ID NOs: 393 and 394, respectively; SEQ ID NOs: 395 and 396, respectively; SEQ ID NOs: 397 and 398, respectively; SEQ ID NOs: 399 and 400, respectively; SEQ ID NOs: 401 and 402, respectively; or SEQ ID NOs: 403 and 404, respectively.

In some aspects, provided herein is an antigen-binding domain that specifically binds to human CD98hc, wherein the antigen-binding domain comprises a VH and a VL, wherein the VH comprises the amino acid sequence of SEQ ID NO:363, 6, 8, 10, 12, 14, 16, 18, 20, 22, 24, 26, 28, 30, 32, 34, 36, 38, 40, 42, 44, 46, 48, 194, 196, 198, 200, 202, 204, 206, 208, 210, 212, 214, 216, 218, 220, 222, 224, 355, 357, 359, 361, 365, 367, 369, 371, 373, 375, 377, 379, 381, 383, 385, 387, 389, 401, or 403.

In some aspects, provided herein is an antigen-binding domain that specifically binds to human CD98hc, wherein the antigen-binding domain comprises a VH and a VL, wherein the VL comprises the amino acid sequence of SEQ ID NO: 364, 7, 9, 11, 13, 15, 17, 19, 21, 23, 25, 27, 29, 31, 33, 35, 37, 39, 41, 43, 45, 47, 49, 195, 197, 199, 201, 203, 205, 207, 209, 211, 213, 215, 217, 219, 221, 223, 225, 356, 358, 360, 362, 366, 368, 370, 372, 374, 376, 378, 380, 382, 384, 386, 388, 400, 402, or 404.

In some aspects, an antigen-binding domain provided herein comprises a VH and VL comprising the amino acid sequences of: SEQ ID NOs: 363 and 364, respectively; SEQ ID NOs: 6 and 7, respectively; SEQ ID NOs: 8 and 9, respectively; SEQ ID NOs: 10 and 11, respectively; SEQ ID NOs: 12 and 13, respectively; SEQ ID NOs: 14 and 15, respectively; SEQ ID NOs: 16 and 17, respectively; SEQ ID NOs: 18 and 19, respectively; SEQ ID NOs: 20 and 21, respectively; SEQ ID NOs: 22 and 23, respectively; SEQ ID NOs: 24 and 25, respectively; SEQ ID NOs: 26 and 27, respectively; SEQ ID NOs: 28 and 29, respectively; SEQ ID NOs: 30 and 31, respectively; SEQ ID NOs: 32 and 33, respectively; SEQ ID NOs: 34 and 35, respectively; SEQ ID NOs: 36 and 37, respectively; SEQ ID NOs: 38 and 39, respectively; SEQ ID NOs: 40 and 41, respectively; SEQ ID NOs: 42 and 43, respectively; SEQ ID NOs: 44 and 45, respectively; SEQ ID NOs: 46 and 47, respectively; SEQ ID NOs: 48 and 49, respectively; SEQ ID NOs: 194 and 195, respectively; SEQ ID NOs: 196 and 197, respectively; SEQ ID NOs: 198 and 199, respectively; SEQ ID NOs: 200 and 201, respectively; SEQ ID NOs: 202 and 203, respectively; SEQ ID NOs: 204 and 205, respectively; SEQ ID NOs: 206 and 207, respectively; SEQ ID NOs: 208 and 209, respectively; SEQ ID NOs: 210 and 211, respectively; SEQ ID NOs: 212 and 213, respectively; SEQ ID NOs: 214 and 215, respectively; SEQ ID NOs: 216 and 217, respectively; SEQ ID NOs: 218 and 219, respectively; SEQ ID NOs: 220 and 221, respectively; SEQ ID NOs: 222 and 223, respectively; SEQ ID NOs: 224 and 225, respectively; SEQ ID NOs: 355 and 356, respectively; SEQ ID NOs: 357 and 358, respectively; SEQ ID NOs: 359 and 360, respectively; SEQ ID NOs: 361 and 362, respectively; SEQ ID NOs: 365 and 366, respectively; SEQ ID NOs: 367 and 368, respectively; SEQ ID NOs: 369 and 370, respectively; SEQ ID NOs: 371 and 372, respectively; SEQ ID NOs: 373 and 374, respectively; SEQ ID NOs: 375 and 376, respectively; SEQ ID NOs: 377 and 378, respectively; SEQ ID NOs: 379 and 380, respectively; SEQ ID NOs: 381 and 382, respectively; SEQ ID NOs: 383 and 384, respectively; SEQ ID NOs: 385 and 386, respectively; SEQ ID NOs: 387 and 388, respectively; SEQ ID NOs: 389 and 390, respectively; SEQ ID NOs: 391 and 392, respectively; SEQ ID NOs: 393 and 394, respectively; SEQ ID NOs: 395 and 396, respectively; SEQ ID NOs: 397 and 398, respectively; SEQ ID NOs: 399 and 400, respectively; SEQ ID NOs: 401 and 402, respectively; or SEQ ID NOs: 403 and 404, respectively.

In some aspects, the antigen-binding domain is capable of crossing the blood brain barrier (BBB). In some aspects, the antigen-binding domain binds to cynomolgus monkey CD98hc. In some aspects, the antigen-binding domain is internalized in blood-brain barrier epithelial cells. In some aspects, the blood-brain barrier epithelial cells are HCMEC/D3 cells.

In some aspects, the antigen-binding domain binds human CD98hc with an affinity between 500 nM and 10 μM. In some aspects, the antigen binding domain binds human CD98hc with an affinity between 50 nM and 500 nM. In some aspects, the antigen binding domain binds human CD98hc with an affinity between 1 nM and 50 nM. In some aspects, the antigen-binding domain binds to human CD98hc with an ELISA OD450 of at least 0.45 and/or binds to cynomolgus monkey CD98hc with an ELISA OD450 of at least 0.45. In some aspects, the antigen-binding domain binds human CD98hc with an affinity of 3.1 nM to 210 nM. In some aspects, the antigen-binding domain binds to cynomolgus CD98hc with an affinity of 3.2 nM to 1.5 μM. In some aspects, the affinity is measured by high throughput surface plasmon resonance (SPR) detection.

In some aspects, the antigen-binding domain does not reduce cell-surface expression of CD98hc on HCMEC/D3 cells by more than 20% relative to cell-surface expression of CD98hc on HCMEC/D3 cells treated with an isotype control. In some aspects, the antigen-binding domain does not increase cell-surface expression of CD98hc on HCMEC/D3 cells by more than 50% relative to cell-surface expression of CD98hc on HCMEC/D3 cells treated with an isotype control.

In some aspects, the antigen-binding domain accumulates at least 1.5-fold or at least 2-fold more than an isotype control in vessel-depleted mouse brain. In some aspects, the antigen-binding domain has at least a 5-fold increase in brain:serum concentration ratio over an isotype control 24 hours after administration to a mouse.

In some aspects, the antigen-binding domain comprises a VH and VL comprising the amino acid sequences of SEQ ID NOs: 46 and 47, respectively; SEQ ID NOs: 367 and 368, respectively; SEQ ID NOs: 369 and 370, respectively; SEQ ID NOs: 371 and 372, respectively; SEQ ID NOs: 373 and 374, respectively; SEQ ID NOs: 375 and 376, respectively; SEQ ID NOs: 377 and 378, respectively; SEQ ID NOs: 379 and 380, respectively; SEQ ID NOs: 381 and 382, respectively; SEQ ID NOs: 383 and 384, respectively; SEQ ID NOs: 385 and 386, respectively; SEQ ID NOs: 387 and 388, respectively; SEQ ID NOs: 389 and 390, respectively; SEQ ID NOs: 391 and 392, respectively; SEQ ID NOs: 393 and 394, respectively; SEQ ID NOs: 395 and 396, respectively; SEQ ID NOs: 397 and 398, respectively; SEQ ID NOs: 399 and 400, respectively; SEQ ID NOs: 401 and 402, respectively; or SEQ ID NOs: 403 and 404, respectively. In some aspects, the antigen-binding domain comprises a VH and VL comprising the amino acid sequences of SEQ ID NOs: 14 and 15, respectively.

In some aspects, the antigen-binding domain comprises a VH and a VL on a single polypeptide chain. In some aspects, the antigen-binding domain comprises a single-chain fragment variable (scFv). In some aspects, the scFv is in the orientation VH-linker-VL. In some aspects, the scFv is in the orientation VL-linker-VH. In some aspects, the linker is about 5 to about 25 amino acids, is about 5 to about 20 amino acids, is about 10 to about 25 amino acids, or is about 10 to about 20 amino acids. In some aspects, the linker comprises the amino acid sequence of GGSEGKSSGSGSESKSTGGS (SEQ ID NO:182) or GGGGSGGGGSGGGGSGGGGS (SEQ ID NO:337). In some aspects, the scFv comprises the amino acid sequence of any one of SEQ ID NOs: 318-336. In some aspects, the antigen-binding domain comprises a VH on a first polypeptide and a VL on a second polypeptide. In some aspects, the antigen-binding domain is a murine, chimeric, humanized, or human antigen-binding domain. In some aspects, the antigen-binding domain is a humanized antigen-binding domain.

In some aspects, provided herein is an antigen-binding domain that specifically binds to human CD98hc, wherein the antigen-binding domain is a VHH comprises (i) the VH CDR1, VH CDR2, and VH CDR3 of the antigen-binding domain provided herein or (ii) the VH of the antigen-binding domain provided herein, optionally wherein the VHH is capable of crossing the blood brain barrier (BBB).

In some aspects, provided herein is a fusion protein comprising the antigen-binding domain provided herein and a heterologous protein or peptide. In some aspects, provided herein the heterologous protein or peptide comprises the amino acid sequence of beta-secretase 1 (BACE1), Abeta, epidermal growth factor receptor (EGFR), human epidermal growth factor receptor 2 (HER2), tau, apolipoprotein, apolipoprotein E (ApoE), apolipoprotein E4 (ApoE4), alpha-synuclein, CD20, huntingtin, prion protein (PrP), leucine rich repeat kinase 2 (LRRK2), parkin, presenilin 1, presenilin 2, glucocerebrosidase (GCase or GBA), progranulin (PGRN), Prosaposin (PSAP), Glycoprotein nonmetastatic protein B (GPNMB), gamma secretase, death receptor 6 (DR6), amyloid precursor protein (APP), p75 neurotrophin receptor (p75NTR), caspase 6, sortilin (SORT), triggering receptor expressed on myeloid cells 2 (TREM2), CD33 or sialic acid binding Ig-like lectin 3 (Siglec3), sialic acid binding Ig-like lectin 5 (Siglec5), sialic acid binding Ig-like lectin 7 (Siglec7), sialic acid binding Ig-like lectin 9 (Siglec9), Paired immunoglobin like type 2 receptor alpha (PILRA), Membrane Spanning 4-Domains A4A (MS4A4A), Membrane Spanning 4-Domains A 6A (MS4A6A), or Transmembrane Protein 106B (TMEM106b), clusterin (APOJ), Reelin, ubiquitin protein ligase E3A (UBE3A), Tripeptidyl Peptidase 1 (CLN2/TPP1), Alpha-L-Iduronidase (IDUA), Iduronate 2-Sulfatase (IDS), glucosamine (N-acetyl)-6-sulfatase (GNS), heparan-alpha-glucosaminide N-acetyltransferase (HGSNAT), and N-acetyl-alpha-glucosaminidase (NAGLU), N-sulfoglucosamine sulfohydrolase (SGSH), or a portion thereof.

In some aspects, provided herein is an antibody comprising the antigen-binding domain provided herein. In some aspects, provided herein is an antibody or antigen-binding fragment thereof that binds to the same human CD98hc epitope as the antigen-binding domain provided herein. In some aspects, provided herein is an antibody or antigen-binding fragment thereof that competitively inhibits binding of the antigen-binding domain provided herein to human CD98hc.

In some aspects, provided herein is a multi-specific protein comprising a first antigen-binding domain that is the antigen-binding domain provided herein linked to a second antigen-binding domain. In some aspects, the second antigen-binding domain specifically binds to a CNS antigen. In some aspects, provided herein is a multi-specific protein comprising the antigen-binding domain provided herein linked to an antibody or antigen-binding fragment thereof. In some aspects, the antibody or antigen-binding fragment thereof specifically binds to a CNS antigen. In some aspects, the antibody or antigen-binding fragment thereof comprises a heavy chain constant region. In some aspects, the antigen-binding domain provided herein is linked, optionally via an amino acid linker, to the C-terminus of the heavy chain constant region. In some aspects, the multi-specific protein is bispecific. In some aspects, the multi-specific protein is bivalent, trivalent, or tetravalent. In some aspects, the multi-specific protein is bivalent.

In some aspects, provided herein is a multi-specific protein is trivalent, optionally wherein the trivalent protein comprises one of the antigen-binding domain that binds to human CD98hc and two antigen-binding domains that bind to a CNS antigen.

In some aspects, provided herein is a multi-specific protein is tetravalent, optionally wherein the tetravalent protein comprises two of the antigen-binding domains that bind to human CD98hc and two antigen-binding domains that bind to a CNS antigen.

In some aspects, provided herein is a multi-specific protein that is trivalent and bi-specific and comprises the antigen-binding domain provided herein linked to an antibody that binds to a CNS antigen, wherein the antibody comprises two heavy chains and two light chains, and wherein the antigen-binding domain is an scFv linked, optionally via an amino acid linker, to the C-terminus of one of the two antibody heavy chains.

In some aspects, provided herein is a multi-specific protein that is tetravalent and bi-specific and comprises two antigen-binding domains provided herein, and an antibody that binds to a CNS antigen, wherein the antibody comprises two heavy chains and two light chains, wherein each of the two antigen-binding domains is an scFv, Fab, or VHH, wherein one of the two antigen-binding domains is linked, optionally via an amino acid linker, to the C-terminus of one of the antibody heavy chains, and wherein the other antigen-binding domain is linked, optionally via an amino acid linker, to the C-terminus of the other antibody heavy chain.

In some aspects, the the antibody or antigen-binding fragment thereof comprises a constant region comprising a knob mutation and a constant region comprising a hole mutation. In some aspects, the antigen-binding domain is linked, optionally via an amino acid linker, to the constant region comprising a hole mutation. In some aspects, the antigen-binding domain is linked, optionally via an amino acid linker, to the constant region comprising a knob mutation. In some aspects, the amino acid linker is a glycine-serine linker, optionally wherein the glycine-serine linker comprises the amino acid sequence (GGGGS)x3 (SEQ ID NO:183). In some aspects, the amino acid linker is a glycine-serine linker, optionally wherein the glycine-serine linker comprises the amino acid sequence (GGSGG)x3 (SEQ ID NO:338).

In some aspects, the CNS antigen is a brain antigen. In some aspects, the CNS antigen is not CD98hc.

In some aspects, the antibody or antigen-binding fragment thereof comprises a mutation that reduces effector function, optionally wherein the mutation that reduces effector function comprises (i) L234A, L235A, and/or P331S and/or (ii) N325S and/or L328F, and/or (iii) P329G or P329S. In some aspects, the antibody or antigen-binding fragment thereof comprises a constant region comprising a knob mutation and a mutation that reduces effector function, optionally wherein the mutation that reduces effector function comprises (i) L234A, L235A, and/or P331S and/or (ii) N325S and/or L328F, and/or (iii) P329G or P329S. In some aspects, the antibody or antigen-binding fragment thereof comprises a constant region comprising a hole mutation and a mutation that reduces effector function, optionally wherein the mutation that reduces effector function comprises (i) L234A, L235A, and/or P331S and/or (ii) N325S and/or L328F, and/or (iii) P329G or P329S.

In some aspects, the the antibody or antigen-binding fragment thereof is an IgG antibody or antigen-binding fragment thereof. In some aspects, the IgG antibody or antigen-binding fragment thereof is an IgG1 antibody or antigen-binding fragment thereof or an IgG4 antibody or antigen-binding fragment thereof.

In some aspects, the multi-specific protein binds human CD98hc with an equilibrium dissociation constant (KD) of about 3 nM to about 225 nM and/or binds cynomolgus monkey CD98hc with a KD of about 3 nM to about 225 nM.

In some aspects, the multi-specific protein is internalized in blood-brain barrier epithelial cells greater than 10-fold as compared to internalization by an isotype control, optionally wherein the blood-brain barrier epithelial cells are HCMEC/D3 cells. In some aspects, the multi-specific protein does not reduce cell-surface expression of CD98hc on HCMEC/D3 cells by more than 20% relative to cell-surface expression of CD98hc on HCMEC/D3 cells treated with an isotype control. In some aspects, the multi-specific protein does not increase cell-surface expression of CD98hc on HCMEC/D3 cells by more than 50% relative to cell-surface expression of CD98hc on HCMEC/D3 cells treated with an isotype control. In some aspects, the multi-specific protein accumulates at least 1.5-fold or at least 2-fold more than an isotype control in vessel-depleted mouse brain. In some aspects, the multi-specific protein has at least a 5-fold increase in brain:serum concentration ratio over an isotype control 24 hours after administration to a mouse.

In some aspects, the the CNS antigen is beta-secretase 1 (BACE1), Abeta, epidermal growth factor receptor (EGFR), human epidermal growth factor receptor 2 (HER2), tau, apolipoprotein, apolipoprotein E (ApoE), apolipoprotein E4 (ApoE4), alpha-synuclein, CD20, huntingtin, prion protein (PrP), leucine rich repeat kinase 2 (LRRK2), parkin, presenilin 1, presenilin 2, □-glucocerebrosidase (GCase or GBA), progranulin (PGRN), Prosaposin (PSAP), gamma secretase, death receptor 6 (DR6), amyloid precursor protein (APP), p75 neurotrophin receptor (p75NTR), caspase 6, sortilin (SORT), triggering receptor expressed on myeloid cells 2 (TREM2), CD33 or sialic acid binding Ig-like lectin 3 (Siglec3), sialic acid binding Ig-like lectin 5 (Siglec5), sialic acid binding Ig-like lectin 7 (Siglec7), sialic acid binding Ig-like lectin 9 (Siglec9), sialic acid binding Ig-like lectin 11 (Siglec11), glycoprotein nonmetastatic melanoma protein B (GPNMB), Paired immunoglobin like type 2 receptor alpha (PILRA), Membrane Spanning 4-Domains A4A (MS4A4A), Membrane Spanning 4-Domains A 6A (MS4A6A), MS4A4E, Transmembrane Protein 106B (TMEM106b), ubiquitin protein ligase E3A (UBE3A), CR1, ABCA1, ABCA7, HLA-DR1, HLA-DR5, IL1RAP, TREML2, IL-34, SORL1, or ADAM1. In some aspects, the CNS antigen is MS4A4A, optionally wherein (i) the antigen-binding domain, antibody, or antigen-binding domain that binds to MS4A4A comprises a VH comprising the amino acid sequence of SEQ ID NO:407 and/or a VL comprising the amino acid sequence of SEQ ID NO:405; and/or (ii) the antigen-binding that binds to human CD98hc comprises the amino acid sequence of SEQ ID NO:316.

In some aspects, the multi-specific protein comprises the amino acid sequences of SEQ ID NOs: 405-410.

In some aspects, the fusion protein provided herein, the antibody or antigen-binding fragment thereof provided herein, or the multi-specific protein provided herein is capable of crossing the BBB. In some aspects, the fusion protein, antibody or antigen-binding fragment thereof, or multi-specific protein provided herein is linked to an imaging agent.

In some aspects, provided herein is a composition comprising a first polynucleotide, a second polynucleotide, and a third polynucleotide, wherein the first, second, and third polynucleotides encode the multi-specific protein provided herein, wherein the first polynucleotide encodes a first heavy chain, the second polynucleotide encodes a second heavy chain and the antigen-binding domain that specifically binds to human CD98hc, and the third polynucleotide encodes a light chain.

In some aspects, provided herein is a composition comprising a first polynucleotide, a second polynucleotide, and a third polynucleotide, wherein the first, second, and third polynucleotides encode a multi-specific protein provided herein, wherein the first polynucleotide encodes a first heavy chain and a first antigen-binding domain that specifically binds to human CD98hc, the second polynucleotide encodes a second heavy chain and a second antigen-binding domain that specifically binds to human CD98, and the third polynucleotide encodes a light chain, optionally wherein the first and second antigen-binding domains that bind to human CD98hc comprise the same amino acid sequence.

In some aspects, the first heavy chain comprises a knob mutation and the second heavy chain comprises a hole mutation.

In some aspects, the ratio of the first, second, and third polynucleotides is about 1:3:6.

In some aspects, the first heavy chain comprises a hole mutation and the second heavy chain comprises a knob mutation.

In some aspects, provided herein is a composition comprising a first polynucleotide and a second polynucleotide, wherein the first and second polynucleotides encode the multi-specific protein provided herein, wherein the first polynucleotide encodes a heavy chain and the antigen-binding domain that bind to human CD98hc, and wherein the second polynucleotide encodes a light chain.

In some aspects, provided herein is a host cell comprising a composition provided herein.

In some aspects, provided herein is an isolated polynucleotide comprising a nucleic acid molecule encoding the heavy chain of the antigen-binding domain provided herein. In some aspects, provided herein, is an isolated polynucleotide comprising a nucleic acid molecule encoding the light chain variable region of the antigen-binding domain provided herein.

In some aspects, provided herein is an isolated vector comprising the polynucleotide provided herein. In some aspects, provided herein is an isolated vector comprising a nucleic acid molecule encoding the heavy chain variable region of the antigen-binding domain provided herein and a nucleic acid molecule encoding the light chain variable region provided herein.

In some aspects, provided herein is a host cell comprising a polynucleotide provided herein or a vector provided herein. In some aspects, the host cell is selected from the group consisting of E. coli, Pseudomonas, Bacillus, Streptomyces, yeast, CHO, YB/20, NSO, PER-C6, HEK-293T, NIH-3T3, HeLa, BHK, Hep G2, SP2/0, R1.1, B-W, L-M, COS 1, COS 7, BSC1, BSC40, BMT10 cell, plant cell, insect cell, and human cell in tissue culture.

In some aspects, provided herein is a method of producing an antigen-binding domain or multi-specific protein comprising culturing a host cell provided herein so that the antigen-binding domain or multi-specific protein is produced, optionally wherein the method further comprises isolating the antigen-binding domain or multi-specific protein from the culture.

In some aspects, provide herein is an isolated antigen-binding domain or multi-specific protein thereof produced by a method provided herein.

In some aspects, provided herein is a pharmaceutical composition comprising (i) an antigen-binding domain, fusion protein, antibody or antigen-binding fragment thereof, or multi-specific protein provided herein and (ii) a pharmaceutically acceptable carrier. In some aspects, the concentration of the fusion protein, antibody or an antigen-binding fragment thereof, or multi-specific protein is increased in the brain following administration to a subject as compared to an isotype control. In some aspects, administration increases delivery of fusion protein, antibody or antigen-binding fragment thereof, multi-specific protein, or pharmaceutical composition into the brain by at least 50%, at least 100%, at least 200%, at least 500% or at least 1000% as compared to an isotype control.

In some aspects, provided herein is a method of treating a neurological disease or disorder in a subject comprising administering a fusion protein, antibody or antigen-binding fragment thereof, multi-specific protein, or pharmaceutical composition provided herein to the subject. In some aspects, the administration increases delivery of fusion protein, antibody or antigen-binding fragment thereof, multi-specific protein, or pharmaceutical composition into the brain by at least 50%, at least 100%, at least 200%, at least 500% or at least 1000% as compared to an isotype control. In some aspects, the administration increases delivery of fusion protein, antibody or antigen-binding fragment thereof, multi-specific protein, or pharmaceutical composition into the frontal cortex, the entorhinal cortex and/or the hippocampus. In some aspects, the neurological disease or disorder is selected from a neuropathy disorder, a neurodegenerative disease, cancer, an ocular disease disorder, a seizure disorder, a lysosomal storage disease, amyloidosis, a viral or microbial disease, ischemia, a behavioral disorder, and CNS inflammation. In some aspects, the neurological disease or disorder is selected from Alzheimer's disease (AD), Huntington's disease, dystonia, ataxia, Bell's palsy, stroke, dementia, Lewy body dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, encephalitis, traumatic brain injury, and limbic-predominant age-related TDP-43 encephalopathy (LATE). In some aspects, the dementia is frontotemporal dementia (FTD). In some aspects, the neurological disease or disorder is Alzheimer's disease. In some aspects, the Alzheimer's disease is early onset Alzheimer's disease, prodromal Alzheimer's disease, mild Alzheimer's disease, or late onset Alzheimer's disease. In some aspects, the neurological disease or disorder is Parkinson's disease. In some aspects, the neurological disease or disorder is frontal temporal epilepsy. In some aspects, the neurological disease or disorder is autism. In some aspects, the neurological disease or disorder is lissencephaly.

In some aspects, provided herein is a method of treating a lysosomal storage disease in a subject, comprising administering the fusion protein provided herein. In some aspects, the lysosomal storage disease is selected from Gaucher disease, Ceroid lipofuscinosis (Batten disease), Mucopolysaccharidosis (MPS) Type I, MPS Type II and MPS Type III.

In some aspects, provided herein is a method of transporting a fusion protein, antibody or an antigen-binding fragment thereof, or multi-specific protein across the BBB of a subject, comprising administering to the subject a fusion protein, antibody or antigen-binding fragment thereof, multi-specific protein, or pharmaceutical composition provided herein to the subject. In some aspects, the concentration of the fusion protein, antibody or an antigen-binding fragment thereof, or multi-specific protein is increased in the brain following administration as compared to an isotype control. In some aspects, the concentration of the fusion protein, antibody or antigen-binding fragment thereof, multi-specific protein, or pharmaceutical composition in the brain is increased by at least 50%, at least 100%, at least 200%, at least 500% or at least 1000% as compared to an isotype control. In some aspects, administration of the fusion protein, antibody or an antigen-binding fragment thereof, or multi-specific protein does not result in reticulocyte count reduced in the subject by more than 10%, as compared to administration of an isotype control. In some aspects, administration of the fusion protein, antibody or an antigen-binding fragment thereof, or multi-specific protein does not result in reticulocyte count reduction in the subject, as compared to an isotype control.

In some aspects, provided herein is a method of increasing the concentration of a CNS binding antigen in the CSF of a subject, comprising administering a multi-specific protein provided herein to the subject, wherein the concentration of the CNS binding antigen is increased as compared to administering the CNS binding antigen alone to the subject.

In some aspects, provided herein is a method of imaging a CNS antigen within a subject, comprising administering to the subject a fusion protein, antibody or antigen-binding fragment thereof, or multi-specific protein provided herein and locating the imaging agent within the subject.

In some aspects, provided herein is a method of detecting a CNS antigen in vitro, comprising contacting an in vitro sample with a fusion protein, antibody or antigen-binding fragment thereof, or multi-specific protein provided herein and locating the imaging agent within the sample.

In some aspects, provided herein is a use of a fusion protein, antibody or antigen-binding fragment thereof, or multi-specific protein, or composition provided herein in the method provided herein.

In some aspects, provided herein is a fusion protein, antibody or antigen-binding fragment thereof, multi-specific protein, or composition provided herein for use in the method provided herein.

It is to be understood that one, some, or all of the properties of the various aspects described herein can be combined to form other aspects of the present disclosure. These and other aspects of the disclosure will be immediately apparent to one of skill in the art. These and other aspects of the disclosure are further described by the detailed description that follows.

### BRIEF DESCRIPTION OF THE DRAWINGS

FIG. 1A shows a 2+1 bispecific antibody.

FIG. 1B shows a 2+2 bispecific antibody with 2 scFvs with the same amino acid sequence.

FIG. 1C shows a 2+2 bispecific antibody with two scFvs with different amino acid sequences.

FIG. 2 shows fold changes of CD98hc surface levels on hCMEC/D3 cells after treatment with 2+1 anti-CD98hc bispecific antibodies as compared to untreated cells, evaluated via FACS. See Example 16.

FIG. 3 shows CD98hc total protein levels on hCMEC/D3 cells after treatment with 2+1 anti-CD98hc bispecific antibodies, evaluated via Western blot. See Example 16.

FIG. 4 shows the brain penetration of 2+1 anti-CD98hc bispecific antibodies in hCD98hc-mice, as measured by antibody level in vessel-depleted brain 24 hrs after peripheral injection. Antibody CD98hc.04.064 has an approximately 2-fold increase over control. Antibody CD98hc.04.063 has an approximately 1.5-fold increase over control. See Example 17.

FIG. 5 shows the serum PK of 2+1 anti-CD98hc bispecific antibodies in hCD98hc^{-/-} mice. Brain penetrating antibodies have the highest serum clearance rate. See Example 17.

FIG. 6 shows the brain/serum ratio of 2+1 anti-CD98hc bispecific antibodies in hCD98hc^{-/-} mice 24 hrs after peripheral injection. See Example 17.

FIG. 7 shows a modest antibody-dependent cellular cytotoxicity (ADCC) response of 2+1 anti-CD98hc bispecific antibodies against a BBB cell line. See Example 20.

FIG. 8 shows antibody levels in vessel-depleted brain fractions of huCD98hc knock-in mice after dosing with 2_+1 anti-CD98hc bispecific antibodies and a matched control (having the same Fab and Fc domain but no scFv that specifically binds to huCD98hc). Antibody levels are shown as fold change over the matched control. See Example 22.

FIG. 9 shows antibody levels in the serum of huCD98hc knock-in mice after dosing with 2_+1 anti-CD98hc bispecific antibodies and a matched control (having the same Fab and Fc domain but no scFv that specifically binds to huCD98hc). See Example 22.

FIG. 10 shows the activity of a 2+1 anti-CD98hc bispecific antibody in an in vitro STREM2 assay. See Example 24.

FIG. 11 shows absolute reticulocyte counts in non-human primates (NHP) following administration of a 2+1 anti-CD98hc bispecific antibody. See Example 26.

FIG. 12 shows serum and CSF levels of a 2+1 anti-CD98hc bispecific antibody after a first and second dosing. See Example 27.

FIG. 13 shows antibody concentration in NHP brain fractions following administration of a 2+1 anti-CD98hc bispecific antibody. See Example 28.

FIG. 14 shows soluble TREM2 levels in serum and CSF of NHPs following administration of a 2+1 anti-CD98hc bispecific antibody. See Example 29.

FIG. 15 shows levels of CSF-1 in the CSF of NHPs following administration of a 2+1 anti-CD98hc bispecific antibody. See Example 29.

### DETAILED DESCRIPTION OF THE PRESENT DISCLOSURE

The present disclosure relates to an antigen-binding domain that specifically binds to human CD98 heavy chain (CD98hc), and antibodies, and antigen-binding fragments thereof comprising such antigen-binding domains, methods of making and using such antigen-binding domains, antibodies, and antigen-binding fragments thereof; pharmaceutical compositions comprising such antigen-binding domains, antibodies, and antigen-binding fragments thereof; nucleic acids encoding such antigen-binding domains, antibodies, and antigen-binding fragments thereof; and host cells comprising nucleic acids encoding such antigen-binding domains, antibodies, and antigen-binding fragments thereof.

The techniques and procedures described or referenced herein are generally well understood and commonly employed using conventional methodology by those skilled in the art, such as, for example, the widely utilized methodologies such as those described in Sambrook et al. Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003); Monoclonal Antibodies: A Practical Approach (P. Shepherd and C. Dean, eds., Oxford University Press, 2000).

### Definitions

The terms “central nervous system” and “CNS” refer to the complex of nerve tissues that control bodily function and includes the brain and spinal cord.

The terms “blood brain barrier” and “BBB” refer to a network of brain capillary endothelial cells that are closely sealed by tight junctions and characterized by low levels of non-specific paracellular and transcellular transport.

A “central nervous system antigen” or “CNS antigen” is an antigen expressed in the CNS, including the brain, which can be targeted with an antibody or small molecule. Examples of such antigens include, without limitation: beta-secretase 1 (BACE1), amyloid beta (Abeta), epidermal growth factor receptor (EGFR), human epidermal growth factor receptor 2 (HER2), tau, apolipoprotein E4 (ApoE4), alpha-synuclein, CD20, huntingtin, prion protein (PrP), leucine rich repeat kinase 2 (LRRK2), parkin, presenilin 1, presenilin 2, β-glucocerebrosidase (GCase or GBA), progranulin (PGRN), Prosaposin (PSAP), gamma secretase, death receptor 6 (DR6), amyloid precursor protein (APP), p75 neurotrophin receptor (p75NTR), interleukin 6 receptor (IL6R), TNF receptor 1 (TNFR1), interleukin 1 beta (IL1 (3)), caspase 6, sortilin (SORT), triggering receptor expressed on myeloid cells 2 (TREM2), CD33 or sialic acid binding Ig-like lectin 3 (Siglec3), sialic acid binding Ig-like lectin 5 (Siglec5), sialic acid binding Ig-like lectin 7 (Siglec7), sialic acid binding Ig-like lectin 9 (Siglec9), glycoprotein nonmetastatic melanoma protein B (GPNMB), Paired immunoglobin like type 2 receptor alpha (PILRA), Membrane Spanning 4-Domains A4A (MS4A4A), Membrane Spanning 4-Domains A 6A (MS4A6A), ubiquitin protein ligase E3A (UBE3A), or Transmembrane Protein 106B (TMEM106b).

A “brain antigen” is a CNS antigen expressed in the brain.

The terms “CD98hc,” “CD98hc polypeptide,” and “CD98hc protein” are used interchangeably herein to refer to any native CD98hc from any vertebrate source, including mammals such as primates (e.g., humans and cynomolgus monkeys (cynos)) and rodents (e.g., mice and rats), unless otherwise indicated. CD98hc is also referred to as 4F2 cell-surface antigen heavy chain, 4F2hc, 4F2 heavy chain antigen, lymphocyte activation antigen 4F2 large subunit, solute carrier family 3 member 2, and CD98. CD98hc protein is encoded by the SLC3A2 gene and is part of the large amino acid transporter (LAT) complex. In some aspects, the term encompasses both wild-type sequences and naturally occurring variant sequences, e.g., splice variants or allelic variants. In some aspects, the term encompasses “full-length,” unprocessed CD98hc, as well as any form of CD98hc that results from processing in the cell. In some aspects, the CD98hc is human CD98hc. As used herein, the term “human CD98hc” refers to a polypeptide with the amino acid sequence of SEQ ID NO:435.

(SEQ ID NO: 435)
MELQPPEASIAVVSIPRQLPGSHSEAGVQGLSAGDDSELGSHCVAQTGL
ELLASGDPLPSASQNAEMIETGSDCVTQAGLQLLASSDPPALASKNAEV
TGTMSQDTEVDMKEVELNELEPEKQPMNAASGAAMSLAGAEKNGLVKIK
VAEDEAEAAAAAKFTGLSKEELLKVAGSPGWVRTRWALLLLFWLGWLGM
LAGAVVIIVRAPRCRELPAQKWWHTGALYRIGDLQAFQGHGAGNLAGLK
GRLDYLSSLKVKGLVLGPIHKNQKDDVAQTDLLQIDPNFGSKEDFDSLL
QSAKKKSIRVILDLTPNYRGENSWFSTQVDTVATKVKDALEFWLQAGVD
GFQVRDIENLKDASSFLAEWQNITKGFSEDRLLIAGTNSSDLQQILSLL
ESNKDLLLTSSYLSDSGSTGEHTKSLVTQYLNATGNRWCSWSLSQARLL
TSFLPAQLLRLYQLMLFTLPGTPVFSYGDEIGLDAAALPGQPMEAPVML
WDESSFPDIPGAVSANMTVKGQSEDPGSLLSLFRRLSDQRSKERSLLHG
DFHAFSAGPGLFSYIRHWDQNERFLVVLNFGDVGLSAGLQASDLPASAS
LPAKADLLLSTQPGREEGSPLELERLKLEPHEGLLLRFPYAA

As used herein, the terms “antibody” and “immunoglobulin” are used interchangeably and refer to an antibody molecule that recognizes and specifically binds to a target, such as a protein, polypeptide, peptide, carbohydrate, polynucleotide, lipid, or combinations of the foregoing (e.g., a glycoprotein), through at least one antigen recognition site within the variable region of the immunoglobulin molecule. The term “antibody” encompasses monoclonal antibodies, chimeric antibodies, humanized antibodies, human antibodies, multi-specific (e.g., bi-specific) antibodies, and any other immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g., IgG1, IgG2, IgG3, IgG4, IgA1, and IgA2), based on the identity of their heavy-chain constant regions referred to as alpha, delta, epsilon, gamma, and mu, respectively. The different classes of antibodies have different and well known subunit structures and three-dimensional configurations. For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, CT, 1994, page 71 and Chapter 6.

The terms “anti-(′D) 98hc antibody,” “antibody that binds to CD98hc,” and “antibody that specifically binds (′D98hc” refer to an antibody that is capable of binding CD98hc with sufficient affinity such that the antibody is useful as a diagnostic and/or therapeutic agent in targeting CD98hc. In one aspect, the extent of binding of an anti-CD98hc antibody to an unrelated, non-CD98hc polypeptide is less than about 10% of the binding of the antibody to CD98hc as measured, e.g., by a radioimmunoassay (RIA). In certain aspects, an antibody that binds to CD98hc has a dissociation constant (KD) of <10 μM, <1 μM, <100 nM, <10 nM, <1 nM, <0.1 nM, <0.01 nM, or <0.001 nM (e.g., 10⁻⁸ M or less, e.g., from 10⁻⁸ M to 10⁻¹³ M, e.g., from 10⁻⁹M to 10⁻¹³ M). In certain aspects, an anti-CD98hc antibody binds to an epitope of CD98hc that is conserved among CD98hc from different species.

The term “antibody fragment” refers to a portion of an antibody. An “antigen-binding fragment” of an antibody refers to a portion of an antibody that binds to an antigen. An antigen-binding fragment of an antibody can comprise the antigenic determining regions of an antibody (e.g., the complementarity determining regions (CDRs)). Examples of antigen-binding fragments of antibodies include, but are not limited to Fab, Fab′, F(ab′)2, and Fv fragments, linear antibodies, and single chain antibodies. An antigen-binding fragment of an antibody can be monovalent or multi-valent (e.g., bi-valent). An antigen-binding fragment of an antibody can be monospecific or multi-specific (e.g., bi-specific.) An antigen-binding fragment of an antibody can be derived from any animal species, such as rodents (e.g., mouse, rat, or hamster) and humans or can be artificially produced.

An “antigen-binding domain” or “antigen-binding region” refers to a monovalent portion of an antibody that binds to an antigen. An “antigen-binding domain” can comprise the antigenic determining regions of an antibody (e.g., the complementarity determining regions (CDRs)). An antibody or antigen-binding fragment thereof (including mono-specific and multi-specific (e.g., bi-specific) antibodies or antigen-binding fragments thereof can comprise an antigen-binding domain.

The terms “anti-CD98hc antigen-binding domain,” “antigen-binding domain that binds to CD98hc,” “anti-CD98hc antigen-binding region,” “antigen-binding region that binds to CD98hc,” and “CD98hc binding domain” refer to an antigen-binding domain that binds to CD98hc with sufficient affinity such that the antigen-binding domain is useful for targeting CD98hc and/or useful as a diagnostic agent, a therapeutic agent, or for transporting a molecule or compound across the BBB. In one aspect, the extent of binding of an anti-CD98hc antigen-binding domain to an unrelated, non-CD98hc polypeptide is less than about 10% of the binding of the antigen-binding domain to CD98hc as measured, e.g., by a radioimmunoassay (RIA). In certain aspects, an antibody that binds to CD98hc has a dissociation constant (KD) of <0.1 μM, <1 μM, <10 μM, <100 nM, <10 nM, <1 nM, <0.1 nM, <0.01 nM, or <0.001 nM (e.g., 10⁻⁸ M or less, e.g., from 10⁻⁸ M to 10⁻¹³ M, e.g., from 10⁻⁹ M to 10⁻¹³ M). In certain aspects, an anti-CD98hc antigen-binding domain binds to an epitope of CD98hc that is conserved among CD98hc from different species.

The terms “full-length antibody,” “intact antibody” or “whole antibody” are used interchangeably to refer to an antibody in its substantially intact form, as opposed to an antibody fragment. Specifically, whole antibodies include those with heavy and light chains including an Fc region. The constant regions can be native sequence constant regions (e.g., human native sequence constant regions) or amino acid sequence variants thereof. In some cases, the intact antibody can have one or more effector functions.

“Native IgG antibodies” are usually heterotetrameric glycoproteins of about 150,000 Daltons, composed of two identical light (“L”) chains and two identical heavy (“H”) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

Papain digestion of antibodies produces two identical antigen-binding fragments, called “Fab” fragments, and a residual “F′c” fragment, a designation reflecting the ability to crystallize readily. The Fab fragment consists of an entire light chain along with the variable region domain of the heavy chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both heavy chains held together by disulfides. The effector functions of antibodies are determined by sequences in the Fc region, the region which is also recognized by Fc receptors (FcR) found on certain types of cells.

“Fv” is the minimum antibody fragment which comprises a complete antigen-recognition and -binding site. This fragment consists of a dimer of one heavy- and one light-chain variable region domain in tight, non-covalent association. From the folding of these two domains emanates six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sky” or “scFv” are antibody fragments that comprise the VH and VL antibody domains connected into a single polypeptide chain. In some aspects, the scFv polypeptide further comprises a polypeptide linker between the V_{H} and V_{L} domains, which enables the sFv to form the desired structure for antigen binding.

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the variable domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains.

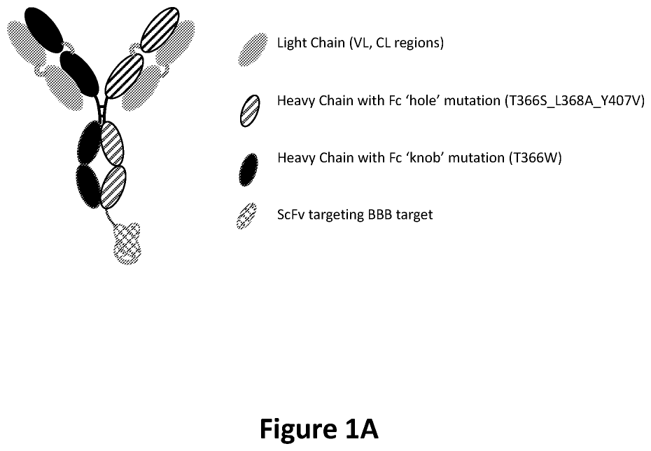

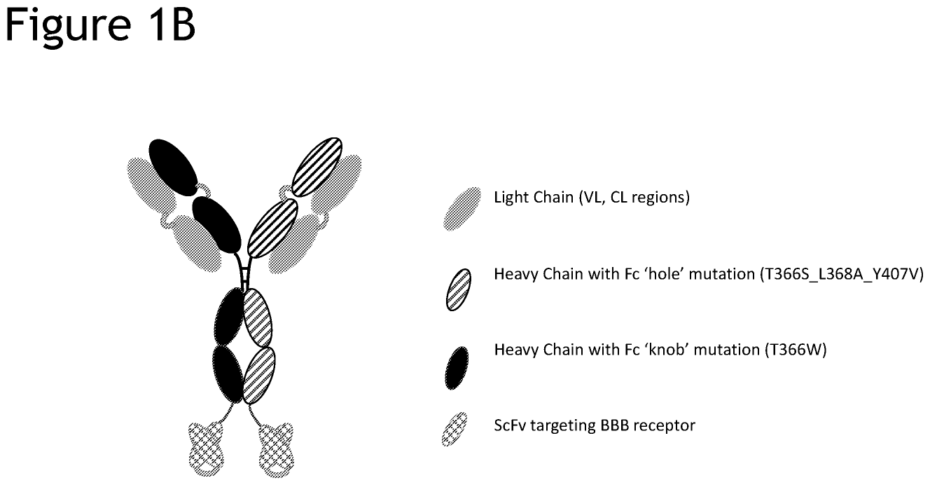

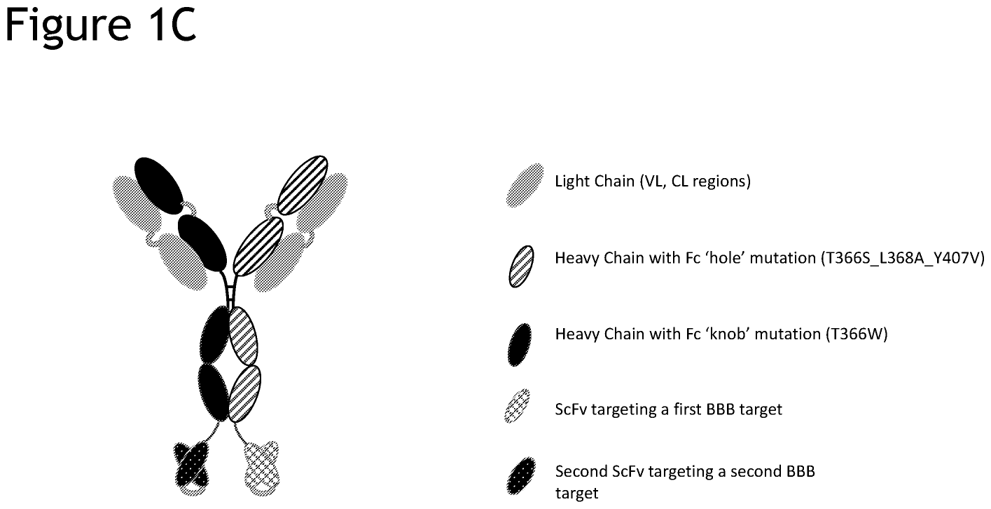

As used herein, a “2+1 antibody format” refers to a trivalent, bi-specific antibody format comprising (i) a single antigen-binding domain that binds to human CD98hc and (ii) an antibody, wherein the antibody comprises two heavy chains and two light chains; wherein the single antigen-binding domain that binds to human CD98hc is linked to the C-terminus of one of the two antibody heavy chains. This format is exemplified in FIG. 1A.

As used herein, a “2+2 antibody format” refers to a tetravalent, bi-specific antibody format comprising (i) two antigen-binding domains that bind to human CD98hc and (ii) an antibody, wherein the antibody comprises two heavy chains and two light chains; wherein one antigen-binding domain that binds to human CD98hc is linked to the C-terminus of one of the two antibody heavy chains, and the other antigen-binding domain that binds to human CD98hc is linked to the C-terminus of the other of the two antibody heavy chains. The two antigen-binding domains that bind to human CD98hc can comprise the same amino acid sequence. This format is displayed in FIG. 1B. In some aspects, the two scFv antigen-binding domains that bind to human CD98hc can comprise different amino acid sequences. This format is displayed in FIG. 1C.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in a sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In some aspects, the variable region is a human variable region. In some aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) framework regions (FRs). The term “Kabat numbering” and like terms are recognized in the art and refer to a system of numbering amino acid residues in the heavy and light chain variable regions of an antibody or an antigen-binding fragment thereof. In certain aspects, CDRs can be determined according to the Kabat numbering system (see, e.g., Kabat E A & Wu T T (1971) Ann NY Acad Sci 190:382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDRH1), amino acid positions 50 to 65 (CDRH2), and amino acid positions 95 to 102 (CDRH3). Using the Kabat numbering system, CDRs within an antibody light chain molecule are typically present at amino acid positions 24 to 34 (CDRL1), amino acid positions 50 to 56 (CDRL2), and amino acid positions 89 to 97 (CDRL3). In some aspects, the CDRs of the antibodies described herein have been determined according to the Kabat numbering scheme. Chothia refers instead to the location of the structural loops (Chothia and Lesk, J. Mol. Biol. 196:901-917 (1987)). The end of the Chothia CDRH1 loop, when numbered using the Kabat numbering convention, varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software. In some aspects, the CDRs can be “contact” CDRs. The “contact” CDRs are based on an analysis of the available complex crystal structures. The residues from each of these CDRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| H1 | H31-H35 | H26-H35 | H26-H32 | H30-H35 (Chothia |
| H2 | H50-H65 | H50-H58 | H53-H55 | H47-H58 |
| H3 | H95-H102 | H95-H102 | H96-H101 | H93-H101 |

CDRs can comprise “extended CDRs” as follows: 24-36 or 24-34 (L1), 46-56 or 50-56 (L2), and 89-97 or 89-96 (L3) in the VL, and 26-35 (H1), 50-65 or 49-65 (H2), and 93-102, 94-102, or 95-102 (H3) in the VH. The variable-domain residues are numbered according to Kabat et al., supra, for each of these extended-CDR definitions.

The terms “VH” and “\′H domain” are used interchangeably to refer to the heavy chain variable region of an antibody.

As used herein, the term “heavy chain” when used in reference to an antibody can refer to any distinct type, e.g., alpha (α), delta (δ), epsilon (ε), gamma (γ), and mu (μ), based on the amino acid sequence of the constant region, which give rise to IgA, IgD, IgE, IgG, and IgM classes of antibodies, respectively, including subclasses of IgG, e.g., IgG1, IgG2, IgG3, and IgG4. Heavy chain amino acid sequences are well known in the art. In some aspects, the heavy chain is a human heavy chain.

The terms “VL” and “VL domain” are used interchangeably to refer to the light chain variable region of an antibody.

As used herein, the term “light chain” when used in reference to an antibody can refer to any distinct type, e.g., kappa (κ) or lambda (λ) based on the amino acid sequence of the constant regions. Light chain amino acid sequences are well known in the art. In some aspects, the light chain is a human light chain.

As used herein, the term “constant region” is a region of an antibody that is not the variable region of the antibody, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain. In certain aspects, an antibody or antigen-binding fragment comprises a constant region or portion thereof that is sufficient for antibody-dependent cell-mediated cytotoxicity (ADCC).

A “constant domain” means a domain within a constant region that is capable of forming an immunoglobulin fold. Constant domains include the CH1, CH2, CH3, and CL domains.

The term “monoclonal” when referring to an antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody or antibody portion, and any other modified immunoglobulin molecule comprising an antigen recognition site. Furthermore, a “monoclonal” antibody or antigen-binding fragment thereof refers to such antibodies and antigen-binding fragments thereof made in any number of manners including but not limited to by hybridoma, phage selection, recombinant expression, and transgenic animals.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g., mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g., murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementarity determining regions (CDRs) are replaced by residues from the CDRs of molecule originating from a non-human species (e.g., mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). The humanized antibody or antigen-binding fragment thereof can be further modified by the substitution of additional residues either in the Fv framework region and/or within the replaced non-human residues to refine and optimize the specificity, affinity, and/or capability of the antibody or antigen-binding fragment thereof. In general, the humanized antibody or antigen-binding fragment thereof will comprise VH and VL that comprise substantially all of at least one, and typically two or three, of the CDR regions that correspond to the non-human immunoglobulin, whereas all or substantially all of the FR regions are those of a human immunoglobulin consensus sequence. The humanized antibody or antigen-binding fragment thereof can also comprise at least a portion of an immunoglobulin constant region or Fc region, typically that of a human immunoglobulin. Examples of methods used to generate humanized antibodies are described in U.S. Pat. No. 5,225,539; Roguska et al., Proc. Natl. Acad. Sci., USA, 91 (3): 969-973 (1994), and Roguska et al., Protein Eng. 9 (10): 895-904 (1996). In some aspects, a “humanized antibody” is a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

“Framework” or “FR” residues are those variable-domain residues other than the CDR residues as herein defined.

An “acceptor human framework” as used herein is a framework comprising the amino acid sequence of a V_{L} or V_{H} framework derived from a human immunoglobulin framework or a human consensus framework. An acceptor human framework “derived from” a human immunoglobulin framework or a human consensus framework can comprise the same amino acid sequence thereof, or it can comprise pre-existing amino acid sequence changes. In some aspects, the number of pre-existing amino acid changes are 10 or less, 9 or less, 8 or less, 7 or less, 6 or less, 5 or less, 4 or less, 3 or less, or 2 or less. Where pre-existing amino acid changes are present in a VH, in some aspects those changes occur at only three, two, or one of positions 71H, 73H and 78H; for instance, the amino acid residues at those positions can by 71A, 73T and/or 78A. In some aspects, the VL acceptor human framework is identical in sequence to the V_{L} human immunoglobulin framework sequence or human consensus framework sequence.

A “human consensus framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, MD (1991). Examples include for the VL, the subgroup can be subgroup kappa I, kappa II, kappa III or kappa IV as in Kabat et al., supra. Additionally, for the VH, the subgroup can be subgroup I, subgroup II, or subgroup III as in Kabat et al., supra.

An “amino-acid modification” at a specified position, e.g., of an antibody of the present disclosure, refers to the substitution or deletion of the specified residue, or the insertion of at least one amino acid residue adjacent the specified residue. Insertion “adjacent” to a specified residue means insertion within one to two residues thereof. The insertion can be N-terminal or C-terminal to the specified residue. In some aspects, an amino acid modification is a substitution.

Antibody “effector functions” refer to those biological activities attributable to the Fc region (a native sequence Fc region or amino acid sequence variant Fc region) of an antibody, and vary with the antibody isotype.

The term “Fc region” or “fragment crystallizable region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region can be removed, for example, during production or purification of the antibody, or by recombinantly engineering the nucleic acid encoding a heavy chain of the antibody. Accordingly, a composition of intact antibodies can comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, in some aspects one or more amino acid substitution(s). In some aspects, the variant Fc region has at least one amino acid substitution compared to a native sequence Fc region or to the Fc region of a parent polypeptide, e.g., from about one to about ten amino acid substitutions, and in some aspects from about one to about five amino acid substitutions in a native sequence Fc region or in the Fc region of the parent polypeptide. In some aspects, the variant Fc region possesses at least 80% homology with a native sequence Fc region and/or with an Fc region of a parent polypeptide, at least 90% homology therewith, or at least 95% homology therewith.

“Fc receptor” or “FcR” describes a receptor that binds to the Fc region of an antibody. In some aspects, an FcR is a native sequence human FcR. In some aspects, a FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. Other FcRs, including those to be identified in the future, are encompassed by the term “FcR” herein. FcRs can also increase the serum half-life of antibodies.

“Binding affinity” generally refers to the strength of the sum total of non-covalent interactions between a single binding site of a molecule (e.g., an antibody or antigen-binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen-binding fragment thereof and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody or antigen-binding fragment thereof to an antigen, and k_{off} refers to the dissociation rate constant of, e.g., an antibody or antigen-binding fragment thereof from an antigen. The kₒₙ and k_{off} can be determined by techniques known to one of ordinary skill in the art, such as BIAcore® or KinExA.

With regard to the binding of an antibody to a target molecule, the term “specific binding” or “specifically binds” or is “specific for” a particular polypeptide or an epitope on a particular polypeptide target means binding that is measurably different from a non-specific interaction. Specific binding can be measured, for example, by determining binding of a molecule compared to binding of a control molecule. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a KD for the target of about any of 10⁻⁴ or lower, 10⁻⁵ M or lower, 10⁻⁶ M or lower, 10⁻⁷ M or lower, 10⁻⁸ M or lower, 10.9 M or lower, 10⁻¹⁰ M or lower, 10⁻¹¹ M or lower, 10⁻¹² M or lower or a KD in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and KD values are inversely related. A high affinity for an antigen is measured by a low KD value. In some aspects, the term “specific binding” refers to binding where a molecule binds to a particular polypeptide or epitope on a particular polypeptide without substantially binding to any other polypeptide or polypeptide epitope.

The term “linker” or “linked” refers to the covalent linkage between two polypeptides or two heterologous molecules. In some aspects, a linker is a chemical linker. In some aspects, the linker comprises a peptide bond, and the two polypeptides or two heterologous molecules are linked to each other either directly to or via one or more additional amino acids. A glycine linker is one that comprises one or more glycines but no other amino acids, e.g., GGGG (SEQ ID NO: 351). A glycine-rich linker is one that comprises one or more glycines and can contain other amino acids as long as glycine is the predominant species in the linker e.g., GGGNGG, wherein N is any amino acid (SEQ ID NO:352). A glycine-serine linker is one which contains both glycine and serine in any proportion, e.g., GGGS (SEQ ID NO:353). Similarly, a proline linker is one that comprises one or more prolines but no other amino acids. A proline-rich linker is one that comprises one or more prolines and can contain other amino acids so long as proline is the predominant species in the linker.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

The term “epitope” includes any determinant capable of being bound by an antibody. An epitope is a region of an antigen that is bound by an antibody that targets that antigen, and when the antigen is a polypeptide, includes specific amino acids that directly contact the antibody. Most often, epitopes reside on polypeptides, but in some instances, can reside on other kinds of molecules, such as nucleic acids. Epitope determinants can include chemically active surface groupings of molecules such as amino acids, sugar side chains, phosphoryl or sulfonyl groups, and can have specific three-dimensional structural characteristics, and/or specific charge characteristics. Generally, antibodies specific for a particular target antigen will preferentially recognize an epitope on the target antigen in a complex mixture of polypeptides and/or macromolecules.

An antibody that “binds to the same epitope” as a reference antibody refers to an antibody that contacts the same amino acid residues on the antigen as the reference antibody. The ability of an antibody to bind to the same epitope as a reference antibody can be determined using peptide scanning mutagenesis or high throughput alanine scanning mutagenesis. In the latter methodology, a comprehensive mutation library of antigen, or a portion thereof (e.g., the extracellular domain), can be generated by mutating each individual amino acid residue to alanine (or if the amino acid residue is alanine, then to another residue such as serine) and testing each mutant for binding to a target antibody or antigen-binding fragment thereof.

An antibody is said to “competitively inhibit” binding of a reference antibody to a given epitope if it preferentially binds to that epitope or an overlapping epitope such that it blocks, to some degree, binding of the reference antibody to the epitope. Competitive inhibition can be determined by any method known in the art, for example, competition ELISA assays. An antibody can be said to competitively inhibit binding of the reference antibody to a given epitope by at least 90%, at least 80%, at least 70%, at least 60%, or at least 50%.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells or compositions include those which have been purified to a degree that they are no longer in a form in which they are found in nature. In some aspects, an antibody, polynucleotide, vector, cell, or composition which is isolated is substantially pure.

As used herein, “substantially pure” refers to material which is at least 50% pure (i.e., free from contaminants), at least 90% pure, at least 95% pure, at least 98% pure, or at least 99% pure.

The term “expression system” refers to one or more nucleic acid molecules comprising coding sequence and control sequence(s) in operable linkage, along with a host cell and/or other in vitro transcription and translation machinery, such that one or more proteins encoded by the nucleic acid molecule(s) are capable of being produced.

The term “vector,” as used herein, is intended to refer to a nucleic acid molecule capable of transporting another nucleic acid to which it has been linked. One type of vector is a “plasmid,” which refers to a circular double stranded DNA into which additional DNA segments can be ligated. Another type of vector is a phage vector. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” can be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

“Carriers” as used herein include pharmaceutically acceptable carriers, excipients, or stabilizers that are nontoxic to the cell or mammal being exposed thereto at the dosages and concentrations employed.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

The terms “administer,” “administering,” “administration,” and the like, as used herein, refer to methods that can be used to deliver a drug, e.g., an anti-human antibody or antigen-binding fragment thereof, to the desired site of biological action.

An “effective amount” refers to at least an amount effective, at dosages and for periods of time necessary, to achieve the desired therapeutic result. An effective amount can be provided in one or more administrations. An effective amount is also one in which any toxic or detrimental effects of the treatment are outweighed by the therapeutically beneficial effects. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount of drug, compound, or pharmaceutical composition is an amount sufficient to accomplish therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” can be considered in the context of administering one or more therapeutic agents, and a single agent can be considered to be given in an effective amount if, in conjunction with one or more other agents, a desirable result can be or is achieved.

As used herein, the terms “subject” and “patient” are used interchangeably. The subject can be a mammal such as a non-human animal (e.g., cow, pig, horse, cat, dog, rat, mouse, monkey or other primate, etc.). In some aspects, the subject is a cynomolgus monkey. In some aspects, the subject is a human.

As used herein, administration “in conjunction” or “in combination” with another compound or composition includes simultaneous administration and/or administration at different times. Administration in conjunction also encompasses administration as a co-formulation or administration as separate compositions, including at different dosing frequencies or intervals, and using the same route of administration or different routes of administration. In some aspects, administration in conjunction is administration as a part of the same treatment regimen.

A “neurological disorder” as used herein refers to a disease or disorder which affects the CNS and/or which has an etiology in the CNS. Exemplary CNS diseases or disorders include, but are not limited to, neuropathy, amyloidosis, cancer, an ocular disease or disorder, viral or microbial infection, inflammation, ischemia, neurodegenerative disease, seizure, behavioral disorders, and a lysosomal storage disease.

A “Lysosomal storage disorder” or (LSD) as used herein refers to an inherited metabolic disease characterized by the accumulation of substrates, such as undigested or partially digested macromolecules, in excess in various cells of organs, which ultimately results in cellular dysfunction and clinical abnormalities. LSDs have been defined as deficiencies in lysosomal function generally classified by the accumulated substrate and include sphingolipidoses, oligosaccharidoses, mucolipidoses, mucopolysaccharidoses, lipoprotein storage disorders, neuronal ceroid lipofuscinoses, and others. LSDs may also include other deficiencies or defects in proteins that result in accumulation of macromolecules, such as proteins necessary for normal post-translational modification of lysosomal enzymes, or proteins important for proper lysosomal trafficking. LSDs are diseases caused by defects in single genes. Enzyme defects cause nearly seventy percent of the LSDs, and the rest are defects in enzyme activator or associated proteins.

“Protein replacement therapy” or “PRT” refers to a medical treatment that supplements or replaces a protein in a patient in whom that particular protein is deficient or absent.

An “enzyme replacement therapy enzyme” or “ERT enzyme” refers to an enzyme that is deficient in a lysosomal storage disorder. An “ERT enzyme variant” refers to a functional variant, including allelic and splice variants, of a wild-type ERT enzyme or a fragment thereof, where the ERT enzyme variant has at least 50%, at least 55%, at least 60%, at least 65%, at least 70%, at least 75%, at least 80%, at least 85%, at least 90%, or at least 95% of the activity of the corresponding wild-type ERT enzyme or fragment thereof, e.g., when assayed under identical conditions. A “catalytically active fragment” of an ERT enzyme refers to a portion of a full-length ERT enzyme or a variant thereof, where the catalytically active fragment has at least 50%, at least 55%, at least 60%, at least 65%, at least 70%, at least 75%, at least 80%, at least 85%, at least 90%, or at least 95% of the activity of the corresponding full-length ERT enzyme or variant thereof, e.g., when assayed under identical conditions.

As used herein, the terms “about” and “approximately,” when used to modify a numeric value or numeric range, indicate that deviations of up to 10% above and down to 10% below the value or range remain within the intended meaning of the recited value or range. It is understood that wherever aspects are described herein with the language “about” or “approximately” a numeric value or range, otherwise analogous aspects referring to the specific numeric value or range are also provided.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that wherever aspects are described herein with the language “comprising,” otherwise analogous aspects described in terms of “consisting of” and/or “consisting essentially of” are also provided. In this disclosure, “comprises,” “comprising,” “containing” and “having” and the like can mean “includes,” “including,” and the like; “consisting essentially of” or “consists essentially of” are open-ended, allowing for the presence of more than that which is recited so long as basic or novel characteristics of that which is recited is not changed by the presence of more than that which is recited, but excludes prior art aspects.

### Anti-CD98hc Antigen-Binding Domains

Provided herein are antigen-binding domains that specifically bind to human CD98hc.

Such antigen-binding domains can be capable of crossing the blood brain barrier (BBB) and capable of transporting other agents (e.g., therapeutically active agents) associated with the antigen-binding domain across the BBB. Accordingly, in some aspects, provided herein are antigen-binding domains that specifically bind to human CD98hc that are capable of being internalized in BBB epithelial cells, such as HCMEC/D3 cells.

In some aspects, an antigen-binding domain that specifically binds to human CD98hc comprises the six CDRs of an antibody listed in Tables 9 and 10 (i.e., the three VH CDRs of the antibody listed in Table 9 and the three VL CDRs of the same antibody listed in Table 10) or an antibody listed in Tables 14 and 15 (i.e., the three VH CDRs of the antibody listed in Table 14 and the three VL CDRs of the same antibody listed in Table 15), or the six CDRs of an antibody listed in Table 23 or 26.

In some aspects, an antigen-binding domain that specifically binds to human CD98hc comprises the six CDRs of an antibody listed in Table 8, 13, 22 or 25. In some aspects, the CDRs of such an antigen-binding domain can be determined according to the Chothia numbering scheme, which refers to the location of immunoglobulin structural loops (see, e.g., Chothia C & Lesk A M, (1987), J Mol Biol 196:901-917; Al-Lazikani B et al., (1997) J Mol Biol 273:927-948; Chothia C et al., (1992) J Mol Biol 227:799-817; Tramontano A et al., (1990) J Mol Biol 215 (1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In some aspects, an antigen-binding domain that specifically binds to human CD98hc comprises the six Chothia CDRs of an antibody listed in Table 8, 13, 22 or 25. In some aspects, such as an antigen-binding domain that specifically binds to human CD98hc comprises one or more CDRs, in which the Chothia and Kabat CDRs have the same amino acid sequence. In some aspects, provided herein are antigen-binding domains that specifically binds to human CD98hc and comprise combinations of Kabat CDRs and Chothia CDRs.

In some aspects, the CDRs of an antigen-binding domain that specifically binds to human CD98hc can be determined according to MacCallum R M et al., (1996) J Mol Biol 262:732-745. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In some aspects, provided herein are antigen-binding domains that specifically bind to human CD98hc and comprise VH and VL CDRs of an antibody listed in Table 8,13, 22 or 25 as determined by the method in MacCallum R M et al.

In some aspects, the CDRs of an antigen-binding domain that specifically binds to human CD98hc can be determined according to the AbM numbering scheme, which refers to AbM hypervariable regions, which represent a compromise between the Kabat CDRs and Chothia structural loops, and are used by Oxford Molecular's AbM antibody modeling software (Oxford Molecular Group, Inc.). In some aspects, provided herein are antigen-binding domains that specifically bind to human CD98hc and comprise VH and VL CDRs of an antibody listed in Table 8, 13, 22 or 25as determined by the AbM numbering scheme.

In some aspects, an antigen-binding domain that specifically binds to human CD98hc comprises the six IMGT CDRs of an antibody listed in Table 8, 13, 22 or 25according to the IMGT numbering system as described in Lefranc M-P, (1999) The Immunologist 7:132-136 and Lefranc M-P et al., (1999) Nucleic Acids Res 27:209-212. According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

In some aspects, an antigen-binding domain that specifically binds to human CD98hc provided herein is described by its VL domain alone, or its VH domain alone, or by its 3 VL CDRs alone, or its 3 VH CDRs alone. See, for example, Rader C et al., (1998) PNAS 95:8910-8915, which is incorporated herein by reference in its entirety, describing the humanization of the mouse anti-αvβ3 antibody by identifying a complementing light chain or heavy chain, respectively, from a human light chain or heavy chain library, resulting in humanized antibody variants having affinities as high or higher than the affinity of the original antibody. See also Clackson T et al., (1991) Nature 352:624-628, which is incorporated herein by reference in its entirety, describing methods of producing antibodies that bind a specific antigen by using a specific VL domain (or VH domain) and screening a library for the complementary variable domains. The screen produced 14 new partners for a specific VH domain and 13 new partners for a specific VL domain, which were strong binders, as determined by ELISA. See also Kim S J & Hong H J, (2007) J Microbiol 45:572-577, which is incorporated herein by reference in its entirety, describing methods of producing antibodies that bind a specific antigen by using a specific VH domain and screening a library (e.g., human VL library) for complementary VL domains; the selected VL domains in turn could be used to guide selection of additional complementary (e.g., human) VH domains.

In some aspects, an antigen-binding domain that specifically binds to human CD98hc comprises the VH of an antibody listed in Table 8, 13, 22 or 25.

In some aspects, an antigen-binding domain that specifically binds to human CD98hc comprises the VL of antibody listed in Table 8, 13, 22 or 25.

In some aspects, an antigen-binding domain that specifically binds to human CD98hc comprises the VH and the VL of an antibody listed in Table 8 (i.e., the VH of the antibody listed in Table 8 and the VL of the same antibody listed in the Table 8) or Table 13 (i.e., the VH of the antibody listed in Table 13 and the VL of the same antibody listed in the Table 13) or Table 22 or Table 25.

In some aspects, an antigen-binding domain that specifically binds to human CD98hc comprises (i) a VH comprising an amino acid sequence that is at least 80% identical to a VH amino acid sequence of an antibody in Table 8, 13, 22 or 25 and (ii) a VL comprising an amino acid sequence that is at least 80% identical to the VL amino acid sequence of the same antibody in Table 8, 13, 22 or 25. In some aspects, the antigen-binding domain that specifically binds to human CD98hc also comprises the CDRs of the antibody in Table 8, 13, 22 or 25 (e.g., the non-identical amino acids in the VH and/or VL are outside of the CDRs).

In some aspects, an antigen-binding domain that specifically binds to human CD98hc comprises (i) a VH comprising an amino acid sequence that is at least 85% identical to a VH amino acid sequence of an antibody in Table 8, 13, 22 or 25 and (ii) a VL comprising an amino acid sequence that is at least 85% identical to the VL amino acid sequence of the same antibody in Table 8, 13, 22 or 25. In some aspects, the antigen-binding domain that specifically binds to human CD98hc also comprises the CDRs of the antibody in Table 8, 13, 22 or 25 (e.g., the non-identical amino acids in the VH and/or VL are outside of the CDRs).

In some aspects, an antigen-binding domain that specifically binds to human CD98hc comprises (i) a VH comprising an amino acid sequence that is at least 90% identical to a VH amino acid sequence of an antibody in Table 8, 13, 22 or 25 and (ii) a VL comprising an amino acid sequence that is at least 90% identical to the VL amino acid sequence of the same antibody in Table 8, 13, 22 or 25. In some aspects, the antigen-binding domain that specifically binds to human CD98hc also comprises the CDRs of the antibody in Table 8, 13, 22 or 25 (e.g., the non-identical amino acids in the VH and/or VL are outside of the CDRs).

In some aspects, an antigen-binding domain that specifically binds to human CD98hc comprises (i) a VH comprising an amino acid sequence that is at least 95% identical to a VH amino acid sequence of an antibody in Table 8, 13, 22 or 25 and (ii) a VL comprising an amino acid sequence that is at least 95% identical to the VL amino acid sequence of the same antibody in Table 8, 13, 22 or 25. In some aspects, the antigen-binding domain that specifically binds to human CD98hc also comprises the CDRs of the antibody in Table 8, 13, 22 or 25 (e.g., the non-identical amino acids in the VH and/or VL are outside of the CDRs).

In some aspects, an antigen-binding domain that specifically binds to human CD98hc comprises (i) a VH comprising an amino acid sequence that is at least 96% identical to a VH amino acid sequence of an antibody in Table 8, 13, 22 or 25 and (ii) a VL comprising an amino acid sequence that is at least 96% identical to the VL amino acid sequence of the same antibody in Table 8, 13, 22 or 25. In some aspects, the antigen-binding domain that specifically binds to human CD98hc also comprises the CDRs of the antibody in Table 8, 13, 22 or 25 (e.g., the non-identical amino acids in the VH and/or VL are outside of the CDRs).

In some aspects, an antigen-binding domain that specifically binds to human CD98hc comprises (i) a VH comprising an amino acid sequence that is at least 97% identical to a VH amino acid sequence of an antibody in Table 8, 13, 22 or 25 and (ii) a VL comprising an amino acid sequence that is at least 97% identical to the VL amino acid sequence of the same antibody in Table 8, 13, 22 or 25. In some aspects, the antigen-binding domain that specifically binds to human CD98hc also comprises the CDRs of the antibody in Table 8, 13, 22 or 25 (e.g., the non-identical amino acids in the VH and/or VL are outside of the CDRs).

In some aspects, an antigen-binding domain that specifically binds to human CD98hc comprises (i) a VH comprising an amino acid sequence that is at least 98% identical to a VH amino acid sequence of an antibody in Table 8, 13, 22 or 25 and (ii) a VL comprising an amino acid sequence that is at least 98% identical to the VL amino acid sequence of the same antibody in Table 8, 13, 22 or 25. In some aspects, the antigen-binding domain that specifically binds to human CD98hc also comprises the CDRs of the antibody in Table 8, 13, 22 or 25 (e.g., the non-identical amino acids in the VH and/or VL are outside of the CDRs).

In some aspects, an antigen-binding domain that specifically binds to human CD98hc comprises (i) a VH comprising an amino acid sequence that is at least 99% identical to a VH amino acid sequence of an antibody in Table 8, 13, 22 or 25 and (ii) a VL comprising an amino acid sequence that is at least 99% identical to the VL amino acid sequence of the same antibody in Table 8, 13, 22 or 25. In some aspects, the antigen-binding domain that specifically binds to human CD98hc also comprises the CDRs of the antibody in Table 8, 13, 22 or 25 (e.g., the non-identical amino acids in the VH and/or VL are outside of the CDRs).

In some aspects, provided herein is an antigen-binding domain that binds to the same CD98hc epitope as an antibody comprising a VH amino acid sequence of an antibody in Table 8, 13, 22 or 25 and a VL amino acid sequence of the same antibody in Table 8, 13, 22 or 25.

In some aspects, provided herein is an antigen-binding domain that competitively inhibits binding to CD98hc of as an antibody comprising a VH amino acid sequence of an antibody in Table 8, 13, 22 or 25 and a VL amino acid sequence of the same antibody in Table 8, 13, 22 or 25.

In some aspects, an antigen-binding domain that specifically binds to human CD98hc comprises a VH and a VL on a single polypeptide chain (e.g., a VH and VL in Table 8, 13, 22 or 25). In some aspects, the antigen-binding domain comprises an scFv. The scFv can comprise a VH that is N-terminal to a VL or a VL that is N-terminal to a VH. The scFv can comprise a linker, e.g., between a VH and a VL. Accordingly, the scFv can be in the orientation VH-linker-VL or VL-linker-VH. Such a linker can be about 5 to about 25 amino acids in length. Such a linker can be about 5 to about 20 amino acids in length. Such a linker can be about 10 to about 25 amino acids in length. Such a linker can be about 10 to about 20 amino acids in length. Such a linker can be, e.g., a glycine linker, a glycine-rich linker, or a glycine-serine linker. Such a linker can comprise the amino acid sequence of GGSEGKSSGSGSESKSTGGS (SEQ ID NO: 182). Such a linker can comprise the amino acid sequence of

(SEQ ID NO: 337)
GGGGSGGGGSGGGGSGGGGS.

In some aspects, an antigen-binding domain that specifically binds to human CD98hc comprises a VH on a first polypeptide and a VL on a second polypeptide (e.g., a Fab).

In some aspects, an antigen-binding domain that specifically binds to human CD98hc comprises the antigen-binding fragment of a heavy chain only antibody (e.g., a VHH or nanobody).

In some aspects, an antigen-binding domain that specifically binds to human CD98hc is a murine antigen-binding domain. In some aspects, an antigen-binding domain that specifically binds to human CD98hc is a chimeric antigen-binding domain. In some aspects, an antigen-binding domain that specifically binds to human CD98hc is a humanized antigen-binding domain. In some aspects, an antigen-binding domain that specifically binds to human CD98hc is a human antigen-binding domain

In some aspects, an antigen-binding domain provided herein that specifically binds to human CD98hc also binds to cynomolgus monkey CD98hc.

In some aspects, an antigen-binding domain provided herein specifically binds to human CD98hc with an affinity of no more than 250 nM (e.g., 10 μM to 250 nM, 5 μM to 250 nM, 1 μM to 250 nM, 1 nM to 250 nM or 3 nM to 250 nM), an affinity of no more than 200 nM (e.g. 10 μM to 200 nM, 5 μM to 200 nM, 1 μM to 200 nM, 1 nM to 200 nM or 3 nM to 200 nM), or an affinity of no more than 150 nM (e.g., 10 μM to 150 nM, 5 μM to 150 nM, 1 μM to 150 nM, 1 nM to 150 nM or 3 nM to 150 nM), optionally wherein the affinity is measured using surface plasmon resonance. Surface plasmon resonance can be measured, e.g., using the Carterra LSA platform. In some aspects, an antigen-binding domain provided herein that specifically binds to human CD98hc with an affinity of 0.1 μM to 10 μM, 0.1 μM to 100 μM, 0.1 μM to 100 μM, 0.1 μM to 1 nM, 1 μM to 10 μM, 1 μM to 100 μM, 1 μM to 1 nM, 1 μM to 10 nM, 1 μM to 100 nM, 1 μM to 150 nM, or 1 μM to 250 nM.

In some aspects, an antigen-binding domain provided herein that specifically binds to human CD98hc binds to human CD98hc with an ELISA OD₄₅₀ of at least 0.45. In some aspects, an antigen-binding domain provided herein that specifically binds to human CD98hc binds to cynomolgus CD98hc with an ELISA OD₄₅₀ of at least 0.45. In some aspects, an antigen-binding domain provided herein that specifically binds to human CD98hc binds to human CD98hc with an ELISA OD₄₅₀ of at least 0.45 and binds to cynomolgus CD98hc with an ELISA OD₄₅₀ of at least 0.45.

In some aspects, an antigen-binding domain provided herein that specifically binds to human CD98hc binds to cynomolgus CD98hc with an affinity of no more than 250 nM (e.g., 10 μM to 250 nM, 5 μM to 250 nM, 1 μM to 250 nM, 10 μM to 250 nM, 1 nM to 250 nM or 3 nM to 250 nM), an affinity of no more than 200 nM (e.g., 10 μM to 200 nM, 5 μM to 200 nM, 1 M to 200 nM, 10 μM to 250 nM, 1 nM to 200 nM or 3 nM to 200 nM), or an affinity of no more than 150 nM (e.g., 10 μM to 150 nM, 5 M to 150 nM, 1 μM to 150 nM, 10 μM to 250 nM, 1 nM to 150 nM or 3 nM to 150 nM), optionally wherein the affinity is measured using surface plasmon resonance. Surface plasmon resonance can be measured, e.g., using the Carterra LSA platform. In some aspects, an antigen-binding domain provided herein that specifically binds to cyno CD98hc binds to cyno CD98hc with an affinity of 1 μM to 100 μM, 1 μM to 1 nM, 1 μM to 10 nM, 1 μM to 100 nM, 1 μM to 150 nM, or 1 μM to 250 nM.

In some aspects, an antigen-binding domain provided herein that specifically binds to human CD98hc binds to each of human CD98hc and cynomolgus CD98hc with an affinity of no more than 250 nM (e.g., 1 μM to 250 nM, 1 nM to 250 nM or 3 nM to 250 nM), an affinity of no more than 200 nM (e.g., 1 μM to 200 nM, 1 nM to 200 nM or 3 nM to 200 nM), or an affinity of no more than 150 nM (e.g., 1 μM to 150 nM, 1 nM to 150 nM or 3 nM to 150 nM), optionally wherein the affinity is measured using surface plasmon resonance. Surface plasmon resonance can be measured, e.g., using the Carterra LSA platform. In some aspects, an antigen-binding domain provided herein that specifically binds to human CD98hc binds to each of human CD98hc and cynomolgus CD98hc with an affinity of 1 μM to 100 μM, 1 μM to 1 nM, 1 μM to 10 nM, 1 μM to 100 nM, 1 μM to 150 nM, or 1 μM to 250 nM.

In some aspects, an antigen-binding domain provided herein that specifically binds to human CD98hc binds to human CD98hc with an affinity of 3.1 nM to 210 nM. In some aspects, an antigen-binding domain provided herein that specifically binds to human CD98hc binds to human CD98hc with an affinity of 18 nM to 35 nM. In some aspects, an antigen-binding domain provided herein that specifically binds to human CD98hc binds to human CD98hc with an affinity of 12 nM to 34 nM. In some aspects, In some aspects, an antigen-binding domain provided herein that specifically binds to human CD98hc binds to cynomolgus CD98hc with an affinity of 3.2 to 145 nm. In some aspects, an antigen-binding domain provided herein that specifically binds to human CD98hc binds to cynomolgus CD98hc with an affinity of 340 nm to 1.5 M. In some aspects, an antigen-binding domain provided herein that specifically binds to human CD98hc binds to cynomolgus CD98hc with an affinity of 120 nm to 880 nm. In some aspects, an antigen-binding domain provided herein that specifically binds to human CD98hc binds to human CD98hc with an affinity of 3.1 nM to 210 nM and binds to cynomolgus CD98hc with an affinity of 3.2 nM to 145 nM, optionally wherein the affinity is measured using surface plasmon resonance. Surface plasmon resonance can be measured, e.g., using the Carterra LSA platform.

In some aspects, an antigen-binding domain provided herein that specifically binds to human CD98hc does not reduce cell surface expression of CD98hc on HCMED/D3 cells by more than 20% relative to cell surface expression of CD98hc on HCMEC/D3 cells treated with an isotype control. Cell surface expression can be measured, e.g., using Western blot or FACS.

In some aspects, an antigen-binding domain provided herein that specifically binds to human CD98hc does not increase cell surface expression of CD98hc on HCMED/D3 cells by more than 50% relative to cell surface expression of CD98hc on HCMEC/D3 cells treated with an isotype control. Cell surface expression can be measured, e.g., using Western blot or FACS.

In some aspects, an antigen-binding domain provided herein that specifically binds to human CD98hc does not reduce cell surface expression of CD98hc on HCMED/D3 cells by more than 20% relative to cell surface expression of CD98hc on HCMEC/D3 cells treated with an isotype control and does not increase cell surface expression of CD98hc on HCMED/D3 cells by more than 50% relative to cell surface expression of CD98hc on HCMEC/D3 cells treated with an isotype control. Cell surface expression can be measured, e.g., using Western Blot or FACS.

In some aspects, an antigen-binding domain provided herein that specifically binds to human CD98hc accumulates at least 1.5-fold more than an isotype control in vessel-depleted human CD98hc knock-in mouse brain after peripheral injection. In some aspects, an antigen-binding domain provided herein that specifically binds to human CD98hc accumulates at least 1-fold more than an isotype control in vessel-depleted mouse brain.

In some aspects, an antigen-binding domain provided herein that specifically binds to human CD98hc has at least a 5-fold increase in brain:serum concentration ratio over an isotype control 24 hours after administration to a mouse.

Also provided herein are antigen-binding domains that bind to the same epitope of CD98hc as a CD98hc antigen-binding domain provided herein. Also provided herein are antigen-binding domains that competitively inhibit binding to CD98hc of CD98hc antigen-binding domain provided herein.

### Agents Comprising Anti-CD98hc Antigen-Binding Domains

Provided herein are agents (e.g., fusion proteins, multi-specific (e.g., bispecific) proteins, antibodies, antigen-binding fragments thereof, etc.) comprising an antigen-binding domain that specifically binds to human CD98hc.

### Fusion Proteins

In some aspects, a fusion protein provided herein comprises an antigen-binding domain that specifically binds to human CD98hc and a heterologous protein or polypeptide. In some aspects, the heterologous protein is a protein or polypeptide or fragment thereof useful in protein replacement therapy (PRT). In some aspects, the heterologous polypeptide is an enzyme (e.g., an enzyme for use in enzyme replacement therapy (ERT)) or a catalytically active fragment thereof. In some aspects, the heterologous polypeptide is an ERT enzyme or an ERT enzyme variant, or a catalytically active fragment thereof. In some aspects, the heterologous polypeptide in a fusion protein provided herein is a growth factor. In some aspects, the heterologous polypeptide in a fusion protein provided herein is a decoy receptor. In some aspects, the heterologous polypeptide in a fusion protein provided herein is progranulin (PGRN), prosaposin (PSAP), or survival motor neuron protein (SMN). In some aspects, the heterologous protein is an enzyme selected from ubiquitin protein ligase E3A (UBE3A), α-L Iduronidase (IDUA), Iduronate-2-sulphatase (IDS), N-acetylgalactoslamine-6-sulphatase (GALNS), N-sulfoglucosamine sulfohydrolase (SGSH), N-acetylgalactosamine-4-sulphatase (arylsulphatase B; ARSB), acid sphingomyelinase (ASM), β-glucocerebrosidase (GCase or GBA), galactosylceramide beta-galactosidase, glucosylceramidase, beta-hexosaminidase A, beta-hexosaminidase B, arylsulphatase A, beta-galactosidase, acid ceramidase, alpha-glucosidase, lysosomal acid lipase, lysosomal protease, a synthetic enzyme replacement thereof, such as larodinase, idursulphase, elosulphase alpha or galsuphase, or a variant thereof, or a catalytically active fragment thereof. In some aspects, the heterologous protein is a protein or an enzyme selected from clusterin (APOJ), Reelin, Tripeptidyl Peptidase 1 (CLN2/TPP1), glucosamine (N-acetyl)-6-sulfatase (GNS), heparan-alpha-glucosaminide N-acetyltransferase (HGSNAT), and N-acetyl-alpha-glucosaminidase (NAGLU), α-L Iduronidase (IDUA), Iduronate-2-sulphatase (IDS), N-acetylgalactoslamine-6-sulphatase (GALNS), N-sulfoglucosamine sulfohydrolase (SGSH), N-acetylgalactosamine-4-sulphatase (arylsulphatase B; ARSB), acid sphingomyelinase (ASM), β-glucocerebrosidase (GCase or GBA), galactosylceramide beta-galactosidase, glucosylceramidase, beta-hexosaminidase A, beta-hexosaminidase B, arylsulphatase A, beta-galactosidase, acid ceramidase, alpha-glucosidase, lysosomal acid lipase, lysosomal protease, a synthetic enzyme replacement thereof, such as larodinase, idursulphase, elosulphase alpha or galsuphase, or a variant thereof, or a catalytically active fragment thereof. In some aspects, the heterologous protein in the fusion protein is N-terminal to the antigen-binding domain that specifically binds to human CD98hc. In some aspects, the heterologous protein or polypeptide in the fusion protein is C-terminal to the antigen-binding domain that specifically binds to human CD98hc. In some aspects, the heterologous protein or polypeptide and the antigen-binding domain that specifically binds to human CD98hc are directly connected via a peptide bond. In some aspects, the heterologous fusion protein and the antigen-binding domain that specifically binds to human CD98hc are connected via a linker, e.g., a peptide linker. In some aspects, the fusion protein comprises an antigen-binding domain and a heterologous protein or polypeptide and an Fc portion. In some aspects, the antigen-binding domain and the heterologous protein or polypeptide are linked to the N-terminus of the Fc portion of the fusion protein. In other aspects, the antigen-binding domain is linked to the N-terminus of the Fc portion and the heterologous protein or polypeptide is linked to the C-terminus of the Fc portion of the fusion protein. In other aspects, the antigen-binding domain is linked to the C-terminus of the Fc portion and the heterologous protein or polypeptide is linked to the N-terminus of the Fc portion of the fusion protein.

### Bispecific and Multispecific Proteins

In some aspects, an antibody or antigen-binding fragment thereof provided herein comprises an antigen-binding domain that specifically binds to human CD98hc. In some aspects, an antibody or antigen-binding fragment thereof comprises an antigen-binding domain that specifically binds to human CD98hc and an antigen-binding domain that specifically binds to a CNS antigen or a brain antigen. In some aspects, the CNS antigen or brain antigen is not CD98hc. Also provided herein are antibodies or antigen-binding fragments thereof that bind to the same epitope of CD98hc as a CD98hc antigen-binding domain provided herein. Also provided herein are antibodies or antigen-binding fragments thereof that competitively inhibit binding to CD98hc of a CD98hc antigen-binding domain provided herein.

In some aspects, a multi-specific protein provided herein comprises a first antigen-binding domain that binds to human CD98hc and a second antigen-binding domain. The first antigen-binding domain that binds to human CD98hc can be any antigen-binding domain that binds to human CD98hc provided herein. The second antigen-binding domain can be an antigen-binding domain that specifically binds to a CNS antigen or a brain antigen. In some aspects, the CNS antigen or brain antigen is not CD98hc.

In some aspects, a multi-specific protein provided herein comprises an antigen-binding domain that binds to human CD98hc linked to an antibody or antigen-binding fragment thereof. The antibody or antigen-binding fragment thereof can bind a CNS antigen or brain antigen. In some aspects, the CNS antigen or brain antigen is not CD98hc. In some aspects, such a multi-specific protein can be in a 2+1 antibody format (as shown in FIG. 1A) or a 2+2 antibody format (FIGS. 1B and 1C).

In some aspects, a multi-specific protein provided herein comprises a CD98hc antigen-binding domain that is an scFv linked to an antibody that binds to a CNS antigen, wherein the antibody comprises two heavy chains and two light chains. In some aspects, the scFv is linked to the C-terminus of one of the two antibody heavy chains, e.g., via a protein linker.

In some aspects, the multi-specific protein comprises 1) an antigen-binding domain that bindsCD98hc, 2) a second antigen-binding domain that binds a different CNS or brain antigen, and 3) an Fc region, wherein the CD98hc antigen-binding domain and the second antigen-binding domain are connected or linked to an Fc region of the multi-specific protein. In other aspects, the multi-specific protein comprises 1) an antigen-binding domain that comprises a heavy chain variable region and bindsCD98hc, 2) a second antigen binding domain that comprises a heavy-chain variable region and binds a different CNS or brain antigen, and 3) an Fc region, wherein the CD98hc antigen-binding domain and the second antigen-binding domain are connected or linked to the Fc region of the multi-specific protein. In some aspects, the multi-specific protein comprises an antigen-binding domain that bindsCD98hc, a second antigen-binding domain that binds a different CNS or brain antigen, and an Fc region. In some aspects, the CD98hc antigen-binding domain and the second antigen-binding domain are connected or linked to the N-terminus of the Fc portion of the multi-specific protein. In other aspects, the CD98hc antigen-binding domain is connected or linked to the N-terminus of an Fc portion of the multi-specific protein and the second antigen-binding domain is linked to the C-terminus of the Fc portion of the multi-specific protein. In other aspects, the CD98hc antigen-binding domain is connected or linked to the C-terminus of an Fc portion of the multi-specific protein and the second antigen-binding domain is linked to the N-terminus of the Fc portion of the multi-specific protein.

In some aspects, a multi-specific protein provided herein comprises two copies of a CD98hc antigen-binding domain that is an scFv and an antibody that binds to a CNS antigen, wherein the antibody comprises two heavy chains and two light chains, wherein one of the two copies of the antigen-binding domain is linked to the C-terminus of one of the antibody heavy chains, and wherein the other copy of the antigen-binding domain is linked to the C-terminus of the other antibody heavy chain. In some aspects, the scFvs are linked to heavy chains via a protein linker.

As provided herein, a fusion protein, antibody, or antigen-binding fragment thereof, or multi-specific protein provided herein can be multi-specific, e.g., bi-specific. Many different formats and uses of bi-specific binding molecules are known in the art (reviewed in, e.g., Kontermann; Drug Discov Today, 2015 July; 20 (7): 838-47; MAbs, 2012 March-April; 4 (2): 182-97). A bispecific protein according to the present invention is not limited to any particular bispecific format or method of producing it. Accordingly, bispecific proteins of the present disclosure can include various configurations having a first antigen-binding domain that binds to human CD98hc and a second antigen-binding domain, e.g., that binds to a CNS antigen or a brain antigen.

Examples of bispecific molecules that can be used in the present disclosure include, e.g., (i) a single antibody that has two arms comprising different antigen-binding domains; (ii) a single chain antibody that has specificity to two different epitopes, e.g., via two scFvs linked in tandem by an extra peptide linker; (iii) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig.TM.) Molecule, In: Antibody Engineering, Springer Berlin Heidelberg (2010)); (iv) a chemically-linked bispecific (Fab′) 2 fragment; (v) a Tandab, which is a fusion of two single chain diabodies resulting in a tetravalent bispecific antibody that has two binding sites for each of the target antigens; (vi) a flexibody, which is a combination of scFvs with a diabody resulting in a multivalent molecule; (vii) a so-called “dock and lock” molecule, based on the “dimerization and docking domain” in Protein Kinase A, which, when applied to Fabs, can yield a trivalent bispecific binding protein consisting of two identical Fab fragments linked to a different Fab fragment; (viii) a so-called Scorpion molecule, comprising, e.g., two scFvs fused to both termini of a human Fab-arm; and (ix) a diabody. Other examples of antibody structures are described in WO2019/246288, which is incorporated by reference.

In some aspects, a fusion protein, antibody or antigen-binding fragment thereof, or multi-specific protein provided herein is multivalent (e.g., bivalent). In some aspects, a fusion protein, antibody or antigen-binding fragment thereof, or multi-specific protein provided herein is trivalent (e.g., in the 2+1 antibody format). In some aspects, a trivalent format comprises a single CD98hc antigen-binding domain provided herein and two antigen-binding domains that bind to a CNS antigen or a brain antigen. The two antigen-binding domains that bind to a CNS antigen or a brain antigen can comprise the same amino acid sequence or can comprise different amino acid sequences. In some aspects, the CD98hc antigen-binding domain is an scFv. In some aspects, the CD98hc antigen-binding domain is a VHH.

In some aspects, a fusion protein, antibody or antigen-binding fragment thereof, or multi-specific protein provided herein is tetravalent (e.g., in the 2+2 antibody format). In some aspects, a tetravalent format comprises two CD98hc antigen-binding domains provided herein and two antigen-binding domains that bind to a CNS antigen or a brain antigen. The two CD98hc antigen-binding domains can comprise the same amino acid sequence or can comprise different amino acid sequences. In some aspects, the two CD98hc antigen-binding domains comprise the same amino acid sequence. In some aspects, one or both of the CD98hc antigen-binding domains is an scFv. The two antigen-binding domains that bind to a CNS antigen or a brain antigen can comprise the same amino acid sequence or can comprise different amino acid sequences.

A fusion protein, antibody or antigen-binding fragment thereof, or multi-specific protein provided herein can comprise a linker, e.g., linking a CD98hc antigen-binding domain to a heterologous protein, antibody or antigen-binding fragment thereof, or other antigen-binding domain. The linker can be e.g., a glycine linker, a glycine-rich linker, or a glycine-serine linker. The linker can comprise the amino acid sequence (GGGGS)x3 (SEQ ID NO:183). The linker can comprise the amino acid sequence (GGSGG)x3 (SEQ ID NO:338). The linker can comprise the amino acid sequence GGSGG (no repeats) (SEQ ID NO:354). The linker can be 1 to 20 amino acids in length.

A fusion protein, antibody or antigen-binding fragment thereof, or multi-specific protein provided herein can comprise a constant region. In some aspects, a CD98hc antigen-binding domain provided herein is linked to the constant region, e.g., the C-terminus of the constant region. In some aspects, a constant domain is a human constant domain. In some aspects, a constant domain is a murine, rat, rabbit, or monkey (e.g., cynomolgus) constant domain. The constant region can be a heavy chain constant region. The constant region can be a human constant region. The constant region can be a human heavy chain constant region. The constant region can be an IgG constant region. The constant region can be an IgG1 constant region. The constant region can be an IgG2 constant region. The constant region can be an IgG4 constant region. The constant region can be a human IgG constant region. The constant region can be a human IgG1 constant region. The constant region can be a human IgG2 constant region. The constant region can be a human IgG4 constant region.

In some aspects a fusion protein, antibody or antigen-binding fragment thereof, or multi-specific protein provided herein comprises a heavy chain and a light chain. With respect to the heavy chain, in some aspects, the heavy chain of an antigen-binding protein described herein can be an alpha (α), delta (δ), epsilon (ε), gamma (γ) or mu (μ) heavy chain. In some aspects, the heavy chain can comprise a human alpha (α), delta (δ), epsilon (ε), gamma (γ) or mu (μ) heavy chain. In some aspects, the heavy chain comprises a human gamma (γ) heavy chain constant region. In some aspects, the heavy chain of comprises the amino acid sequence of an IgG1 heavy chain constant region. In some aspects, the heavy chain comprises the amino acid sequence of an IgG2 (e.g., IgG2a or IgG2b) heavy chain constant region. In some aspects, the heavy chain comprises the amino acid sequence of an IgG4 heavy chain constant region. With respect to the light chain, in some aspects, the light chain is a kappa light chain. In some aspects, the light chain is a lambda light chain. In some aspects, the light chain is a human kappa light chain or a human lambda light chain.

In some aspects, a fusion protein, antibody or antigen-binding fragment thereof, or multi-specific protein provided herein comprises constant regions comprising the amino acid sequences of the constant regions of an IgG, IgE, IgM, IgD, IgA, or IgY immunoglobulin molecule, or a human IgG, IgE, IgM, IgD, IgA, or IgY immunoglobulin molecule. In some aspects, a fusion protein, antibody or antigen-binding fragment thereof, or multi-specific protein provided herein comprises constant regions comprising the amino acid sequences of the constant regions of an IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule. In some aspects, the constant regions comprise the amino acid sequences of the constant regions of a human IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule.

Non-limiting examples of human constant region sequences have been described in e.g., U.S. Pat. No. 5,693,780 and Kabat E A et al., (1991) Sequences of Proteins of Immunological Interest, Fifth Edition, U.S. Department of Health and Human Services, NIH Publication No. 91-3242).

In some aspects, a constant region provided herein comprises a knob mutation. In some aspects, a constant region provided herein comprises a hole mutation. Accordingly, in some aspects, a fusion protein, antibody or antigen-binding fragment thereof, or multi-specific protein provided herein can comprise a constant region comprising a knob mutation and a constant region comprising a hole mutation.

### FC Domains

A fusion protein, antibody or antigen-binding fragment thereof, or multi-specific protein provided herein can comprise an Fc domain or fragment thereof. In some aspects, an Fc domain is of IgG class, the IgM class, or the IgA class. In some aspects, an Fc domain or fragment thereof is an IgG Fc domain or fragment thereof. In some aspects, an Fc domain or fragment thereof is a human IgG Fc domain or fragment thereof. In some aspects, an Fc domain or fragment thereof is a human IgG1 Fc domain or fragment thereof. In some aspects, an Fc domain or fragment thereof is a human IgG2 Fc domain or fragment thereof. In some aspects, an Fc domain or fragment thereof is a human IgG4 Fc domain or fragment thereof.

In some aspects provided herein, a fusion protein, antibody or antigen-binding fragment thereof, or multi-specific protein provided comprises a modified Fc domain or fragment thereof. In some aspects, the modified Fc domain or fragment thereof is a modified IgG1 Fc comprising one or more modifications. For example, in some aspects, the IgG1 modified Fc comprises one or more amino acid substitutions (e.g., relative to a wild-type Fc region of the same isotype). In some aspects, the one or more amino acid substitutions are selected from N297A (Bolt S et al. (1993) Eur J Immunol 23:403-411), D265A (Shields et al. (2001) R. J. Biol. Chem. 276, 6591-6604), L234A, L235A (Hutchins et al. (1995) Proc Natl Acad Sci USA, 92:11980-11984; Alegre et al., (1994) Transplantation 57:1537-1543. 31; Xu et al., (2000) (′ell Immunol, 200:16-26), G237A (Alegre et al. (1994) Transplantation 57:1537-1543. 31; Xu et al. (2000) Cell Immunol, 200:16-26), C226S, C229S, E233P, L234V, L234F, L235E (McEarchern et al., (2007) Blood, 109:1185-1192), P331S (Sazinsky et al., (2008) Proc Natl Acad Sci USA 2008, 105:20167-20172), S267E, L328F, A330L, M252Y, S254T, E430G, and/or T256E, where the amino acid position is according to the EU numbering convention. In some aspects, the bispecific antibody comprises the amino acid substitutions L234A, L235A, and P331S (LALAPS) accordingly to EU numbering. In some aspects of any of the modified IgG1 Fc, the Fc comprises N325S and L328F mutations according to EU numbering. In some aspects of any of the modified IgG1 Fc, the Fc comprises P329G or P329S according to EU numbering.

In some aspects provided herein, a fusion protein, antibody or antigen-binding fragment thereof, or multi-specific protein provided herein is a bi-specific fusion protein, antibody or antigen-binding fragment thereof, or multi-specific protein. Bi-specific molecules include, e.g., a kappa-lambda body, a dual-affinity re-targeting molecule (DART), a knob-in-hole antibody, a strand-exchange engineered domain body (SEEDbody), and a DuoBody. In some aspects, a bispecific fusion protein, antibody or antigen-binding fragment thereof, or multi-specific protein provided herein comprises a knob mutation and a hole mutation. In some aspects, the knob mutation comprises the amino acid substitution T366W according to EU numbering. In some aspects, the hole mutation comprises the amino acids substitutions T366S, L368A, and Y407V according to EU numbering.

In some aspects provided herein, a fusion protein, antibody or antigen-binding fragment thereof, or multi-specific protein provided herein comprises a mutation to promote heterodimerization of Fc regions. In some aspects, a dimerized Fc region of a bispecific provided herein is formed by Fc regions that contain amino acid mutations, substitutions, additions, or deletions to promote heterodimerization in which different polypeptides comprising different Fc regions can dimerize to yield a heterodimer configuration. In some aspects, a bispecific of the present disclosure comprises a first Fc sequence comprising a first CH3 region, and a second Fc sequence comprising a second CH3 region, wherein the sequences of the first and second CH3 regions are different and are such that the heterodimeric interaction between said first and second CH3 regions is stronger than each of the homodimeric interactions of said first and second CH3 regions

Methods to promote heterodimerization of Fc regions include amino acid deletions, additions, or substitutions of the amino acid sequence of the Fc region, such as by including a set of “knob-into-hole” deletions, additions, or substitutions or including amino acid deletions, additions, or substitutions to effect electrostatic steering of the Fc to favor attractive interactions among different polypeptide chains. Methods for promoting heterodimerization of complementary Fc polypeptides have been previously described in, for example, Ridgway et al, 1996, Protein Eng, 9:617-621; Merchant et al, 1998, Nature Biotechnol, 16:677-681; Moore et al, 2011, MAbs, 3:546-557; Von Kreudenstein et al, 2013, 5:646-654; Gunasekaran et al, 2010, J Biol Chem, 285:19637-19464; Leaver-Fay et al, 2016, Structure, 24:641-651; Ha et al, 2016, Frontiers in Immunology, 7:1; Davis et al, 2010, Protein Eng Des Sel, 23:195-202; WO1996/027011; WO1998/050431; WO2006/028936; WO2009/089004; WO2011/143545; WO2014/067011; WO2012/058768; WO2018/027025; US2014/0363426; US2015/0307628; US2018/0016354; US2015/0239991; US2017/0058054; U.S. Pat. Nos. 5,731,168; 7,183,076; 9,701,759; 9,605,084; 9,650,446; 8,216,805; 8,765,412; and 8,258,268.

In some aspects, complementary Fc polypeptides of an Fc heterodimer include a mutation to alter charge polarity across the Fc dimer interface such that co-expression of electrostatically matched Fc regions support favorable attractive interactions, thereby promoting desired Fc heterodimer formation; whereas unfavorable repulsive charge interactions suppress unwanted Fc homodimer formation (Guneskaran et al, 2010, J Biol Chem, 285:19637-19646). When co-expressed in a cell, association between the polypeptide chains is possible but the chains do not substantially self-associate due to charge repulsion.

Additionally, complementary Fc polypeptides of an Fc heterodimer include “knob-into-hole” configurations to promote heterodimerization of two Fc polypeptides. “Knob-into-hole” technology is described in e.g., U.S. Pat. Nos. 5,731,168; 7,695,936; 8,216,805; 8,765,412; Ridgway et al., Prot Eng 9, 617-621 (1996); and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). The protuberance and cavity can be made by altering the nucleic acid encoding the polypeptides, e.g., by site-specific mutagenesis, or by peptide synthesis. In some aspects, a knob modification comprises the amino acid substitution T366W in one of the two subunits of the Fc domain, and the hole modification comprises the amino acid substitutions T366S, L368A and Y407V in the other one of the two subunits of the Fc domain. In some aspects, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc domain, thus further stabilizing the dimer (Carter, J Immunol Methods 248, 7-15 (2001)). Thus, in such configurations, a first Fc polypeptide comprises amino acid modifications to form the “knob” and a second Fc polypeptide comprises amino acid modifications to form the “hole” thus forming an Fc heterodimer comprising complementary Fc polypeptides.

Exemplary paired amino acid modifications of complementary Fc polypeptides of an Fc heterodimeric configuration are set forth below in the table below (EU numbering).

TABLE 1
Exemplary paired Fc modifications for heterodimeric Fc domains

| First Fc polypeptide | Second Fc polypeptide |
|---|---|
| T366Y | Y407T |
| T366W | T366S/L368W/Y407V |
| T366W | T366S/L368A/Y407V |
| T366W/S354C | T366S/L368A/Y407V/Y349C |
| T350V/L351Y/F405A/Y407V | T350V/T366L/K392L/T394W |
| K360D/D399M/Y407A | E345R/Q347R/T366V/K409V |
| K409D/K392D | D399K/E356K |
| K360E/K409W | Q347R/D399V/F405T |
| L360E/K409W/Y349C | Q347R/D399V/F405T/S354C |
| K370E/K409W | E357N/D399V/F405T |

Some agents provided herein comprise antigen-binding fragments of antibodies. Antigen-binding fragments of antibodies include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that can be bivalent and/or bispecific. See, for example, EP404097; WO 1993/01161; Hudson et al. Nat. Med. 9:129-134 (2003). Triabodies and tetrabodies are also described in Hudson et al. Nat. Med. 9:129-134 (2003). Single-domain antibodies are antibody fragments comprising all or a portion of the heavy chain variable domain or all or a portion of the light chain variable domain of an antibody. Some aspects, a single-domain antibody is a human single-domain antibody (see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

As provided herein, a fusion protein, antibody or antigen-binding fragment thereof, or multi-specific protein provided herein can be chimeric. Certain chimeric antibodies are described, e.g., in U.S. Pat. No. 4,816,567. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody.

As provided herein, a fusion protein, antibody or antigen-binding fragment thereof, or multi-specific protein provided herein can be humanized. Typically, a non-human antibody is humanized to reduce immunogenicity to humans, while retaining the specificity and affinity of the parental non-human antibody. In some aspects, a humanized antibody is substantially non-immunogenic in humans. In some aspects, a humanized antibody has substantially the same affinity for a target as an antibody from another species from which the humanized antibody is derived. See, e.g., U.S. Pat. Nos. 5,530,101, 5,693,761; 5,693,762; and 5,585,089. In some aspects, amino acids of an antibody variable domain that can be modified without diminishing the native affinity of the antigen-binding domain while reducing its immunogenicity are identified. See, e.g., U.S. Pat. Nos. 5,766,886 and 5,869,619. Generally, a humanized antibody comprises one or more variable domains in which CDRs (or portions thereof) are derived from a non-human antibody, and framework regions (FRs) (or portions thereof) are derived from human antibody sequences. A humanized antibody can comprise at least a portion of a human constant region. In some aspects, some FR residues in a humanized antibody are substituted with corresponding residues from a non-human antibody (e.g., the antibody from which the CDR residues are derived), for example, to restore or improve antibody specificity or affinity.

Humanized antibodies and methods of making them are reviewed, for example, in Almagro et al. Front. Biosci. 13:161 9-1633 (2008), and are further described, e.g., in U.S. Pat. Nos. 5,821,337, 7,527,791, 6,982,321, and 7,087,409. Human framework regions that can be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA 89:4285 (1992); and Presta et al., J. Immunol. 151:2623 (1993)); human mature (somatically mutated) framework regions or human germline framework regions (see, e.g., Almagro and Fransson Front. Biosci. 13:1619-1633 (2008)); and framework regions derived from screening FR libraries (see, e.g., Baca et al. J. Biol. Chem. 272:10678-10684 (1997) and Rosok et al. J. Biol. Chem. 271:22611-22618 (1996)).

As provided herein, a fusion protein, antibody or antigen-binding fragment thereof, or multi-specific protein provided herein can be human. Human antibodies can be produced using various techniques known in the art. Human antibodies are described generally in van Dijk et al. Curr. Opin. Pharmacol. 5:368-74 (2001) and Lonberg Curr. Opin. Immunol. 20:450-459 (2008).

Human antibodies can be prepared by administering an immunogen to a transgenic animal that has been modified to produce intact human antibodies or intact antibodies with human variable regions in response to antigenic challenge. One can engineer mouse strains deficient in mouse antibody production with large fragments of the human Ig loci in anticipation that such mice would produce human antibodies in the absence of mouse antibodies. Large human Ig fragments can preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains can yield high affinity fully human antibodies against any antigen of interest, including human antigens. Using the hybridoma technology, antigen-specific human MAbs with the desired specificity can be produced and selected. Certain exemplary methods are described in U.S. Pat. No. 5,545,807, EP 546073, and EP 546073. See also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals can be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol. 133:3001 (1984) and Boerner et al. J. Immunol. 147:86 (1991)). Human antibodies generated via human B-cell hybridoma technology are also described in Li et al. Proc. Natl. Acad. Sci. USA, 1 03:3557-3562 (2006). Additional methods include those described, for example, in U.S. Pat. No. 7,189,826 (describing production of monoclonal human IgM antibodies from hybridoma cell lines). Human hybridoma technology (Trioma technology) is also described in Vollmers et al. Histology and Histopathology 20 (3): 927-937 (2005) and Vollmers et al. Methods and Findings in Experimental and Clinical Pharmacology 27 (3): 185-91 (2005). Human antibodies can also be generated by isolating Fv clone variable domain sequences selected from human-derived phage display libraries. Such variable domain sequences can then be combined with a desired human constant domain. Techniques for selecting human antibodies from antibody libraries are described below.

In some aspects provided herein, an antibody is a human antibody isolated by in vitro methods and/or screening combinatorial libraries for antibodies with the desired activity or activities. Suitable examples include but are not limited to phage display (CAT, Morphosys, Dyax, Biosite/Medarex, Xoma, Symphogen, Alexion (formerly Proliferon), Affimed) ribosome display (CAT), yeast display (Adimab), and the like. In certain phage display methods, repertoires of VH and VL genes are separately cloned by polymerase chain reaction (PCR) and recombined randomly in phage libraries, which can then be screened for antigen-binding phage as described in Winter et al. Ann. Rev. Immunol. 12:433-455 (1994). For example, a variety of methods are known in the art for generating phage display libraries and screening such libraries for antibodies possessing the desired binding characteristics. See also Sidhu et al. J. Mol. Biol. 338 (2): 299-310, 2004; Lee et al. J. Mol. Biol. 340 (5): 1073-1093, 2004; Fellouse Proc. Natl. Acad. Sci. USA 101 (34): 12467-12472 (2004); and Lee et al. J. Immunol. Methods 284 (-2): 1 19-132 (2004). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al. EMBO J. 12:725-734 (1993). Finally, naive libraries can also be made synthetically by cloning unrearranged V-gene segments from stem cells, and using PCR primers comprising random sequence to encode the highly variable CDR3 regions and to accomplish rearrangement in vitro, as described by Hoogenboom et al. J. Mol. Biol., 227:381-388, 1992. Patent publications describing human antibody phage libraries include, for example: U.S. Pat. No. 5,750,373, and US Patent Publication Nos. 2007/0292936 and 2009/0002360. Antibodies isolated from human antibody libraries are considered human antibodies or human antibody fragments herein.

As provided herein, a fusion protein, antibody or antigen-binding fragment thereof, or multi-specific protein provided herein can comprise an antigen-binding domain that binds to a CNS antigen or a brain antigen. In some aspects, the CNS antigen or brain antigen can be beta-secretase 1 (BACE1), Abeta, epidermal growth factor receptor (EGFR), human epidermal growth factor receptor 2 (HER2), tau, apolipoprotein, apolipoprotein E (ApoE), apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), caspase 6, sortilin (SORT), triggering receptor expressed on myeloid cells 2 (TREM2), CD33 or sialic acid binding Ig-like lectin 3 (Siglec3), Membrane Spanning 4-Domains A4A (MS4A4A), Membrane Spanning 4-Domains A 6A (MS4A6A), or Transmembrane Protein 106B (TMEM106b). In some aspects, the CNS antigen or brain antigen can be beta-secretase 1 (BACE1), Abeta, epidermal growth factor receptor (EGFR), human epidermal growth factor receptor 2 (HER2), tau, apolipoprotein, apolipoprotein E (ApoE), apolipoprotein E4 (ApoE4), alpha-synuclein, CD20, huntingtin, prion protein (PrP), leucine rich repeat kinase 2 (LRRK2), parkin, presenilin 1, presenilin 2, β-glucocerebrosidase (GCase or GBA), progranulin (PGRN), Prosaposin (PSAP), ubiquitin protein ligase E3A (UBE3A), gamma secretase, death receptor 6 (DR6), amyloid precursor protein (APP), p75 neurotrophin receptor (p75NTR), caspase 6, sortilin (SORT), triggering receptor expressed on myeloid cells 2 (TREM2), CD33, sialic acid binding Ig-like lectin 3 (Siglec3), sialic acid binding Ig-like lectin 5 (Siglec5), sialic acid binding Ig-like lectin 7 (Siglec7), sialic acid binding Ig-like lectin 9 (Siglec9), sialic acid binding Ig-like lectin 11 (Siglec11), glycoprotein nonmetastatic melanoma protein B (GPNMB), Paired immunoglobin like type 2 receptor alpha (PILRA), Membrane Spanning 4-Domains A4A (MS4A4A), Membrane Spanning 4-Domains A 6A (MS4A6A), MSA4A4E, Transmembrane Protein 106B (TMEM106b), CR1, ABCA1, ABCA7, HLA-DR1, HLA-DR5, IL1RAP, TREML2, IL-34, SORL1, and ADAM1.

In some aspects, the CNS or brain antigen is on a cancer cell within the central nervous system. In some aspects, the CNS or brain antigen is a cell surface target on a hematological cancer cell selected from B7H3, BCMA, CD125, CD166, CD19, CD20, CD205, CD22, CD25, CD30, CD37, CD39, CD73, and CD79b. In some aspects, the CNS or brain antigen is a tumor cell target selected from siglec-3 or CD33, siglec-5, siglec-7, siglec-9, siglec 14, PILRA, IL18-BP, MerTK, ACKR1, ALK, AXL, CD25, CD44v6, CD46, CD56 (NCAM), CDH6 (cadherin 6), CEACAM 5 (CD66E), EGFR, EGFR viii, ETBR, FGFR (1-4), Folate Receptor alpha, GAL-3BP (galectin binding protein), GD2, GD3, GloboH (globohexasylceramide), gp100, gpNMB, HER2, HER3, HER4, IGFR1, KIT, LIV1A, LRRC15 (leucine rich repeat containing 15), MET, NaPi2B, PDL1, PMEL17, PRAME, PSMA, PTK7 (CCK4; colon carcinoma kinase), RON, ROR1, TF (tissue factor), and TROP2.

As provided herein, a fusion protein, antibody or antigen-binding fragment thereof, or multi-specific protein provided herein can comprise an antigen-binding domain that binds to a CNS antigen or a brain antigen. As provided herein, a multi-specific protein provided herein can comprise an antigen-binding domain that binds to a CNS antigen or a brain antigen. The antigen-binding domain that binds to a CNS antigen or a brain antigen can comprise a VH and a VL. Exemplary CNS antigen-binding VH and VL sequences are provided below. Additional VH and VL and antigen-binding domain sequences are found in US2017/0224702, US 2018/0002433, US 2021/0236634, and US 2021/0238265, each of which is herein incorporated by reference in its entirety.

TABLE 2
Exemplary CNS Antigens

| CNS | | |
|---|---|---|
| Antigen | VH | VL |
| BACE1 | EVQLVESGGGLVQPGGSLRL | DIQMTQSPSSLSASVGDRVTITCRASQD |
| | SCAASGFTFSGYAIHWVRQA | VSTAVAWYQQKPGKAPKLLIYSASFLY |
| | PGKGLEWVGWISPAGGSTD | SGVPSRFSGSGSGTDFTLTISSLQPEDFA |
| | YADSVKGRFTISADTSKNTA | TYYCQQSYTTPPTFGQGTKVEIKR (SEQ |
| | YLQMNSLRAEDTAVYYCAR | ID NO: 340) |
| | GPFSPWVMDYWGQGTLVTV | |
| | SS (SEQ ID NO: 339) | |
| BACE1 | EVQLVESGGGLVQPGGSLRL | DIQMTQSPSSLSASVGDRVTITCRASQS |
| | SCAASGFNFYYSSIHWVRQA | VSSAVAWYQQKPGKAPKLLIYSASSLY |
| | PGKGLEWVASISPYSGYTSY | SGVPSRFSGSGSGTDFTLTISSLQPEDFA |
| | ADSVKGRFTISADTSKNTAY | TYYCQQYSYSPFTFGQGTKVEIKR (SEQ |
| | LQMNSLRAEDTAVYYCARQ | ID NO: 342) |
| | PTHYYYYAKGYKAMDYWG | |
| | QGTLVTVSS (SEQ ID | |
| | NO: 341) | |
| Alpha- | EVQLLESGGGLVQPGGSLRL | EIVLTQSPGTLSLSPGERATLSCRASQSV |
| synuclein | SCAASGFTFSRFTMTWVRQA | SRSYLAWYQQKPGQAPRLLIYGASSRA |
| | PGKGLEWVSAISGSGGGTSY | TGIPDRFSGSGSGTDFTLTVSRLEPEDFA |
| | ADSVKGRLTVSRDNSKNTL | VYYCQQYGSSPWTFGQGTKVEIK (SEQ |
| | YLQMNSLRAEDTAVYYCAK | ID NO: 344) |
| | NWAPFDYWGQGTLVTVSS | |
| | (SEQ ID NO: 343) | |
| Alpha- | EVQLLESGGGLVQTGGSLRL | EIVLTQSPGTLSLSPGERATLSCRASQSV |
| synuclein | SCAASGFTFSSYAMTWVRQ | SSSYLAWYQQKPGQAPRLLIYGASSRA |
| | APGKGLEWVSAIRSNGDRTD | TGIPDRFSGSGSGTDFTLTISRLEPEDFA |
| | YADSVKGRFTISRDNSQNTL | VYYCQQYGSSPWTFGQGTKVEIK (SEQ |
| | YLQMNSLRAEDTAVYYCAK | ID NO: 346) |
| | NWAPFDSWGQGTLVTVSS | |
| | (SEQ ID NO: 345) | |
| Abeta | QVQLVQSGAEVKKPGSSVK | DIVMTQTPLSLSVTPGQPASISCKSSQSL |
| | VSCKASGYDFTRYYINWVR | LYSRGKTYLNWLLQKPGQSPQLLIYAV |
| | QAPGQGLEWMGWINPGSGN | SKLDSGVPDRFSGSGSGTDFTLKISRVE |
| | TKYNEKFKGRVTITADESTS | AEDVGVYYCVQGTHYPFTFGQGTKLEI |
| | TAYMELSSLRSEDTAVYYCA | K (SEQ ID NO: 348) |
| | REGITVYWGQGTTVTVSS | |
| | (SEQ ID NO: 347) | |
| Tau | EVQLVESGGGLVQPGGSLKL | SYELTQPPSVSVSPGQTARISCFGDTLPK |
| | SCAASGFNFNISAIHWVRQA | QYTYWYQQKPGQAPVLVIYKDTERPSG |
| | SGKGLEWVGRIRSKSHNYAT | IPERFSGSSSGTTVTLTISGVQAEDEADY |
| | LYAASLKGRFTLSRDDSRNT | YCLSADNSATWVFGGGTKVTVL (SEQ |
| | AYLQMSSLQTEDMAVYYCT | ID NO: 350) |
| | VLSANYDTFDYWGQGTLVT | |
| | VSS (SEQ ID NO: 349) | |

As provided herein, a fusion protein, antibody or antigen-binding fragment thereof, or multi-specific protein provided herein can be capable of crossing the BBB as a result of the fact that the anti-CD98 antigen-binding domain in the fusion protein, antibody or antigen-binding fragment thereof, or multi-specific protein is capable of crossing the BBB.

In some aspects, a fusion protein, antibody or antigen-binding fragment thereof, or multi-specific protein provided herein is internalized in blood-brain barrier epithelial cells greater than 10-fold as compared to internalization by an isotype control. The blood-brain barrier endothelial cells can be, e.g., HCMEC/D3 cells.

In some aspects, a fusion protein, antibody or antigen-binding fragment thereof, or multi-specific protein provided herein does not reduce cell-surface expression of CD98hc on HCMEC/D3 cells by more than 20% relative to cell-surface expression of CD98hc on HCMEC/D3 cells treated with an isotype control. Cell surface expression can be measured, e.g., using Western blot or FACS.

In some aspects, a fusion protein, antibody or antigen-binding fragment thereof, or multi-specific protein provided herein does not increase cell-surface expression of CD98hc on HCMEC/D3 cells by more than 50% relative to cell-surface expression of CD98hc on HCMEC/D3 cells treated with an isotype control. Cell surface expression can be measured, e.g., using Western blot or FACS.

In some aspects, a fusion protein, antibody or antigen-binding fragment thereof, or multi-specific protein provided herein does not reduce cell-surface expression of CD98hc on HCMEC/D3 cells by more than 20% relative to cell-surface expression of CD98hc on HCMEC/D3 cells treated with an isotype control and does not increase cell-surface expression of CD98hc on HCMEC/D3 cells by more than 50% relative to cell-surface expression of CD98hc on HCMEC/D3 cells treated with an isotype control. Cell surface expression can be measured, e.g., using Western blot or FACS.

In some aspects, a fusion protein, antibody or antigen-binding fragment thereof, or multi-specific protein provided herein accumulates at least 1.5-fold more than an isotype control in vessel-depleted mouse brain. In some aspects, a fusion protein, antibody or antigen-binding fragment thereof, or multi-specific protein provided herein accumulates at least 2-fold more than an isotype control in vessel-depleted mouse brain.

In some aspects, a fusion protein, antibody or antigen-binding fragment thereof, or multi-specific protein provided herein has at least a 5-fold increase in brain:serum concentration ratio over an isotype control 24 hours after administration to a mouse.

In some aspects, a fusion protein, antibody or antigen-binding fragment thereof, or multi-specific protein provided herein binds human CD98hc with an equilibrium dissociation constant (K_{D}) of about 3 nM to about 225 nM. In some aspects, a fusion protein, antibody or antigen-binding fragment thereof, or multi-specific protein provided herein binds cynomolgus monkey CD98hc with a K_{D} of about 3 nM to about 225 nM. In some aspects, a fusion protein, antibody or antigen-binding fragment thereof, or multi-specific protein provided herein binds human CD98hc with an equilibrium dissociation constant (K_{D}) of about 3 nM to about 225 nM and binds cynomolgus monkey CD98hc with a K_{D} of about 3 nM to about 225 nM.

### Polynucleotides and Methods of Making Anti-CD98hc Antigen-Binding Domains and Agents Comprising the Same

In some aspects, provided herein are polynucleotides comprising a nucleotide sequence encoding an antigen-binding domain that specifically bind to human CD98hc, fusion protein, antibody, antigen-binding fragment thereof, or multi-specific protein described herein or a domain thereof described herein, and vectors, e.g., vectors comprising such polynucleotides for recombinant expression in host cells (e.g., E. coli and mammalian cells).

In some aspects, a polynucleotide provided herein comprises a nucleic acid molecule encoding the heavy chain of an antigen-binding domain that specifically bind to human CD98hc provided herein. In some aspects, a polynucleotide provided herein comprises a nucleic acid molecule encoding the light chain of an antigen-binding domain that specifically bind to human CD98hc provided herein. In some aspects, a polynucleotide provided herein comprises a nucleic acid molecule encoding the heavy chain of an antigen-binding domain that specifically bind to human CD98hc provided herein and a nucleic acid molecule encoding the light chain of an antigen-binding domain that specifically bind to human CD98hc provided herein.

In some aspects, combinations or compositions of polynucleotides are provided herein. In some aspects, a combination or composition comprises a first polynucleotide, a second polynucleotide, and a third polynucleotide, wherein the first, second, and third polynucleotides encode a multi-specific protein provided herein, e.g., wherein the first polynucleotide encodes a first heavy chain, the second polynucleotide encodes a second heavy chain and an antigen-binding domain that specifically binds to human CD98hc provided herein, and the third polynucleotide encodes a light chain. In some aspects, the antigen-binding domains that bind to human CD98hc is an scFv. In some aspects, the first heavy chain comprises a knob mutation and the second heavy chain comprises a hole mutation. In some aspects, the first heavy chain comprises a hole mutation and the second heavy chain comprises a knob mutation.

In some aspects, a combination or composition comprises a first polynucleotide, a second polynucleotide, and a third polynucleotide, wherein the first, second, and third polynucleotides encode a multi-specific protein provided herein, wherein the first polynucleotide encodes a first heavy chain and a first antigen-binding domain that specifically binds to human CD98hc, the second polynucleotide encodes a second heavy chain and a second antigen-binding domain that specifically binds to human CD98, and the third polynucleotide encodes a light chain. In some aspects, the first and second antigen-binding domains that bind to human CD98hc comprise the same amino acid sequence. In some aspects, the first and second antigen-binding domains that bind to human CD98hc comprise different amino acid sequences. In some aspects, the first and/or second antigen-binding domains that bind to human CD98hc are scFvs. In some aspects, the first heavy chain comprises a knob mutation and the second heavy chain comprises a hole mutation. In some aspects, the first heavy chain comprises a hole mutation and the second heavy chain comprises a knob mutation.

In some aspects, a combination or composition comprises a first polynucleotide and a second polynucleotide, wherein the first and second polynucleotides encode a multi-specific protein provided herein, wherein the first polynucleotide encodes a heavy chain and an antigen-binding domain that bind to human CD98hc provided herein, and wherein the second polynucleotide encodes a light chain.

Also provided herein are polynucleotides comprising a nucleotide sequence encoding an antigen-binding domain that specifically bind to human CD98hc, fusion protein, antibody, antigen-binding fragment thereof, or multi-specific protein described herein, or a domain thereof described herein, that are optimized, e.g., by codon/RNA optimization, replacement with heterologous signal sequences, and/or elimination of mRNA instability elements. Methods to generate optimized nucleic acids for recombinant expression by introducing codon changes (e.g., a codon change that encodes the same amino acid due to the degeneracy of the genetic code) and/or eliminating inhibitory regions in the mRNA can be carried out by adapting the optimization methods described in, e.g., U.S. Pat. Nos. 5,965,726; 6, 174,666; 6,291,664; 6,414,132; and 6,794,498, accordingly.

A polynucleotide comprising a nucleotide sequence encoding an antigen-binding domain that specifically bind to human CD98hc, fusion protein, antibody, antigen-binding fragment thereof, or multi-specific protein described herein, or a domain thereof described herein, can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising, e.g., the sequence encoding the light chain and/or heavy chain of an antigen-binding domain, antibody, or antigen-binding fragment thereof. The amplified nucleic acids can be cloned into vectors for expression in host cells and for further cloning, for example, to generate an antigen-binding domain that specifically bind to human CD98hc, fusion protein, antibody, antigen-binding fragment thereof, or multi-specific protein described herein, or a domain thereof described herein.

Polynucleotides provided herein can be, e.g., in the form of RNA or in the form of DNA. DNA includes cDNA, genomic DNA, and synthetic DNA, and DNA can be double-stranded or single-stranded. If single stranded, DNA can be the coding strand or non-coding (anti-sense) strand. In some aspects, the polynucleotide is a cDNA or a DNA lacking one more endogenous introns. In some aspects, a polynucleotide is a non-naturally occurring polynucleotide. In some aspects, a polynucleotide is recombinantly produced. In some aspects, the polynucleotides are isolated. In some aspects, the polynucleotides are substantially pure.

In some embodiments, polynucleotides provided herein are in the form of RNA. In some embodiments, polynucleotides provided herein are in the form of RNA encoding a fusion protein provided herein. In some embodiments, a polynucleotide provided herein is a synthetic messenger RNA (mRNA). In some embodiments, the synthetic mRNA has at least one nucleoside modification. In some embodiments, the at least one nucleoside modification is selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some aspects, provided herein are polynucleotides encoding a fusion protein comprising an antigen-binding protein provided herein and a heterologous polypeptide. In some aspects, the heterologous polypeptide comprises an antigen binding domain that binds to beta-secretase 1 (BACE1), Abeta, epidermal growth factor receptor (EGFR), human epidermal growth factor receptor 2 (HER2), tau, apolipoprotein, apolipoprotein E (ApoE), apolipoprotein E4 (ApoE4), alpha-synuclein, CD20, huntingtin, prion protein (PrP), leucine rich repeat kinase 2 (LRRK2), parkin, presenilin 1, presenilin 2, β-glucocerebrosidase (GCase or GBA), progranulin (PGRN), Prosaposin (PSAP), gamma secretase, death receptor 6 (DR6), amyloid precursor protein (APP), p75 neurotrophin receptor (p75NTR), caspase 6, sortilin (SORT), triggering receptor expressed on myeloid cells 2 (TREM2), CD33 or sialic acid binding Ig-like lectin 3 (Siglec3), sialic acid binding Ig-like lectin 5 (Siglec5), sialic acid binding Ig-like lectin 7 (Siglec7), sialic acid binding Ig-like lectin 9 (Siglec9), glycoprotein nonmetastatic melanoma protein B (GPNMB), Paired immunoglobin like type 2 receptor alpha (PILRA), Membrane Spanning 4-Domains A4A (MS4A4A), Membrane Spanning 4-Domains A 6A (MS4A6A), or Transmembrane Protein 106B (TMEM106b), or a portion thereof. In some aspects, the heterologous polypeptide comprises an antigen binding domain that binds to ubiquitin protein ligase E3A (UBE3A). In some aspects, provided herein are polynucleotides encoding a fusion protein comprising an antigen-binding domain provided herein and a heterologous polypeptide. In some aspects, the heterologous polypeptide comprises the amino acid sequence of beta-secretase 1 (BACE1), Abeta, epidermal growth factor receptor (EGFR), human epidermal growth factor receptor 2 (HER2), tau, apolipoprotein, apolipoprotein E (ApoE), apolipoprotein E4 (ApoE4), alpha-synuclein, CD20, huntingtin, prion protein (PrP), leucine rich repeat kinase 2 (LRRK2), parkin, presenilin 1, presenilin 2, β-glucocerebrosidase (GBA), progranulin (PGRN), Prosaposin (PSAP), gamma secretase, death receptor 6 (DR6), amyloid precursor protein (APP), p75 neurotrophin receptor (p75NTR), caspase 6, sortilin (SORT), triggering receptor expressed on myeloid cells 2 (TREM2), CD33 or sialic acid binding Ig-like lectin 3 (Siglec3), sialic acid binding Ig-like lectin 5 (Siglec5), sialic acid binding Ig-like lectin 7 (Siglec7), sialic acid binding Ig-like lectin 9 (Siglec9), glycoprotein nonmetastatic melanoma protein B (GPNMB), Paired immunoglobin like type 2 receptor alpha (PILRA), Membrane Spanning 4-Domains A4A (MS4A4A), Membrane Spanning 4-Domains A 6A (MS4A6A), or Transmembrane Protein 106B (TMEM106b), or a portion thereof. In some aspects, the multispecific protein comprises an antigen binding domain that binds to ubiquitin protein ligase E3A (UBE3A). In some embodiments, the polynucleotide is mRNA (e.g., synthetic mRNA).

In some embodiments, disclosed herein are polynucleotides encoding a fusion protein disclosed herein comprising a heterologous polypeptide. In some aspects, the heterologous polypeptide is an ERT enzyme or an ERT enzyme variant, or a catalytically active fragment thereof. In some aspects, the heterologous polypeptide comprises β-glucocerebrosidase (GBA), progranulin (PGRN), Prosaposin (PSAP), or a catalytically active fragment thereof. In some aspects, the heterologous polypeptide in a fusion protein provided herein is a growth factor. In some aspects, the heterologous polypeptide in a fusion protein provided herein is a decoy receptor. In some aspects, the heterologous polypeptide in a fusion protein provided herein is progranulin (PGRN), prosaposin (PSAP), or survival motor neuron protein (SMN). In some aspects, the heterologous protein is an enzyme selected from α-L Iduronidase (IDUA), Iduronate-2-sulphatase (IDS), N-acetylgalactoslamine-6-sulphatase (GALNS), N-sulfoglucosamine sulfohydrolase (SGSH), N-acetylgalactosamine-4-sulphatase (arylsulphatase B; ARSB), acid sphingomyelinase (ASM), β-glucocerebrosidase (GCase or GBA), galactosylceramide beta-galactosidase, glucosylceramidase, beta-hexosaminidase A, beta-hexosaminidase B, arylsulphatase A, beta-galactosidase, acid ceramidase, alpha-glucosidase, lysosomal acid lipase, lysosomal protease, a synthetic enzyme replacement thereof, such as larodinase, idursulphase, elosulphase alpha or galsuphase, or a variant thereof, or a catalytically active fragment thereof. In some aspects, the heterologous protein is an enzyme selected from clusterin (APOJ), Reelin, ubiquitin protein ligase E3A (UBE3A), Tripeptidyl Peptidase 1 (CLN2/TPP1), glucosamine (N-acetyl)-6-sulfatase (GNS), heparan-alpha-glucosaminide N-acetyltransferase (HGSNAT), and N-acetyl-alpha-glucosaminidase (NAGLU), α-L Iduronidase (IDUA), Iduronate-2-sulphatase (IDS), N-acetylgalactoslamine-6-sulphatase (GALNS), N-sulfoglucosamine sulfohydrolase (SGSH), N-acetylgalactosamine-4-sulphatase (arylsulphatase B; ARSB), acid sphingomyelinase (ASM), β-glucocerebrosidase (GCase or GBA), galactosylceramide beta-galactosidase, glucosylceramidase, beta-hexosaminidase A, beta-hexosaminidase B, arylsulphatase A, beta-galactosidase, acid ceramidase, alpha-glucosidase, lysosomal acid lipase, lysosomal protease, a synthetic enzyme replacement thereof, such as larodinase, idursulphase, elosulphase alpha or galsuphase, or a variant thereof, or a catalytically active fragment thereof α-L Iduronidase (IDUA), Iduronate-2-sulphatase (IDS), N-acetylgalactoslamine-6-sulphatase (GALNS), N-sulfoglucosamine sulfohydrolase (SGSH), N-acetylgalactosamine-4-sulphatase (arylsulphatase B; ARSB), acid sphingomyelinase (ASM), β-glucocerebrosidase (GCase or GBA), galactosylceramide beta-galactosidase, glucosylceramidase, beta-hexosaminidase A, beta-hexosaminidase B, arylsulphatase A, beta-galactosidase, acid ceramidase, alpha-glucosidase, lysosomal acid lipase, lysosomal protease, a synthetic enzyme replacement thereof, such as larodinase, idursulphase, elosulphase alpha or galsuphase, or a variant thereof, or a catalytically active fragment thereof. In some embodiments, the polynucleotide is mRNA (e.g., synthetic mRNA).

In certain aspects, provided herein are vectors (e.g., expression vectors) comprising polynucleotides comprising nucleotide sequences encoding an antigen-binding domain that specifically bind to human CD98hc, fusion protein, antibody, antigen-binding fragment thereof, or multi-specific protein described herein, or a domain thereof described herein, for recombinant expression in a host cell, e.g., in a mammalian host cell. A vector for the production of the antigen-binding domain that specifically bind to human CD98hc, fusion protein, antibody, antigen-binding fragment thereof, or multi-specific protein described herein, or a domain thereof described herein, can be produced, e.g., by recombinant DNA technology using techniques well known in the art. These methods include, for example, in vitro recombinant DNA techniques, synthetic techniques, and in vivo genetic recombination. Also provided are replicable vectors comprising a nucleotide sequence encoding an antigen-binding domain that specifically bind to human CD98hc, fusion protein, antibody, antigen-binding fragment thereof, or multi-specific protein described herein, or a domain thereof described herein, operably linked to a promoter. Such vectors can, for example, include the nucleotide sequence encoding the constant region of an antigen-binding domain, antibody or antigen-binding fragment thereof (see, e.g., International Publication Nos. WO 86/05807 and WO 89/01036; and U.S. Pat. No. 5,122,464), and variable domains of the antigen-binding domain, antibody or antigen-binding fragment thereof can be cloned into such a vector for expression of the entire heavy, the entire light chain, or both the entire heavy and light chains. In some embodiments, the vector is gene therapy vector (e.g., an AAV or lentiviral vector).

In certain aspects, provided herein are expression systems comprising polynucleotides comprising nucleotide sequences encoding an antigen-binding domain that specifically bind to human CD98hc, fusion protein, antibody, antigen-binding fragment thereof, or multi-specific protein described herein, or a domain thereof described herein. An expression system can be included on a vector. An expression system can also be integrated into a host cell chromosome. In some aspects, an expression system is a cell free expression system. In some aspects, an expressions system comprises a host cell comprising a polynucleotide and/or vector provided herein.

Accordingly, also provided herein are cells, e.g., host cells, comprising polynucleotides and/or vectors for recombinantly expressing an antigen-binding domain that specifically bind to human CD98hc, fusion protein, antibody, antigen-binding fragment thereof, or multi-specific protein described herein, or a domain thereof described herein. In some aspects, for the expression of double-chained antigen-binding proteins, vectors encoding both the heavy and light chains, individually, can be co-expressed in the host cell for expression of the entire immunoglobulin. In some aspects, a host cell contains two different vectors, a first vector comprising a polynucleotide encoding a heavy chain of an antigen-binding protein described herein, and a second vector comprising a polynucleotide encoding a light chain of an antigen-binding protein. In some aspects, a first host cell comprises a first vector comprising a polynucleotide encoding a heavy chain, and a second host cell comprises a second vector comprising a polynucleotide encoding a light chain. In some aspects, provided herein is a population of host cells comprising such first host cell and such second host cell.

In some aspects, provided herein are methods for producing an antigen-binding domain that specifically binds to human CD98hc, fusion protein, antibody, antigen-binding fragment thereof, or multi-specific protein described herein, or a domain thereof described herein in a host cell. In some aspects, provided herein are methods for producing a single chain antigen-binding domain that specifically binds to human CD98hc, an Fc domain, and a heterologous protein or polypeptide, as described herein in a host cell. In some aspects, provided herein are methods for producing a single chain antigen-binding domain that specifically binds to human CD98hc, an Fc domain, and a second antigen-binding domain, as described herein in a host cell. An expression vector can be transferred to a cell (e.g., host cell) by conventional techniques, and the resulting cells can then be cultured by conventional techniques to produce an antigen-binding domain that specifically binds to human CD98hc, fusion protein, antibody, antigen-binding fragment thereof, or multi-specific protein described herein, or a domain thereof described herein.

A variety of host-expression vector systems can be utilized to express an antigen-binding domain that specifically bind to human CD98hc, fusion protein, antibody, antigen-binding fragment thereof, or multi-specific protein described herein, or a domain thereof described herein (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antigen-binding domain that specifically bind to human CD98hc, fusion protein, antibody, antigen-binding fragment thereof, or multi-specific protein described herein, or a domain thereof described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing coding sequences; or mammalian cell systems (e.g., COS (e.g., COS1 or COS), CHO, BHK, MDCK, HEK 293, NSO, PER.C6, VERO, CRL7030, HsS78Bst, HeLa, and NIH3T3, HEK-293T, HepG2, SP210, R1.1, B-W, L-M, BSC1, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In some aspects, cells for expressing an antigen-binding domain that specifically bind to human CD98hc, fusion protein, antibody, antigen-binding fragment thereof, or multi-specific protein described herein, or a domain thereof described herein are CHO cells, for example CHO cells from the CHO GS System™ (Lonza). In some aspects, cells for expressing an antigen-binding domain that specifically bind to human CD98hc, fusion protein, antibody, antigen-binding fragment thereof, or multi-specific protein described herein, or a domain thereof described herein as described herein are human cells, e.g., human cell lines. In some aspects, a mammalian expression vector is pOptiVEC™ or pcDNA3.3. In some aspects, bacterial cells, such as Escherichia coli, or eukaryotic cells (e.g., mammalian cells) are used for the expression of an antigen-binding domain that specifically bind to human CD98hc, fusion protein, antibody, antigen-binding fragment thereof, or multi-specific protein described herein, or a domain thereof described herein. For example, mammalian cells such as Chinese hamster ovary (CHO) cells in conjunction with a vector such as the major intermediate early gene promoter element from human cytomegalovirus is an effective expression system for antibodies (Foecking M K & Hofstetter H (1986) Gene 45:101-105; and Cockett M I et al., (1990) Biotechnology 8:662-667). In some aspects, an antigen-binding domain that specifically bind to human CD98hc, fusion protein, antibody, antigen-binding fragment thereof, or multi-specific protein described herein, or a domain thereof described herein is produced by CHO cells or NSO cells.

In addition, a host cell strain can be chosen which modulates the expression of the inserted sequences, or modifies and processes the gene product in the specific fashion desired. Such modifications (e.g., glycosylation) and processing (e.g., cleavage) of protein products can contribute to the function of the protein. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NSO (a murine myeloma cell line that does not endogenously produce any immunoglobulin chains), CRL7030, COS (e.g., COS1 or COS), PER.C6, VERO, HsS78Bst, HEK-293T, HepG2, SP210, R1.1, B-W, L-M, BSC1, BSC40, YB/20, BMT10 and HsS78Bst cells.

Once an antigen-binding domain that specifically bind to human CD98hc, fusion protein, antibody, antigen-binding fragment thereof, or multi-specific protein described herein, or a domain thereof described herein has been produced by recombinant expression, it can be purified by any method known in the art for purification, for example, by chromatography (e.g., ion exchange, affinity, particularly by affinity for the specific antigen after Protein A, and sizing column chromatography), centrifugation, differential solubility, or by any other standard technique for the purification of proteins. Further, the antigen-binding domain that specifically bind to human CD98hc, fusion protein, antibody, antigen-binding fragment thereof, or multi-specific protein described herein, or a domain thereof described herein can be fused to heterologous polypeptide sequences to facilitate purification.

In some aspects, once an antigen-binding domain that specifically binds to human CD98hc, fusion protein, antibody, antigen-binding fragment thereof, or multi-specific protein described herein is isolated or purified. Generally, an isolated Once an antigen-binding domain that specifically bind to human CD98hc, fusion protein, antibody, antigen-binding fragment thereof, or multi-specific protein described herein is one that is substantially free of other proteins. For example, in some aspects, a preparation of Once an antigen-binding domain that specifically bind to human CD98hc, fusion protein, antibody, antigen-binding fragment thereof, or multi-specific protein described herein is substantially free of cellular material and/or chemical precursors.

### Pharmaceutical Compositions

Provided herein are compositions comprising an antigen-binding domain that specifically binds to human CD98hc, fusion protein, antibody, antigen-binding fragment thereof, or multi-specific protein described herein. In some aspects, the antigen-binding domain that specifically bind to human CD98hc, fusion protein, antibody, antigen-binding fragment thereof, or multi-specific protein having the desired degree of purity is present in a formulation comprising, e.g., a physiologically acceptable carrier, excipient or stabilizer (Remington's Pharmaceutical Sciences (1990) Mack Publishing Co., Easton, PA). Acceptable carriers, excipients, or stabilizers are nontoxic to recipients at the dosages and concentrations employed. Formulations suitable for parenteral administration include aqueous and non-aqueous, isotonic sterile injection solutions, which can comprise antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

In some aspects, a pharmaceutical composition comprises an antigen-binding domain that specifically bind to human CD98hc, fusion protein, antibody, antigen-binding fragment thereof, or multi-specific protein described herein, and a pharmaceutically acceptable carrier (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Pharmaceutical compositions described herein are, in some aspects, for use as a medicament. The compositions to be used for in vivo administration can be sterile. This is readily accomplished by filtration through, e.g., sterile filtration membranes.

Also provided herein are pharmaceutical compositions comprising a polynucleotide encoding an antigen-binding domain that specifically binds to human CD98hc, a fusion protein, an antibody or antigen-binding fragment thereof, or multi-specific protein described herein. In some embodiments, the polynucleotide is RNA. In some embodiments, the polynucleotide is synthetic mRNA. In some embodiments, the pharmaceutical composition comprising a polynucleotide further comprises a lipid-based transfection reagent.

A pharmaceutical composition described herein can be used to exert a biological effect(s) in vivo or in vitro. For example, a pharmaceutical composition described herein can be used to cross a blood brain barrier, e.g., in a subject.

In some aspects, a pharmaceutical composition provided herein is used to treat diseases or conditions such as a neuropathy disorder, a neurodegenerative disease, cancer, an ocular disease disorder, a seizure disorder, a lysosomal storage disease, amyloidosis, a viral or microbial disease, ischemia, a behavioral disorder, and CNS inflammation. In some aspects, a pharmaceutical composition provided herein is used to treat diseases or conditions such as Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, and traumatic brain injury. In some aspects, a pharmaceutical composition provided herein is used to treat frontotemporal dementia.

In some aspects, a pharmaceutical composition provided herein is formulated for intravenous administration. In some aspects, a pharmaceutical composition provided herein is formulated for subcutaneous administration.

### Methods of Using Anti-CD98hc Antigen-Binding Domains and Agents Comprising the Same

Antigen-binding domains that specifically bind to human CD98hc, fusion proteins, antibodies, antigen-binding fragments thereof, and multi-specific proteins comprising such antigen-binding domains as provided herein can advantageously be transported across a blood brain barrier. Accordingly, provided herein are methods of administering or transporting an antigen-binding protein that specifically binds to human CD98hc or a fusion protein, antibody, antigen-binding fragment thereof, or multi-specific protein comprising an antigen-binding protein that specifically binds to human CD98hc across the blood brain barrier of a subject comprising administering to the subject an antigen-binding protein that specifically binds to human CD98hc or a fusion protein, antibody, antigen-binding fragment thereof, or multi-specific protein comprising an antigen-binding protein that specifically binds to human CD98hc.

In view of the ability of antigen-binding domains that specifically bind to human CD98hc, fusion proteins, antibodies, antigen-binding fragments thereof, and multi-specific proteins comprising such antigen-binding domains as provided herein to be transported across a blood brain barrier, they can be used to treat a neurological disease or disorder. In some aspects, a method of treating a neurological disease or disorder in a subject comprises administering to the subject an antigen-binding protein that specifically binds to human CD98hc or a fusion protein, antibody, antigen-binding fragment thereof, or multi-specific protein comprising an antigen-binding protein that specifically binds to human CD98hc. The neurological disease or disorder can be, for example, a neuropathy disorder, a neurodegenerative disease, cancer, an ocular disease disorder, a seizure disorder, a lysosomal storage disease, amyloidosis, a viral or microbial disease, ischemia, a behavioral disorder, or CNS inflammation. The neurological disease or disorder can be, for example, a neurodegenerative disease (such as Lewy body disease, postpoliomyelitis syndrome, Shy-Draeger syndrome, olivopontocerebellar atrophy, Parkinson's disease, Gaucher disease, multiple system atrophy, striatonigral degeneration, spinocerebellar ataxia, spinal muscular atrophy), a tauopathy (such as Alzheimer disease and supranuclear palsy), a prion disease (such as bovine spongiform encephalopathy, scrapie, Creutz-feldt-Jakob syndrome, kuru, Gerstmann-Straussler-Scheinker disease, chronic wasting disease, and fatal familial insomnia), bulbar palsy, motor neuron disease, a nervous system heterodegenerative disorders (such as Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (such as Pick's disease, and spinocerebellar ataxia), cancer of the CNS and/or brain (such as glioblastoma or brain metastases resulting from cancer elsewhere in the body), Alzheimer's disease (AD), stroke, dementia, muscular dystrophy (MD), multiple sclerosis (MS), amyotrophic lateral sclerosis (ALS), limbic-predominant age-related TDP-43 encephalopathy (LATE), cystic fibrosis, Angelman's syndrome, Liddle syndrome, Parkinson's disease, Pick's disease, Paget's disease, cancer, or traumatic brain injury. In some aspects, the neurological disease or disorder is dementia. In some aspects, the neurological disease or disorder is frontotemporal dementia. In some aspects, the neurological disease or disorder is Alzheimer's disease. In some aspects, the neurological disease or disorder is Parkinson's disease. In some aspects, the neurological disease or disorder is frontal temporal epilepsy. In some embodiments, the neurological disease or disorder is autism. In some aspects, the neurological disease or disorder is lissencephaly.

In some aspects, provided herein is a method of treating a lysosomal storage disease with a fusion protein disclosed herein. In some aspects, the lysosomal storage disease is selected from Gaucher disease, Ceroid lipofuscinosis (Batten disease), Mucopolysaccharidosis (MPS) Type I, MPS Type II and MPS Type III.

Antigen-binding domains that specifically bind to human CD98hc, fusion proteins, antibodies, antigen-binding fragments thereof, and multi-specific proteins comprising such antigen-binding domains as provided herein can be used to detect an antigen (e.g., a CNS antigen or a brain antigen). Antigen-binding domains that specifically bind to human CD98hc and fusion proteins, antibodies, antigen-binding fragments thereof, and multi-specific proteins comprising such antigen-binding domains for such purposes can be labeled. Exemplary labels include, for example, radioisotopes (e.g., 6+CU) and fluorescent labels. Accordingly, methods of detecting an antigen using an antigen-binding protein that specifically binds to human CD98hc or a fusion protein, antibody, antigen-binding fragment thereof, or multi-specific protein comprising an antigen-binding protein that specifically binds to human CD98hc described herein are provided. In some aspects, a method of detecting an antigen in the CNS (e.g., brain) of a subject comprises administering an antigen-binding protein that specifically binds to human CD98hc or a fusion protein, antibody, antigen-binding fragment thereof, or multi-specific protein comprising an antigen-binding protein that specifically binds to human CD98hc to the antigen in the CNS (e.g., brain). Such methods can further comprise, e.g., performing Positron emission tomography (PET) imaging on the subject. In some aspects, disclosed herein is a method of detecting a CNS antigen in vitro, comprising contacting an in vitro sample with a fusion protein, antibody or antigen-binding fragment thereof, or multi-specific protein disclosed herein and locating the imaging agent within the sample.

Antigen-binding domains that specifically bind to human CD98hc and fusion proteins, antibodies, antigen-binding fragments thereof, and multi-specific proteins comprising such antigen-binding domains as provided herein can be used for prognostic, diagnostic, monitoring, and/or screening applications, including in vivo applications well known and standard to the skilled artisan and based on the present description. In some aspects, provided herein is an antigen-binding protein that specifically binds to human CD98hc or a fusion protein, antibody, antigen-binding fragment thereof, or multi-specific protein comprising an antigen-binding protein that specifically binds to human CD98hc for use as a diagnostic. In some aspects, the antigen-binding protein that specifically binds to human CD98hc or a fusion protein, antibody, antigen-binding fragment thereof, or multi-specific protein comprising an antigen-binding protein that specifically binds to human CD98hc comprises a detectable label.

All references cited herein, including patent applications and publications, are hereby incorporated by reference in their entirety.

The present disclosure will be more fully understood by reference to the following Examples. They should not, however, be construed as limiting the scope of the present disclosure. All citations throughout the disclosure are hereby expressly incorporated by reference.

### EXAMPLES

### Example 1: Production of Avi-His Tagged Variants of CD98 Heavy Chain (CD98hc)

To generate Avi-His tagged variants of the extra-cellular domain (ECD) of human, cynomolgus macaque (cyno), and mouse CD98 heavy chain (CD98hc), the following methods were used. Mammalian expression of human, cyno and mouse variants of BBB receptor antigens (SEQ ID NO:1-3) was performed by cloning synthetic genes based on cDNA into mammalian expression vectors, followed by transient transfection and expression in Expi293 cells. Constructs included a heterologous signal peptide and C-terminal Avi-His tag to allow for purification and biotinylation. Briefly, plasmids encoding the antigens were transfected using the Expifectamine 293 Transfection kit (ThermoFisher A14524) according to the manufacturer's specifications. Five days after transfection, the culture supernatants were harvested, clarified by centrifugation, and purified using HisPur Ni-NTA resin (Thermo Scientific 88223) in a drip-column format. 200 mL of culture supernatant was filtered using 0.2 μm filtration unit and 3 mL of resin slurry in PBS was added to the filtered supernatant. The sample was incubated overnight with shaking at 4° C., and on the following day, the beads were loaded onto 20 mL drip columns, washed with 10 mL of His-Select wash buffer (Millipore Sigma H5288) and eluted with 5 mL of His-Pur elution buffer (Millipore Sigma H5413). Eluate was buffer exchanged into PBS using Amicon Ultra-15 centrifugal filter units (Millipore UFC9010). Quantification of the antibody concentration was determined by measuring the absorbance at 280 nm using the Nanodrop 8000 (ThermoFisher). Purity of the antigens was determined by SDS-PAGE. The antigens were analyzed with size exclusion chromatography (SEC) for aggregation. Some antigens were biotinylated using a BirA biotin-protein ligase kit (AVIDITY), according to the manufacturer's instructions.

TABLE 3
Avi-His tagged variants of CD98hc ECD

| SEQ | | |
|---|---|---|
| ID | | |
| NO | Description | Sequence |
| 1 | huCD98hc | RAPRCRELPAQKWWHTGALYRIGDLQAFQGHGAGNLAGLKGRLDYLSSLKVKGLVLG |
| | ECD Avi- | PIHKNQKDDVAQTDLLQIDPNFGSKEDFDSLLQSAKKKSIRVILDLTPNYRGENSWF |
| | His | STQVDTVATKVKDALEFWLQAGVDGFQVRDIENLKDASSFLAEWQNITKGFSEDRLL |
| | | IAGTNSSDLQQILSLLESNKDLLLTSSYLSDSGSTGEHTKSLVTQYLNATGNRWCSW |
| | | SLSQARLLTSFLPAQLLRLYQLMLFTLPGTPVFSYGDEIGLDAAALPGQPMEAPVML |
| | | WDESSFPDIPGAVSANMTVKGQSEDPGSLLSLFRRLSDORSKERSLLHGDFHAFSAG |
| | | PGLFSYIRHWDQNERFLVVLNFGDVGLSAGLQASDLPASASLPAKADLLLSTQPGRE |
| | | EGSPLELERLKLEPHEGLLLRFPYAAGGGLNDIFEAQKIEWHEHHHHHHHHH |
| 2 | Cyno | RAPRCRELPAQKWWHTGALYRIGDLQAFQGHGSGNLAGLKGRLDYLSSLKVKGLVLG |
| | CD98hc | PLHKNQKDDVAQTDLLQIDPNFGSKEDFDNLLQSAKKKSIRVILDLTPNYRGENLWF |
| | ECD Avi- | STQVDSVATKVKDALEFWLQAGVDGFQVRDIENLKDASSFLAEWENITKGFSEDRLL |
| | His | IAGTNSSDLQQIVSLLESNKDLLLTSSYLSDSSFTGEHTKSLVTQYLNATGNRWCSW |
| | | SLSQAGLLTSFLPAQLLRLYQLMLFTLPGTPVFSYGDEIGLKAAALPGQPVEAPVML |
| | | WDESSFPDIPGAVSANMTVKGQSEDPGSLLSLFRQLSDQRSKERSLLHGDFHTFSSG |
| | | PGLFSYIRHWDQNERFLVVLNFGDVGLSAGLQASDLPASASLPTKADLVLSTQPGRE |
| | | EGSPLELERLKLEPHEGLLLRFPYVAGGGLNDIFEAQKIEWHEHHHHHHHHH |
| 3 | muCD98hc | APRCRELPVQRWWHKGALYRIGDLQAFVGRDAGGIAGLKSHLEYLSTLKVKGLVLGP |
| | ECD Avi- | IHKNQKDEINETDLKQINPTLGSQEDFKDLLQSAKKKSIHIILDLTPNYQGQNAWFL |
| | His | PAQADIVATKMKEALSSWLQDGVDGFQFRDVGKLMNAPLYLAEWQNITKNLSEDRLL |
| | | IAGTESSDLQQIVNILESTSDLLLTSSYLSNSTFTGERTESLVTRFLNATGSQWCSW |
| | | SVSQAGLLADFIPDHLLRLYQLLLFTLPGTPVFSYGDELGLQGALPGQPAKAPLMPW |
| | | NESSIFHIPRPVSLNMTVKGQNEDPGSLLTQFRRLSDLRGKERSLLHGDFHALSSSP |
| | | DLFSYIRHWDQNERYLVVLNFRDSGRSARLGASNLPAGISLPASAKLLLSTDSARQS |
| | | REEDTSLKLENLSLNPYEGLLLQFPFVAGGGLNDIFEAQKIEWHEHHHHHHHHH |

### Example 2: Production of CHO Cell Lines Over-Expressing CD98hc

Several cell lines were generated for screening the binding of anti-CD98hc antibodies, as described herein. Briefly, pLenti-EF1a constructs each expressing full length human CD98hc and mouse CD98hc (SEQ ID NOs: 4-5, respectively) were used to generate CHO cells stably expressing human CD98hc and mouse CD98hc. Lentiviral constructs (Genecopoeia) were used to express human CD98hc with puromycin selection and mouse CD98hc with GFP as a reporter. Lentivirus was generated from transfection of 293T cells using the ViraSafe™ Lentiviral Packaging System (CellBiolabs VPK-206). Subsequently, the supernatant containing lentivirus was used to transduce Chinese Hamster Ovary (CHO) cells. At 2 days post transduction, puromycin was added to the medium as a selection pressure for the human CD98hc. The mouse CD98hc was FACS sorted for GFP positive cells on the FACS Aria system (BD Biosciences). The resulting CHO cells stably expressing human CD98hc and mouse CD98hc were analyzed for cell surface expression by flow cytometry.

TABLE 4
Full Length CD98hc Sequences

| SEQ | | |
|---|---|---|
| ID | | |
| NO | Description | Sequence |
| 4 | Full length | MELQPPEASIAVVSIPRQLPGSHSEAGVQGLSAGDDSELGSHCVAQTGLELLASGDP |
| | human | LPSASQNAEMIETGSDCVTQAGLQLLASSDPPALASKNAEVTGTMSQDTEVDMKEVE |
| | CD98hc | LNELEPEKQPMNAASGAAMSLAGAEKNGLVKIKVAEDEAEAAAAAKFTGLSKEELLK |
| | | VAGSPGWVRTRWALLLLFWLGWLGMLAGAVVIIVRAPRCRELPAQKWWHTGALYRIG |
| | | DLQAFQGHGAGNLAGLKGRLDYLSSLKVKGLVLGPIHKNQKDDVAQTDLLQIDPNFG |
| | | SKEDFDSLLQSAKKKSIRVILDLTPNYRGENSWFSTQVDTVATKVKDALEFWLQAGV |
| | | DGFQVRDIENLKDASSFLAEWQNITKGFSEDRLLIAGTNSSDLQQILSLLESNKDLL |
| | | LTSSYLSDSGSTGEHTKSLVTQYLNATGNRWCSWSLSQARLLTSFLPAQLLRLYQLM |
| | | LFTLPGTPVFSYGDEIGLDAAALPGQPMEAPVMLWDESSFPDIPGAVSANMTVKGQS |
| | | EDPGSLLSLFRRLSDQRSKERSLLHGDFHAFSAGPGLFSYIRHWDQNERFLVVLNFG |
| | | DVGLSAGLQASDLPASASLPAKADLLLSTQPGREEGSPLELERLKLEPHEGLLLRFP |
| | | YAA |
| 5 | Full length | MSQDTEVDMKDVELNELEPEKQPMNAADGAAAGEKNGLVKIKVAEDETEAGVKFTGL |
| | mouse | SKEELLKVAGSPGWVRTRWALLLLFWLGWLGMLAGAVVIIVRAPRCRELPVQRWWHK |
| | CD98hc | GALYRIGDLQAFVGRDAGGIAGLKSHLEYLSTLKVKGLVLGPIHKNQKDEINETDLK |
| | | QINPTLGSQEDFKDLLQSAKKKSIHIILDLTPNYQGQNAWFLPAQADIVATKMKEAL |
| | | SSWLQDGVDGFQFRDVGKLMNAPLYLAEWQNITKNLSEDRLLIAGTESSDLQQIVNI |
| | | LESTSDLLLTSSYLSNSTFTGERTESLVTRFLNATGSQWCSWSVSQAGLLADFIPDH |
| | | LLRLYQLLLFTLPGTPVFSYGDELGLQGALPGQPAKAPLMPWNESSIFHIPRPVSLN |
| | | MTVKGQNEDPGSLLTQFRRLSDLRGKERSLLHGDFHALSSSPDLFSYIRHWDQNERY |
| | | LVVLNFRDSGRSARLGASNLPAGISLPASAKLLLSTDSARQSREEDTSLKLENLSLN |
| | | PYEGLLLQFPFVA |

### Example 3: Generation of CD98hc Humanized Mice

Humanized mouse lines expressing human extracellular domains of CD98hc were generated (Taconic Biosciences GmbH (Germany)). CRISPR was used to replace the mouse ECD with the human version, while retaining the mouse intracellular and transmembrane portions under the control of the mouse promotor. Brain slices from these mice were evaluated by IHC and/or Western Blot to confirm expression and localization of the human ECD in vivo (data not shown).

### Example 4: Generation of Anti-CD98hc Hybridoma Antibodies

In order to obtain antibodies against CD98hc, the following procedures were used to generate hybridomas. BALB/c mice or Sprague Dawley rats (Charles River Laboratories, Wilmington, MA) were immunized twice a week by subcutaneous or intraperitoneal injections of purified extracellular domain polypeptides of human, cyno, and/or mouse CD98hc (obtained as described above in Example 1) with or without adjuvant. After a total of 6-8 injections and three days following the final boost, the lymph nodes were harvested from the mice for hybridoma cell line generation.

Sera from the animals were analyzed for reactivity to CD98hc by FACS on CHO cells overexpressing human or mouse CD98hc and by ELISA against human, cyno and mouse CD98hc Avi-His polypeptides (as described below). Lymphocytes from animals whose sera demonstrated strong binding to CHO cells overexpressing human or mouse CD98hc were isolated and fused with myeloma fusion partners to produce mouse myeloma cells via electrofusion (Hybrimune, BTX, Holliston, MA) and incubated at 37° C., 5% CO2, overnight in Clonacell-HY Medium C (Stemcell Technologies, Vancouver, BC, Canada, Cat #03803).

The following day, the fused cells were centrifuged and resuspended in Clonacell-HY Medium E (Stemcell Technologies, Cat #03805) with HAT (Sigma Aldrich, Cat #H0262). The cells were seeded into T225 flasks and grown for 6 days at 37° C., 5% CO2, and then the hybridoma libraries were cryopreserved. After thawing the mouse hybridoma libraries, the cells were resuspended into 10 ml of ClonaCell-HY Medium C with anti-mouse IgG Fc-FITC (Jackson ImmunoResearch, West Grove, PA) and then gently mixed with 90 ml of methylcellulose-based ClonaCell-HY Medium D (Stemcell Technologies, Cat #03804) containing HAT components. The cells were plated into Nunc OmniTrays (Thermo Fisher Scientific, Rochester, NY) and allowed to grow at 37° C., 5% CO2 for 11 days. The Clonepix 2 (Molecular Devices, Sunnyvale, CA) system was used to select and transfer IgG positive colonies into 96-well plates with high glucose DMEM culture media containing 10% Fetal Cone II serum (Hyclone SH30066.03, Cytiva, Malborough, MA), 1× GlutaMAX (Gibco 35050061, Thermo Fisher Scientific, Waltham, MA) and 20% Clonacell-HY Medium E (Stemcell Technologies, Cat #03805). This method was performed for two hybridoma campaigns (Nos. 3 and 4).

Alternatively, the fused hybridomas were recovered overnight, resuspended in ClonaCell-HY Medium C with anti-mouse IgG Fc-FITC and mixed into Clonacell-HY Medium D. The cells were plated, grown, and picked using the Clonepix 2 system as described above. This method was performed for two additional hybridoma campaigns (Nos. 1 and 2).

In total, four hybridoma campaigns were conducted, and a total of 898 IgG positive hybridoma clones were isolated. After six days of culture, tissue culture supernatants from the hybridomas were screened by FACS on CHO cells overexpressing human or mouse CD98hc (as described below).

### Example 5: Screening of Anti-CD98hc Antibody Hybridoma Supernatants by FACS

The IgG positive hybridoma supernatants (898 in total) were initially screened for their ability to differentially bind CHO cells overexpressing human or mouse CD98hc cells compared to CHO parental cells by FACS. Overexpressing cells were harvested, washed, and labeled with various concentrations and combinations of CellTrace cell proliferation dyes CFSE and Violet (ThermoFisher, Cat #C34554 and Cat #34557, respectively) to create uniquely barcoded cell populations. Barcoded cells (5×10⁴ of each cell population) were aliquoted into 96-well U-bottom plates and incubated with 50 μl of hybridoma cell culture supernatant or 1 μg/ml of commercially available purified mouse anti-human CD98hc monoclonal antibody (Sigma Aldrich, Cat #SAB 4700503) and rat anti-mouse CD98hc antibody (Bio-Rad Laboratories, Hercules, CA, Cat #MCA2684) on ice for 30 minutes. After this primary incubation, the supernatants were removed via centrifugation, the cells were washed twice with 175 μl of ice-cold FACS buffer (PBS+1% FBS+2 mM EDTA), and the cells were then further incubated on ice for 20 minutes with anti-mouse IgG Fc-allophycocyanin (APC) or anti-rat IgG Fc-APC (Jackson Labs, Cat #115-136-071 and Cat #112-136-071, respectively), diluted 1:1000 in FACS buffer. Following this secondary antibody incubation, the cells were again washed twice with ice-cold FACS buffer and resuspended in a final volume of 50 μl of FACS buffer containing 0.25 μl/well propidium iodide (BD Biosciences, Cat #556463). Binding intensity on cells was analyzed using the FACS Canto system (BD Biosciences), with sorting gates drawn to exclude dead (i.e., propidium iodide-positive) cells. For each barcoded cell population, the ratio of APC Mean Fluorescence Intensity (MFI) divided by secondary antibody MFI was calculated for each anti-CD98hc hybridoma supernatant tested and reported in Table 5.

From the four hybridoma campaigns, a total of 121 hybridoma clones displayed an MFI ratio greater than 2-fold for binding to CHO cells stably overexpressing human CD98hc. 103 of these clones also bound with an MFI ratio greater than 5-fold for binding to HCMEC/D3 endothelial cells endogenously expressing human CD98hc. None of the hybridoma clones showed binding to CHO cells stably overexpressing mouse CD98hc. Accordingly, 121 of the 898 clones were determined to be “FACS positive,” and 104 of these FACS positive clones had unique CD98hc sequences. Data are shown in Table 3 for a subset of the FACS positive clones.

TABLE 5
FACS MFI values of unique anti-CD98hc antibodies
identified from hybridoma campaigns

| | MFI ratio over secondary antibody | | | |
|---|---|---|---|---|
| | | CHO- | CHO- | |
| | HCMEC/ | human | mouse | CHO |
| Antibody | D3 | CD98hc | CD98hc | parental |
| CD98HC.03.002 | 232.8 | 28.5 | 1.0 | 1.1 |
| CD98HC.03.004 | 189.4 | 34.4 | 1.0 | 1.0 |
| CD98HC.03.010 | 190.4 | 25.8 | 1.0 | 1.0 |
| CD98HC.03.031 | 198.3 | 38.9 | 1.2 | 1.2 |
| CD98HC.04.003 | 13.8 | 3.2 | 1.1 | 1.3 |
| CD98HC.04.004 | 157.4 | 31.0 | 1.1 | 1.3 |
| CD98HC.04.005 | 46.1 | 8.5 | 1.2 | 1.4 |
| CD98HC.04.006 | 197.1 | 39.0 | 1.1 | 1.3 |
| CD98HC.04.009 | 130.9 | 18.5 | 1.1 | 1.4 |
| CD98HC.04.010 | 150.9 | 26.5 | 1.1 | 1.3 |
| CD98HC.04.011 | 192.2 | 31.0 | 1.1 | 1.4 |
| CD98HC.04.012 | 154.7 | 28.3 | 1.1 | 1.3 |
| CD98HC.04.013 | 123.5 | 21.2 | 1.1 | 1.3 |
| CD98HC.04.018 | 334.7 | 50.3 | 1.1 | 1.3 |
| CD98HC.04.022 | 102.0 | 16.6 | 1.1 | 1.3 |
| CD98HC.04.045 | 4.0 | 2.4 | 1.2 | 1.4 |
| CD98HC.04.046 | 147.1 | 27.7 | 1.2 | 1.4 |
| CD98HC.04.047 | 129.2 | 30.2 | 1.2 | 1.4 |
| CD98HC.04.054 | 218.4 | 52.0 | 1.2 | 1.4 |
| CD98HC.04.058 | 158.4 | 33.0 | 1.2 | 1.4 |
| CD98HC.04.062 | 91.8 | 25.1 | 1.3 | 1.5 |
| CD98HC.04.063 | 124.2 | 34.4 | 1.2 | 1.4 |
| CD98HC.04.064 | 277.2 | 46.6 | 1.2 | 1.4 |
| CD98HC.04.065 | 161.8 | 28.3 | 1.2 | 1.4 |
| Sigma Mouse anti- | 225.1 | 41.1 | 1.0 | 1.0 |
| huCD98 antibody | | | | |
| Biorad Rat anti-mouse | 1.0 | 1.0 | 44.3 | 1.0 |
| CD98 antibody | | | | |
| Secondary antibody | 1.0 | 1.0 | 1.0 | 1.0 |
| only | | | | |

### Example 6: Screening of Anti-CD98hc Antibody Hybridoma Supernatants by Recombinant CD98hc Protein Binding Assay

Hybridoma culture supernatants from 121 hybridomas (obtained from Example 5) were screened for their ability to bind Avi-His-tagged human, cyno, and mouse CD98hc (prepared as described in Example 1) and compared to binding to Avi-His-tagged human TfR, a control protein. Briefly, 96-well polystyrene plates were coated with 5 μg/ml of streptavidin (Thermo Fisher, Cat #PI21125) in coating buffer (0.05M carbonate buffer, pH 9.6, Sigma, Cat #C3041) overnight at 4° C. Coated plates were then blocked with ELISA diluent (PBS+0.5% BSA+0.05% Tween20) for one hour. Blocking buffer was removed and human, cyno, or mouse Avi-His-tagged CD98hc and Avi-His-tagged human TfR polypeptides were added at 1 μg/ml in ELISA diluent and captured for 1 hour at room temperature. After washing three times with 300 μl of PBST (PBS+0.05% Tween20, Thermo 28352), the hybridoma cell culture supernatants or 1 μg/ml of commercially available purified mouse anti-human CD98hc monoclonal antibody (Sigma Aldrich, Cat #SAB 4700503) and rat anti-mouse CD98hc antibody (Bio-Rad Laboratories, Hercules, CA, Cat #MCA2684) were added (50 μl/well) to each well. After 30 minutes of incubation at room temperature, the plates were washed three times with 300 μl of PBST. Anti-mouse IgG Fc-HRP or anti-rat IgG Fc-HRP (Jackson Immunoresearch, Cat #115-035-071 and 112-036-071, respectively) secondary antibodies were diluted 1:5000 in ELISA diluent, added to each well at 50 μl/well, and incubated for 30 minutes at room temperature with shaking. After a final set of washes (3×300 μl in PBST), 50 μl/well of BioFx TMB substrate (Surmodics, Eden Praire, MN, Cat #TMBW-1000-01) was added to the wells. The reaction was then quenched after 5-10 mins with 50 μl/well of 2N sulfuric acid. The plates were read for absorbance at 450 nm on a SpectraMax M5 (Molecular Devices, Sunnyvale, CA) using SoftMax Pro software. ELISA data for selected antibodies is shown in Table 4 below.

From this hybridoma supernatant screen, 120 out of 121 anti-CD98hc hybridoma clones had OD₄₅₀ values greater than 0.45 to human and cyno CD98hc. One hybridoma clone, CD98HC.02.020, bound to mouse CD98hc.

TABLE 6
ELISA OD₄₅₀ values of unique anti-CD98hc antibodies
identified from hybridoma campaigns 3 and 4.

| | | | | Human TfR-AVI- |
|---|---|---|---|---|
| | Human CD98- | Cyno CD98- | Mouse CD98-AVI- | His |
| Antibody | AVI-His | AVI-His | His | (control) |
| CD98HC.03.002 | 1.5282 | 1.6882 | 0.0802 | 0.073 |
| CD98HC.03.004 | 1.4923 | 1.3988 | 0.0938 | 0.0905 |
| CD98HC.03.010 | 1.0045 | 1.7732 | 0.0625 | 0.0588 |
| CD98HC.03.031 | 1.0612 | 1.4761 | 0.0843 | 0.0873 |
| CD98HC.04.001 | 1.5861 | 1.8463 | 0.0684 | 0.0637 |
| CD98HC.04.002 | 1.234 | 1.8413 | 0.064 | 0.0609 |
| CD98HC.04.003 | 1.6046 | 2.0468 | 0.0565 | 0.0601 |
| CD98HC.04.006 | 1.2401 | 1.7052 | 0.13 | 0.1291 |
| CD98HC.04.009 | 0.9913 | 1.4182 | 0.0688 | 0.0743 |
| CD98HC.04.010 | 1.4805 | 1.9673 | 0.0907 | 0.1015 |
| CD98HC.04.011 | 1.163 | 1.7618 | 0.067 | 0.0705 |
| CD98HC.04.012 | 1.6385 | 1.7342 | 0.0786 | 0.074 |
| CD98HC.04.013 | 1.1704 | 2.0437 | 0.0717 | 0.0667 |
| CD98HC.04.018 | 1.6427 | 1.9671 | 0.079 | 0.0717 |
| CD98HC.04.022 | 1.3613 | 1.8919 | 0.0554 | 0.0533 |
| CD98HC.04.045 | 1.5109 | 1.3407 | 0.0764 | 0.1033 |
| CD98HC.04.046 | 1.3811 | 1.876 | 0.0817 | 0.0719 |
| CD98HC.04.047 | 1.089 | 1.5648 | 0.103 | 0.0949 |
| CD98HC.04.055 | 1.3175 | 1.6149 | 0.1696 | 0.1282 |
| CD98HC.04.058 | 1.3419 | 1.7318 | 0.1206 | 0.0976 |
| CD98HC.04.062 | 1.6086 | 1.6286 | 0.0607 | 0.0561 |
| CD98HC.04.063 | 1.6636 | 1.9109 | 0.0738 | 0.0629 |
| CD98HC.04.064 | 1.6971 | 1.765 | 0.073 | 0.0639 |
| CD98HC.04.065 | 1.3531 | 1.7036 | 0.0984 | 0.1001 |
| Sigma Mouse anti- | 1.1114 | 1.5655 | 0.0577 | 0.0593 |
| huCD98 | | | | |
| Biorad Rat anti-mouse | 0.0971 | 0.1406 | 1.9103 | 0.1005 |
| CD98 | | | | |
| Secondary Ab only | 0.0536 | 0.0576 | 0.0484 | 0.0486 |

### Example 7: In Vitro Internalisation of Anti-CD98kc Antibodies into a Blood-Brain Barrier Endothelial Cell Line

Supernatants from the 121 anti-CD98hc hybridoma clones were purified using ProPlus Phytip columns (Biotage, Uppsala, Sweden, Cat #PTH 91-20-07) on a Hamilton STAR platform (Hamilton Company, Reno, NV). Briefly, antibodies from the supernatants were captured by protein A coupled on resin-packed tips, washed twice with PBS, eluted with Pierce IgG elution buffer (ThermoFisher, Cat #21004), and neutralized with 1M Tris-HCl pH8 to a final pH of 6.0. The concentrations of the purified antibodies were determined by measuring the absorbance at 280 nm using the Nanodrop 8000 (ThermoFisher). Hybridoma purified antibodies were then tested for their ability to internalize into endothelial cells.

Internalization into endothelial cells at the blood-brain barrier is the first stage of transcytosis across the BBB and into the brain. To identify anti-CD98hc antibodies with ability to internalize, HCMEC/D3 cells were seeded at 2.5*10\hat{ }4 cells/well in a black wall clear bottom 96-well plate (#3904, Corning). Next day, cells were treated with 6 μg/ml of anti-CD98hc antibodies pre-conjugated with an equal concentration of pHrodo-Red labeling reagent (Z25612, Invitrogen) in 100 μl of culture medium (EBM2, Lonza). A human IgG isotype and an anti-CD98hc reference antibody with known internalization ability were included in the assay as negative and positive controls, respectively.

Plates were then placed into an IncuCyte machine (live-cell analysis system), and images were captured every 2 hours over a 24 hour time course. Images were then processed and analyzed using IncuCyte software. Internalization data at the 24 hour timepoint (pHrodo Red positive area μm²/image) is shown in Table 5 as relative fold-change to isotype hIgG1.

TABLE 7
Internalization fold-change of purified unique anti-CD98hc antibodies
identified from hybridoma campaigns relative to isotype hIgG1

| | Fold-change |
|---|---|
| | relative to |
| Antibody | isotype hIgG1 |
| CD98hc.03.002 | 10.9 |
| CD98hc.03.004 | 45.4 |
| CD98hc.03.010 | 32.5 |
| CD98hc.03.030 | 8.5 |
| CD98hc.03.031 | 350.0 |
| CD98hc.04.003 | 10.9 |
| CD98hc.04.006 | 29.1 |
| CD98hc.04.009 | 41.0 |
| CD98hc.04.010 | 21.2 |
| CD98hc.04.011 | 9.9 |
| CD98hc.04.012 | 206.4 |
| CD98hc.04.013 | 38.5 |
| CD98hc.04.018 | 34.5 |
| CD98hc.04.022 | 36.5 |
| CD98hc.04.045 | 92.4 |
| CD98hc.04.046 | 24.4 |
| CD98hc.04.047 | 13.2 |
| CD98hc.04.054 | 23.1 |
| CD98hc.04.056 | 161.0 |
| CD98hc.04.058 | 16.4 |
| CD98hc.04.061 | 38.3 |
| CD98hc.04.062 | 22.2 |
| CD98hc.04.063 | 12.8 |
| CD98hc.04.064 | 175.3 |
| CD98hc.04.065 | 24.2 |
| Reference Anti-CD98hc | 25.2 |
| Antibody | |

### Example 8: Molecular Cloning of Anti-CD98hc Antibodies

Anti-CD98hc antibodies from the 121 hybridomas described above were cloned as follows. Anti-CD98hc antibodies were selected based on FACS positive data (described above in Example 5). 1-2×10⁵ hybridoma cells were harvested, washed with PBS, and resuspended in 200 μl of RNAlater (Invitrogen, Cat #. AM7021). Samples were stored at −80° C. and sent to Abterra Biosciences (San Diego, CA) for sequencing. Briefly, RNA was extracted and cDNA synthesis was performed. The variable regions of IgG/IgM, IgK, and IgL were amplified using proprietary primers in a 5′ RACE strategy. Hybridoma variable region amplicons were sequenced on the Illumina MiSeq platform (Illumina, San Diego, CA). Reads from the hybridomas were processed through Abterra's Reptor analysis pipeline. Exemplary unique amino acid sequences of the variable heavy chains and variable light chains of anti-CD98hc antibodies are provided below in Table 8. Tables 9 and 10 provide the CDR sequences (Kabat) for each of the unique VH and VL sequences in Table 8.

TABLE 8
Unique VH/VL sequences derived from hybridoma campaigns

| Antibody | VH | VL |
|---|---|---|
| CD98HC.03.002 | DVQLVESGGGLVQPGGSRKLSCA | DIQMTQTTSSLSASLGDRVTISCR |
| | ASGFTFSTFGMHWVRQAPEKGLE | ASQDISNYLNWYQQKPDGTVKLLI |
| | WVAYISGGSTNIYYADTVKGRFT | YYTSRLHSGVPSRFSGSGSGTDYS |
| | ISRDNPKNTLFLQMTSLRSEDTA | LTISNLEQEDIATYFCQQGSTLPR |
| | MYYCARETTLLATNFDYWGQGTT | TFGGGTKLEIK |
| | LTVSS | (SEQ ID NO: 7) |
| | (SEQ ID NO: 6) | |
| CD98HC.03.004 | DVQLQESGPGLVKPSQSLSLTCT | QAVVTQESALTTSPGETVTLTCRS |
| | VTGYSITNNYAWNWIRRFPGNKL | STGAVTTSNFANWVQEKPDHLFTG |
| | EWMAYISYSGYTSYNPSLKSRIS | LIRGSNNRAPGVPARFSGSLIGDK |
| | ITRDTSKNQFFLQLNSVTTEDTA | AALTITGARTEDEAIYFCALWYSD |
| | TYYCTGYFYYYGGGSFDYWGQGT | HWVFGGGTKLTVL |
| | TLTVSS | (SEQ ID NO: 9) |
| | (SEQ ID NO: 8) | |
| CD98HC.03.010 | EVQLQQSGTELVKPGASVKLSCT | QIVLTQSPAIMSASPGEKVTLTCS |
| | ASGLDIKDTYLHWVKQRPEQGLE | ASSSVSSSYLYWYQQKPGSSPKLW |
| | WIGKIDPANGNTKYNPKFQGKAT | IYSTSNLASGVPARFSGSGSGTSY |
| | ITADTSSNTAYLQLSSLTSEDTA | SLTISSMEAEDAASYFCHQWSRYP |
| | VHYCTTPYYSGSSYGDYWGQGTT | LTFGAGTKLELK |
| | LTVSS | (SEQ ID NO: 11) |
| | (SEQ ID NO: 10) | |
| CD98HC.03.031 | QVQLQQSGAELVRPGVSVKISCK | DIVMTQSQRFMSTSVGDGVRITCK |
| | ASGYTFTDYAMHWVKQSHAKSLE | ASQNVRTAVAWYQQKPGQSPKALI |
| | WIGVISTYYGDTTYNQKFKDKAT | NLAPNRHTGVPDRFTARGSGTDFT |
| | MTVDKSSSTAYLELARLTSEDSA | LTISNVQSEDLADYFCLQHWNYPL |
| | IYYCARVWLLSYAMDNWGQGTSV | TFGAGTKLELK |
| | TVSS | (SEQ ID NO: 13) |
| | (SEQ ID NO: 12) | |
| CD98HC.04.003 | QVQLQQSGAELAKPGASLKMSCK | DIQMTQTTSSLSASLGDRVTISCR |
| | ASGYTFTDYYIHWVKQRPGQDLE | ASQDISNYLNWYQQKPDGTVKLLI |
| | WIGYINPNSAYTDSNQKFKDKAT | YYTSRLHSGVPSRFSGSGSGADYS |
| | LTADKSSNTAYMQLSSLTSEDSA | LTISNLQQEDIATYFCQQGDTLPY |
| | VYYCARKGHFYAPSFAYWGQGTL | TFGGGTKLEIE |
| | VTVSA | (SEQ ID NO: 15) |
| | (SEQ ID NO: 14) | |
| CD98HC.04.006 | EVNLVESGGDLVQPGGSLRLSCA | DIVMSQSPSSLAVSVGETVTMSCK |
| | TSGFTFTDYFMSWVRQPPGKALE | SSQSLLYSSNQKNYLAWYQQKPGQ |
| | WLGFIRNKAYGYTTEYSASVKGR | SPKLLIYWGSTRESGVPDRFTGSG |
| | FTISRDNSQSIFYLQMNTLRLED | SGTDFTLTISSVKAEDLAVYYCQH |
| | SATYYCARGPFISTAPFAYWGQG | YYGYPWTFGGGTKLEIK |
| | TLVTVSA | (SEQ ID NO: 17) |
| | (SEQ ID NO: 16) | |
| CD98HC.04.009 | QVQLKESGPGLVAPSQSLSITCT | ENVLTQSPASMSASPGEKVTMTCR |
| | VSGFSLTSYGVHWVRQPPGKGLE | ASSSVSSSYLHWYQQKSGASPKLW |
| | WLGVIWAGGSTYYNSALMSRLSI | IYSTSNLASGVPARFSGSGSGTSY |
| | SKDNAKSQVLLKMSSLQTDDTAM | SLTISSVEAEDAATYYCQQYSGYP |
| | YYCARDNYGGVYLDVWGAGTTVT | LTFGSGTKLEIK |
| | VSS | (SEQ ID NO: 19) |
| | (SEQ ID NO: 18) | |
| CD98HC.04.010 | QVQLQQSGAELMKPGASVKISCK | QIVLTQSPAIMSASPGEKVTITCS |
| | ATGYTFSTYWIEWVKQRPGHGLE | ASSSVSYMFWFQQRPGTSPKLWIY |
| | WIGEILPGSGTTTYNEKFKGKAT | TTSNLASGVPARFSGSGSGTSYSL |
| | FTADTSSNTAYMQLSSLTSEDSA | TISRMEAEDAATYYCQQKNNYPFT |
| | VYYCARRTVYYRYEGIDYWGQGT | FGSGTKLEIK |
| | TLTVSS | (SEQ ID NO: 21) |
| | (SEQ ID NO: 20) | |
| CD98HC.04.011 | DVQLQESGPDLVKPSQSLSLTCS | DIQMTQSSSYLSVSLGDRVTITCK |
| | VTVYSFTSVYSWHWIRQFPGNKL | ASDHINNWLAWYQQKPGNAPRLLI |
| | EWMGYIHDSGTTNYNPSLKSRIS | TGAVNLETGVPSRFSGSGSGKDYT |
| | VTRDTSKNQFFLQLNSVTTEDTA | LSITSLQTEDVATYYCQQYWSIPR |
| | TYYCARGRPYGYDGFAYWGQGTL | TFGGGTNLEIK |
| | VTVSA | (SEQ ID NO: 23) |
| | (SEQ ID NO: 22) | |
| CD98HC.04.012 | QVQLQQSGADLVRPGDSVKISCK | NIMMTQSPSSLAVSAGEKVTMNCK |
| | GSGYTFTDNTLHWVKQSHAKSLE | SSQSVLSSSNQKNYLAWYQQKPGQ |
| | WIGVISPYYGDASYNQKFKGKAT | SPKLLIYWASTRESGVPARFTGSG |
| | MTVDKSSSTAYMELARLTSEDSA | SGTDFSLTITSVQAEDLAVYYCLQ |
| | IYYCARVGGRLRLGYYFDHWGQG | YLSSYTFGGGTKLEIK |
| | TTLTVSS | (SEQ ID NO: 25) |
| | (SEQ ID NO: 24) | |
| CD98HC.04.013 | EVQLVESGGDLGMPGGSLKLSCA | DIVMSQSPSSLAVSVGEKVTLTCK |
| | ASGFALSGYDMSWVRQTPDKRLE | SSQSLLYSGNQKNYLAWYQQNPGQ |
| | WVAYLSSGGGSTYYPYTVKGRFT | SPKLLIYWASTRESGVPDRFTGSG |
| | ISRDNAQNTLYLQMSSLKSEDTA | SGTDFTLTISSVKAEDLAVYYCQQ |
| | IYYCARHSEVRPWFGYWGQGTLV | YYTYPPTFGAGTKLGLK |
| | TVSA | (SEQ ID NO: 27) |
| | (SEQ ID NO: 26) | |
| CD98HC.04.018 | QIQLVQSGPELKKPGETVKISCK | QIVLTQSPAIMSASLGERVTMTCT |
| | ASGYTFRNYGMNWVKQAPGKGLK | ASSSVSSSYLYWYQQKPGSSPKLW |
| | WMGWINTYTGEPTYAEDFKGRFA | IYSIFNLASGVPARFSGSGSGTSY |
| | FSLETSASTAYLQINKLKNEDEA | SLTISSMEAEDAATYYCHQYHRSP |
| | TYFCARGEAHWYFDVWGAGTTVT | PTFGGGTKLEIK |
| | VSS | (SEQ ID NO: 29) |
| | (SEQ ID NO: 28) | |
| CD98HC.04.022 | EVQLQQSGAELVKPGASVKLSCT | DILMTQSPSSMSVSLGDTVTITCH |
| | ASGFNIKDTFMHWMKQRPEQGLE | ASQGISSNIGWLQQKPGRSFKGLI |
| | WIGRIDPANANTRYDPKFQGKAT | YHGSNLEDGVPSRFTGSGSGTDYS |
| | ITTDTSSNTAYLQLSSLTSEDTA | LTISGLESEDFADYYCVQYAQFPR |
| | VYYCARDVYYDVWGAGTTVTVSS | TFGGGTKLEIQ |
| | (SEQ ID NO: 30) | (SEQ ID NO: 31) |
| CD98HC.04.045 | QIQLVQSGPELKKPGETVKISCK | DIQLTQSPSSLSASLGERVSLTCR |
| | ASGYSITNFGMNWVKQAPGKGLK | ASQEISGYLSWLQQKPDGTIKRLI |
| | WMGWVNTYTGEPTYADDFKGRFA | YAASTLDSGVPKRFSGSRSGSDYS |
| | FSLETSASTASLQINNLKNEDTA | LTISSLESEDFADYYCLQYASYPR |
| | TYFCTRSVITYTMDYWGQGTSVT | TFGGGTKVEIK |
| | VSS | (SEQ ID NO: 33) |
| | (SEQ ID NO: 32) | |
| CD98HC.04.046 | QIQLVQSGPVLKKPGETVKISCK | QIVLTQSPAIMSASLGERVTMTCI |
| | ASGYTFTNFGMNWVKQAPGKGLK | VSSSVSSSYLYWYQQKPGSSPKLW |
| | WMGWIDTYTREPTYVDDFKGRFA | IYSTSILASGVPGRFSGSGSGTSY |
| | FSLETSVTTAYLQINDLKNEDSA | SLTISSMEAEDAATYYCHQYHRSP |
| | TYFCARGESHWYFDVWGAGTTVT | PTFGGGTKLEIK |
| | VSS | (SEQ ID NO: 35) |
| | (SEQ ID NO: 34) | |
| CD98HC.04.047 | EVQLQESGPSLVKPSQTLSLTCS | QIVLTQSPAIMSASPGEKVTISCS |
| | VTGDSITSGYWNWIRKFPGNKLE | ASSSVGYMYWLQQKPGSSPKPWIY |
| | YMGYISYSGSTYYNPSLKGRVSI | RTSILASGVPARFSGSGSGTSYSL |
| | TRDTSKNQYYLQLNSVTTEDTAT | TISSMEAEDAATYYCQQYQSYPLT |
| | YYCQRHVIYDYTWFAYWGQGTLV | FGAGTKLELK |
| | TVSA | (SEQ ID NO: 37) |
| | (SEQ ID NO: 36) | |
| CD98HC.04.054 | QVQLQQSAAELARPGASVKMSCE | DIVMTQSQKFMSTSFGDRVDITCK |
| | TSGYTFTRYTMNWVKQRPGQGLD | ASQNVGSAVAWYQQKPGQSPKLLI |
| | WIGYINPSSGYAEYNQKFRGKTT | YSASNRYTGVPDRFTGSGSGTDFT |
| | LTADKSSSTAYIQLNSLTSEDSA | LTINNMQSADLADYFCQQYNFYPL |
| | VYYCVRYDGNYVYWGQGTTLTVS | TFGAGTKLDLK |
| | S | (SEQ ID NO: 39) |
| | (SEQ ID NO: 38) | |
| CD98HC.04.058 | QVQLQQSGAELVRPGVSVKISCK | NIMMTQSPSSLAVSAGEKVTMNCK |
| | ASGYTFTDHTMHWVKQSHARSLE | SSQTVLYSSNQKNYLAWYQQKPGQ |
| | WIGVISTYFGDASYNPKFKGKAT | SPKLLIYWASTRESGVPDRFTGSG |
| | MTVDKSSNTAYMELARLTSEDSA | SGTDFTLTISSVQVEDLAVYYCHQ |
| | IYYCARVGGRLRLGYYFDYWGQG | YLSSYTFGGGTKLEIK |
| | TTLTVSS | (SEQ ID NO: 41) |
| | (SEQ ID NO: 40) | |
| CD98HC.04.062 | EVQLVESGGDLVKPGGSLKLSCT | DIVMSQSPSSLAVSVGEKVTMSCK |
| | ASRFAFSTYDMSWVRQTPEKRLE | SSQSLLYSGNQKTYLAWYQQKPGQ |
| | WVAYISSGGGSTYFPDTVKGRFT | SPKPLIYWASTRESGIPDRFTGSG |
| | ISRDNAKNTLYLQMSSLKSEDTA | SGTDFTLTISSVKAEDLAVYYCQQ |
| | MYYCARQDGHYPWFPYWGQGTLV | YYSYPPTFGAGTKLELK |
| | TVSA | (SEQ ID NO: 43) |
| | (SEQ ID NO: 42) | |
| CD98HC.04.063 | EVKLEESGGGLVQPGGSMILSCV | DIQMTQSSSYLSVSLGNRVTITCK |
| | ASGFTFSNSWMNWVRQSPEKGLE | ASDHIKNWLAWFQQKPGNAPRLLI |
| | WVAEIRLRSNNYATHYAESVKGR | SGATILETGVPSRFSGSGSGKDYT |
| | FTISRDDSKSTVYLQMNNLRAED | LSITSLQTEDVATYYCQQYWSTPY |
| | TGIYYCSRGDPGAYWGQGTLVTV | TFGGGTKLEIK |
| | SA | (SEQ ID NO: 45) |
| | (SEQ ID NO: 44) | |
| CD98HC.04.064 | QVQLQQPGAELVRPGASVKLSCK | DIVMTQSPSSLAMSVGQKVTMSCK |
| | ASGYTFTSYWMNWVRQRPGQGLE | SSQSLLNNINQKNYLAWYQQKPGQ |
| | WIGLIDPSDSETHYNQMFKDRAT | SPKLLVYFASTRESGVPDRFIGSG |
| | LTVDKSSSTAYMQLSSLTSADPA | SGTDFTLTINTVQAEDLADYFCQQ |
| | VYYCARASYGKGYFDYWGQGTTL | HYSSPFTFGGGTKLEIK |
| | TVSS | (SEQ ID NO: 47) |
| | (SEQ ID NO: 46) | |
| CD98HC.04.065 | EVQLQESGPSLVKPSQTLSLTCS | QIVLTQSPAIMSASPGEKVTISCS |
| | VTGDSITSGYWNWIRKFPGNKLE | ASSSVHYMYWFQQKPGSSPKPWIY |
| | YMGYISYSGRTYYNPSLKSRVSI | RTSILASGVPDRFSGSGSGTSYSL |
| | TRDTSKNQYYLQLNSVTTEDTAT | TISSMEAEDAATYYCQQYQSYPLT |
| | YYCQRHVIYDYTWFAYWGQGTLV | FGAGTKLELK |
| | TVSA | (SEQ ID NO: 49) |
| | (SEQ ID NO: 48) | |

TABLE 9
CDR Heavy Chain sequences derived from hybridoma campaigns

| Antibody | CDR-H1 | CDR-H2 | CDR-H3 |
|---|---|---|---|
| CD98HC.03.002 | TFGMH | YISGGSTNIYYADTV | ETTLLATNFDY |
| | (SEQ ID NO: 50) | KG | (SEQ ID NO: 52) |
| | | (SEQ ID NO: 51) | |
| CD98HC.03.004 | NNYAWN | YISYSGYTSYNPSLKS | YFYYYGGGSFDY |
| | (SEQ ID NO: 53) | (SEQ ID NO: 54) | (SEQ ID NO: 55) |
| CD98HC.03.010 | DTYLH | KIDPANGNTKYNPKF | PYYSGSSYGDY |
| | (SEQ ID NO: 56) | QG | (SEQ ID NO: 58) |
| | | (SEQ ID NO: 57) | |
| CD98HC.03.031 | DYAMH | VISTYYGDTTYNQKF | VWLLSYAMDN |
| | (SEQ ID NO: 59) | KD | (SEQ ID NO: 61) |
| | | (SEQ ID NO: 60) | |
| CD98HC.04.003 | DYYIH | YINPNSAYTDSNQKF | KGHFYAPSFAY |
| | (SEQ ID NO: 62) | KD | (SEQ ID NO: 64) |
| | | (SEQ ID NO: 63) | |
| CD98HC.04.006 | DYFMS | FIRNKAYGYTTEYSA | GPFISTAPFAY |
| | (SEQ ID NO: 65) | SVKG | (SEQ ID NO: 67) |
| | | (SEQ ID NO: 66) | |
| CD98HC.04.009 | SYGVH | VIWAGGSTYYNSAL | DNYGGVYLDV |
| | (SEQ ID NO: 68) | MS | (SEQ ID NO: 70) |
| | | (SEQ ID NO: 69) | |
| CD98HC.04.010 | TYWIE | EILPGSGTTTYNEKF | RTVYYRYEGIDY |
| | (SEQ ID NO: 71) | KG | (SEQ ID NO: 73) |
| | | (SEQ ID NO: 72) | |
| CD98HC.04.011 | SVYSWH | YIHDSGTTNYNPSLK | GRPYGYDGFAY |
| | (SEQ ID NO: 74) | S | (SEQ ID NO:76) |
| | | (SEQ ID NO: 75) | |
| CD98HC.04.012 | DNTLH | VISPYYGDASYNQKF | VGGRLRLGYYFD |
| | (SEQ ID NO: 77) | KG | H |
| | | (SEQ ID NO: 78) | (SEQ ID NO:79) |
| CD98HC.04.013 | GYDMS | YLSSGGGSTYYPYTV | HSEVRPWFGY |
| | (SEQ ID NO: 80) | KG | (SEQ ID NO:82) |
| | | (SEQ ID NO: 81) | |
| CD98HC.04.018 | NYGMN | WINTYTGEPTYAEDF | GEAHWYFDV |
| | (SEQ ID NO: 83) | KG | (SEQ ID NO: 85) |
| | | (SEQ ID NO: 84) | |
| CD98HC.04.022 | DTFMH | RIDPANANTRYDPKF | DVYYDV |
| | (SEQ ID NO: 86) | QG | (SEQ ID NO: 88) |
| | | (SEQ ID NO: 87) | |
| CD98HC.04.045 | NFGMN | WVNTYTGEPTYADD | SVITYTMDY |
| | (SEQ ID NO: 89) | FKG | (SEQ ID NO: 91) |
| | | (SEQ ID NO: 90) | |
| CD98HC.04.046 | NFGMN | WIDTYTREPTYVDDF | GESHWYFDV |
| | (SEQ ID NO: 92) | KG | (SEQ ID NO: 94) |
| | | (SEQ ID NO: 93) | |
| CD98HC.04.047 | SGYWN | YISYSGSTYYNPSLK | HVIYDYTWFAY |
| | (SEQ ID NO: 95) | G | (SEQ ID NO: 97) |
| | | (SEQ ID NO: 96) | |
| CD98HC.04.054 | RYTMN | YINPSSGYAEYNQKF | YDGNYVY |
| | (SEQ ID NO: 98) | RG | (SEQ ID NO: 100) |
| | | (SEQ ID NO: 99) | |
| CD98HC.04.058 | DHTMH | VISTYFGDASYNPKF | VGGRLRLGYYFD |
| | (SEQ ID NO: 101) | KG | Y |
| | | (SEQ ID NO: 102) | (SEQ ID NO: 103) |
| CD98HC.04.062 | TYDMS | YISSGGGSTYFPDTV | QDGHYPWFPY |
| | (SEQ ID NO: 104) | KG | (SEQ ID NO: 106) |
| | | (SEQ ID NO: 105) | |
| CD98HC.04.063 | NSWMN | EIRLRSNNYATHYAE | GDPGAY |
| | (SEQ ID NO: 107) | SVKG | (SEQ ID NO: 109) |
| | | (SEQ ID NO: 108) | |
| CD98HC.04.064 | SYWMN | LIDPSDSETHYNQMF | ASYGKGYFDY |
| | (SEQ ID NO: 110) | KD | (SEQ ID NO: 112) |
| | | (SEQ ID NO: 111) | |
| CD98HC.04.065 | SGYWN | YISYSGRTYYNPSLK | HVIYDYTWFAY |
| | (SEQ ID NO: 113) | S | (SEQ ID NO: 115) |
| | | (SEQ ID NO: 114) | |

TABLE 10
CDR Light Chain sequences derived from hybridoma campaigns

| Antibody | CDR-L1 | CDR-L2 | CDR-L3 |
|---|---|---|---|
| CD98HC.03.002 | RASQDISNYLN | YTSRLHS | QQGSTLPRT |
| | (SEQ ID NO: 116) | (SEQ ID NO:117) | (SEQ ID NO: 118) |
| CD98HC.03.004 | RSSTGAVTTSNFAN | GSNNRAP | ALWYSDHWV |
| | (SEQ ID NO: 119) | (SEQ ID NO: 120) | (SEQ ID NO: 121) |
| CD98HC.03.010 | SASSSVSSSYLY | STSNLAS | HQWSRYPLT |
| | (SEQ ID NO: 122) | (SEQ ID NO: 123) | (SEQ ID NO: 124) |
| CD98HC.03.031 | DYAMH | VISTYYGDTTYNQKF | VWLLSYAMDN |
| | (SEQ ID NO: 125) | KD | (SEQ ID NO: 127) |
| | | (SEQ ID NO: 126) | |
| CD98HC.04.003 | DYYIH | YINPNSAYTDSNQKF | KGHFYAPSFAY |
| | (SEQ ID NO: 128) | KD | (SEQ ID NO: 130) |
| | | (SEQ ID NO: 129) | |
| CD98HC.04.006 | DYFMS | FIRNKAYGYTTEYSA | GPFISTAPFAY |
| | (SEQ ID NO: 131) | SVKG | (SEQ ID NO: 133) |
| | | (SEQ ID NO: 132) | |
| CD98HC.04.009 | RASSSVSSSYLH | STSNLAS | QQYSGYPLT |
| | (SEQ ID NO: 134) | (SEQ ID NO: 135) | (SEQ ID NO: 136) |
| CD98HC.04.010 | SASSSVSYMF | TTSNLAS | QQKNNYPFT |
| | (SEQ ID NO: 137) | (SEQ ID NO: 138) | (SEQ ID NO: 139) |
| CD98HC.04.011 | KASDHINNWLA | GAVNLET | QQYWSIPRT |
| | (SEQ ID NO: 140) | (SEQ ID NO: 141) | (SEQ ID NO: 142) |
| CD98HC.04.012 | KSSQSVLSSSNQKN | WASTRES | LQYLSSYT |
| | YLA | (SEQ ID NO: 144) | (SEQ ID NO: 145) |
| | (SEQ ID NO: 143) | | |
| CD98HC.04.013 | KSSQSLLYSGNQKN | WASTRES | QQYYTYPPT |
| | YLA | (SEQ ID NO: 147) | (SEQ ID NO: 148) |
| | (SEQ ID NO: 146) | | |
| CD98HC.04.018 | TASSSVSSSYLY | SIFNLAS | HQYHRSPPT |
| | (SEQ ID NO: 149) | (SEQ ID NO: 150) | (SEQ ID NO: 151) |
| CD98HC.04.022 | HASQGISSNIG | HGSNLED | VQYAQFPRT |
| | (SEQ ID NO: 152) | (SEQ ID NO: 153) | (SEQ ID NO: 154) |
| CD98HC.04.045 | RASQEISGYLS | AASTLDS | LQYASYPRT |
| | (SEQ ID NO: 155) | (SEQ ID NO: 156) | (SEQ ID NO: 157) |
| CD98HC.04.046 | IVSSSVSSSYLY | STSILAS | HQYHRSPPT |
| | (SEQ ID NO: 158) | (SEQ ID NO: 159) | (SEQ ID NO: 160) |
| CD98HC.04.047 | SASSSVGYMY | RTSILAS | QQYQSYPLT |
| | (SEQ ID NO: 161) | (SEQ ID NO: 162) | (SEQ ID NO: 163) |
| CD98HC.04.054 | KASQNVGSAVA | SASNRYT | QQYNFYPLT |
| | (SEQ ID NO: 164) | (SEQ ID NO: 165) | (SEQ ID NO: 166) |
| CD98HC.04.058 | KSSQTVLYSSNQKN | WASTRES | HQYLSSYT |
| | YLA | (SEQ ID NO: 168) | (SEQ ID NO: 169) |
| | (SEQ ID NO: 167) | | |
| CD98HC.04.062 | KSSQSLLYSGNQKT | WASTRES | QQYYSYPPT |
| | YLA | (SEQ ID NO: 171) | (SEQ ID NO: 172) |
| | (SEQ ID NO: 170) | | |
| CD98HC.04.063 | KASDHIKNWLA | GATILET | QQYWSTPYT |
| | (SEQ ID NO: 173) | (SEQ ID NO: 174) | (SEQ ID NO: 175) |
| CD98HC.04.064 | KSSQSLLNNINQKN | FASTRES | QQHYSSPFT |
| | YLA | (SEQ ID NO: 177) | (SEQ ID NO: 178) |
| | (SEQ ID NO: 176) | | |
| CD98HC.04.065 | SASSSVHYMY | RTSILAS | QQYQSYPLT |
| | (SEQ ID NO: 179) | (SEQ ID NO: 180) | (SEQ ID NO: 181) |

### Example 9: Reformatting Hybridoma Antibodies as 2+1 Bispecific Antibodies

Antibodies were selected for formatting into a 2+1 bispecific format based on various criteria: 1) the antibodies covered a broad range of affinities based on ELISA and FACS binding assays, 2) the antibodies covered a broad range of internalization activity, 3) no outstanding high-risk modification sites were identified in the CDRs, and 4) the antibodies were phylogenetically diverse from each other within the hybridoma sequences obtained. Cross-reactivity of the antibodies between human and cyno CD98hc was also used as a criteria for selection. From the 121 anti-CD98hc antibodies, a panel of 24 anti-CD98hc hybridoma antibodies were selected based on these criteria for reformatting into scFvs in a VH-linker-VL format.

An IgG isotype antibody with no target specificity (“inert isotype control antibody”) with knob-hole mutations in the constant regions of the heavy chains was used for formatting into a 2+1 bispecific antibody. An anti-CD98hc scFv (VH and VL) was appended to the IgG isotype antibody via a linker at the C-terminus of the constant region with the “hole” mutation (“hole side” of the IgG isotype antibody). The IgG isotype antibody attached to a scFv is called a 2+1 bispecific antibody, as shown in FIG. 1A. The 20 amino acid linker connecting the VH and VL domains within the scFv was GGSEGKSSGSGSESKSTGGS (SEQ ID NO:182) (Bird et al, Science 1988. October 21; 242 (4877): 423-6), and the linker connecting the C-terminus of the Fc ‘hole’ domain to the scFv was (GGGGS)x3 (SEQ ID NO:183). DNA encoding the 2+1 bispecific antibodies were prepared by gene synthesis and cloned into the expression vector pcDNA3.4 (ThermoFisher).

The scFv (VH and VL) sequences were formatted into an expression construct of the heavy chain of the IgG with the “hole” mutation (“heavy chain-hole” construct) using the framework in SEQ ID NO:184. The corresponding heavy chain-knob and the light chain expression constructs were also generated (SEQ ID NOs: 186-187). In some instances, the “heavy chain-hole” construct included a set of mutations (H435R, Y436F) to minimize binding to Protein A (Tustian et al., MAbs. May-June 2016; 8 (4): 828-38) (SEQ ID NO:185). In some instances, the heavy chain-hole and the heavy chain-knob also included mutations to reduce effector function, such as L234A/L235A/P331S (LALAPS) or N325S/L328F (NSLF) (SEQ ID NOs: 188-193).

An example structure of a 2+1 bispecific antibody comprised the following components: 1) Isotype control hIgG1 wildtype antibody with a knob (T366W) mutation and a hole mutation (T366S_L368A_Y407V) in the constant regions, 2) a (G4S)x3 linker between the “hole side” of the hIgG1 antibody and a scFv, 3) a VH sequence of the scFv, 4) a 20 amino acid linker sequence between the VH and VL of the scFv, and 5) a VL sequence of the scFv. Table 11 shows example heavy chain with various knob or hole mutations and light chain sequences.

TABLE 11
2 + 1 Bispecific antibody sequences

| SEQ | | |
|---|---|---|
| ID | | |
| NO. | Description | Sequence |
| 184 | Isotype control heavy | EVRLLESGGGLVQPGGSLRLSCAASGFTFSNYAMGWVRQAPGKGLEW |
| | chain wt hIgG1 holescFv | VSAISGSGGSTYYADSVKGRFTTSRDDSKNALYLQMNSLRAEDTAVY |
| | (T366S_L368A_Y407V)- | YCARGGPGWYAADVWGQGTTVTVSSASTKGPSVFPLAPSSKSTSGGT |
| | (G4S)3 linker-VH- | AALGCLVKDYFPEPVTVSWNSGALTSGVHTFPAVLQSSGLYSLSSVV |
| | 20AA linker-VL | TVPSSSLGTQTYICNVNHKPSNTKVDKKVEPKSCDKTHTCPPCPAPE |
| | | LLGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNKA |
| | | LPAPIEKTISKAKGQPREPQVYTLPPSRDELTKNQVSLSCAVKGFYP |
| | | SDIAVEWESNGQPENNYKTTPPVLDSDGSFFLVSKLTVDKSRWQQGN |
| | | VFSCSVMHEALHNHYTQKSLSLSPGK-GGGGSGGGGSGGGGS-X1- |
| | | GGSEGKSSGSGSESKSTGGS-X2, wherein X1 is the |
| | | amino acid sequence of any one of SEQ ID NOs: 6, |
| | | 8, 10, 12, 14, 16, 18, 20, 22, 24, 26, 28, 30, |
| | | 32, 34, 36, 38, 40, 42, 44, 46, 48, 194, 196, |
| | | 198, 200, 202, 204, 206, 208, 210, 212, 214, |
| | | 216, 218, 220, 222, 224, 355, 357, 359, 361, |
| | | 363, 365, 367, 369, 371, 373, 375, 377, 379, |
| | | 381, 383, 385, 387, 389, 391, 393, 395, 397, |
| | | 399, 401, and 403, and wherein X2 is the amino |
| | | acid sequence of any one of SEQ ID NOs: SEQ ID |
| | | NO: 7, 9, 11, 13, 15, 17, 19, 21, 23, 25, 27, |
| | | 29, 31, 33, 35, 37, 39, 41, 43, 45, 47, 49, |
| | | 195, 197, 199, 201, 203, 205, 207, 209, 211, |
| | | 213, 215, 217, 219, 221, 223, 225, 356, 358, |
| | | 360, 362, 364, 366, 368, 370, 372, 374, 376, |
| | | 378, 380, 382, 384, 386, 388, 390, 392, 394, |
| | | 396, 398, 400, 402, and 404. |
| 185 | Isotype control heavy | EVRLLESGGGLVQPGGSLRLSCAASGFTFSNYAMGWVRQAPGKGLEW |
| | chain wt hIgG1 hole scFv | VSAISGSGGSTYYADSVKGRFTTSRDDSKNALYLQMNSLRAEDTAVY |
| | (T366S_L368A_Y407V)- | YCARGGPGWYAADVWGQGTTVTVSSASTKGPSVFPLAPSSKSTSGGT |
| | (G4S)3 linker-VH- | AALGCLVKDYFPEPVTVSWNSGALTSGVHTFPAVLQSSGLYSLSSVV |
| | 20AA linker-VL with | TVPSSSLGTQTYICNVNHKPSNTKVDKKVEPKSCDKTHTCPPCPAPE |
| | mutations (H435R, | LLGGPSVFLFPPKPKDTLMISRTPEVTCVVVDVSHEDPEVKFNWYVD |
| | Y436F) | GVEVHNAKTKPREEQYNSTYRVVSVLTVLHQDWLNGKEYKCKVSNKA |
| | | LPAPIEKTISKAKGQPREPQVYTLPPSRDELTKNQVSLSCAVKGFYP |
| | | SDIAVEWESNGQPENNYKTTPPVLDSDGSFFLVSKLTVDKSRWQQGN |
| | | VFSCSVMHEALHNRFTQKSLSLSPGK-GGGGSGGGGSGGGGS-X1- |
| | | GGSEGKSSGSGSESKSTGGS-X2, wherein X1 is the |
| | | amino acid sequence of any one of SEQ ID NOs: 6, |
| | | 8, 10, 12, 14, 16, 18, 20, 22, 24, 26, 28, 30, |
| | | 32, 34, 36, 38, 40, 42, 44, 46, 48, 194, 196, |
| | | 198, 200, 202, 204, 206, 208, 210, 212, 214, |
| | | 216, 218, 220, 222, and 224, 355, 357, 359, |
| | | 361, 363, 365, 367, 369, 371, 373, 375, 377, |
| | | 379, 381, 383, 385, 387, 389, 391, 393, 395, |
| | | 397, 399, 401, and 403, and wherein X2 is the |
| | | amino acid sequence of any one of SEQ ID |
| | | NOs: SEQ ID NO: 7, 9, 11, 13, 15, 17, 19, 21, 23, |
| | | 25, 27, 29, 31, 33, 35, 37, 39, 41, 43, 45, 47, |
| | | 49, 195, 197, 199, 201, 203, 205, 207, 209, |
| | | 211, 213, 215, 217, 219, 221, 223, 225, 356, |
| | | 358, 360, 362, 364, 366, 368, 370, 372, 374, |
| | | 376, 378, 380, 382, 384, 386, 388, 390, 392, |
| | | 394, 396, 398, 400, 402, and 404 |
| 186 | Isotype control heavy | EVRLLESGGGLVQPGGSLRLSCAASGFTFSNYAMGWVRQAPGKGLEW |
| | chain hIgG1 wt knob | VSAISGSGGSTYYADSVKGRFTTSRDDSKNALYLQMNSLRAEDTAVY |
| | | YCARGGPGWYAADVWGQGTTVTVSSASTKGPSVFPLAPSSKSTSGGT |
| | | AALGCLVKDYFPEPVTVSWNSGALTSGVHTFPAVLQSSGLYSLSSVV |
| | | TVPSSSLGTQTYICNVNHKPSNTKVDKKVEPKSCDKTHTCPPCPAPE |
| | | LLGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNKA |
| | | LPAPIEKTISKAKGQPREPQVYTLPPSRDELTKNQVSLWCLVKGFYP |
| | | SDIAVEWESNGQPENNYKTTPPVLDSDGSFFLYSKLTVDKSRWQQGN |
| | | VFSCSVMHEALHNHYTQKSLSLSPGK |
| 187 | Isotype control light | DIQMTQSPSSLSASVGDRVTITCRASQSISSYLNWYQQKPGKAPKLL |
| | chain | IYAASSLQSGVPSRFSGSGSGTDFTLTISSLQPEDFATYYCQQADLP |
| | | AFAFGGGTKVEIKRTVAAPSVFIFPPSDEQLKSGTASVVCLLNNFYP |
| | | REAKVQWKVDNALQSGNSQESVTEQDSKDSTYSLSSTLTLSKADYEK |
| | | HKVYACEVTHQGLSSPVTKSFNRGEC |
| 188 | Isotype control heavy | EVRLLESGGGLVQPGGSLRLSCAASGFTFSNYAMGWVRQAPGKGLEW |
| | chain hIgG1 LALAPS | VSAISGSGGSTYYADSVKGRFTTSRDDSKNALYLQMNSLRAEDTAVY |
| | hole scFv | YCARGGPGWYAADVWGQGTTVTVSSASTKGPSVFPLAPSSKSTSGGT |
| | (T366S_L368A_Y407V) | AALGCLVKDYFPEPVTVSWNSGALTSGVHTFPAVLQSSGLYSLSSVV |
| | (L234A_L235A_P331S)- | TVPSSSLGTQTYICNVNHKPSNTKVDKKVEPKSCDKTHTCPPCPAPE |
| | (G4S)3 linker-VH-20AA | AAGGPSVFLFPPKPKDTLMISRTPEVTCVVVDVSHEDPEVKFNWYVD |
| | linker-VL | GVEVHNAKTKPREEQYNSTYRVVSVLTVLHQDWLNGKEYKCKVSNKA |
| | | LPASIEKTISKAKGQPREPQVYTLPPSRDELTKNQVSLSCAVKGFYP |
| | | SDIAVEWESNGQPENNYKTTPPVLDSDGSFFLVSKLTVDKSRWQQGN |
| | | VFSCSVMHEALHNHYTQKSLSLSPGK-GGGGSGGGGSGGGGS-X1- |
| | | GGSEGKSSGSGSESKSTGGS-X2, wherein X1 is the |
| | | amino acid sequence of any one of SEQ ID NOs: 6, |
| | | 8, 10, 12, 14, 16, 18, 20, 22, 24, 26, 28, 30, |
| | | 32, 34, 36, 38, 40, 42, 44, 46, 48, 194, 196, |
| | | 198, 200, 202, 204, 206, 208, 210, 212, 214, |
| | | 216, 218, 220, 222, and 224, 355, 357, 359, |
| | | 361, 363, 365, 367, 369, 371, 373, 375, 377, |
| | | 379, 381, 383, 385, 387, 389, 391, 393, 395, |
| | | 397, 399, 401, and 403, and wherein X2 is the |
| | | amino acid sequence of any one of SEQ ID |
| | | NOs: SEQ ID NO: 7, 9, 11, 13, 15, 17, 19, 21, 23, |
| | | 25, 27, 29, 31, 33, 35, 37, 39, 41, 43, 45, 47, |
| | | 49, 195, 197, 199, 201, 203, 205, 207, 209, |
| | | 211, 213, 215, 217, 219, 221, 223, 225, 356, |
| | | 358, 360, 362, 364, 366, 368, 370, 372, 374, |
| | | 376, 378, 380, 382, 384, 386, 388, 390, 392, |
| | | 394, 396, 398, 400, 402, and 404 |
| 189 | Isotype control heavy | EVRLLESGGGLVQPGGSLRLSCAASGFTFSNYAMGWVRQAPGKGLEW |
| | chain hIgG1 LALAPS | VSAISGSGGSTYYADSVKGRFTTSRDDSKNALYLQMNSLRAEDTAVY |
| | hole scFv | YCARGGPGWYAADVWGQGTTVTVSSASTKGPSVFPLAPSSKSTSGGT |
| | (T366S_L368A_Y407V) | AALGCLVKDYFPEPVTVSWNSGALTSGVHTFPAVLQSSGLYSLSSVV |
| | (L234A_L235A_P331S)- | TVPSSSLGTQTYICNVNHKPSNTKVDKKVEPKSCDKTHTCPPCPAPE |
| | (G4S)3 linker-VH-20AA | AAGGPSVFLFPPKPKDTLMISRTPEVTCVVVDVSHEDPEVKFNWYVD |
| | linker-VL with mutations | GVEVHNAKTKPREEQYNSTYRVVSVLTVLHQDWLNGKEYKCKVSNKA |
| | (H435R, Y436F) | LPASIEKTISKAKGQPREPQVYTLPPSRDELTKNQVSLSCAVKGFYP |
| | | SDIAVEWESNGQPENNYKTTPPVLDSDGSFFLVSKLTVDKSRWQQGN |
| | | VFSCSVMHEALHNRFTQKSLSLSPGK-GGGGSGGGGSGGGGS-X1- |
| | | GGSEGKSSGSGSESKSTGGS-X2, wherein X1 is the |
| | | amino acid sequence of any one of SEQ ID NOs: 6, |
| | | 8, 10, 12, 14, 16, 18, 20, 22, 24, 26, 28, 30, |
| | | 32, 34, 36, 38, 40, 42, 44, 46, 48, 194, 196, |
| | | 198, 200, 202, 204, 206, 208, 210, 212, 214, |
| | | 216, 218, 220, 222, and 224, 355, 357, 359, |
| | | 361, 363, 365, 367, 369, 371, 373, 375, 377, |
| | | 379, 381, 383, 385, 387, 389, 391, 393, 395, |
| | | 397, 399, 401, and 403, and wherein X2 is the |
| | | amino acid sequence of any one of SEQ ID |
| | | NOs: SEQ ID NO: 7, 9, 11, 13, 15, 17, 19, 21, 23, |
| | | 25, 27, 29, 31, 33, 35, 37, 39, 41, 43, 45, 47, |
| | | 49, 195, 197, 199, 201, 203, 205, 207, 209, |
| | | 211, 213, 215, 217, 219, 221, 223, 225, 356, |
| | | 358, 360, 362, 364, 366, 368, 370, 372, 374, |
| | | 376, 378, 380, 382, 384, 386, 388, 390, 392, |
| | | 394, 396, 398, 400, 402, and 404 |
| 190 | Isotype control heavy | EVRLLESGGGLVQPGGSLRLSCAASGFTFSNYAMGWVRQAPGKGLEW |
| | chain hIgG1 LALAPS | VSAISGSGGSTYYADSVKGRFTTSRDDSKNALYLQMNSLRAEDTAVY |
| | knob | YCARGGPGWYAADVWGQGTTVTVSSASTKGPSVFPLAPSSKSTSGGT |
| | | AALGCLVKDYFPEPVTVSWNSGALTSGVHTFPAVLQSSGLYSLSSVV |
| | | TVPSSSLGTQTYICNVNHKPSNTKVDKKVEPKSCDKTHTCPPCPAPE |
| | | AAGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSNKA |
| | | LPASIEKTISKAKGQPREPQVYTLPPSRDELTKNQVSLWCLVKGFYP |
| | | SDIAVEWESNGQPENNYKTTPPVLDSDGSFFLYSKLTVDKSRWQQGN |
| | | VFSCSVMHEALHNHYTQKSLSLSPGK |
| 191 | Isotype control heavy | EVRLLESGGGLVQPGGSLRLSCAASGFTFSNYAMGWVRQAPGKGLEW |
| | chain hIgG1 NSLF hole | VSAISGSGGSTYYADSVKGRFTTSRDDSKNALYLQMNSLRAEDTAVY |
| | scFv | YCARGGPGWYAADVWGQGTTVTVSSASTKGPSVFPLAPSSKSTSGGT |
| | (T366S_L368A_Y407V) | AALGCLVKDYFPEPVTVSWNSGALTSGVHTFPAVLQSSGLYSLSSVV |
| | (N325S_L328F)-(G4S)3 | TVPSSSLGTQTYICNVNHKPSNTKVDKKVEPKSCDKTHTCPPCPAPE |
| | linker-VH-20AA linker- | LLGGPSVFLFPPKPKDTLMISRTPEVTCVVVDVSHEDPEVKFNWYVD |
| | VL | GVEVHNAKTKPREEQYNSTYRVVSVLTVLHQDWLNGKEYKCKVSSKA |
| | | FPAPIEKTISKAKGQPREPQVYTLPPSRDELTKNQVSLSCAVKGFYP |
| | | SDIAVEWESNGQPENNYKTTPPVLDSDGSFFLVSKLTVDKSRWQQGN |
| | | VFSCSVMHEALHNHYTQKSLSLSPGK-GGGGSGGGGSGGGGS-X1- |
| | | GGSEGKSSGSGSESKSTGGS-X2, wherein X1 is the |
| | | amino acid sequence of any one of SEQ ID NOs: 6, |
| | | 8, 10, 12, 14, 16, 18, 20, 22, 24, 26, 28, 30, |
| | | 32, 34, 36, 38, 40, 42, 44, 46, 48, 194, 196, |
| | | 198, 200, 202, 204, 206, 208, 210, 212, 214, |
| | | 216, 218, 220, 222, and 224, 355, 357, 359, |
| | | 361, 363, 365, 367, 369, 371, 373, 375, 377, |
| | | 379, 381, 383, 385, 387, 389, 391, 393, 395, |
| | | 397, 399, 401, and 403, and wherein X2 is the |
| | | amino acid sequence of any one of SEQ ID |
| | | NOs: SEQ ID NO: 7, 9, 11, 13, 15, 17, 19, 21, 23, |
| | | 25, 27, 29, 31, 33, 35, 37, 39, 41, 43, 45, 47, |
| | | 49, 195, 197, 199, 201, 203, 205, 207, 209, |
| | | 211, 213, 215, 217, 219, 221, 223, 225, 356, |
| | | 358, 360, 362, 364, 366, 368, 370, 372, 374, |
| | | 376, 378, 380, 382, 384, 386, 388, 390, 392, |
| | | 394, 396, 398, 400, 402, and 404 |
| 192 | Isotype control heavy | EVRLLESGGGLVQPGGSLRLSCAASGFTFSNYAMGWVRQAPGKGLEW |
| | chain hIgG1 NSLF hole | VSAISGSGGSTYYADSVKGRFTTSRDDSKNALYLQMNSLRAEDTAVY |
| | scFv | YCARGGPGWYAADVWGQGTTVTVSSASTKGPSVFPLAPSSKSTSGGT |
| | (T366S_L368A_Y407V) | AALGCLVKDYFPEPVTVSWNSGALTSGVHTFPAVLQSSGLYSLSSVV |
| | (N325S_L328F)-(G4S)3 | TVPSSSLGTQTYICNVNHKPSNTKVDKKVEPKSCDKTHTCPPCPAPE |
| | linker-VH-20AA linker- | LLGGPSVFLFPPKPKDTLMISRTPEVTCVVVDVSHEDPEVKFNWYVD |
| | VL mutations (H435R, | GVEVHNAKTKPREEQYNSTYRVVSVLTVLHQDWLNGKEYKCKVSSKA |
| | Y436F) | FPAPIEKTISKAKGQPREPQVYTLPPSRDELTKNQVSLSCAVKGFYP |
| | | SDIAVEWESNGQPENNYKTTPPVLDSDGSFFLVSKLTVDKSRWQQGN |
| | | VFSCSVMHEALHNRFTQKSLSLSPGK-GGGGSGGGGSGGGGS-X1- |
| | | GGSEGKSSGSGSESKSTGGS-X2, wherein X1 is the |
| | | amino acid sequence of any one of SEQ ID NOs: 6, |
| | | 8, 10, 12, 14, 16, 18, 20, 22, 24, 26, 28, 30, |
| | | 32, 34, 36, 38, 40, 42, 44, 46, 48, 194, 196, |
| | | 198, 200, 202, 204, 206, 208, 210, 212, 214, |
| | | 216, 218, 220, 222, and 224, 355, 357, 359, |
| | | 361, 363, 365, 367, 369, 371, 373, 375, 377, |
| | | 379, 381, 383, 385, 387, 389, 391, 393, 395, |
| | | 397, 399, 401, and 403, and wherein X2 is the |
| | | amino acid sequence of any one of SEQ ID |
| | | NOs: SEQ ID NO: 7, 9, 11, 13, 15, 17, 19, 21, 23, |
| | | 25, 27, 29, 31, 33, 35, 37, 39, 41, 43, 45, 47, |
| | | 49, 195, 197, 199, 201, 203, 205, 207, 209, |
| | | 211, 213, 215, 217, 219, 221, 223, 225, 356, |
| | | 358, 360, 362, 364, 366, 368, 370, 372, 374, |
| | | 376, 378, 380, 382, 384, 386, 388, 390, 392, |
| | | 394, 396, 398, 400, 402, and 404 |
| 193 | Isotype control heavy | EVRLLESGGGLVQPGGSLRLSCAASGFTFSNYAMGWVRQAPGKGLEW |
| | chain hlgG1 NSLF knob | VSAISGSGGSTYYADSVKGRFTTSRDDSKNALYLQMNSLRAEDTAVY |
| | (T366W) | YCARGGPGWYAADVWGQGTTVTVSSASTKGPSVFPLAPSSKSTSGGT |
| | (N325S_L328F) | AALGCLVKDYFPEPVTVSWNSGALTSGVHTFPAVLQSSGLYSLSSVV |
| | | TVPSSSLGTQTYICNVNHKPSNTKVDKKVEPKSCDKTHTCPPCPAPE |
| | | LLGGPSVFLFPPKPKDTLMISRTPEVTCVVVDVSHEDPEVKFNWYVD |
| | | GVEVHNAKTKPREEQYNSTYRVVSVLTVLHQDWLNGKEYKCKVSSKA |
| | | FPAPIEKTISKAKGQPREPQVYTLPPSRDELTKNQVSLWCLVKGFYP |
| | | SDIAVEWESNGQPENNYKTTPPVLDSDGSFFLYSKLTVDKSRWQQGN |
| | | VFSCSVMHEALHNHYTQKSLSLSPGK |

To generate the 2+1 bispecific antibodies, transient transfection of Expi293 cells (Invitrogen) was performed according to manufacturer's instructions. For 20 mL culture, 20 μg of total DNA consisting of 3 expression plasmids (heavy chain-hole-scFv sequence, heavy chain-knob sequence, and light chain sequence) were used. The molar ratio of the heavy chain-knob, the heavy chain-hole-scFv, and the light chain plasmids were optimized to 1:3:6 to achieve high purity of the 2+1 bispecific antibodies. The cell culture supernatant was harvested at 5 days post transfection. Clarified supernatants were purified using drip column with MabSelect Sure resin (Cytiva), washed with PBS, and eluted with pH 3.5 elution buffer and neutralized with Tris-HCl to a final pH of 5.5-6.0. Neutralized eluates containing the antibodies were dialyzed into PBS. Quantification of the antibody concentration was determined by measuring the absorbance at 280 nm using the Nanodrop 8000 (ThermoFisher) or Lunatic (Unchained Labs). Purity of the 2+1 bispecific antibodies was determined by SDS-PAGE. The 2+1 bispecific antibodies were analyzed with size exclusion chromatography (SEC) for aggregation. Next, 22 out of 24 antibodies were produced and purified as 2+1 bispecific antibodies. 22 of the 2+1 bispecific antibodies were analyzed via ELISA, FACS and internalization to confirm retention of binding in the 2+1 format (data not shown).

### Example 10: Generation and Screening of Anti-CD98hc Antibodies Using Single B-Cell Cloning

Anti-CD98hc antibodies were also generated via B-cell cloning technology. BALB/c mice (Charles River Laboratories, Wilmington, MA) were immunized twice a week by subcutaneous or intraperitoneal injections of purified extracellular domain polypeptides of human, cyno, and/or mouse CD98hc (as described above in Example 1) with or without adjuvant. After a total of 14 injections and three days following the final boost, the lymph nodes and spleens were harvested from the mice whose sera demonstrated strong binding by FACS to CHO cells overexpressing human or mouse CD98hc.

The tissues were processed into single cell suspensions and enriched for B-cells using a negative selection magnetic bead kit (Stemcell Technologies, Vancouver, BC, Canada, Cat #19844). Cells were blocked with anti-mouse CD16/CD32 (2.4G2, catalog #553142, BD Biosciences, San Jose, CA) and an unlabeled irrelevant Avi-His tagged protein. Cells were then stained with huCD98hc Avi-His protein labeled with AF488 and AF647 using Lightening link kits (Novus Biologics, catalog #ab236553, 336-0010, Littletown, CO), anti-mouse IgG BV605 (Poly4053, Biolegend, catalog #405327, San Diego, CA), and anti-mouse IgM BV786 (II/41, BD Bioscience, catalog #743328). Cells were washed and subsequently stained with anti-mouse CD19 BV421 (6D5, Biolegend, catalog #115537), anti-mouse CD138 BV421 (281-2, Biolegend, catalog #142508), anti-human CD3 PE (UCHT1, Biolegend, catalog #100206). Finally, viability dye 7-AAD (BD Biosciences, catalog #559925) was added prior to sorting. CD19-positive and/or CD138-positive plasmablast and memory B-cell populations that were IgM-negative, IgG-positive and human CD98hc-positive were bulk sorted using a FACS ARIA II cell sorter (BD Biosciences).

Sorted cells were processed for single cell B-cell receptor sequencing using the Chromium Single Cell 5′ Gel Beads v2 and loaded into 3 wells of a K chip for formation of Gel Beads-in-emulsion (GEMs) in the Chromium controller (10× Genomics, Pleasanton, CA, Cat #1000266 and 1000287). After GEM clean up, GEM-RT products were purified, and cDNA was amplified for antibody heavy and light chains using the Chromium Single Cell Mouse BCR Amplification Kit (10× Genomics, Cat #1000255). GEX and VDJ libraries were prepared following the manufacturer's instructions and assessed with an Agilent BioAnalyzer High Sensitivity DNA kit and quantified by qPCR (KAPA Library Quantification Kit, Cat #07960140001, Roche Sequencing, Wilmington, MA). Libraries were dual index sequenced using an Illumina 150-cycle kit on an Illumina MiSeq or NovaSeq (Illumina, San Diego, CA). Sequencing data was processed with the 10× Genomics Cell Ranger mkfastq, count, and vdj pipelines. The resulting VDJ sequences were post-processed to identify unique IgG pairs.

450 unique sequences were identified, and from these a panel of 94 sequences with diverse CDRH3 sequences were selected to synthesize and express as chimeras in the 2+1 bispecific antibody format.

To generate the 2+1 bispecific antibodies, transient transfection of Expi293 cells (Invitrogen) were performed in 96-well deep well blocks according to the manufacturer's instructions. For 0.8 mL culture in each well of the 96 deep well block, 0.8 μg of total DNA consisting of three expression plasmids (heavy chain-hole-scFv, heavy chain-knob, and light chain) were used. The molar ratio of the three types of plasmids (heavy chain-knob, heavy chain-hole-scFv, and light chain plasmids) were optimized to 1:3:6 to achieve high purity of 2+1 bispecific antibodies. The cell culture supernatant was harvested at 5 days post transfection. Clarified supernatants were purified using ProPlus Phytip columns on a Hamilton STAR platform as described in Example 7. The concentrations of the purified antibodies were determined by measuring the absorbance at 280 nm using the Nanodrop 8000 (ThermoFisher).

Purified 2+1 anti-CD98hc bispecific antibodies were screened for binding to human and cyno CD98hc by ELISA as in Example 6. Purified 2+1 bispecific antibodies were screened at concentrations ranging from 1-30 μg/mL. ELISA binding to human and cyno CD98 and control cyno TfR AVI-His tagged proteins as a ratio to secondary antibody alone were calculated, and values are listed in Table 10A. Purified 2+1 bispecific antibodies were also screened for their ability to bind CHO cells overexpressing human or mouse CD98hc or cells endogenously expressing human CD98hc by FACS. Antibodies were incubated with transfected CHO, parental CHO, or HCMEC/D3, washed and then stained with goat anti human IgG Fc (Jackson Immunoresearch, 109-136-098) and labeled with fixable viability eFluor 450 (eBioscience, catalog 65-0863-14). Data was acquired on a FACS Canto (BD Biosciences). The ratio of mean fluorescence intensity of binding to transfected cell lines over parental CHO is indicated in Table 12B. The ratio of binding to endogenously expressing human brain endothelial cells over secondary only is also in Table 12B. 47 out of 94 of the 2+1 anti-CD98hc bispecific antibodies screened were positive by ELISA with signal at least 5-fold higher than the secondary alone, and 16 were ELISA and FACS positive which FACS binding increased 3-fold or more compared to non-expressing cells. Sequences for the 2+1 anti-CD98hc anti-CD98hc bispecific antibodies that were ELISA and FACS binding are shown in Table 13. CDR sequences (Kabat) are shown in Table 14 and Table 15.

TABLE 12A
ELISA Characterization of 2 + 1 Anti-CD98hc Bispecific
Antibodies Derived From Single B-cell Cloning

| | Hu- | cyno | |
|---|---|---|---|
| | CD98hc/ | CD98hc/ | cyno Tfr/ |
| Antibody | 2nd only | 2nd only | 2nd only |
| CD98hc.05.006 | 38 | 16 | 1 |
| CD98hc.05.007 | 38 | 31 | 1 |
| CD98hc.05.046 | 25 | 6 | 1 |
| CD98hc.05.052 | 27 | 19 | 1 |
| CD98hc.05.059 | 32 | 19 | 1 |
| CD98hc.05.081 | 25 | 6 | 1 |
| CD98hc.05.126 | 36 | 14 | 1 |
| CD98hc.05.144 | 24 | 6 | 1 |
| CD98hc.05.153 | 10 | 1 | 1 |
| CD98hc.05.166 | 31 | 4 | 1 |
| CD98hc.05.168 | 28 | 9 | 1 |
| CD98hc.05.188 | 17 | 1 | 1 |
| CD98hc.05.193 | 28 | 10 | 1 |
| CD98hc.05.207 | 23 | 7 | 1 |
| CD98hc.05.208 | 5 | 1 | 1 |
| CD98hc.05.226 | 25 | 6 | 1 |
| Anti-TfR 2 + 1 iso control | 1 | 1 | 11 |
| Secondary antibody only | 1 | 1 | 1 |

TABLE 12B
FACS Characterization of Anti-CD98hc 2 + 1 Bispecific
Antibodies Derived From Single B-cell Cloning

Ratio MFI IgG APC

| | CHO | |
|---|---|---|
| | huCD98 / | |
| | CHO | HCMEC/ |
| Antibody | parental | 2nd ab only |
| CD98Hc.05.006 | 31 | 603 |
| CD98Hc.05.007 | 24 | 403 |
| CD98Hc.05.046 | 9 | 98 |
| CD98Hc.05.052 | 24 | 272 |
| CD98Hc.05.059 | 21 | 139 |
| CD98Hc.05.081 | 8 | 136 |
| CD98Hc.05.126 | 30 | 316 |
| CD98Hc.05.144 | 32 | 405 |
| CD98Hc.05.153 | 6 | 80 |
| CD98Hc.05.166 | 9 | 60 |
| CD98Hc.05.168 | 18 | 204 |
| CD98Hc.05.188 | 16 | 260 |
| CD98Hc.05.193 | 23 | 526 |
| CD98Hc.05.207 | 17 | 215 |
| CD98Hc.05.208 | 3 | 33 |
| CD98Hc.05.226 | 24 | 490 |
| Positive Control | 40 | 1095 |
| Ab170 | | |
| Anti-TfR 2 + 1 | 1 | 50 |
| isotype control | | |

TABLE 13
Antibody Sequences of Anti-CD98hc 2 + 1 Bispecific Antibodies From Single B-cell
Cloning

| Antibody | | |
|---|---|---|
| Name | Heavy Chain Variable Region | Light Chain Variable Region |
| CD98hc.05.006 | EVKLVESGGNLVQPGGSLKLSCAASGFTFS | DIVMSQSPSSLTVSVGAKIILGCKSSQSLLY |
| | GYSMSWVRQTPDKRLEWVAYIGNGGDSTYY | SGNQKNYLAWYQQKPGQSPKLLIYWASTRES |
| | PYTVKGRFTISRDNAKNTLYLQMNSLKSED | GVPDRFTGSGSGTDFTLTISSVKAEDVAVYY |
| | SAMYYCARQYVNYPFFDYWGQGTTLTVSS | CQQYYTYPPAFGAGTKLEIK |
| | (SEQ ID NO: 194) | (SEQ ID NO: 195) |
| CD98hc.05.007 | DVKLVESGGDLVKPGGSLKLSCAASGFTFS | DILMTQSPSSMSASLGDTVSITCHASQGISS |
| | SYSMSWVRQTPEKRLEWVATITSGGSYTYF | NIGWLQQKPGKSFKGLIYRGTNLEDGVPSRF |
| | PDSVKGRFTISRDNAKNTLYLQVSNLKSED | SGSGSGADYSLTISSLESEDFADYYCVQYAH |
| | TAMYYCTRGPSFGNYLDYWGQGTTLTVSS | FPYTFGGGTKLEIK |
| | (SEQ ID NO: 196) | (SEQ ID NO: 197) |
| CD98hc.05.046 | EVQLVESGGGLVKPGGSLKLSCAASGFTFS | DVVMTQTPLSLPVSLGNQASISCRSSQSLVH |
| | DYYMYWVRQTPEKRLEWVATISDGGSYTYY | SNGNTFFHWYLQKPGQSPKLLIYKVSNRFSG |
| | PDSVKGRFTISRDNAKNNLYLQMSSLKSED | VPDRFSGSGSGTDFTLKISRVEAEDLGLYFC |
| | TAMYYCARDRAYYYGSSYGFGYWGQGTLVT | SQTTHVPPTFGGGTKLEIK |
| | VSS | (SEQ ID NO: 199) |
| | (SEQ ID NO: 198) | |
| CD98hc.05.052 | DMQLQESGPGLVKPSQSLSLTCTVTGYSIT | QFVLTQSPAIMSASPGEKVTISCSASSSVSY |
| | SDYAWNWIRQFPGNKLEWMGFISYSGTTSY | MFWYQQKPGSSPKPWISHTSNLASGVPARFS |
| | TPSLKRRISITRDTSKNQFFLRLNSVTPED | GSGSGTSYSLTISNMEAEDAATYYCQQFHDY |
| | TATYYCAKSAFYGNVWFAYWGQGTLVTVSS | PWTFGGGTKLEIK |
| | (SEQ ID NO: 200) | (SEQ ID NO: 201) |
| CD98hc.05.059 | EIQLQQSGPELVKPGASVKVSCKASGYSFN | QIVLTQSPAIMSASLGERVTMTCTASSSVTS |
| | MFWVKQSHGKSLEWIGHIDPFNGDTSYSPN | SYLYWYQQKPGSSPKLWIYSTSNLASGVPPR |
| | FEDRASLTVDRSSNTAYMHLNSLTSEDSAV | FSGSGSGTSYSLTISSMEAEDAATYHCQQYH |
| | YYCARSVGAHWYFDVWGAGTTVTVSS | RSPATFGAGTKLEIK |
| | (SEQ ID NO: 202) | (SEQ ID NO: 203) |
| CD98hc.05.081 | EVQLQQSRPELVKPGASVKMSCKASGYTFT | DIVMTQFPATLSVTPGDRVSLSCRASQSISN |
| | SYVMHWVKQKPGQGLEWIGYISPYNGDTKY | YLHWYQQKSHESPRLLIKYASQSISGIPSRF |
| | NEKFKGKATLTSDISSTTAYMELSSLTSED | SGSGSGSDFTLSINSVEPEDVGVYYCQNGHS |
| | SGVYYCARPGSSPSWFAYWGQGTLVTVSS | FPYMYTFGGGTKLEIK |
| | (SEQ ID NO: 204) | (SEQ ID NO: 205) |
| CD98hc.05.126 | EVKLVESGGNLVQPGGSLKLSCAASGFTFS | DFVMSQSPSSLTVSVGAKVIMSCKSSQSLLY |
| | GYPMSWVRRTPDKRLEWVAYIGSGGDSTYY | SGNQKNYLAWYQQKPGQSPKLLIYWASTRES |
| | PYAVKGRFTISRDNAKNTLYLQMNSLKSED | GVPDRFTGSGSGTDFTLTISSVKAEDLAVYY |
| | SAMYYCARQYDNYPFFDYWGQGTTLTVSS | CQQYYTYPPTFGAGTKLEIK |
| | (SEQ ID NO: 206) | (SEQ ID NO: 207) |
| CD98hc.05.144 | EVQLVESGGALVKPGGSLKLSCAASGFAFS | DFVMSQSPSSLTVSVGEKVTMTCKSSQSLLY |
| | SFDMSWVRQTPERRVEWVAYISGGGGSTYH | SYTQKNYLAWYQQKPGQSPNLLIYWASTRES |
| | PDTVKGRFAISRDNAKNTLYLHMNSLKSED | GVPDRFAGSGSGTDFTLTISSVEAEDLAVYY |
| | TAMYYCVRQGSRPWFAYWGQGTLVTVSS | CQQYSNYPPTFGGGTKLEIK |
| | (SEQ ID NO: 208) | (SEQ ID NO: 209) |
| CD98hc.05.153 | EVQLVESGGGLVKPGGSLKLSCAASGFDFS | DIVMSQSPSFLDVSVGEKVAMTCKSIQSLLY |
| | NCDMSWVRQTPEKRLEWVAYISNSGDSTYF | SGNQKNYLAWYQQKPGQSPKVLIYWASTRES |
| | PHTVKGRFTISRDNAKNALYLQMSSLKSED | GVPDRFTGSGSGTDFTLTISSVKAEDLAVYY |
| | TAMYYCARQGGHYPWFVYWGQGTLVTVSS | CQQYYTYPPTFGGGTKLEIK |
| | (SEQ ID NO: 210) | (SEQ ID NO: 211) |
| CD98hc.05.166 | EVQLLQSGAELVKPGASVKLSCTASGFNIL | DIVMTQSHKFMSTSLGDRVSITCKASQGVFT |
| | DTYIHWVMQRPEQGLEWIGRIDPANGHVKY | AVAWYQQKPGQSPKLLIYSASYRYTGVPDRF |
| | DPKFQGKATITADTSSNTAYLQLSSLTSED | TGSGSGTDFTFTISSVQAEDLAVYYCQQHYS |
| | TAVYYCASPFTTVVATNIYYYGLGYWGHGT | TPWTFGGGTKLEIK |
| | SVTVSS | (SEQ ID NO: 213) |
| | (SEQ ID NO: 212) | |
| CD98hc.05.168 | EVQLVESGGGLVKPGGSLKLSCAASGFAFS | DIVMSQSPSSLAVSVGEKVTVSCKSSQNLLY |
| | TYDMSWVRQTPEKRLEWVAYISSGGDTTYY | SSNQKNYLAWYQQKPGQSPKLLIYWASTRES |
| | PDTVKGRFTISRDNAKNTLYLQMSSLKSED | GVPDRFTGSGSGTDFTLTISSVKAEDLAVYY |
| | SAIYYCARQDYRPYFAYWGQGTLVTVSS | CQQYSYYPPTFGGGTKLEIK |
| | (SEQ ID NO: 214) | (SEQ ID NO: 215) |
| CD98hc.05.188 | QVQLQQPGAELVRPGASVKLSCKASGYTFT | DIVMTQSPSSLAMSVGQKVTMSCKSSQSLLN |
| | SYWMSWVKQRPGQGLEWIGFITPSDSEALY | SNNQKNYLAWYQQKPGQSPKLLVYFSSTRES |
| | SQMFKDKATLTVDKSSSTAHLQLSSLTSED | GVPDRFIGSGSGTDFTLTITSVQAEDLADYF |
| | SAVYYCARWSYGNFAAMDYWGQGTSVTVSS | CQQHYSTPLTFGAGTKLEIK |
| | (SEQ ID NO: 216) | (SEQ ID NO: 217) |
| CD98hc.05.193 | EVQLVESGGGLVQPGGSLKVSCTTSGFTFS | DIVMSQSPSSLAVSVGEKVTMTCKSSQSLLY |
| | DFDMSWIRQTPEKRLEWVAYISSGGGSTYF | SSNQKNYLAWYQQKPGQSPKLLIYWASTRES |
| | PDTVKGRFTISRDNAKSTLYLQMSSLKSED | GVPDRFTGSGSGTDFTLTISSVKAEDLALYY |
| | TAMYYCARHVGVRPWFDYWGQGTLVTVSS | CQQYHYYPPTFGAGTKLEIK |
| | (SEQ ID NO: 218) | (SEQ ID NO: 219) |
| CD98hc.05.207 | EVQLVESGGGLVKPGGSLKISCAASGFAFS | DIVMSQSPSSLAVSVGEKVTLSCKSSQSLFY |
| | SYDMSWVRQTPEKRLEWVAYISSAGGSTYF | SGNQKTYLAWYQQKPGQSPKLLIYWTSTRES |
| | PGTVKGRFTISRDNAKNTLYLQMRSLKSED | GVPDRFTGSGSGTDFTLTISSVKAEDLAVYF |
| | TAMYYCARHENGHYPFFDFWGQGTTLTVSS | CQQYYTYPPTFGAGTKLEIK |
| | (SEQ ID NO: 220) | (SEQ ID NO: 221) |
| CD98hc.05.208 | QVQLQQSGPELVRPGVSVKISCKSSGYAFT | DIVMTQSPSSLAISVGQKVTMSCKSSQSLLN |
| | DYAVHWVRQSRAMSLEWIGIISTYSGNIDY | RNNQKNYLAWYQQKPGQSPKLLLYFTSTRES |
| | NQQFKGKATMTVDKSSSTVYMELARLTSED | GVPDRFIGSGSGTDFTLIISSVQAEDLADYF |
| | SAIYFCARGGRHYYGSYYYAFDYWGQGTSV | CQQHYSTPPTFGGGTKLEIK |
| | TVSS | (SEQ ID NO: 223) |
| | (SEQ ID NO: 222) | |
| CD98hc. 05.226 | EVQLVESGGGLGKPGGSLKLSCAASGFAFS | DIVMSQSPSSLAVSVGEKITMTCKSSQSLLY |
| | SYDMSWVRQTPEKRLDWVAYISSGGGGTYY | SGSQKNYLAWYQQKPGQSPKLLIYWASTRES |
| | PDTVKGRFTISRDNAKNTLYLQMSSLKSED | GVPDRFTGSGSGTDFTLTISSVKAEDLAVYY |
| | TAIYYCARHVGVRPWFGYWGQGTLVTVSS | CQQYSYYPPTFGAGTKLEIK |
| | (SEQ ID NO: 224) | (SEQ ID NO: 225) |

TABLE 14
Heavy Chain CDR Sequences of Anti-CD98hc 2 + 1 Bispecific Antibodies from
Single B-cell Cloning

| Antibody | CDR-H1 | CDR-H2 | CDR-H3 |
|---|---|---|---|
| CD98hc.05.006 | GYSMS | YIGNGGDSTYYPYTVKG | QYVNYPFFDY |
| | (SEQ ID NO: 226) | (SEQ ID NO: 227) | (SEQ ID NO: 228) |
| CD98hc.05.007 | SYSMS | TITSGGSYTYFPDSVKG | GPSFGNYLDY |
| | (SEQ ID NO: 229) | (SEQ ID NO: 230) | (SEQ ID NO: 231) |
| CD98hc.05.046 | DYYMY | TISDGGSYTYYPDSVKG | DRAYYYGSSYGFGY |
| | (SEQ ID NO: 232) | (SEQ ID NO: 233) | (SEQ ID NO: 234) |
| CD98hc.05.052 | SDYAWN | FISYSGTTSYTPSLKR | SAFYGNVWFAY |
| | (SEQ ID NO: 235) | (SEQ ID NO: 236) | (SEQ ID NO: 237) |
| CD98hc.05.059 | SFNMF | HIDPFNGDTSYSPNFED | SVGAHWYFDV |
| | (SEQ ID NO: 238) | (SEQ ID NO: 239) | (SEQ ID NO: 240) |
| CD98hc.05.081 | SYVMH | YISPYNGDTKYNEKFKG | PGSSPSWFAY |
| | (SEQ ID NO: 241) | (SEQ ID NO: 242) | (SEQ ID NO: 243) |
| CD98hc.05.126 | GYPMS | YIGSGGDSTYYPYAVKG | QYDNYPFFDY |
| | (SEQ ID NO: 244) | (SEQ ID NO: 245) | (SEQ ID NO: 246) |
| CD98hc.05.144 | SFDMS | YISGGGGSTYHPDTVKG | QGSRPWFAY |
| | (SEQ ID NO: 247) | (SEQ ID NO: 248) | (SEQ ID NO: 249) |
| CD98hc.05.153 | NCDMS | YISNSGDSTYFPHTVKG | QGGHYPWFVY |
| | (SEQ ID NO: 250) | (SEQ ID NO: 251) | (SEQ ID NO: 252) |
| CD98hc.05.166 | DTYIH | RIDPANGHVKYDPKFQG | PFTTVVATNIYYYGLG |
| | (SEQ ID NO: 253) | (SEQ ID NO: 254) | Y |
| | | | (SEQ ID NO: 255) |
| CD98hc.05.168 | TYDMS | YISSGGDTTYYPDTVKG | QDYRPYFAY |
| | (SEQ ID NO: 256) | (SEQ ID NO: 257) | (SEQ ID NO: 258) |
| CD98hc.05.188 | SYWMS | FITPSDSEALYSQMFKD | WSYGNFAAMDY |
| | (SEQ ID NO: 259) | (SEQ ID NO: 260) | (SEQ ID NO: 261) |
| CD98hc.05.193 | DFDMS | YISSGGGSTYFPDTVKG | HVGVRPWFDY |
| | (SEQ ID NO: 262) | (SEQ ID NO: 263) | (SEQ ID NO: 264) |
| CD98hc.05.207 | SYDMS | YISSAGGSTYFPGTVKG | HENGHYPFFDF |
| | (SEQ ID NO: 265) | (SEQ ID NO: 266) | (SEQ ID NO: 267) |
| CD98hc.05.208 | DYAVH | IISTYSGNIDYNQQFKG | GGRHYYGSYYYAFDY |
| | (SEQ ID NO: 268) | (SEQ ID NO: 269) | (SEQ ID NO: 270) |
| CD98hc.05.226 | SYDMS | YISSGGGGTYYPDTVKG | HVGVRPWFGY |
| | (SEQ ID NO: 265) | (SEQ ID NO: 271) | (SEQ ID NO: 272) |

TABLE 15
Light Chain CDR Sequences of Anti-CD98hc 2 + 1 Bispecific Antibodies from
Single B-cell Cloning

| Antibody | CDR-L1 | CDR-L2 | CDR-L3 |
|---|---|---|---|
| CD98hc.05.006 | KSSQSLLYSGNQKNYL | WASTRES | QQYYTYPPA |
| | A | (SEQ ID NO: 274) | (SEQ ID NO: 275) |
| | (SEQ ID NO: 273) | | |
| CD98hc.05.007 | HASQGISSNIG | RGTNLED | VQYAHFPYT |
| | (SEQ ID NO: 276) | (SEQ ID NO: 277) | (SEQ ID NO: 278) |
| CD98hc.05.046 | RSSQSLVHSNGNTFFH | KVSNRFS | SQTTHVPPT |
| | (SEQ ID NO: 279) | (SEQ ID NO: 280) | (SEQ ID NO: 281) |
| CD98hc.05.052 | SASSSVSYMF | HTSNLAS | QQFHDYPWT |
| | (SEQ ID NO: 282) | (SEQ ID NO: 283) | (SEQ ID NO: 284) |
| CD98hc.05.059 | TASSSVTSSYLY | STSNLAS | QQYHRSPAT |
| | (SEQ ID NO: 285) | (SEQ ID NO: 286) | (SEQ ID NO: 287) |
| CD98hc.05.081 | RASQSISNYLH | YASQSIS | QNGHSFPYMYT |
| | (SEQ ID NO: 288) | (SEQ ID NO: 289) | (SEQ ID NO: 290) |
| CD98hc.05.126 | KSSQSLLYSGNQKNYL | WASTRES | QQYYTYPPT |
| | A | (SEQ ID NO: 274) | (SEQ ID NO: 291) |
| | (SEQ ID NO: 273) | | |
| CD98hc.05.144 | KSSQSLLYSYTQKNYL | WASTRES | QQYSNYPPT |
| | A | (SEQ ID NO: 274) | (SEQ ID NO: 293) |
| | (SEQ ID NO: 292) | | |
| CD98hc.05.153 | KSIQSLLYSGNQKNYLA | WASTRES | QQYYTYPPT |
| | (SEQ ID NO: 294) | (SEQ ID NO: 274) | (SEQ ID NO: 291) |
| CD98hc.05.166 | KASQGVFTAVA | SASYRYT | QQHYSTPWT |
| | (SEQ ID NO: 295) | (SEQ ID NO: 296) | (SEQ ID NO: 297) |
| CD98hc.05.168 | KSSQNLLYSSNQKNYL | WASTRES | QQYSYYPPT |
| | A | (SEQ ID NO: 274) | (SEQ ID NO: 299) |
| | (SEQ ID NO: 298) | | |
| CD98hc.05.188 | KSSQSLLNSNNQKNYL | FSSTRES | QQHYSTPLT |
| | A | (SEQ ID NO: 301) | (SEQ ID NO: 302) |
| | (SEQ ID NO: 300) | | |
| CD98hc.05.193 | KSSQSLLYSSNQKNYL | WASTRES | QQYHYYPPT |
| | A | (SEQ ID NO: 274) | (SEQ ID NO: 304) |
| | (SEQ ID NO: 303) | | |
| CD98hc.05.207 | KSSQSLFYSGNQKTYL | WTSTRES | QQYYTYPPT |
| | A | (SEQ ID NO: 306) | (SEQ ID NO: 291) |
| | (SEQ ID NO: 305) | | |
| CD98hc.05.208 | KSSQSLLNRNNQKNYL | FTSTRES | QQHYSTPPT |
| | A | (SEQ ID NO: 308) | (SEQ ID NO: 309) |
| | (SEQ ID NO: 307) | | |
| CD98hc.05.226 | KSSQSLLYSGSQKNYL | WASTRES | QQYSYYPPT |
| | A | (SEQ ID NO: 274) | (SEQ ID NO: 299) |
| | (SEQ ID NO: 310) | | |

### Example 11: ELISA Binding of 2+1 Anti-CD98hc Bispecific Antibodies

To determine binding retention of reformatted 2+1 anti-CD98hc bispecific antibodies by ELISA, high binding ELISA plates (Thermo Fisher) were coated with streptavidin (1 μg/ml, Thermo Fisher) overnight at 4° C. Plates were then washed three times with phosphate buffered saline with 0.05% tween-20 (PBST) and incubated with block buffer (PBS+1% BSA, 1 h, room temperature). Plates were washed three times again with PBST and incubated with biotinylated human, cyno, and mouse CD98hc and an irrelevant antigen, human transferrin receptor (1 μg/ml, 1 h, room temperature). Plates were washed three times again with PBST and incubated with anti-CD98hc antibodies (1 μg/ml, 1 h, room temperature). Plates were washed three times again with PBST and incubated with a secondary detection antibody, anti-human horseradish peroxidase conjugated antibody (1:5000 dilution, 30 min, room temperature). Plates were washed three times one last time and incubated with TMB-ELISA Substrate Solution (Thermo Fisher) and then quenched with H₂SO₄. Anti-CD98hc binding was then analyzed by quantifying optical density at 450 nm (OD₄₅₀) using the SpectraMax M5 (Molecular Devices). The binding characteristics of the various 2+1 anti-CD98hc bispecific antibodies to CD98hc (human, cyno, mouse) and control human TfR are summarized below in Table 16, shown as fold-change relative to isotype hIgG. All 2+1 anti-CD98hc antibodies showed binding specificity to human CD98hc compared to mouse CD98hc and the control, and cross-reactive binding to cyno CD98hc to varying degrees.

TABLE 16
ELISA OD 450 values of reformatted 2
+ 1 anti-CD98hc bispecific antibodies

| | | | | Human |
|---|---|---|---|---|
| | Human | Cyno | Mouse | TfR-AVI- |
| 2 + 1 Bispecific | CD98hc- | CD98hc- | CD98hc- | His |
| Antibody | AVI-His | AVI-His | AVI-His | (control) |
| CD98hc.03.002 | 25.0 | 24.1 | 1.6 | 1.3 |
| CD98hc.03.004 | 28.5 | 25.1 | 4.0 | 1.5 |
| CD98hc.03.010 | 25.7 | 21.2 | 1.2 | 1.2 |
| CD98hc.03.031 | 19.6 | 18.5 | 1.7 | 1.4 |
| CD98hc.04.003 | 30.1 | 28.0 | 1.1 | 1.1 |
| CD98hc.04.006 | 27.9 | 24.4 | 2.3 | 2.3 |
| CD98hc.04.009 | 14.9 | 10.8 | 1.1 | 1.8 |
| CD98hc.04.010 | 27.7 | 29.2 | 1.2 | 2.3 |
| CD98hc.04.011 | 2.7 | 2.1 | 1.4 | 1.2 |
| CD98hc.04.012 | 29.2 | 21.4 | 1.5 | 1.3 |
| CD98hc.04.013 | 23.8 | 23.6 | 2.0 | 1.6 |
| CD98hc.04.018 | 23.6 | 22.7 | 1.1 | 1.1 |
| CD98hc.04.022 | 19.6 | 12.6 | 1.1 | 1.2 |
| CD98hc.04.045 | 21.9 | 20.1 | 1.1 | 1.1 |
| CD98hc.04.046 | 16.2 | 17.2 | 1.1 | 1.1 |
| CD98hc.04.047 | 9.8 | 4.1 | 1.3 | 1.3 |
| CD98hc.04.054 | 25.0 | 30.7 | 1.4 | 1.2 |
| CD98hc.04.058 | 26.7 | 16.8 | 3.9 | 3.0 |
| CD98hc.04.062 | 26.0 | 23.4 | 2.6 | 2.2 |
| CD98hc.04.063 | 11.1 | 9.1 | 1.2 | 1.1 |
| CD98hc.04.064 | 26.5 | 17.3 | 1.1 | 1.1 |
| CD98hc.04.065 | 16.4 | 7.4 | 2.0 | 2.0 |

### Example 12: FACS Binding of 2+1 Anti-CD98hc Bispecific Antibodies

To determine binding retention of 2+1 anti-CD98hc bispecific antibodies by FACS, antibodies were evaluated for binding to HCMEC/D3 cells (a cell line derived from human brain endothelial cells), overexpressing human and mouse CD98hc CHO cell lines, and parental CHO cells for non-specific binding. Cells were seeded in tissue culture plates (50,000 cells/well) and washed and resuspended in FACS buffer (PBS+2% BSA+1 mM EDTA). The 2+1 anti-CD98hc bispecific antibodies were then incubated with cells (5 μg/ml, 1 hr, on ice) and washed twice with FACS buffer. Cells were then incubated with allophycocyanin conjugated anti-mouse secondary antibody (Jackson Immunoresearch) (1:1000, 30 min, on ice) and then washed twice with FACS buffer. Next, cells were resuspended in FACS buffer supplemented with 0.5% propidium iodide (Thermo Fisher). Anti-CD98hc binding was then analyzed on the iQue flow cytometer (IntelliCyt) by measuring the Mean Fluorescent Intensity (MFI). FACS data are summarized below in Table 17, shown as MFI fold-change relative to isotype hIgG1 control. All 2+1 anti-CD98hc antibodies showed binding specificity to hCMEC/D3 cells and to CHO-huCD98hc cell lines to a lesser degree.

TABLE 17
FACS MFI Fold-Change of Reformatted 2
+ 1 anti-CD98hc Bispecific Antibodies

| 2 + 1 Bispecific | hCMEC/ | CHO- | CHO- | CHO |
|---|---|---|---|---|
| Antibody | D3 | huCD98hc | mCD98hc | parental |
| CD98hc.03.002 | 14.5 | 4.7 | 0.6 | 0.8 |
| CD98hc.03.004 | 127.5 | 43.7 | 1.0 | 0.9 |
| CD98hc.03.010 | 6.9 | 2.8 | 1.2 | 0.8 |
| CD98hc.03.031 | 59.2 | 17.6 | 1.4 | 0.9 |
| CD98hc.04.003 | 5.3 | 3.0 | 0.5 | 0.6 |
| CD98hc.04.006 | 80.5 | 13.0 | 1.5 | 0.9 |
| CD98hc.04.009 | 1.5 | 0.9 | 0.3 | 0.7 |
| CD98hc.04.010 | 42.2 | 16.0 | 0.6 | 0.6 |
| CD98hc.04.011 | 27.5 | 4.4 | 0.7 | 0.8 |
| CD98hc.04.012 | 96.7 | 30.9 | 1.2 | 0.8 |
| CD98hc.04.013 | 82.1 | 26.4 | 1.3 | 0.9 |
| CD98hc.04.018 | 53.4 | 14.8 | 0.7 | 0.8 |
| CD98hc.04.022 | 17.9 | 5.6 | 1.1 | 0.8 |
| CD98hc.04.045 | 1.5 | 0.9 | 0.4 | 0.7 |
| CD98hc.04.046 | 5.6 | 3.9 | 0.8 | 0.6 |
| CD98hc.04.047 | 7.5 | 2.9 | 0.6 | 0.7 |
| CD98hc.04.054 | 114.7 | 30.6 | 1.8 | 0.9 |
| CD98hc.04.058 | 26.3 | 11.8 | 3.8 | 1.6 |
| CD98hc.04.062 | 136.1 | 40.3 | 1.6 | 1.1 |
| CD98hc.04.063 | 39.8 | 6.1 | 1.0 | 0.9 |
| CD98hc.04.064 | 108.3 | 29.5 | 0.4 | 0.7 |
| CD98hc.04.065 | 25.1 | 7.4 | 2.1 | 1.2 |

### Example 13: In Vitro Internalization of 2+1 Anti-CD98hc Bispecific Antibodies into a Blood-Brain Barrier Endothelial Cell Line

Internalization of 2+1 anti-CD98hc bispecific antibodies into hCMEC/D3 cells was determined as described above in Example 7. An anti-TfR hIgG1 antibody was included as a positive control, and an isotype hIgG1 antibody was included as a negative control. The fold-change of total integrated intensity of pHrodo-red signal per well relative to isotype hIgG1 was calculated and summarized in Table 18 below. Table 168shows that 22 of the 2+1 anti-CD98hc bispecific antibodies that retained target binding also showed internalization. All 2+1 anti-CD98hc antibodies were internalized into hCMEC/D3 cells at varying degrees.

TABLE 18
Internalization values of 2 + 1 anti-CD98hc bispecific antibodies

| | Total integrated |
|---|---|
| 2 + 1 Bispecific | intensity fold- |
| Antibody | change |
| CD98hc.03.002 | 338.5 |
| CD98hc.03.004 | 342.8 |
| CD98hc.03.010 | 81.4 |
| CD98hc.03.031 | 16.8 |
| CD98hc.04.003 | 275.1 |
| CD98hc.04.006 | 626.2 |
| CD98hc.04.009 | 349.0 |
| CD98hc.04.010 | 520.1 |
| CD98hc.04.011 | 310.5 |
| CD98hc.04.012 | 330.2 |
| CD98hc.04.013 | 426.2 |
| CD98hc.04.018 | 809.9 |
| CD98hc.04.022 | 142.0 |
| CD98hc.04.045 | 251.2 |
| CD98hc.04.046 | 340.6 |
| CD98hc.04.047 | 260.2 |
| CD98hc.04.054 | 330.4 |
| CD98hc.04.058 | 878.1 |
| CD98hc.04.062 | 568.5 |
| CD98hc.04.063 | 466.2 |
| CD98hc.04.064 | 130.8 |
| CD98hc.04.065 | 250.8 |
| anti-TfR hIgG1 | 231.2 |
| isotype hIgG1 | 1.0 |

### Example 14: Epitope Binning of 2+1 Anti-CD98hc Bispecific Antibodies

Epitope binning analysis was performed on 2+1 anti-CD98hc bispecific antibodies by performing a classical sandwich experiment using a Carterra LSA instrument (Carterra, Salt Lake City, UT). Briefly, an HC200M was used for the kinetic evaluations, then tested in a binning assay, in which the immobilized antibodies were tested for their ability to form sandwich pairs with recombinant huCD98hc Avi-His and injected antibodies. For each cycle, 200 nM of huCD98hc Avi-His was injected over the chip for five minutes, followed by a test antibody (diluted to 40 μg/ml in running buffer) for five minutes, then by two 30-second injections of Pierce™ IgG Elution Buffer (ThermoScientific), for regeneration. Data were processed and analyzed using Epitope high-throughput binning analysis software (Carterra). Antibodies that were able to bind antigen captured by an immobilized antibody were designated as “sandwich” or “pairing” antibodies, and these antibodies were assigned into a different epitope bin from that of the immobilized antibody. A matrix of pairing and non-pairing antibodies was constructed from the binding results of these experiments, which allowed for an epitope bin landscape of the 2+1 anti-CD98hc bispecific antibodies to be generated. Some of antibodies have overlapping binning profiles suggesting that they may recognize adjacent, but not completely overlapping epitopes. Slight heterogeneity within bins is indicated by adding letters such as a, b, c, and d. For antibodies that appear to overlap two epitope bins, the numbers are designated with an underscore to indicate the overlapping bins. The epitope bins are summarized in Table 19. “Uniques” mean the 2+1 anti-CD98hc bispecific antibody did not share an epitope with any other antibodies (tested in the panel).

TABLE 19
Epitope bins of 2 + 1 anti-CD98hc bispecific antibodies

| Bin | Sample ID |
|---|---|
| 1a | CD98hc.04.009, −.010, −.013, −.018, .064 |
| 1b | CD98hc.04.046, −.022 |
| 1c | CD98hc.04.054 |
| 1d | CD98hc.04.062 |
| le | CD98hc.03.010 |
| 1f | CD98hc.04.047, −.065 |
| 1_2 | CD98hc.04.011 |
| 1_2d | CD98hc.04.063 |
| 2a | CD98hc.04.003 |
| 2b | CD98hc.04.045, CD98hc.03.002 |
| 2c | CD98hc.04.012 |
| 2d | CD98hc.03.031, CD98hc.04.058 |
| Uniques | CD98hc.03.004, CD98hc.04.006 |

### Example 15: Binding Kinetics of 2+1 Anti-CD98hc Bispecific Antibodies

Binding kinetics of 2+1 anti-CD98hc bispecific antibodies (from the hybridoma campaigns) to human and cyno CD98hc Avi-His were evaluated using a Carterra LSA instrument (Carterra, Salt Lake City, UT). The 2+1 anti-CD98hc bispecific antibodies were prepared in duplicates by diluting 50-fold into 10 mM Acetate, pH 5.0 (Carterra) to give final concentration of 20 μg/mL. An HC200M sensor chip (Carterra) was activated using the single channel flow cell with a 7-minute injection of a 1:1:1 mixture of 100 mM MES pH 5.5, 100 mM sulfo-NHS, 400 mM EDC (all reconstituted in MES pH 5.5; 100 μl of each mixed in vial immediately before running assay). After switching to the multi-channel array flow cell, the antibodies were injected over the activated chip in four 96-spot arrays for 15 minutes each. The remaining unconjugated active groups on the chip were then blocked by injecting 1M Ethanolamine pH 8.5 (Carterra) for 7 minutes using the single channel flow cell. The resulting sensor chip contained eight spots for each antibody, at four different densities. Two independent experiments were performed as follows, resulting in an N of between one and eight determinations for each antibody. Spots that yielded less than 20 RU of analyte binding were excluded from further analysis.

After priming with running buffer HBS-EP+ (Teknova) with 0.5 mg/ml BSA (MP Biomedicals), the immobilized 2+1 anti-CD98hc bispecific antibodies were tested for their ability to bind to several forms of CD98hc Avi-His, including human and cyno orthologs described above. Estimates of affinity were generated by injecting each analyte over the entire antibody array using the single channel flow cell. CD98hc Avi-His analytes were diluted with running buffer, in a series of six, three-fold serial dilutions starting from 600 nM for human and cyno CD98hc Avi-His. Analytes were injected for 5 minutes, and dissociation was followed for 10 minutes. After each analyte injection, antibodies were regenerated with Pierce IgG Elution Buffer (ThermoScientific). Data were processed and analyzed using NextGenKIT high-throughput kinetics analysis software (Carterra).

The equilibrium dissociation constants (KD) were calculated from the fitted association and dissociation rate constants (k-on and k-off) for 2+1 anti-CD98hc bispecific antibodies of the present disclosure. The values were combined, means and standard deviation calculated, and graphs prepared using GraphPad Prism. The KD values are summarized in Table 20 below. Some antibodies, as indicated in the table, displayed heterogeneous binding profiles that do not fit to a 1:1 binding model, thus their rate constants could not be determined.

TABLE 20
Equilibrium dissociation constants (K_{D}) for 2 + 1 anti-CD98hc bispecific antibodies

| | huCD98hc Avi-His | | | cyno CD98hc Avi-His | | |
|---|---|---|---|---|---|---|
| Antibody | Mean KD (M) | SD | N | Mean KD (M) | SD | N |
| CD98hc.03.002 | 1.9E−08 | 2.1E−09 | 8 | 3.1E−08 | 6.9E−09 | 8 |
| CD98hc.03.004 | 9.1E−09 | 2.8E−09 | 5 | 4.8E−08 | 1.4E−08 | 5 |
| CD98hc.03.010 | 4.1E−08 | 6.8E−09 | 5 | 8.0E−08 | 1.3E−08 | 4 |
| CD98hc.03.031 | 6.8E−08 | 2.0E−08 | 3 | 1.5E−07 | 5.0E−08 | 3 |
| CD98hc.04.003 | 1.9E−08 | 1.7E−09 | 7 | 1.4E−08 | 1.7E−09 | 7 |
| CD98hc.04.006 | 1.8E−08 | 2.9E−09 | 2 | 1.2E−08 | NA | 1 |
| CD98hc.04.009 | heterogeneous | heterogeneous | NA | heterogeneous | heterogeneous | NA |
| CD98hc.04.010 | 2.1E−08 | 1.4E−08 | 8 | 5.2E−09 | 2.8E−09 | 7 |
| CD98hc.04.012 | 1.7E−08 | 2.6E−09 | 3 | 1.4E−07 | 3.2E−08 | 3 |
| CD98hc.04.013 | 3.2E−08 | 2.1E−09 | 7 | 7.6E−08 | 1.6E−08 | 8 |
| CD98hc.04.018 | heterogeneous | heterogeneous | NA | 2.9E−08 | 4.5E−09 | 8 |
| CD98hc.04.022 | 2.2E−08 | 3.5E−09 | 8 | 3.8E−08 | 4.9E−09 | 8 |
| CD98hc.04.045 | 8.1E−08 | NA | 1 | 5.5E−08 | 1.5E−09 | 2 |
| CD98hc.04.046 | 7.0E−08 | NA | 1 | 3.9E−08 | 7.2E−09 | 6 |
| CD98hc.04.047 | 4.7E−08 | 1.4E−08 | 5 | NB | NB | NA |
| CD98hc.04.054 | 1.6E−08 | 4.5E−09 | 3 | 5.3E−09 | 3.1E−09 | 3 |
| CD98hc.04.058 | 2.5E−08 | 4.2E−09 | 2 | NB | NB | NA |
| CD98hc.04.062 | 2.3E−08 | 4.1E−09 | 6 | 4.9E−08 | 7.9E−09 | 7 |
| CD98hc.04.063 | 7.8E−08 | 1.0E−08 | 2 | heterogeneous | heterogeneous | NA |
| CD98hc.04.064 | 2.6E−08 | 3.9E−09 | 8 | heterogeneous | heterogeneous | NA |
| CD98hc.04.065 | NB | NB | NA | NB | NB | NA |
| CD98hc.05.006 | 4.3E−09 | 3.2E−09 | 2 | 2.0E−08 | 3.9E−09 | 4 |
| CD98hc.05.007 | 3.1E−09 | NA | 1 | 3.8E−09 | 2.8E−09 | 2 |
| CD98hc.05.046 | NB | NB | NA | NB | NB | NA |
| CD98hc.05.052 | 1.3E−08 | 1.3E−09 | 2 | 4.1E−09 | NA | 1 |
| CD98hc.05.059 | NB | NB | NA | NB | NB | NA |
| CD98hc.05.081 | heterogeneous | heterogeneous | NA | 8.0E−08 | NA | 1 |
| CD98hc.05.126 | 7.5E−09 | 1.4E−09 | 2 | 2.3E−08 | 1.6E−09 | 3 |
| CD98hc.05.142 | heterogeneous | heterogeneous | NA | 3.2E−09 | 1.2E−09 | 2 |
| CD98hc.05.144 | 1.5E−08 | NA | 1 | 6.3E−08 | 1.2E−08 | 2 |
| CD98hc.05.153 | NB | NB | NA | NB | NB | NA |
| CD98hc.05.165 | 2.1E−07 | NA | 1 | 5.5E−09 | 3.6E−09 | 4 |
| CD98hc.05.166 | 9.1E−09 | 3.8E−10 | 4 | 3.9E−08 | 5.0E−09 | 4 |
| CD98hc.05.167 | 4.4E−09 | 2.6E−09 | 4 | 1.4E−08 | 2.9E−09 | 4 |
| CD98hc.05.168 | 2.0E−08 | 5.1E−09 | 4 | 1.0E−07 | 3.1E−08 | 4 |
| CD98hc.05.176 | 1.7E−07 | NA | 1 | 4.1E−08 | NA | 1 |
| CD98hc.05.180 | 5.2E−09 | NA | 1 | 1.6E−08 | NA | 1 |
| CD98hc.05.182 | 1.9E−07 | NA | 1 | heterogeneous | heterogeneous | NA |
| CD98hc.05.188 | 2.7E−08 | 4.4E−09 | 4 | heterogeneous | heterogeneous | NA |
| CD98hc.05.191 | heterogeneous | heterogeneous | NA | heterogeneous | heterogeneous | NA |
| CD98hc.05.193 | 1.1E−08 | 9.2E−10 | 2 | 2.2E−08 | 1.2E−08 | 3 |
| CD98hc.05.207 | 4.2E−08 | 2.8E−10 | 2 | 1.2E−07 | 1.1E−08 | 2 |
| CD98hc.05.226 | 2.1E−08 | 4.7E−09 | 3 | 6.1E−08 | 9.3E−09 | 3 |

N = number of determinations; NT = not tested; NA = not applicable; LB = low binding (below limit of measurement); NB = no binding detected.

These results illustrate that 2+1 anti-CD98hc bispecific antibodies exhibit a range of affinities from approximately 3 nM to 200 nM for CD98hc Avi-His binding. In particular, affinity of 2+1 anti-CD98hc bispecific antibodies for binding to human CD98hc Avi-His ranged from 3.1 nM to 210 nM; affinity of anti-CD98hc antibodies of the present disclosure for binding to cyno CD98hc Avi-His ranged from 3.2 nM to 145 nM.

### Example 16: Ability of 2+1 Anti-CD98hc Bispecific Antibodies to Effect CD98hc Levels or Localization

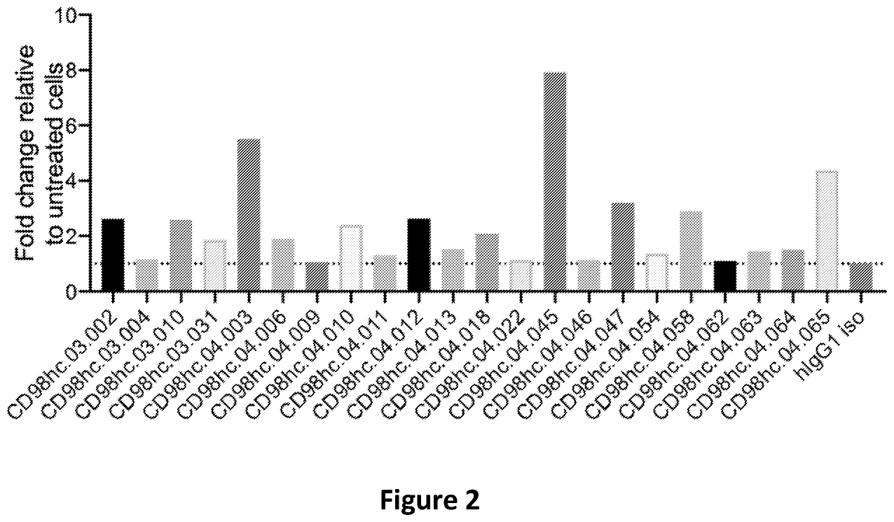

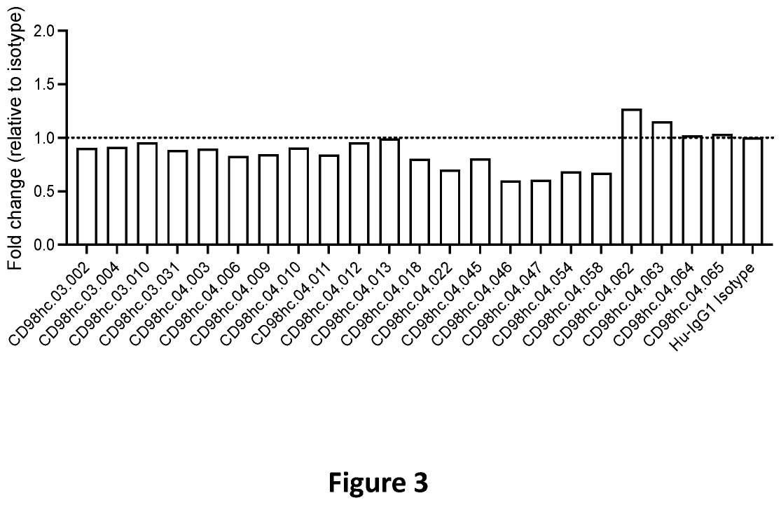

Binding of 2+1 anti-CD98hc bispecific antibodies to their receptor on target cells could dysregulate its cell surface expression or recycling. To assess this, HCMEC/D3 cells (a cell line derived from human brain endothelial cells; Weksler B. et al., FASEB J. 2005; 19 (13): 1872-4) were seeded at 7.5*10\hat{ }4 cells/cm² in tissue culture plates in 100 uL in culture medium (EBM2, Lonza). After 24 hours, cells were treated with 20 μg/ml of 2+1 anti-CD98hc bispecific antibodies for 24 hrs before being analyzed by either flowcytometry (FACS) or western blot. Quantitative FACS data revealed no reduction in cell-surface expression of CD98hc following 2+1 bispecific antibody treatment (FIG. 2). Similarly, the total amount of CD98hc protein in antibody treated cells using quantitative Western blot analysis did not change more than 50% relative to an isotype control (FIG. 3). Notably, cell-surface expression of CD98hc was increased upon treatment with some of these 2+1 anti-CD98hc bispecific antibodies, which can be due a feedback effect induced in cells in response to amino acid deprivation. This phenotype has been reported in cells treated with JPH203 and BCH that inhibit activity of LAT1-CD98hc complex (Maimaiti M. et al., Scientific Reports. (2020) 10:1292; Häfliger et al., Journal of Experimental & Clinical Cancer Research (2018) 37:234). Thus, 2+1 anti-CD98hc bispecific antibodies that significantly increased CD98hc cell surface levels (>2 fold) were excluded from further development processes (except for CD98hc.04.003 because of its other desirable properties (provided in other Examples)).

### Example 17: In-Vivo PK Study of 2+1 Anti-CD98hc Bispecific Antibodies in huCD98hc ECD Mice

Antibody Production Method: Antibodies for in vivo PK studies were generated via transient transfection of ExpiCHO cells (Invitrogen) performed according to manufacturer's instruction. For 400 mL culture, 320 μg of total DNA consisting of 3 expression plasmids (heavy chain-hole-scFv, heavy chain-knob, and light chain) were used. The molar ratio of the 3 plasmids were optimized to achieve high purity of 2+1 anti-CD98hc bispecific antibodies. The cell culture supernatant was harvested at 10 days post transfection. The antibody was purified using protein A affinity chromatography followed by ion exchange chromatography on an AKTA Avant 25 (Cytiva) to remove product-related impurities. The purified antibodies were dialyzed into PBS. Analytical characterization was performed by absorbance at 280 nm, CE-SDS, size-exclusion chromatography, and endotoxin measurement.

Study design: To establish in vivo proof of concept for brain penetration of 2+1 anti-CD98hc bispecific antibodies, a knock-in (KI) mouse was generated, in which the ectodomain region of mouse CD98hc was replaced with that of human CD98hc in one of the two alleles. This was done because the 2+1 anti-CD98hc bispecific antibodies specifically bind to primate CD98hc, but not to mouse CD98hc. A two-dose strategy was used to determine both the brain uptake, as well as peripheral clearance of injected antibodies over time. Briefly, groups (N=3) of heterozygous (huCD98hc+/−KI) mice were dosed with 10 mg/kg of either isotype IgG or 2+1 anti-CD98hc bispecific antibodies at day 1 and 15. Blood samples were then collected from antibody treated animals at various timepoints in serum separator tubes and allowed to clot at room temperature (RT) before centrifugation. The resulting supernatants, representing sera, were subsequently transferred into new tubes and stored at −80° C. until analysis.

Brain sample preparation: Brain tissues were collected from antibody treated mice on day 15 (i.e., 24 hrs after the second antibody injection). Prior to this, mice were anesthetized and underwent cardiac perfusion with 15 ml PBS to clear blood vessels. Brain tissues were then minced and homogenized in 1 ml HBSS buffer (MilliporeSigma #55037C) containing 10 mM HEPES (#15630130, Gibco) using a hand-operated grinder. Subsequently, samples were centrifuged at 1000 g for 5 minutes to pellet the vessel portion. The supernatants, representing the parenchyma, were then transferred into new tubes, and mixed with 10× RIPA buffer (final concentration 1×) including protease and phosphatase inhibitor cocktail (Complete™, Mini Protease Inhibitor Cocktail, Roche, #11836153001). After a 20 minute incubation on a nutator at 4° C., samples were frozen on dry ice and stored at −80° C. until analysis. Vessel fractions were cleared from myelin debris via centrifugation through % 18 Dextran (70 kDa, #31390, Sigma-Aldrich), followed by 2 washes in HBSS buffer. Afterwards, samples were lysed in RIPA buffer (R0278, Sigma-Aldrich) and stored at −80° C. until analysis. The validity of this method was confirmed using WB analysis, in which markers of brain endothelial cells (e.g., CD31 and Claudin-5) were absent in the parenchymal portions (data not shown).

Pharmacokinetic (PK) analysis: Antibody concentrations in serum and brain samples were measured using MSD (Meso Scale Discovery) method. Briefly, 50 μl/well of goat anti-human IgG1 Ab (Jackson ImmunoResearch, #109-005-097) was added to MSD plates at 1 μg/ml in PBS and incubated overnight at 4° C. Plates were then washed 3× with wash buffer (0.05% Tween-20 in 1×PBS), followed by incubation with blocking buffer (wash buffer containing 3% BSA) for 1 hour at RT. Subsequently, serum (1:10000 dilution in PBS) and brain (1:2 dilutions in PBS) samples were added in duplicates to the plates and incubated for 1 hour on a shaker at 500 RPM. A human IgG1 isotype antibody with known concentration was included in the assay to create standard curve. Next, plates were washed 3× with wash buffer, followed by addition of 30 μl/well of Sulfo-Tag goat anti-human antibody (R32AJ-5, Meso Scale Discovery) at 0.2 μg/ml in PBS and 1 hour incubation on shaker at 500 RPM. Finally, after 3× washes with wash buffer, 150 μl/well of 1× READ buffer (R92PC-2, Meso Scale Discovery) was added to the plates, before they are being read using a Sector Imager S600 instrument.

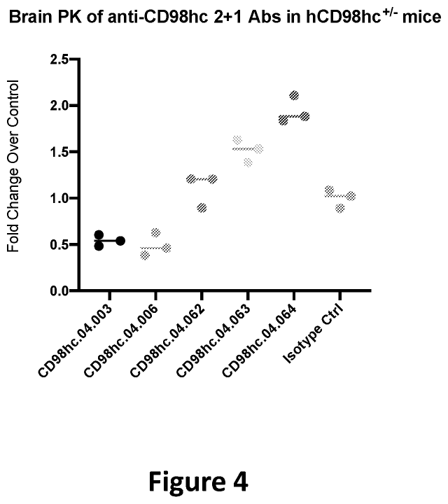

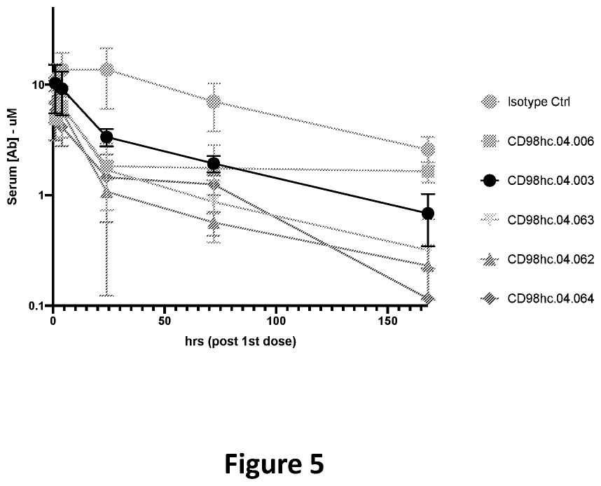

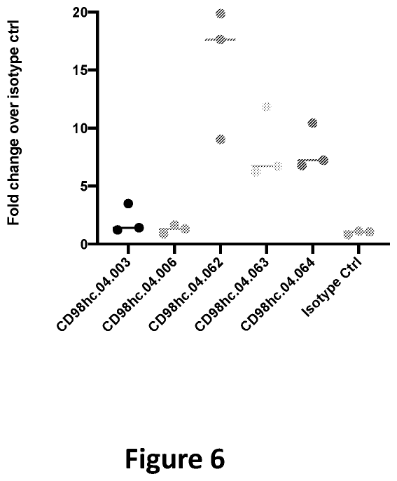

By measuring antibody concentrations in vessel depleted brains, levels of 2+1 anti-CD98hc bispecific antibodies were determined to be up to 2-fold higher than isotype IgG in HET huCD98hc KI mice (FIG. 4). The peripheral PK was determined by measuring antibody concentrations in plasma samples of antibody treated mice (FIG. 5). All 2+1 anti-CD98hc bispecific antibodies tested exhibited higher plasma clearance in HET huCD98hc KI mice compared to isotype IgG, which is consistent with CD98hc-mediated disposition in the periphery. Three of the 2+1 anti-CD98hc bispecific antibodies (CD98hc.04.062, CD98hc.04.063, CD98hc.04.064) tested showed a significant (5-17) fold change in brain/serum ratio at the 24 hour timepoint tested (FIG. 6).

### Example 18: Humanization of 2+1 Anti-CD98hc Bispecific Antibodies

Based on the in vivo mouse data, 2+1 anti-CD98hc bispecific antibody CD98hc.04.064 was chosen for humanization and further development. CD98hc.04.064 was chosen because it had the best brain penetration at 24 hrs post-injection. One of the most common methods of humanizing a non-human antibody is to transplant the CDRs from a non-human antibody onto a human antibody acceptor framework. Frequently, such CDR transplantation results in attenuation or complete loss of affinity of the humanized antibody due to perturbation in its framework. As a result, certain residues from the mouse framework may need to be retained to replace the human residues at the corresponding positions (back mutations) in order to restore attenuated or lost affinity. Therefore, it is crucial to precisely predict the residues to be retained in the context of the selected human antibody germline acceptor framework that can retain functions and paratopes of the humanized antibody. In addition, retained or improved thermal stability and solubility are desired for good manufacturability and downstream development.

Structure-based antibody modeling was applied in the process of humanizing anti-CD98hc mouse monoclonal antibodies (mAbs) utilizing the BioMOE module of MOE (Molecular Operating Environment, Chemical Computing Group, Montreal, Canada). Briefly, VH and VL sequences of the mouse mAb sequence to be humanized were compared to human VL, VH, LJ, HJ functional germline sequences taken from IMGT (http://www.imgt.org/). Pseudo-genes and ORFs were excluded. Per one mouse mAb (query), five most similar VL and five most similar VH germline sequences were selected and combined with the most similar VJ and HJ genes, producing 25 humanized sequences. The CDRs to be transplanted onto the human framework were defined according to the AbM definition (http://www.bioinf.org.uk/abs/#cdrdef).

Two humanized sequences were selected based on the frequency of their VH and VL frameworks in human repertoire. The query and the humanized sequences were used to create Fv homology models. The BioMOE module or the Antibody Modeler module of MOE (Molecular Operating Environment, Chemical Computing Group, Montreal, Canada) was utilized to create Fv homology models. AMBER10: EHT force field was used for energy minimization through the entire antibody homology modeling process. Based on the Fv homology models, molecular descriptors such as interaction energy between VL and VH, coordinate-based isoelctric point (3D pI), hydrophobic patch, and charged surface area were calculated, analyzed, and sorted by scoring metrics provided by MOE. These molecular descriptors were utilized to prioritize the humanized mAbs for downstream experimental procedures, including protein expression, purification, and binding affinity test, and functional assays.

The BioMOE module of MOE provides a tool, Mutation Site Properties, to visualize and classify potential residues for back-mutation. Back-mutation is defined as amino acid substitution which is reverted to the original query sequence replacing the humanized sequence. Using this tool, the original query (reference) was compared individually to the selected humanized variants for both the primary amino acid sequence and the 3D structure of the 3D Fv homology model.

The changes between the reference and the humanized variant were classified based on amino acid type difference, interaction potential with CDR residues, impact potential for VL/VH pairing, and potential change in hydrophobic and charged surface area in and near the CDRs.

Mutations that are near the CDRs or the VL/VH interface, have a significant charge difference or contain strong H-bond interactions were individually evaluated and the significantly disrupting mutations were reverted back to the original query residues. As a result, humanized sequences may contain up to five back mutations. The query mouse mAb (mouse CD98hc.04.064) and the humanized mAbs are listed as scFv in Table 21 below.

TABLE 21
Humanized Anti-CD98hc scFv Sequences

| | |
|---|---|
| mouse | QVQLQQPGAELVRPGASVKLSCKASGYTFTSYWMNWVRQRPGQGLEWIGLIDPSDSETHYNQMFKDRA |
| CD98hc.04.064 | TLTVDKSSSTAYMQLSSLTSADPAVYYCARASYGKGYFDYWGQGTTLTVSSGGSEGKSSGSGSESKST |
| | GGSDIVMTQSPSSLAMSVGQKVTMSCKSSQSLLNNINQKNYLAWYQQKPGQSPKLLVYFASTRESGVP |
| | DRFIGSGSGTDFTLTINTVQAEDLADYFCQQHYSSPFTFGGGTKLEIK |
| | (SEQ ID NO: 311) |
| CD98hc.04. | QVQLVQSGAEVKKPGASVKVSCKASGYTFTSYWMNWVRQAPGQGLEWMGIIDPSDSETHYAQKFQGRV |
| 064.1a | TMTVDKSTSTVYMELSSLRSEDTAVYYCARASYGKGYFDYWGQGTLVTVSSGGSEGKSSGSGSESKST |
| | GGSDIVMTQSPDSLAVSLGERATINCKSSQSLLNNINQKNYLAWYQQKPGQPPKLLIYFASTRESGVP |
| | DRFSGSGSGTDFTLTISSLQAEDVAVYYCQQHYSSPFTFGGGTKVEIK |
| | (SEQ ID NO: 312) |
| CD98hc.04. | QVQLVQSGAEVKKPGASVKVSCKASGYTFTSYWMNWVRQAPGQGLEWMGIIDPSDSETHYAQKFQGRV |
| 064.1b | TMTVDKSTSTVYMELSSLRSEDTAVYYCARASYGKGYFDYWGQGTLVTVSSGGSEGKSSGSGSESKST |
| | GGSDIVMTQSPDSLAVSLGERATINCKSSQSLLNNINQKNYLAWYQQKPGQPPKLLIYFASTRESGVP |
| | DRFSGSGSGTDFTLTISSLQAEDVAVYFCQQHYSSPFTFGGGTKVEIK |
| | (SEQ ID NO: 313) |
| CD98hc.04. | QVQLVQSGAEVKKPGASVKVSCKASGYTFTSYWMHWVRQAPGQGLEWMGIIDPSDSETSYAQKFQGRV |
| 064.1c | TMTVDKSTSTVYMELSSLRSEDTAVYYCARASYGKGYFDYWGQGTLVTVSSGGSEGKSSGSGSESKST |
| | GGSDIVMTQSPDSLAVSLGERATINCKSSQSLLNNINQKNYLAWYQQKPGQPPKLLIYFASTRESGVP |
| | DRFSGSGSGTDFTLTISSLQAEDVAVYYCQQHYSSPFTFGGGTKVEIK |
| | (SEQ ID NO: 314) |
| CD98hc.04. | QVQLVQSGAEVKKPGASVKVSCKASGYTFTSYWMNWVRQAPGQGLEWMGIIDPSDSETSYAQKFQGRV |
| 064.1d | TMTVDKSTSTVYMELSSLRSEDTAVYYCARASYGKGYFDYWGQGTLVTVSSGGSEGKSSGSGSESKST |
| | GGSDIVMTQSPDSLAVSLGERATINCKSSQSLLNNINQKNYLAWYQQKPGQPPKLLIYFASTRESGVP |
| | DRFSGSGSGTDFTLTISSLQAEDVAVYYCQQHYSSPFTFGGGTKVEIK |
| | (SEQ ID NO: 315) |
| CD98hc.04. | QVQLVQSGAEVKKPGASVKVSCKASGYTFTSYWMHWVRQAPGQGLEWMGIIDPSDSETHYAQKFQGRV |
| 064.1e | TMTVDKSTSTVYMELSSLRSEDTAVYYCARASYGKGYFDYWGQGTLVTVSSGGSEGKSSGSGSESKST |
| | GGSDIVMTQSPDSLAVSLGERATINCKSSQSLLNNINQKNYLAWYQQKPGQPPKLLIYFASTRESGVP |
| | DRFSGSGSGTDFTLTISSLQAEDVAVYYCQQHYSSPFTFGGGTKVEIK |
| | (SEQ ID NO: 316) |
| CD98hc.04. | QVQLVQSGAEVKKPGASVKVSCKASGYTFTSYWMNWVRQAPGQGLEWMGIIDPSDSETHYAQKFQGRV |
| 064.1f | TMTRDTSTSTVYMELSSLRSEDTAVYYCARASYGKGYFDYWGQGTLVTVSSGGSEGKSSGSGSESKST |
| | GGSDIVMTQSPDSLAVSLGERATINCKSSQSLLNNINQKNYLAWYQQKPGQPPKLLIYFASTRESGVP |
| | DRFSGSGSGTDFTLTISSLQAEDVAVYYCQQHYSSPFTFGGGTKVEIK |
| | (SEQ ID NO: 317) |
| CD98hc.04. | QVQLVQSGAEVKKPGASVKVSCKASGYTFTSYWMNWVRQAPGQGLEWMGIIDPSDSETNYAQKFQGRV |
| 064.4a | TSTVDKSISTAYMELSRLRSDDTVVYYCARASYGKGYFDYWGQGTLVTVSSGGSEGKSSGSGSESKST |
| | GGSDIVMTQSPDSLAVSLGERATINCKSSQSLLNNINQKNYLAWYQQKPGQPPKLLIYFASTRESGVP |
| | DRFSGSGSGTDFTLTISSLQAEDVAVYYCQQHYSSPFTFGGGTKVEIK |
| | (SEQ ID NO: 318) |
| CD98hc.04. | QVQLVQSGAEVKKPGASVKVSCKASGYTFTSYWMNWVRQAPGQGLEWMGIIDPSDSETNYAQKFQGRV |
| 064.4b | TSTVDKSISTAYMELSRLRSDDTVVYYCARASYGKGYFDYWGQGTLVTVSSGGSEGKSSGSGSESKST |
| | GGSDIVMTQSPDSLAVSLGERATINCKSSQSLLNNINQKNYLAWYQQKPGQPPKLLIYFASTRESGVP |
| | DRFSGSGSGTDFTLTISSLQAEDVAVYFCQQHYSSPFTFGGGTKVEIK |
| | (SEQ ID NO: 319) |
| CD98hc.04. | QVQLVQSGAEVKKPGASVKVSCKASGYTFTSYWMHWVRQAPGQGLEWMGIIDPSDSETNYAQKFQGRV |
| 064.4c | TSTVDKSISTAYMELSRLRSDDTVVYYCARASYGKGYFDYWGQGTLVTVSSGGSEGKSSGSGSESKST |
| | GGSDIVMTQSPDSLAVSLGERATINCKSSQSLLNNINQKNYLAWYQQKPGQPPKLLIYFASTRESGVP |
| | DRFSGSGSGTDFTLTISSLQAEDVAVYYCQQHYSSPFTFGGGTKVEIK |
| | (SEQ ID NO: 320) |
| CD98hc.04. | QVQLVQSGAEVKKPGASVKVSCKASGYTFTSYWMHWVRQAPGQGLEWMGIIDPSDSETSYAQKFQGRV |
| 064.4d | TMTRDTSTSTVYMELSSLRSEDTAVYYCARASYGKGYFDYWGQGTLVTVSSGGSEGKSSGSGSESKST |
| | GGSDIQMTQSPSSLSASVGDRVTITCRASQSLLNNINQKNYLAWYQQKPGKAPKLLIYFASSLQSGVP |
| | SRFSGSGSGTDFTLTISSLQPEDFATYYCQQHYSSPFTFGGGTKVEIK |
| | (SEQ ID NO: 321) |

TABLE 22
Humanized Anti-CD98hc Sequences (VH and VL of sc Fv sequences in Table 21)

| Antibody name | VH | VL |
|---|---|---|
| CD98hc.04.064.1a | QVQLVQSGAEVKKPGASVKVSCKAS | DIVMTQSPDSLAVSLGERATINCKSSQSL |
| | GYTFTSYWMNWVRQAPGQGLEWMGI | LNNINQKNYLAWYQQKPGQPPKLLIYFAS |
| | IDPSDSETHYAQKFQGRVTMTVDKS | TRESGVPDRFSGSGSGTDFTLTISSLQAE |
| | TSTVYMELSSLRSEDTAVYYCARAS | DVAVYYCQQHYSSPFTFGGGTKVEIK |
| | YGKGYFDYWGQGTLVTVSS (SEQ ID | (SEQ ID NO: 356) |
| | NO: 355) | |
| CD98hc.04.064.1b | QVQLVQSGAEVKKPGASVKVSCKAS | DIVMTQSPDSLAVSLGERATINCKSSQSL |
| | GYTFTSYWMNWVRQAPGQGLEWMGI | LNNINQKNYLAWYQQKPGQPPKLLIYFAS |
| | IDPSDSETHYAQKFQGRVTMTVDKS | TRESGVPDRFSGSGSGTDFTLTISSLQAE |
| | TSTVYMELSSLRSEDTAVYYCARAS | DVAVYFCQQHYSSPFTFGGGTKVEIK |
| | YGKGYFDYWGQGTLVTVSS | (SEQ ID NO: 358) |
| | (SEQ ID NO: 357) | |
| CD98hc.04.064.1c | QVQLVQSGAEVKKPGASVKVSCKAS | DIVMTQSPDSLAVSLGERATINCKSSQSL |
| | GYTFTSYWMHWVRQAPGQGLEWMGI | LNNINQKNYLAWYQQKPGQPPKLLIYFAS |
| | IDPSDSETSYAQKFQGRVTMTVDKS | TRESGVPDRFSGSGSGTDFTLTISSLQAE |
| | TSTVYMELSSLRSEDTAVYYCARAS | DVAVYYCQQHYSSPFTFGGGTKVEIK |
| | YGKGYFDYWGQGTLVTVSS | (SEQ ID NO: 360) |
| | (SEQ ID NO: 359) | |
| CD98hc.04.064.1d | QVQLVQSGAEVKKPGASVKVSCKAS | DIVMTQSPDSLAVSLGERATINCKSSQSL |
| | GYTFTSYWMNWVRQAPGQGLEWMGI | LNNINQKNYLAWYQQKPGQPPKLLIYFAS |
| | IDPSDSETSYAQKFQGRVTMTVDKS | TRESGVPDRFSGSGSGTDFTLTISSLQAE |
| | TSTVYMELSSLRSEDTAVYYCARAS | DVAVYYCQQHYSSPFTFGGGTKVEIK |
| | YGKGYFDYWGQGTLVTVSS | (SEQ ID NO: 362) |
| | (SEQ ID NO: 361) | |
| CD98hc.04.064.1e | QVQLVQSGAEVKKPGASVKVSCKAS | DIVMTQSPDSLAVSLGERATINCKSSQSL |
| | GYTFTSYWMHWVRQAPGQGLEWMGI | LNNINQKNYLAWYQQKPGQPPKLLIYFAS |
| | IDPSDSETHYAQKFQGRVTMTVDKS | TRESGVPDRFSGSGSGTDFTLTISSLQAE |
| | TSTVYMELSSLRSEDTAVYYCARAS | DVAVYYCQQHYSSPFTFGGGTKVEIK |
| | YGKGYFDYWGQGTLVTVSS | (SEQ ID NO: 364) |
| | (SEQ ID NO: 363) | |
| CD98hc.04.064.1f | QVQLVQSGAEVKKPGASVKVSCKAS | DIVMTQSPDSLAVSLGERATINCKSSQSL |
| | GYTFTSYWMNWVRQAPGQGLEWMGI | LNNINQKNYLAWYQQKPGQPPKLLIYFAS |
| | IDPSDSETHYAQKFQGRVTMTRDTS | TRESGVPDRFSGSGSGTDFTLTISSLQAE |
| | TSTVYMELSSLRSEDTAVYYCARAS | DVAVYYCQQHYSSPFTFGGGTKVEIK |
| | YGKGYFDYWGQGTLVTVSS | (SEQ ID NO: 366) |
| | (SEQ ID NO: 365) | |
| CD98hc.04.064.4a | QVQLVQSGAEVKKPGASVKVSCKAS | DIVMTQSPDSLAVSLGERATINCKSSQSL |
| | GYTFTSYWMNWVRQAPGQGLEWMGI | LNNINQKNYLAWYQQKPGQPPKLLIYFAS |
| | IDPSDSETNYAQKFQGRVTSTVDKS | TRESGVPDRFSGSGSGTDFTLTISSLQAE |
| | ISTAYMELSRLRSDDTVVYYCARAS | DVAVYYCQQHYSSPFTFGGGTKVEIK |
| | YGKGYFDYWGQGTLVTVSS | (SEQ ID NO: 368) |
| | (SEQ ID NO: 367) | |
| CD98hc.04.064.4b | QVQLVQSGAEVKKPGASVKVSCKAS | DIVMTQSPDSLAVSLGERATINCKSSQSL |
| | GYTFTSYWMNWVRQAPGQGLEWMGI | LNNINQKNYLAWYQQKPGQPPKLLIYFAS |
| | IDPSDSETNYAQKFQGRVTSTVDKS | TRESGVPDRFSGSGSGTDFTLTISSLQAE |
| | ISTAYMELSRLRSDDTVVYYCARAS | DVAVYFCQQHYSSPFTFGGGTKVEIK |
| | YGKGYFDYWGQGTLVTVSS | (SEQ ID NO: 370) |
| | (SEQ ID NO: 369) | |
| CD98hc.04.064.4c | QVQLVQSGAEVKKPGASVKVSCKAS | DIVMTQSPDSLAVSLGERATINCKSSQSL |
| | GYTFTSYWMHWVRQAPGQGLEWMGI | LNNINQKNYLAWYQQKPGQPPKLLIYFAS |
| | IDPSDSETNYAQKFQGRVTSTVDKS | TRESGVPDRFSGSGSGTDFTLTISSLQAE |
| | ISTAYMELSRLRSDDTVVYYCARAS | DVAVYYCQQHYSSPFTFGGGTKVEIK |
| | YGKGYFDYWGQGTLVTVSS | (SEQ ID NO: 372) |
| | (SEQ ID NO: 371) | |
| CD98hc.04.064.4d | QVQLVQSGAEVKKPGASVKVSCKAS | DIQMTQSPSSLSASVGDRVTITCRASQSL |
| | GYTFTSYWMHWVRQAPGQGLEWMGI | LNNINQKNYLAWYQQKPGKAPKLLIYFAS |
| | IDPSDSETSYAQKFQGRVTMTRDTS | SLQSGVPSRFSGSGSGTDFTLTISSLQPE |
| | TSTVYMELSSLRSEDTAVYYCARAS | DFATYYCQQHYSSPFTFGGGTKVEIK |
| | YGKGYFDYWGQGTLVTVSS | (SEQ ID NO: 374) |
| | (SEQ ID NO: 373) | |

TABLE 23
Humanized Anti-CD98hc CDR Sequences (Kabat)

| Antibody | CDR-H1 | CDR-H2 | CDR-H3 |
|---|---|---|---|
| mouse | SYWMN (SEQ ID | LIDPSDSETHYNQMFK | ASYGKGYFD |
| CD98hc.04.064 | NO: 110) | D (SEQ ID NO: 111) | Y (SEQ ID |
| | | | NO: 112) |
| CD98hc.04.064.1a | SYWMN (SEQ ID | IIDPSDSETHYAQKFQG | ASYGKGYFD |
| | NO: 110) | (SEQ ID NO: 414) | Y (SEQ ID |
| | | | NO: 112) |
| CD98hc.04.064.1b | SYWMN (SEQ ID | IIDPSDSETHYAQKFQG | ASYGKGYFD |
| | NO: 110) | (SEQ ID NO: 414) | Y (SEQ ID |
| | | | NO: 112) |
| CD98hc.04.064.1c | SYWMH (SEQ ID | IIDPSDSETSYAQKFQG | ASYGKGYFD |
| | NO: 413) | (SEQ ID NO: 415) | Y (SEQ ID |
| | | | NO: 112) |
| CD98hc.04.064.1d | SYWMN (SEQ ID | IIDPSDSETSYAQKFQG | ASYGKGYFD |
| | NO: 110) | (SEQ ID NO: 415) | Y (SEQ ID |
| | | | NO: 112) |
| CD98hc.04.064.1e | SYWMH (SEQ ID | IIDPSDSETHYAQKFQG | ASYGKGYFD |
| | NO: 413) | (SEQ ID NO: 414) | Y (SEQ ID |
| | | | NO: 112) |
| CD98hc.04.064.1f | SYWMN (SEQ ID | IIDPSDSETHYAQKFQG | ASYGKGYFD |
| | NO: 110) | (SEQ ID NO: 414) | Y (SEQ ID |
| | | | NO: 112) |
| CD98hc.04.064.4a | SYWMN (SEQ ID | IIDPSDSETNYAQKFQG | ASYGKGYFD |
| | NO: 110) | (SEQ ID NO: 416) | Y (SEQ ID |
| | | | NO: 112) |
| CD98hc.04.064.4b | SYWMN (SEQ ID | IIDPSDSETNYAQKFQG | ASYGKGYFD |
| | NO: 110) | (SEQ ID NO: 416) | Y (SEQ ID |
| | | | NO: 112) |
| CD98hc.04.064.4c | SYWMH (SEQ ID | IIDPSDSETNYAQKFQG | ASYGKGYFD |
| | NO: 413) | (SEQ ID NO: 416) | Y (SEQ ID |
| | | | NO: 112) |
| CD98hc.04.064.4d | SYWMH (SEQ ID | IIDPSDSETSYAQKFQG | ASYGKGYFD |
| | NO: 413) | (SEQ ID NO: 415) | Y (SEQ ID |
| | | | NO: 112) |
| Antibody | CDR-L1 | CDR-L2 | CDR-L3 |
| mouse | KSSQSLLNNINQKNYL | FASTRES (SEQ ID | QQHYSSPFT |
| CD98hc.04.064 | A (SEQ ID NO: 176) | NO: 177) | (SEQ ID |
| | | | NO: 178) |
| CD98hc.04.064.1a | KSSQSLLNNINQKNYL | FASTRES (SEQ ID | QQHYSSPFT |
| | A (SEQ ID NO: 176) | NO: 177) | (SEQ ID |
| | | | NO: 178) |
| CD98hc.04.064.1b | KSSQSLLNNINQKNYL | FASTRES (SEQ ID | QQHYSSPFT |
| | A (SEQ ID NO: 176) | NO: 177) | (SEQ ID |
| | | | NO: 178) |
| CD98hc.04.064.1c | KSSQSLLNNINQKNYL | FASTRES (SEQ ID | QQHYSSPFT |
| | A (SEQ ID NO: 176) | NO: 177) | (SEQ ID |
| | | | NO: 178) |
| CD98hc.04.064.1d | KSSQSLLNNINQKNYL | FASTRES (SEQ ID | QQHYSSPFT |
| | A (SEQ ID NO: 176) | NO: 177) | (SEQ ID |
| | | | NO: 178) |
| CD98hc.04.064.1e | KSSQSLLNNINQKNYL | FASTRES (SEQ ID | QQHYSSPFT |
| | A (SEQ ID NO: 176) | NO: 177) | (SEQ ID |
| | | | NO: 178) |
| CD98hc.04.064.1f | KSSQSLLNNINQKNYL | FASTRES (SEQ ID | QQHYSSPFT |
| | A (SEQ ID NO: 176) | NO: 177) | (SEQ ID |
| | | | NO: 178) |
| CD98hc.04.064.4a | KSSQSLLNNINQKNYL | FASTRES (SEQ ID | QQHYSSPFT |
| | A (SEQ ID NO: 176) | NO: 177) | (SEQ ID |
| | | | NO: 178) |
| CD98hc.04.064.4b | KSSQSLLNNINQKNYL | FASTRES (SEQ ID | QQHYSSPFT |
| | A (SEQ ID NO: 176) | NO: 177) | (SEQ ID |
| | | | NO: 178) |
| CD98hc.04.064.4c | KSSQSLLNNINQKNYL | FASTRES (SEQ | QQHYSSPFT |
| | A (SEQ ID NO: 176) | ID NO: 177) | (SEQ ID |
| | | | NO: 178) |
| CD98hc.04.064.4d | RASQSLLNNINQKNYL | FASSLQS (SEQ ID | QQHYSSPFT |
| | A (SEQ ID NO: 417) | NO: 418) | (SEQ ID |
| | | | NO: 178) |

### Example 19: Affinity Optimization and Liability Removal of 2+1 Anti-CD98hc Antibodies

Based on characterization of the humanized variants, CD98hc.04.064.4a was chosen for further engineering. Affinity optimization of CD98hc.04.04.064.4a variants were designed by the removal of liability sequences, such as high-risk isomerization sites and oxidation sites, and replacing them with residues that have high frequency at that position in the immunorepertoire. In addition, residues in CDR regions predicted to have strong antigen binding interactions were selected for mutational analysis and evaluation for further affinity maturation. The variants chosen for production and further screening is shown below in Table 24. Selected clones were produced as 2+1 anti-CD98hc bispecific antibodies in the format described above (Example 9).

TABLE 24
Affinity Optimized 2 + 1 Bispecific Antibody Variant Sequences

| 2 + 1 Bispecific | |
|---|---|
| Antibody Name | Sequence |
| CD98hc.04.064. | QVQLVQSGAEVKKPGASVKVSCKASGYTFTSYYMNWVRQAPGQGLEWMGIIDPSESETNYA |
| 4a.1 | QKFQGRVTSTVDKSISTAYMELSRLRSDDTVVYYCARASYGKGYFDYWGQGTLVTVSSGGS |
| | EGKSSGSGSESKSTGGSDIVMTQSPDSLAVSLGERATINCKSSQSLLNNINQKNYLAWYQQ |
| | KPGQPPKLLIYFASTRESGVPDRFSGSGSGTDFTLTISSLQAEDVAVYYCQQHYSSPFTFG |
| | GGTKVEIK |
| | (SEQ ID NO: 322) |
| CD98hc.04.064. | QVQLVQSGAEVKKPGASVKVSCKASGYTFTSYYMNWVRQAPGQGLEWMGIIDPSQSETNYA |
| 4a.2 | QKFQGRVTSTVDKSISTAYMELSRLRSDDTVVYYCARASYGKGYFDYWGQGTLVTVSSGGS |
| | EGKSSGSGSESKSTGGSDIVMTQSPDSLAVSLGERATINCKSSQSLLNNINQKNYLAWYQQ |
| | KPGQPPKLLIYFASTRESGVPDRFSGSGSGTDFTLTISSLQAEDVAVYYCQQHYSSPFTFG |
| | GGTKVEIK |
| | (SEQ ID NO: 323) |
| CD98hc.04.064. | QVQLVQSGAEVKKPGASVKVSCKASGYTFTSYYMNWVRQAPGQGLEWMGIIDPSSSETNYA |
| 4a.3 | QKFQGRVTSTVDKSISTAYMELSRLRSDDTVVYYCARASYGKGYFDYWGQGTLVTVSSGGS |
| | EGKSSGSGSESKSTGGSDIVMTQSPDSLAVSLGERATINCKSSQSLLNNINQKNYLAWYQQ |
| | KPGQPPKLLIYFASTRESGVPDRFSGSGSGTDFTLTISSLQAEDVAVYYCQQHYSSPFTFG |
| | GGTKVEIK |
| | (SEQ ID NO: 324) |
| CD98hc.04.064. | QVQLVQSGAEVKKPGASVKVSCKASGYTFTSYYMNWVRQAPGQGLEWMGIIDPSGSETNYA |
| 4a.4 | QKFQGRVTSTVDKSISTAYMELSRLRSDDTVVYYCARASYGKGYFDYWGQGTLVTVSSGGS |
| | EGKSSGSGSESKSTGGSDIVMTQSPDSLAVSLGERATINCKSSQSLLNNINQKNYLAWYQQ |
| | KPGQPPKLLIYFASTRESGVPDRFSGSGSGTDFTLTISSLQAEDVAVYYCQQHYSSPFTFG |
| | GGTKVEIK |
| | (SEQ ID NO: 325) |
| CD98hc.04.064. | QVQLVQSGAEVKKPGASVKVSCKASGYTFTSYYMNWVRQAPGQGLEWMGIIDPSASETNYA |
| 4a.5 | QKFQGRVTSTVDKSISTAYMELSRLRSDDTVVYYCARASYGKGYFDYWGQGTLVTVSSGGS |
| | EGKSSGSGSESKSTGGSDIVMTQSPDSLAVSLGERATINCKSSQSLLNNINQKNYLAWYQQ |
| | KPGQPPKLLIYFASTRESGVPDRFSGSGSGTDFTLTISSLQAEDVAVYYCQQHYSSPFTFG |
| | GGTKVEIK |
| | (SEQ ID NO: 326) |
| CD98hc.04.064. | QVQLVQSGAEVKKPGASVKVSCKASGYTFTSYYMNWVRQAPGQGLEWMGIIDPSESETNYA |
| 4a.6 | QKFQGRVTSTVDKSISTAYMELSRLRSDDTVVYYCARASYGKGYFDYWGQGTLVTVSSGGS |
| | EGKSSGSGSESKSTGGSDIVMTQSPDSLAVSLGERATINCKSSQSLLENINQKNYLAWYQQ |
| | KPGQPPKLLIYFASTRESGVPDRFSGSGSGTDFTLTISSLQAEDVAVYYCQQHYSSPFTFG |
| | GGTKVEIK |
| | (SEQ ID NO: 327) |
| CD98hc.04.064. | QVQLVQSGAEVKKPGASVKVSCKASGYTFTSYYMNWVRQAPGQGLEWMGIIDPSESETNYA |
| 4a.7 | QKFQGRVTSTVDKSISTAYMELSRLRSDDTVVYYCARASYGKGYFDYWGQGTLVTVSSGGS |
| | EGKSSGSGSESKSTGGSDIVMTQSPDSLAVSLGERATINCKSSQSLLQNINQKNYLAWYQQ |
| | KPGQPPKLLIYFASTRESGVPDRFSGSGSGTDFTLTISSLQAEDVAVYYCQQHYSSPFTFG |
| | GGTKVEIK |
| | (SEQ ID NO: 328) |
| CD98hc.04.064. | QVQLVQSGAEVKKPGASVKVSCKASGYTFTSYYMNWVRQAPGQGLEWMGIIDPSESETNYA |
| 4a.8 | QKFQGRVTSTVDKSISTAYMELSRLRSDDTVVYYCARASYGKGYFDYWGQGTLVTVSSGGS |
| | EGKSSGSGSESKSTGGSDIVMTQSPDSLAVSLGERATINCKSSQSLLSNINQKNYLAWYQQ |
| | KPGQPPKLLIYFASTRESGVPDRFSGSGSGTDFTLTISSLQAEDVAVYYCQQHYSSPFTFG |
| | GGTKVEIK |
| | (SEQ ID NO: 329) |
| CD98hc.04.064. | QVQLVQSGAEVKKPGASVKVSCKASGYTFTSYYMNWVRQAPGQGLEWMGIIDPSESETNYA |
| 4a.9 | QKFQGRVTSTVDKSISTAYMELSRLRSDDTVVYYCARASYGKGYFDYWGQGTLVTVSSGGS |
| | EGKSSGSGSESKSTGGSDIVMTQSPDSLAVSLGERATINCKSSQSLLGNINQKNYLAWYQQ |
| | KPGQPPKLLIYFASTRESGVPDRFSGSGSGTDFTLTISSLQAEDVAVYYCQQHYSSPFTFG |
| | GGTKVEIK |
| | (SEQ ID NO: 330) |
| CD98hc.04.064. | QVQLVQSGAEVKKPGASVKVSCKASGYTFTSYYMNWVRQAPGQGLEWMGIIDPSESETNYA |
| 4a.10 | QKFQGRVTSTVDKSISTAYMELSRLRSDDTVVYYCARASYGKGYFDYWGQGTLVTVSSGGS |
| | EGKSSGSGSESKSTGGSDIVMTQSPDSLAVSLGERATINCKSSQSLLANINQKNYLAWYQQ |
| | KPGQPPKLLIYFASTRESGVPDRFSGSGSGTDFTLTISSLQAEDVAVYYCQQHYSSPFTFG |
| | GGTKVEIK |
| | (SEQ ID NO: 331) |
| CD98hc. 04.064. | QVQLVQSGAEVKKPGASVKVSCKASGYTFTSYYMNWVRQAPGQGLEWMGIIDPSESETNYA |
| 4a.11 | QKFQGRVTSTVDKSISTAYMELSRLRSDDTVVYYCARASYGKGYFDYWGQGTLVTVSSGGS |
| | EGKSSGSGSESKSTGGSDIVMTQSPDSLAVSLGERATINCKSSQSLLHNINQKNYLAWYQQ |
| | KPGQPPKLLIYFASTRESGVPDRFSGSGSGTDFTLTISSLQAEDVAVYYCQQHYSSPFTFG |
| | GGTKVEIK |
| | (SEQ ID NO: 332) |
| CD98hc.04.064. | QVQLVQSGAEVKKPGASVKVSCKASGYTFTSYYMNWVRQAPGQGLEWMGIIDPSESETNYA |
| 4a.12 | QKFQGRVTSTVDKSISTAYMELSRLRSDDTVVYYCARASYGKGYFDYWGQGTLVTVSSGGS |
| | EGKSSGSGSESKSTGGSDIVMTQSPDSLAVSLGERATINCKSSQSLLYNINQKNYLAWYQQ |
| | KPGQPPKLLIYFASTRESGVPDRFSGSGSGTDFTLTISSLQAEDVAVYYCQQHYSSPFTFG |
| | GGTKVEIK |
| | (SEQ ID NO: 333) |
| CD98hc.04.064. | QVQLVQSGAEVKKPGASVKVSCKASGYTFTSYAMNWVRQAPGQGLEWMGIIDPSESETNYA |
| 4a.13 | QKFQGRVTSTVDKSISTAYMELSRLRSDDTVVYYCARASYGKGYFDYWGQGTLVTVSSGGS |
| | EGKSSGSGSESKSTGGSDIVMTQSPDSLAVSLGERATINCKSSQSLLNNINQKNYLAWYQQ |
| | KPGQPPKLLIYFASTRESGVPDRFSGSGSGTDFTLTISSLQAEDVAVYYCQQHYSSPFTFG |
| | GGTKVEIK |
| | (SEQ ID NO: 334) |
| CD98hc.04.064. | QVQLVQSGAEVKKPGASVKVSCKASGYTFTSYFMNWVRQAPGQGLEWMGIIDPSESETNYA |
| 4a.15 | QKFQGRVTSTVDKSISTAYMELSRLRSDDTVVYYCARASYGKGYFDYWGQGTLVTVSSGGS |
| | EGKSSGSGSESKSTGGSDIVMTQSPDSLAVSLGERATINCKSSQSLLNNINQKNYLAWYQQ |
| | KPGQPPKLLIYFASTRESGVPDRFSGSGSGTDFTLTISSLQAEDVAVYYCQQHYSSPFTFG |
| | GGTKVEIK |
| | (SEQ ID NO : 335) |
| CD98hc.04.064. | QVQLVQSGAEVKKPGASVKVSCKASGYTFTSYFMNWVRQAPGQGLEWMGIIDPSASETNYA |
| 4a.16 | QKFQGRVTSTVDKSISTAYMELSRLRSDDTVVYYCARASYGKGYFDYWGQGTLVTVSSGGS |
| | EGKSSGSGSESKSTGGSDIVMTQSPDSLAVSLGERATINCKSSQSLLGNINQKNYLAWYQQ |
| | KPGQPPKLLIYFASTRESGVPDRFSGSGSGTDFTLTISSLQAEDVAVYYCQQHYSSPFTFG |
| | GGTKVEIK |
| | (SEQ ID NO: 336) |

TABLE 25
Affinity Optimized 2+1 Bispecific Antibody Variant Sequences (VH and VL of
sequences in Table 24)

| 2 + 1 Bispecific | | |
|---|---|---|
| Antibody Name | VH | VL |
| CD98hc.04.064. | QVQLVQSGAEVKKPGASVKVSCKASGYT | DIVMTQSPDSLAVSLGERATINCKSSQS |
| 4a.1 | FTSYYMNWVRQAPGQGLEWMGIIDPSES | LLNNINQKNYLAWYQQKPGQPPKLLIYF |
| | ETNYAQKFQGRVTSTVDKSISTAYMELS | ASTRESGVPDRFSGSGSGTDFTLTISSL |
| | RLRSDDTVVYYCARASYGKGYFDYWGQG | QAEDVAVYYCQQHYSSPFTFGGGTKVEI |
| | TLVTVSS | K |
| | (SEQ ID NO: 375) | (SEQ ID NO: 376) |
| CD98hc.04.064. | QVQLVQSGAEVKKPGASVKVSCKASGYT | DIVMTQSPDSLAVSLGERATINCKSSQS |
| 4a.2 | FTSYYMNWVRQAPGQGLEWMGIIDPSQS | LLNNINQKNYLAWYQQKPGQPPKLLIYF |
| | ETNYAQKFQGRVTSTVDKSISTAYMELS | ASTRESGVPDRFSGSGSGTDFTLTISSL |
| | RLRSDDTVVYYCARASYGKGYFDYWGQG | QAEDVAVYYCQQHYSSPFTFGGGTKVEI |
| | TLVTVSS | K |
| | (SEQ ID NO: 377) | (SEQ ID NO: 378) |
| CD98hc.04.064. | QVQLVQSGAEVKKPGASVKVSCKASGYT | DIVMTQSPDSLAVSLGERATINCKSSQS |
| 4a.3 | FTSYYMNWVRQAPGQGLEWMGIIDPSSS | LLNNINQKNYLAWYQQKPGQPPKLLIYF |
| | ETNYAQKFQGRVTSTVDKSISTAYMELS | ASTRESGVPDRFSGSGSGTDFTLTISSL |
| | RLRSDDTVVYYCARASYGKGYFDYWGQG | QAEDVAVYYCQQHYSSPFTFGGGTKVEI |
| | TLVTVSS | K |
| | (SEQ ID NO: 379) | (SEQ ID NO: 380) |
| CD98hc.04.064. | QVQLVQSGAEVKKPGASVKVSCKASGYT | DIVMTQSPDSLAVSLGERATINCKSSQS |
| 4a.4 | FTSYYMNWVRQAPGQGLEWMGIIDPSGS | LLNNINQKNYLAWYQQKPGQPPKLLIYF |
| | ETNYAQKFQGRVTSTVDKSISTAYMELS | ASTRESGVPDRFSGSGSGTDFTLTISSL |
| | RLRSDDTVVYYCARASYGKGYFDYWGQG | QAEDVAVYYCQQHYSSPFTFGGGTKVEI |
| | TLVTVSS | K |
| | (SEQ ID NO: 381) | (SEQ ID NO: 382) |
| CD98hc.04.064. | QVQLVQSGAEVKKPGASVKVSCKASGYT | DIVMTQSPDSLAVSLGERATINCKSSQS |
| 4a.5 | FTSYYMNWVRQAPGQGLEWMGIIDPSAS | LLNNINQKNYLAWYQQKPGQPPKLLIYF |
| | ETNYAQKFQGRVTSTVDKSISTAYMELS | ASTRESGVPDRFSGSGSGTDFTLTISSL |
| | RLRSDDTVVYYCARASYGKGYFDYWGQG | QAEDVAVYYCQQHYSSPFTFGGGTKVEI |
| | TLVTVSS | K |
| | (SEQ ID NO: 383) | (SEQ ID NO: 384) |
| CD98hc.04.064. | QVQLVQSGAEVKKPGASVKVSCKASGYT | DIVMTQSPDSLAVSLGERATINCKSSQS |
| 4a.6 | FTSYYMNWVRQAPGQGLEWMGIIDPSES | LLENINQKNYLAWYQQKPGQPPKLLIYF |
| | ETNYAQKFQGRVTSTVDKSISTAYMELS | ASTRESGVPDRFSGSGSGTDFTLTISSL |
| | RLRSDDTVVYYCARASYGKGYFDYWGQG | QAEDVAVYYCQQHYSSPFTFGGGTKVEI |
| | TLVTVSS | K |
| | (SEQ ID NO: 385) | (SEQ ID NO: 386) |
| CD98hc.04.064. | QVQLVQSGAEVKKPGASVKVSCKASGYT | DIVMTQSPDSLAVSLGERATINCKSSQS |
| 4a.7 | FTSYYMNWVRQAPGQGLEWMGIIDPSES | LLQNINQKNYLAWYQQKPGQPPKLLIYF |
| | ETNYAQKFQGRVTSTVDKSISTAYMELS | ASTRESGVPDRFSGSGSGTDFTLTISSL |
| | RLRSDDTVVYYCARASYGKGYFDYWGQG | QAEDVAVYYCQQHYSSPFTFGGGTKVEI |
| | TLVTVSS | K |
| | (SEQ ID NO: 387) | (SEQ ID NO: 388) |
| CD98hc.04.064. | QVQLVQSGAEVKKPGASVKVSCKASGYT | DIVMTQSPDSLAVSLGERATINCKSSQS |
| 4a.8 | FTSYYMNWVRQAPGQGLEWMGIIDPSES | LLSNINQKNYLAWYQQKPGQPPKLLIYF |
| | ETNYAQKFQGRVTSTVDKSISTAYMELS | ASTRESGVPDRFSGSGSGTDFTLTISSL |
| | RLRSDDTVVYYCARASYGKGYFDYWGQG | QAEDVAVYYCQQHYSSPFTFGGGTKVEI |
| | TLVTVSS | K |
| | (SEQ ID NO: 389) | (SEQ ID NO: 390) |
| CD98hc.04.064. | QVQLVQSGAEVKKPGASVKVSCKASGYT | DIVMTQSPDSLAVSLGERATINCKSSQS |
| 4a.9 | FTSYYMNWVRQAPGQGLEWMGIIDPSES | LLGNINQKNYLAWYQQKPGQPPKLLIYF |
| | ETNYAQKFQGRVTSTVDKSISTAYMELS | ASTRESGVPDRFSGSGSGTDFTLTISSL |
| | RLRSDDTVVYYCARASYGKGYFDYWGQG | QAEDVAVYYCQQHYSSPFTFGGGTKVEI |
| | TLVTVSS | K |
| | (SEQ ID NO: 391) | (SEQ ID NO: 392) |
| CD98hc.04.064. | QVQLVQSGAEVKKPGASVKVSCKASGYT | DIVMTQSPDSLAVSLGERATINCKSSQS |
| 4a.10 | FTSYYMNWVRQAPGQGLEWMGIIDPSES | LLANINQKNYLAWYQQKPGQPPKLLIYF |
| | ETNYAQKFQGRVTSTVDKSISTAYMELS | ASTRESGVPDRFSGSGSGTDFTLTISSL |
| | RLRSDDTVVYYCARASYGKGYFDYWGQG | QAEDVAVYYCQQHYSSPFTFGGGTKVEI |
| | TLVTVSS | K |
| | (SEQ ID NO: 393) | (SEQ ID NO: 394) |
| CD98hc.04.064. | QVQLVQSGAEVKKPGASVKVSCKASGYT | DIVMTQSPDSLAVSLGERATINCKSSQS |
| 4a.11 | FTSYYMNWVRQAPGQGLEWMGIIDPSES | LLHNINQKNYLAWYQQKPGQPPKLLIYF |
| | ETNYAQKFQGRVTSTVDKSISTAYMELS | ASTRESGVPDRFSGSGSGTDFTLTISSL |
| | RLRSDDTVVYYCARASYGKGYFDYWGQG | QAEDVAVYYCQQHYSSPFTFGGGTKVEI |
| | TLVTVSS | K |
| | (SEQ ID NO: 395) | (SEQ ID NO: 396) |
| CD98hc.04.064. | QVQLVQSGAEVKKPGASVKVSCKASGYT | DIVMTQSPDSLAVSLGERATINCKSSQS |
| 4a.12 | FTSYYMNWVRQAPGQGLEWMGIIDPSES | LLYNINQKNYLAWYQQKPGQPPKLLIYF |
| | ETNYAQKFQGRVTSTVDKSISTAYMELS | ASTRESGVPDRFSGSGSGTDFTLTISSL |
| | RLRSDDTVVYYCARASYGKGYFDYWGQG | QAEDVAVYYCQQHYSSPFTFGGGTKVEI |
| | TLVTVSS | K |
| | (SEQ ID NO: 397) | (SEQ ID NO: 398) |
| CD98hc.04.064. | QVQLVQSGAEVKKPGASVKVSCKASGYT | DIVMTQSPDSLAVSLGERATINCKSSQS |
| 4a.13 | FTSYAMNWVRQAPGQGLEWMGIIDPSES | LLNNINQKNYLAWYQQKPGQPPKLLIYF |
| | ETNYAQKFQGRVTSTVDKSISTAYMELS | ASTRESGVPDRFSGSGSGTDFTLTISSL |
| | RLRSDDTVVYYCARASYGKGYFDYWGQG | QAEDVAVYYCQQHYSSPFTFGGGTKVEI |
| | TLVTVSS | K |
| | (SEQ ID NO: 399) | (SEQ ID NO: 400) |
| CD98hc.04.064. | QVQLVQSGAEVKKPGASVKVSCKASGYT | DIVMTQSPDSLAVSLGERATINCKSSQS |
| 4a.15 | FTSYFMNWVRQAPGQGLEWMGIIDPSES | LLNNINQKNYLAWYQQKPGQPPKLLIYF |
| | ETNYAQKFQGRVTSTVDKSISTAYMELS | ASTRESGVPDRFSGSGSGTDFTLTISSL |
| | RLRSDDTVVYYCARASYGKGYFDYWGQG | QAEDVAVYYCQQHYSSPFTFGGGTKVEI |
| | TLVTVSS | K |
| | (SEQ ID NO: 401) | (SEQ ID NO: 402) |
| CD98hc.04.064. | QVQLVQSGAEVKKPGASVKVSCKASGYT | DIVMTQSPDSLAVSLGERATINCKSSQS |
| 4a.16 | FTSYFMNWVRQAPGQGLEWMGIIDPSAS | LLGNINQKNYLAWYQQKPGQPPKLLIYF |
| | ETNYAQKFQGRVTSTVDKSISTAYMELS | ASTRESGVPDRFSGSGSGTDFTLTISSL |
| | RLRSDDTVVYYCARASYGKGYFDYWGQG | QAEDVAVYYCQQHYSSPFTFGGGTKVEI |
| | TLVTVSS | K |
| | (SEQ ID NO: 403) | (SEQ ID NO: 404) |

TABLE 26
Affinity Optimized 2 + 1 Bispecific Antibody CDR Sequences (Kabat)

| Antibody | CDR-H1 | CDR-H2 | CDR-H3 |
|---|---|---|---|
| CD98hc.04.064.4a.1 | SYYMN (SEQ ID | IIDPSESETNYAQKF | ASYGKGYFDY |
| | NO: 419) | QG (SEQ ID NO: 422) | (SEQ ID NO: 112) |
| CD98hc.04.064.4a.2 | SYYMN (SEQ ID | IIDPSQSETNYAQKF | ASYGKGYFDY |
| | NO: 419) | QG (SEQ ID NO: 423) | (SEQ ID NO: 112) |
| CD98hc.04.064.4a.3 | SYYMN (SEQ ID | IIDPSSSETNYAQKF | ASYGKGYFDY |
| | NO: 419) | QG (SEQ ID NO: 424) | (SEQ ID NO: 112) |
| CD98hc.04.064.4a.4 | SYYMN (SEQ ID | IIDPSGSETNYAQKF | ASYGKGYFDY |
| | NO: 419) | QG (SEQ ID NO: 425) | (SEQ ID NO: 112) |
| CD98hc.04.064.4a.5 | SYYMN (SEQ ID | IIDPSASETNYAQKF | ASYGKGYFDY |
| | NO: 419) | QG (SEQ ID NO: 426) | (SEQ ID NO: 112) |
| CD98hc.04.064.4a.6 | SYYMN (SEQ ID | IIDPSESETNYAQKF | ASYGKGYFDY |
| | NO: 419) | QG (SEQ ID NO: 422) | (SEQ ID NO: 112) |
| CD98hc.04.064.4a.7 | SYYMN (SEQ ID | IIDPSESETNYAQKF | ASYGKGYFDY |
| | NO: 419) | QG (SEQ ID NO: 422) | (SEQ ID NO: 112) |
| CD98hc.04.064.4a.8 | SYYMN (SEQ ID | IIDPSESETNYAQKF | ASYGKGYFDY |
| | NO: 419) | QG (SEQ ID NO: 422) | (SEQ ID NO: 112) |
| CD98hc.04.064.4a.9 | SYYMN (SEQ ID | IIDPSESETNYAQKF | ASYGKGYFDY |
| | NO: 419) | QG (SEQ ID NO: 422) | (SEQ ID NO: 112) |
| CD98hc.04.064.4a.10 | SYYMN (SEQ ID | IIDPSESETNYAQKF | ASYGKGYFDY |
| | NO: 419) | QG (SEQ ID NO: 422) | (SEQ ID NO: 112) |
| CD98hc.04.064.4a.11 | SYYMN (SEQ ID | IIDPSESETNYAQKF | ASYGKGYFDY |
| | NO: 419) | QG (SEQ ID NO: 422) | (SEQ ID NO: 112) |
| CD98hc.04.064.4a.12 | SYYMN (SEQ ID | IIDPSESETNYAQKF | ASYGKGYFDY |
| | NO: 419) | QG (SEQ ID NO: 422) | (SEQ ID NO: 112) |
| CD98hc.04.064.4a.13 | SYAMN (SEQ ID | IIDPSESETNYAQKF | ASYGKGYFDY |
| | NO: 420) | QG (SEQ ID NO: 422) | (SEQ ID NO: 112) |
| CD98hc.04.064.4a.15 | SYFMN (SEQ ID | IIDPSESETNYAQKF | ASYGKGYFDY |
| | NO: 421) | QG (SEQ ID NO: 422) | (SEQ ID NO: 112) |
| CD98hc.04.064.4a.16 | SYFMN (SEQ ID | IIDPSASETNYAQKF | ASYGKGYFDY |
| | NO: 421) | QG (SEQ ID NO: 426) | (SEQ ID NO: 112) |
| Antibody | CDR-L1 | CDR-L2 | CDR-L3 |
| CD98hc.04.064.4a.1 | KSSQSLLNNINQKNYL | FASTRES (SEQ ID | QQHYSSPFT |
| | A (SEQ ID NO: 176) | NO: 177) | (SEQ ID NO: 178) |
| CD98hc.04.064.4a.2 | KSSQSLLNNINQKNYL | FASTRES (SEQ ID | QQHYSSPFT |
| | A (SEQ ID NO: 176) | NO: 177) | (SEQ ID NO: 178) |
| CD98hc.04.064.4a.3 | KSSQSLLNNINQKNYL | FASTRES (SEQ ID | QQHYSSPFT |
| | A (SEQ ID NO: 176) | NO: 177) | (SEQ ID NO: 178) |
| CD98hc.04.064.4a.4 | KSSQSLLNNINQKNYL | FASTRES (SEQ ID | QQHYSSPFT |
| | A (SEQ ID NO: 176) | NO: 177) | (SEQ ID NO: 178) |
| CD98hc.04.064.4a.5 | KSSQSLLNNINQKNYL | FASTRES (SEQ ID | QQHYSSPFT |
| | A (SEQ ID NO: 176) | NO: 177) | (SEQ ID NO: 178) |
| CD98hc.04.064.4a.6 | KSSQSLLENINQKNYL | FASTRES (SEQ ID | QQHYSSPFT |
| | A (SEQ ID NO: 427) | NO: 177) | (SEQ ID NO: 178) |
| CD98hc.04.064.4a.7 | KSSQSLLQNINQKNYL | FASTRES (SEQ ID | QQHYSSPFT |
| | A (SEQ ID NO: 428) | NO: 177) | (SEQ ID NO: 178) |
| CD98hc.04.064.4a.8 | KSSQSLLSNINQKNYL | FASTRES (SEQ ID | QQHYSSPFT |
| | A (SEQ ID NO: 429) | NO: 177) | (SEQ ID NO: 178) |
| CD98hc.04.064.4a.9 | KSSQSLLGNINQKNYL | FASTRES (SEQ ID | QQHYSSPFT |
| | A (SEQ ID NO: 430) | NO: 177) | (SEQ ID NO: 178) |
| CD98hc.04.064.4a.10 | KSSQSLLANINQKNYL | FASTRES (SEQ ID | QQHYSSPFT |
| | A (SEQ ID NO: 431) | NO: 177) | (SEQ ID NO: 178) |
| CD98hc.04.064.4a.11 | KSSQSLLHNINQKNYL | FASTRES (SEQ ID | QQHYSSPFT |
| | A (SEQ ID NO: 432) | NO: 177) | (SEQ ID NO: 178) |
| CD98hc.04.064.4a.12 | KSSQSLLYNINQKNYL | FASTRES (SEQ ID | QQHYSSPFT |
| | A (SEQ ID NO: 433) | NO: 177) | (SEQ ID NO: 178) |
| CD98hc.04.064.4a.13 | KSSQSLLNNINQKNYL | FASTRES (SEQ ID | QQHYSSPFT |
| | A (SEQ ID NO: 176) | NO: 177) | (SEQ ID NO: 178) |
| CD98hc.04.064.4a.15 | KSSQSLLNNINQKNYL | FASTRES (SEQ ID | QQHYSSPFT |
| | A (SEQ ID NO: 176) | NO: 177) | (SEQ ID NO: 178) |
| CD98hc.04.064.4a.16 | KSSQSLLGNNINQKN | FASTRES (SEQ ID | QQHYSSPFT |
| | YLA (SEQ ID NO: 434) | NO: 177) | (SEQ ID NO: 178) |

### Example 20: Evaluation of In Vitro Effector Function (CDC and ADCC)

To investigate the mechanism of reticulocyte depletion seen in 2+1 anti-CD98hc bispecific antibodies, the ability of these same antibodies was evaluated to elicit in vitro effector responses, such as complement dependent cytotoxicity (CDC) and antibody-dependent cellular cytotoxicity (ADCC).

CDC method and results: The ability of 2+1 anti-CD98hc bispecific antibodies to drive complement deposition was measured on the hCMEC/D3 cell line, which expresses high levels of both receptors. Target cells were detached, washed 1× in PBS, and diluted to 2×10\hat{ }6 cells/mL in RPMI 1640 media. 50 μL of target cells were aliquoted per well (1×10\hat{ }5 cells per well) in round-bottom 96 well plates (Falcon #351177). To these cells was added 25 μL of 4× antibody diluted in the same media. Cells and antibody were incubated at 37° C. for 15 min, then 25 μL of pooled complement human serum (Innovative Research, IPLA-CSER) was added per well as a complement source and the plates incubated for a further 2 h at 37° C. Afterwards, cells were washed 2× with FACS buffer (PBS+2% FBS+1 mM EDTA) and resuspended in 50 μL of FACS buffer+0.25 μL/well of propidium iodide (Fischer Scientific, BD 556463) prior to analysis on an iQue flow cytometer (IntelliCyt). CDC was analyzed as the proportion of PI+ single cells in each well. In this assay, none of the 2+1 anti-CD98hc bispecific antibodies induced a significant CDC response (data not shown).

ADCC method and results: The ability of 2+1 bispecific antibodies to cause antibody-dependent cellular cytotoxicity (ADCC) was evaluated using an ADCC Reporter Bioassay system from Promega (#G7010). This system relies on an engineered Jurkat T cell line stably expressing the FcgRIIIa receptor (V158 variant) and an NFAT response element driving expression of firefly luciferase. Target cells were diluted in assay buffer (RPMI+4% low IgG Serum) at a concentration of 1.2×10\hat{ }6 per mL and 25 μL of cells (30,000 per well) were aliquoted to the appropriate wells of a 96-well white assay plate. To wells containing cells was added 25 μL of 3× antibody, also diluted in assay buffer. Following addition of antibody to target cells, the provided effector cells (frozen at 2×10\hat{ }7 per mL) were thawed at 37° C., and 630 μL added to 3.6 mL of warmed (37° C.) assay buffer, gently mixed, and 25 μL of effector cells (75000 per well, for an E:T ratio of 2.5) immediately added to the wells containing target cells and antibody. The plate was then incubated at 37° C. and 5% CO2 for 6 hrs to allow for receptor cell activation and luciferase expression. After 6 hrs, the plate was equilibrated to room temperature (15 minutes), while the Bio-Glo Luciferase Assay Reagent was prepared. 75 μL of the Luciferase Assay Reagent were added to each well, the plate was incubated for 10 minutes, and luminescence was measured on a BioTek plate reader.

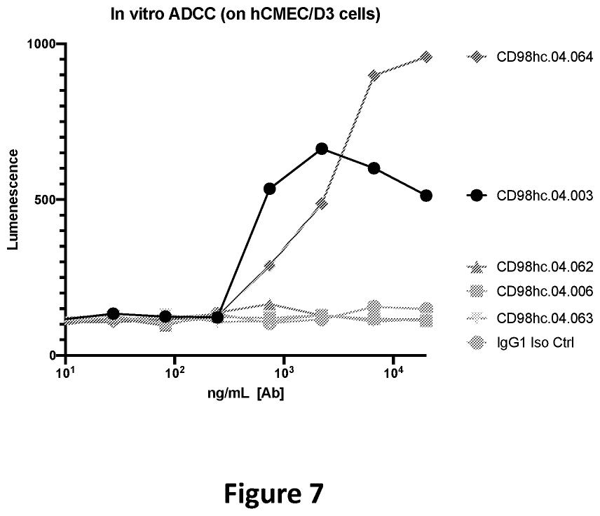

In the wildtype human IgG1 format, certain of the 2+1 bispecific antibodies against CD98hc (CD98hc.04.064 and CD98hc.04.003) were able to drive significant ADCC signal (FIG. 7), indicating an advantage in utilizing inactive Fc.

### Example 21: Affinity Data for 04.064 Humanized Panel

Binding kinetics of humanized anti-CD98hc antibodies were evaluated using the Biacore T200 (Cytiva). Briefly, IgGs were diluted to 10 μg/mL and captured using a Protein A/G (ThermoFisher, #21186) surface on a CM5 chip that was prepared by amine coupling according to the instrument manufacturer's recommendations. The captured antibodies were tested for their ability to bind human and cynomolgus monkey CD98hc as follows.

Recombinant human and cynomolgus CD98hc analytes described in the methods above were diluted in running buffer (HBS-EP+, Teknova, #8022, with 0.5 mg/mL BSA, MP Biomedicals LLC, #820451), to a concentration of 1000 nM, then diluted 3-fold serially to 250, 62.5, and 15.6 nM. Each sample was injected for 2 minutes to allow association, followed by dissociation in buffer alone for 10 minutes. Each sample injection was followed by the regeneration of the chip with three 30-second injections of 10 mM glycine pH 1.7. Fresh antibody was captured at the beginning of each cycle.

Data were analyzed using Biacore evaluation software to generate kinetic constants. The equilibrium dissociation constants (KD) were calculated from the fitted association and dissociation rate constants (k-on and k-off) for each of the anti-CD98hc antibodies. These values are summarized in Table 27 below.

TABLE 27
Equilibrium dissociation constants (KD) for humanized
2 + 1 anti-CD98hc bispecific antibodies

| | huCD98hc Avi-His | cyno CD98hc Avi-His |
|---|---|---|
| Antibody | KD (M) | KD (M) |
| CD98hc.04.064.1d | 3.5E−08 | 1.5E−06 |
| CD98hc.04.064.1e | 2.5E−08 | 7.4E−07 |
| CD98hc.04.064.4d | 1.8E−08 | 3.4E−07 |

This data indicates that the 4.064 humanized panel bound human CD98hc at a range of about 18 nM to 35 nM and cynomolgus CD98hc at a range of about 340 nM to 1.5 μM.

### Example 22: Serum and Brain PK Data of CD98hc.04.064 Humanized Panel after Intravenous Injection into hCD98hc+/+KI Mice

Antibody Production Method: 2+1 antibodies as well as a matched control antibody (same Fab and Fc domain but lacking the scFv targeting CD98hc) for in vivo PK studies were generated via transient transfection of ExpiCHO cells (Invitrogen) performed according to manufacturer's instruction and purified as described above in Example 17.

Study design: For this study, homozygous knock-in (KI) mice (as described in Example 17) were utilized (mice in which the ectodomain region of mouse CD98hc was replaced with that of human CD98hc in both alleles (huCD98hc+/+)). A two-dose study was conducted to determine both the brain uptake, as well as peripheral clearance of the injected antibodies over time. Briefly, groups (N=3) of homozygous (huCD98hc+/+KI) mice were dosed intravenously with 10 mg/kg of either isotype IgG or 2+1 anti-CD98hc bispecific antibody at day 1 and 15. Blood samples were then collected from antibody treated animals at various timepoints in serum separator tubes and allowed to clot at room temperature (RT) before centrifugation. The resulting supernatants, representing sera, were subsequently transferred into new tubes and stored at −80° C. until analysis.

Samples from vessel depleted brain and serum were processed and analyzed as described in Example 17.

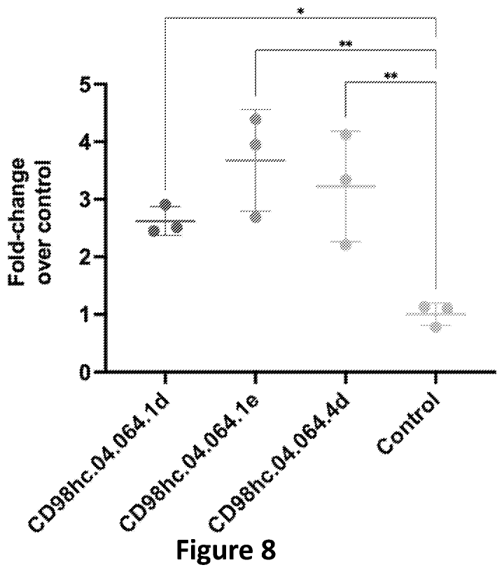

FIG. 8 illustrates antibody levels in vessel-depleted brain fraction of huCD98hc KI+/+ mice, 24 hrs after a 2nd 10 mg/kg dose of 2+1 anti-CD98hc and control antibodies. Antibody levels are shown as fold change over a matched control with the same Fab and Fc but no scFv binding domain to CD98hc. As illustrated in FIG. 8, levels of 2+1 anti-CD98hc bispecific CD98hc.04.064 humanized lead antibodies were determined to be 3-4.5-fold higher than TD1 IgG in the vessel depleted brain fraction of huCD98hc+/+KI mice. The peripheral PK was determined by measuring antibody concentrations in plasma samples of antibody treated mice.

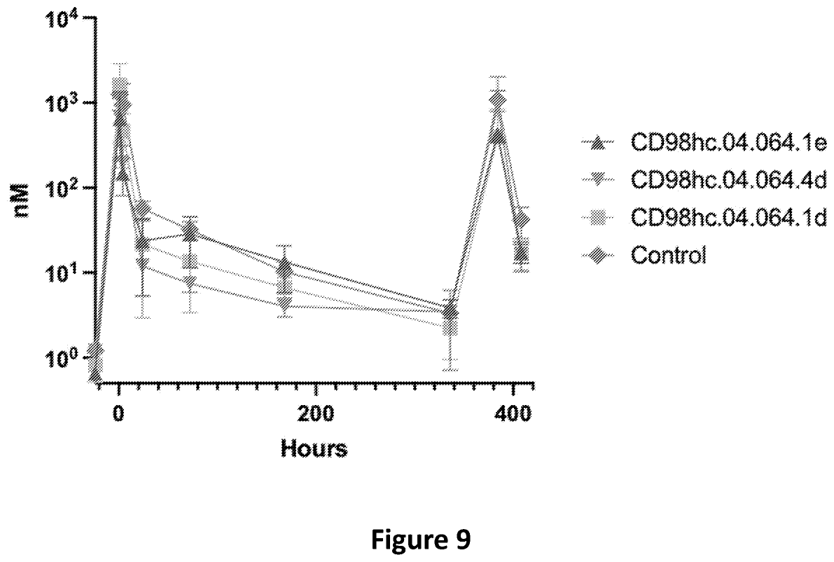

FIG. 9 illustrates antibody levels in the serum of huCD98hc KI+/+ mice, 24 hrs after a 2nd 10 mg/kg dose of 2+1 anti-CD98hc and control antibodies. Antibody levels in vessel-depleted brain fraction of huCD98hc KI+/+ mice, 24 hrs after a 2nd 10 mg/kg dose of 2+1 anti-CD98hc and control antibodies. As illustrated in FIG. 9, all 2+1 anti-CD98hc bispecific 04.064 humanized antibodies tested exhibited similar clearance rates compared to the control IgG in the huCD98hc+/+KI mice. CD98hc.04.064.1e was further engineered.

### Example 23: Affinity Data for Engineered Variants of CD98hc.04.064

Binding kinetics of 2+1 anti-CD98hc bispecific antibodies to recombinant human and cynomolgus CD98hc ECD Avi-His were evaluated using a Biacore T200 instrument (Global Life Sciences Solutions USA LLC, Marlborough, MA). The 2+1 anti-CD98hc bispecific antibodies were prepared by diluting to 50 μg/mL in running buffer HBS-EP+ (Teknova) with 0.5 mg/mL BSA (MP Biomedicals) and captured by a goat anti-human kappa polyclonal antibody (Southern Biotech) surface that was prepared according to the instrument manufacturer's recommendations. Two independent experiments were performed as follows, resulting in an N of between one and three determinations for each antibody. Estimates of affinity were generated by injecting each analyte over captured antibody on Flowcell 2, 3, or 4. CD98hc ECD Avi-His analytes were diluted with running buffer, in a series of five, three-fold serial dilutions starting from 600 nM. Analytes were injected without regeneration, from lowest to highest concentration, for 5 minutes each, and dissociation was followed for 15 minutes. After each analyte injection series, antibodies were regenerated with 10 mM glycine pH 2.0 buffer (Cytiva). Data were processed and analyzed using Biacore T200 BiaEvaluation software (Cytiva).

The equilibrium dissociation constants (K_{D}) were calculated from the fitted association and dissociation rate constants (k-on and k-off) for 2+1 anti-CD98hc bispecific antibodies of the present disclosure binding to human and cynomolgus CD98hc ECD Avi-His. For measurements with N>1, means and standard deviation were calculated and measurements for all samples were combined and graphed using GraphPad Prism. The K_{D} values are summarized in Table 28 below.

TABLE 28
Equilibrium dissociation constants (K_{D}) for 2 + 1 humanized
variants of CD98hc.04.064 in 2 + 1 format.

| | Human CD98hc ECD Avi-His | | | | | |
|---|---|---|---|---|---|---|
| | Mean K_{D} | | | Cyno CD98hc ECD Avi-His | | |
| Antibody | (M) | SD | N | Mean K_{D} (M) | SD | N |
| CD98hc.0 | 3.1E−08 | 8.0E−10 | 3 | 8.8E−07 | NA | 1 |
| 4.064.1a | | | | | | |
| CD98hc.0 | 3.0E−08 | 1.0E−09 | 3 | 3.3E−07 | NA | 1 |
| 04.064.1b | | | | | | |
| CD98hc.0 | 2.4E−08 | 1.7E−09 | 3 | 3.6E−07 | NA | 1 |
| 04.064.1d | | | | | | |
| CD98hc.0 | 1.3E−08 | 5.5E−10 | 3 | 1.2E−07 | 1.5E−08 | 2 |
| 4.064.1e* | | | | | | |
| CD98hc.0 | 3.4E−08 | 5.2E−09 | 2 | 4.3E−07 | NA | 1 |
| 4.064.4a | | | | | | |
| CD98hc.0 | 3.0E−08 | 1.1E−09 | 2 | 6.2E−07 | NA | 1 |
| 4.064.4b | | | | | | |
| CD98hc.0 | 1.7E−08 | 1.0E−09 | 3 | 3.1E−07 | NA | 1 |
| 4.064.4c | | | | | | |
| CD98hc.0 | 1.2E−08 | 7.6E−10 | 3 | 1.6E−07 | NA | 1 |
| 4.064.4d | | | | | | |

N = number of determinations; NA = not applicable;
* Inconsistent results were obtained in an additional equilibrium dissociation constant experiment.

These results illustrate that 2+1 anti-CD98hc bispecific antibodies exhibit a range of affinities from approximately 13 nM to 880 nM for human and cynomolgus CD98hc ECD Avi-His binding. In particular, affinity of 2+1 anti-CD98hc bispecific antibodies for binding to human CD98hc ECD Avi-His ranged from 12 nM to 34 nM, while binding to cyno CD98hc ECD Avi-His ranged from 120 nM to 880 nM.

### Example 24: TD1-CD98hc Bi-Specific Antibody in an In Vitro sTREM2 Assay

In order to test the function of a TD1-CD98hc antibody (that was used as a PK/PD model for CD98hc-mediated antibody delivery into the brain) a sTREM2 assay was performed. Human monocytes were isolated from whole blood using RosetteSep Human monocyte enrichment cocktail (Stemcell technologies) and Ficoll centrifugation per manufacturer protocols. After lysing red blood cells with ACK lysing buffer, monocytes were resuspended in complete media (RPMI, 10% FBS, Pen/Strep, L-glutamine, HEPES, non-essential amino acid, and sodium pyruvate). To obtain macrophages from these isolated monocytes, 50 ng/ml human M-CSF and 8% v/v human serum were added to the cells for 5-7 days.

Macrophages were differentiated from 4 human donors in 2 independent experiments. 105 macrophages per well were stimulated with 1 μg/mL of tested antibody (huIgG1, TD1, or TD1-CD98hc) for 48 hours. Duplicate wells were treated as technical replicates. Supernatants were tested for sTREM2 by MSD platform.

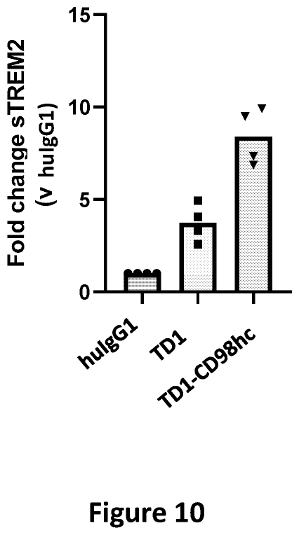

The technical replicates for each donor were averaged, then the fold change compared to NSLF isotype control was calculated and graphed. Each dot on the graph in FIG. 10 represents an individual donor. Matched one way ANOVA was used to calculate statistical significance.

As illustrated in FIG. 10, TD1-CD98hc was found to be active in an in vitro sTREM2 assay compared to hIgG1. TD1-CD98hc also elevated sTREM2 as well or better than TD1.

### Example 25: TD1-CD98hc Bi-Specific Antibody and Method for Intravenous Administration to Non-Human Primates

A bi-specific antibody was generated with: (i) CD98hc.04.064.1e scFV; and (ii) an antibody specific for human MSA4A (TD1), in a 2+1 (hole) format (referred to as “TD1-CD98hc”). The full amino acid sequence of TD1-CD98hc is below:

Variable light chain region:
(SEQ ID NO: 405)
DVVMTQSPLSLPVTLGQPASISCKSSRSLLYSAGKTYLSWFQQRPGQSP
RRLIYLVSKLDSGVPDRFSGSGSGTDFTLKISRVEAEDVGVYYCWQGID
FHQTFGGGTKVEIK.
Constant light chain region:
(SEQ ID NO: 406)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC.
Variable heavy chain region:
(SEQ ID NO: 407)
QVQLVQSGSELKKPGASVKVSCKASGYAFTSYGLSWVRQAPGQGLEWMG
WINTYSGVPTYAQGFTGRFVFSLDTSVSTAYLQISSLKAEDTAVYYCAR
TMADYWGQGTLVTVSS.
CH + Fc #1 (knob, T366W):
(SEQ ID NO: 408)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSSKAFPAPIEKTISKAKGQPREPQVYTLPPSRDELTKNQ
VSLWCLVKGFYPSDIAVEWESNGQPENNYKTTPPVLDSDGSFFLYSKLT
VDKSRWQQGNVFSCSVMHEALHNHYTQKSLSLSPGK.
CH + Fc #2 (hole, T366S, L368A, Y407V):
(SEQ ID NO: 409)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSSKAFPAPIEKTISKAKGQPREPQVYTLPPSRDELTKNQ
VSLSCAVKGFYPSDIAVEWESNGQPENNYKTTPPVLDSDGSFFLVSKLT
VDKSRWQQGNVFSCSVMHEALHNRFTQKSLSLSPGK.
Linker-targeting domain:
(SEQ ID NO: 410)
GGGGSGGGGSGGGGSQVQLVQSGAEVKKPGASVKVSCKASGYTFTSYWM
HWVRQAPGQGLEWMGIIDPSDSETHYAQKFQGRVTMTVDKSTSTVYMEL
SSLRSEDTAVYYCARASYGKGYFDYWGQGTLVTVSSGGSEGKSSGSGSE
SKSTGGSDIVMTQSPDSLAVSLGERATINCKSSQSLLNNINQKNYLAWY
QQKPGQPPKLLIYFASTRESGVPDRFSGSGSGTDFTLTISSLQAEDVAV
YYCQQHYSSPFTFGGGTKVEIK.

The full amino acid sequence of the antibody specific for human MSA4A alone (TD1) is below:

TD1 heavy chain hIgG1 NSLF (N325S, L328F)
(SEQ ID NO: 411)
QVQLVQSGSELKKPGASVKVSCKASGYAFTSYGLSWVRQAPGQGLEWMG
WINTYSGVPTYAQGFTGRFVFSLDTSVSTAYLQISSLKAEDTAVYYCAR
TMADYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYN
STYRVVSVLTVLHQDWLNGKEYKCKVSSKAFPAPIEKTISKAKGQPREP
QVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLS
PGK
TD1 light chain
(SEQ ID NO: 412)
DVVMTQSPLSLPVTLGQPASISCKSSRSLLYSAGKTYLSWFQQRPGQSP
RRLIYLVSKLDSGVPDRFSGSGSGTDFTLKISRVEAEDVGVYYCWQGID
FHQTFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKV
YACEVTHQGLSSPVTKSFNRGEC.

A PK/PD study was conducted in naïve male cynomolgus monkeys (n=9) following intravenous (iv) dosing of 20 mg/kg of each of the 3 antibodies (n=3) on Day 1 and 29 (total of 2 administrations).

TABLE 29
In vivo administration of anti-TD1-CD98hc bispecific antibody

| | Number of | | |
|---|---|---|---|
| | Animals | Test | Dosing |
| Group | (male only) | Article | Route |
| 1 | 3 | huIgG1 | IV |
| | | (Isotype | |
| | | Control) | |
| 2 | 3 | TD 1 | IV |
| 3 | 3 | TD1- | IV |
| | | CD98hc | |

The intravenous doses were administered via a saphenous vein as slow push injection. The target dose level (mg/kg), target dose concentration (mg/mL) and target dose volume (mL/kg) were consistent between Groups 1-3.

### Example 26: Hematological Parameters Following Following Intravenous Administration of TD1-CD98hc Bi-Specific Antibody to Non-Human Primates

At pre-dose and following dose administration on days 3, 8, 15, 29 and 31, blood samples (˜2 mL) for hematology were collected from the femoral vein into tubes containing potassium EDTA. The following parameters in Table 30 were tested:

TABLE 30
Hematology Parameters tested following intravenous
administration of anti-TD1-CD98hc antibody

| Red blood cell (erythrocyte) count | Platelet count |
|---|---|
| Hemoglobin | White blood cell (leukocyte) count |
| Hematocrit | Differential blood cell count (percent & |
| Mean corpuscular volume | absolute) |
| Mean corpuscular hemoglobin | Red cell distribution width |
| Mean corpuscular hemoglobin concentration | Blood smear |
| | Reticulocyte count |

Testing facility normal range for reticulocyte count is 25.7 -122.3 10³/μl.

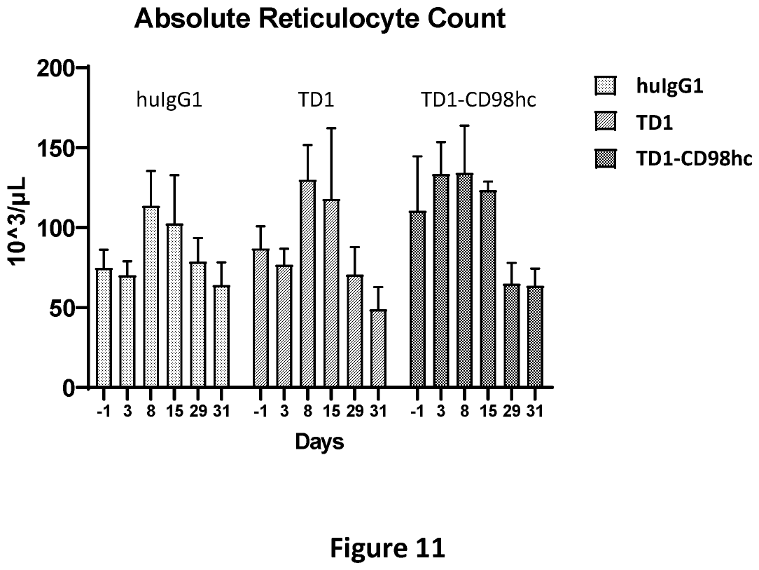

FIG. 11 illustrates absolute reticulocyte counts over the duration of the study.

Overall, no notable changes were observed in hematology parameters, and no consistent reduction in reticulocytes was observed over the course of the study.

### Example 27: CSF Penetrance of TD1-CD98hc Bi-Specific Antibody Following IV Administration in Non-Human Primates

Cerebrospinal fluid (CSF) and serum were collected from the NHPs at various times following the first and second administrations described in Example 25. The serum and CSF were tested as described below.

Sandwich ELISA: 100 μL/well of goat anti-human IgG, Monkey adsorbed-BIOTIN (Southern Biotech; catalog #2049-08) working solution (0.5 μg/mL) was added to a 96-well Pierce™ streptavidin coated high binding plate with SuperBlock™ blocking buffer (ThermoScientific; Reference #15500) and incubated for 1 hour+10 minutes at Room Temperature (RT) on a plate shaker at 350 revolutions per minute (RPM). The serum or CSF quality controls (QC) and test samples were diluted to the minimum required dilution (MRD) in assay buffer (1×TBS, 0.05% Tween-20, and 0.1% BSA) prior to loading onto the assay plate while the standard calibration curve and assay buffer QCs (AB-QCs) were added directly to the assay plate. After the incubation was complete, the plate was washed 3 times with wash buffer (1×PBS and 0.05% Tween-20) using a plate washer and 100 μL/well of the calibration standards, QCs, and MRD-diluted Serum or CSF QCs and test samples were then added to the plate and incubated for 2 hours+15 minutes at room temperature on a plate shaker at 350 RPM. The plate was then washed, and 100 μL/well of goat anti-human IgG, Monkey adsorbed-HRP (Southern Biotech; catalog #2049-05) working solution (0.08 μg/mL) was added to the plate and incubated for 1 hour+10 minutes at room temperature on a plate shaker at 350 RPM. After the HRP incubation, the plate was washed and 100 μL/well of tetramethylbenzidine (TMB) substrate solution (Surmodics Product No. TMBS-1000-01) was added and incubated for 15+10 minutes at room temperature (RT) shielded from light by covering in aluminum foil or placed in a drawer. After the incubation was complete, 100 μL/well Stop Solution (Surmodics Product No. NSTP-1000-01) was added to the plate to stop the reaction. The plate was then placed on a plate shaker at 350 RPM for at least 2 minutes at RT. The plate was then read on a Molecular Devices SpectraMax M5 instrument and a raw optical density (OD450-540) signal was produced from each well. The concentration of the analyte in each unknown sample was determined by interpolation of the concentration using the calibration standard curve. The standard regression was performed by SoftMax Pro (Molecular Devices/Version 7.1) using a 4-Parameter Logistic (4-PL) model with a weighting factor of 1/Y²

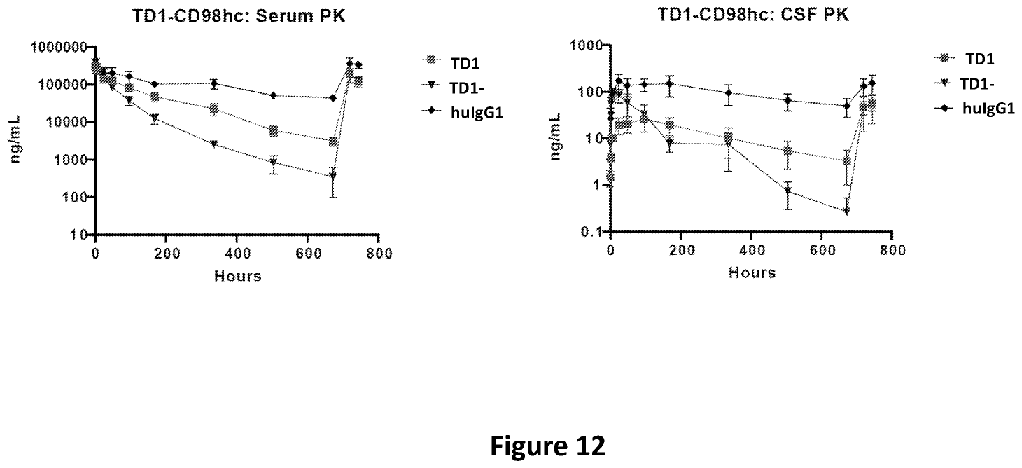

FIG. 12 illustrates that TD1-CD98hc demonstrated increased serum clearance in NHPs as compared to TD1 alone and isotype control. Further, as illustrated in FIG. 12, an increased CSF Cmax in CSF was seen with TD1-CD98hc administration in NHPs, as compared to TD1 alone. This increase was ˜13-fold at the 2 hr timepoint, ˜10-fold at the 6 hr timepoint and ˜4-fold at the 24 hr timepoint after the initial 10 mg/kg dose but waned after the 96 hr timepoint.

### Example 28: Effect of CD98hc Binding Arm on Brain Uptake and Brain Pharmacodynamics in Non-Human Primates

Brain Sample preparation: Brain tissues were collected from the antibody treated cynomolgus monkeys on day 30 (i.e., 48 hours after the second antibody administration) and subsequently frozen. Prior to this, animals were anesthetized and underwent cardiac perfusion with PBS to clear blood vessels. Brain tissues were then thawed and minced and homogenized in HBSS buffer (MilliporeSigma #55037C) containing 10 mM HEPES (#15630130, Gibco) using a hand-operated grinder. Subsequently, samples were centrifuged at 1000 g for 5 minutes to pellet the vessel portion. The supernatants, representing the parenchyma, were then transferred into new tubes, and mixed with 10× RIPA buffer (final concentration 1×) including protease and phosphatase inhibitor cocktail (Complete™, Mini Protease Inhibitor Cocktail, Roche, #11836153001). After 45 minutes incubation on a nutator at 4° C., samples were frozen on dry ice and stored at −80° C. until analysis. Vessel fractions were cleared from myelin debris via centrifugation through 18% Dextran (70 kDa, #31390, Sigma-Aldrich), followed by 2 washes in HBSS buffer. Afterwards, samples were lysed in RIPA buffer (R0278, Sigma-Aldrich) and stored at −80° C. until analysis. We have previously confirmed the validity of this method using Western Blot analysis, in which markers of brain endothelial cells (e.g., CD31 and Claudin-5) were absent in the parenchymal portions (data not shown).

Pharmacokinetic (PK) analysis: Antibody concentrations in brain samples were measured using MSD (Meso Scale Discovery) method. Briefly, 50 μl/well of goat anti-human IgG1 antibody was added to MSD plates at 1 μg/ml in PBS and incubated overnight on a shaker at 500 RPM at 4° C. Plates were then washed 3× with wash buffer (0.05% Tween-20 in 1×PBS), followed by incubation with blocking buffer (wash buffer containing 3% BSA) for 1 hour at RT. Subsequently, brain (diluted in PBS) samples were added in duplicates to the plates and incubated for 2 hours on a shaker at 500 RPM at room temperature. Plates were then washed 3× with wash buffer, followed by addition of 40 μl/well of Sulfo-Tag goat anti-human Ab (R32AJ-5, Meso Scale Discovery) at 0.5 g/ml in PBS and 1 hour incubation on a shaker at 500 RPM. Finally, after 3× washes with wash buffer, 150 μl/well of 1× READ buffer (R92PC-2, Meso Scale Discovery) was added to the plates, before they are being read using a Sector Imager S600 instrument. The standard curve for each treatment was generated using eleven 2-fold serial dilutions (from 100-0.019 ng/ml) of their respective antibody. The curves were fit using a four-parameter logistic regression for calculating antibody concentrations within samples.

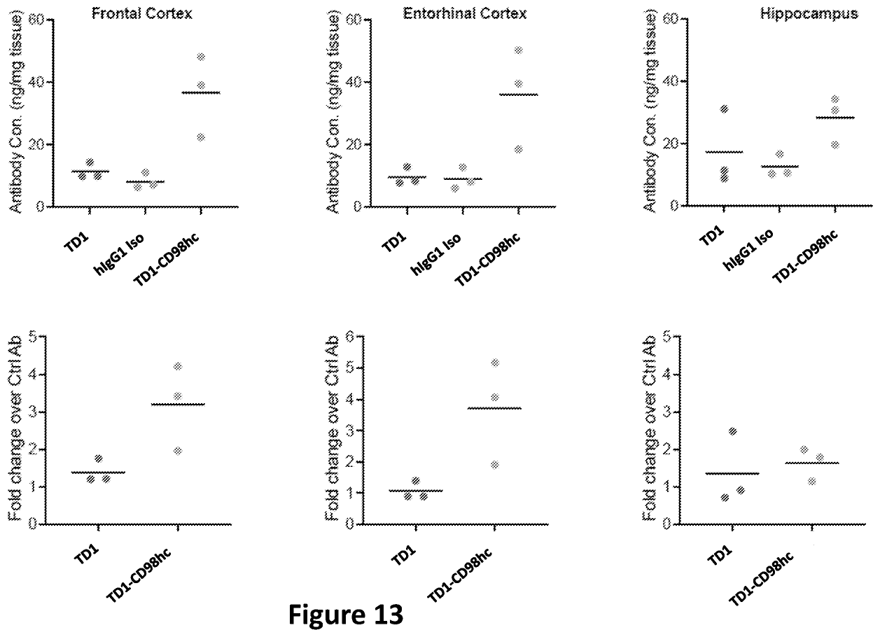

FIG. 13 illustrates that the CD98hc BBB targeting arm was able to increase brain uptake of the TD1-CD98hc compared to TD1 alone up to 3-fold in the frontal cortex, and up to 4-fold in the entorhinal cortex, with no significant increase seen in the hippocampus.

### Example 29: Pharmacodynamic Analysis of TD1-CD98hc Bi-Specific Antibody in NHP Samples

CSF from NHP test subjects, as described in Example 27, were further tested for pharmacodynamic purposes, as described below.

### CSFIR Method Summary (Brain Lysate)

An Enzyme-linked Immunosorbent Assay (ELISA) kit from R&D Systems (Catalog No. DY329) was used to measure the concentration of Colony Stimulating Factor 1 Receptor (CSF1R) in cynomolgus monkey brain lysates. The mouse anti-Human M-CSF R Capture Antibody (R&D Systems, Part #841246) was diluted in 1×PBS (Corning; REF 21-040-CM) to a working concentration of 4 μg/mL and coated at 100 μL/well on a 96-well microplate (Nunc-Immuno Maxisorp; ThermoScientific Catalog No. 446612) and incubated overnight at room temperature without shaking. The next day, the plate was washed 3× with wash buffer (1×PBS and 0.05% Tween-20), blocked with 300 μL of reagent diluent (R&D Systems, Part #841380) and incubated for at least 1 hour without shaking. Human M-CSF R Standards and QCs (R&D Systems, Part #841248) were prepared in Reagent Diluent. Test samples and QCs were diluted at the minimum required dilution (MRD) in Assay Buffer (1×PBS, 1% BSA, and 0.05% Tween-20). The plate was then washed, 100 μL/well of Human M-CSF R Standards, diluted QCs and diluted samples in Assay Buffer were added to the appropriate wells and incubated for 2 hours at room temperature. The plate was washed and 100 μL/well of the 100 ng/mL biotinylated Goat anti-Human M-CSF R detection antibody (R&D Systems, Part #841247) working solution in reagent diluent was added and incubated for 2 hours at room temperature. After the incubation was complete, the plate was washed and 1:200 dilution of Streptavidin-HRP (R&D Systems, Part #890803) in reagent diluent was then added and incubated for 20 minutes at room temperature. The plate was washed for a final time and 100 μL/well of substrate solution (R&D Systems, Parts #895000 and 895001) was added and incubated for 20 minutes at room temperature. After the incubation was complete, 50 μL/well of stop solution (R&D Systems, Part #895032) was added to the plate and mixed thoroughly. The plate was then read on a Molecular Devices SpectraMax M5 instrument and a raw optical density (OD450-540) signal was produced from each well. The concentration of CSFIR in each unknown sample was determined by interpolation against the calibration standard curve. The standard regression was performed by SoftMax Pro (Molecular Devices, version 7.1) using a 4-Parameter Logistic (4-PL) model with a weighting factor of 1/Y².

### CSF1 Method Summary (CSF)

An Electrochemiluminescence (ECL) Assay kit from Meso Scale Discovery (MSD, Catalog #K151XRK-4) was used to measure the concentrations of Colony Stimulating Factor 1 (CSF1) in Cynomolgus Monkey Cerebral Spinal Fluid (CSF) samples. A 96-well MSD GOLD Small Spot Streptavidin Plate (MSD, Catalog #L45SA-1) was coated with 25 μL/well of the biotinylated anti-human CSF1 Antibody (MSD, Catalog #C21XR-3) diluted 1:17.5 in Diluent 100 (MSD, Catalog #R50AA-4) and incubated for 1 hour at room temperature on a plate shaker set at 700 revolutions per minute (RPM).

Standards were prepared in Diluent 43 (MSD, Catalog #R50AG-2) and were further diluted 1:1 in assay buffer prior to loading onto the plate. QCs were prepared in Diluent 43 and were diluted at the MRD in assay buffer. Diluted QCs were further diluted 1:1 with Diluent 43 prior to loading onto the plate. Test samples were diluted at the MRD in assay buffer. Diluted samples were further diluted 1:1 in Diluent 43 prior to loading onto the plate.

After washing, 50 μL of Standards, QCs and test samples diluted in Diluent 43 were added to the appropriate wells and incubated for 1 hour at room temperature on a shaker set at 700 RPM. The plate was then washed and 50 μL of the detection antibody (SULFO-TAG Anti-Human CSF1 Antibody; MSD, Catalog #D21XR-3) diluted 1:100 in Diluent 3 (MSD, Catalog #R50AP-2) was added to each well and incubated for 1 hour at room temperature on a plate shaker set at 700 RPM. After the incubation was complete, the plates were washed and 150 μL/well of MSD GOLD Read Buffer B (MSD, Catalog #R60AM-2) was then added and plates were read on an MSD Sector 600 Imager. In the presence of read buffer, ruthenium produced a chemiluminescent signal when a voltage was applied. The intensity of the signal was proportional to the concentration of CSF1 present in the sample. The CSF1 levels were quantified according to the standard curve utilizing a four-parameter logistic (4-PL) curve fit equation with 1/y² weighting. Data was analyzed using Microsoft Excel and GraphPad Prism 9.0.

### STREM2 Method Summary (Serum, CSF and Brain Lysates)

An Electrochemiluminescence (ECL) Assay on the MSD platform was used to measure the concentration of Triggering Receptor Expressed on Myeloid Cells 2 (TREM2) in Cynomolgus Monkey Serum, Cerebral Spinal Fluid (CSF), and Brain Lysates. A 96-well standard MSD plate (Catalog #L15XA) was coated with 50 μL/well of capture antibody T2-8F11 (generated at Alector), in 1×PBS (Corning; REF 21-040-CM) at 2-8° C. overnight on a plate shaker set at 500 revolutions per minute (RPM). After washing with wash buffer (1×PBS and 0.05% Tween-20), the plate was blocked with 150 μL/well of blocking buffer (1% heat-inactivated high-grade BSA in PBS). The plate was then washed and 50 μL/well of Cynomolgus TREM2-Fc Standards (generated at Alector), QCs and diluted samples in assay buffer (1×PBS, 0.05% Tween-20, 1% BSA) were added to the appropriate wells and incubated for 1 hour at room temperature (RT) on a shaker set at 500 RPM. TREM2 present in the Standards, QCs and samples were bound by the immobilized T2-8F11 capture antibody. After washing away any unbound substances, 50 μL/well of biotinylated goat anti-human TREM2 polyclonal antibody (R&D Systems, Catalog No. BAF1828) diluted in assay buffer at 100 ng/mL were added to wells of the plate and incubated for 1 hour at RT on a shaker set at 500 RPM. After washing the plates, 50 μL of Sulfo-Tag streptavidin (MSD, Catalog No. R32AD-1) solution (0.2 μg/mL) in assay buffer was added and incubated for 30 minutes at RT on a shaker set at 500 RPM. After the incubation was complete, the plates were washed and 150 μL/well of 1× Read buffer (prepared by diluting 4× Read Buffer T with Surfactant (MSD, Catalog #R92TC-1) with deionized water) was added and plates were read on an MSD Sector 600 Imager. In the presence of read buffer, ruthenium produced a chemiluminescent signal when a voltage was applied. The intensity of the signal was proportional to the concentration of TREM2 present in the sample. The standard regression was performed by SoftMax Pro (Molecular Devices, version 7.1) using a 4-Parameter Logistic (4-PL) model with a weighting factor of 1/Y².

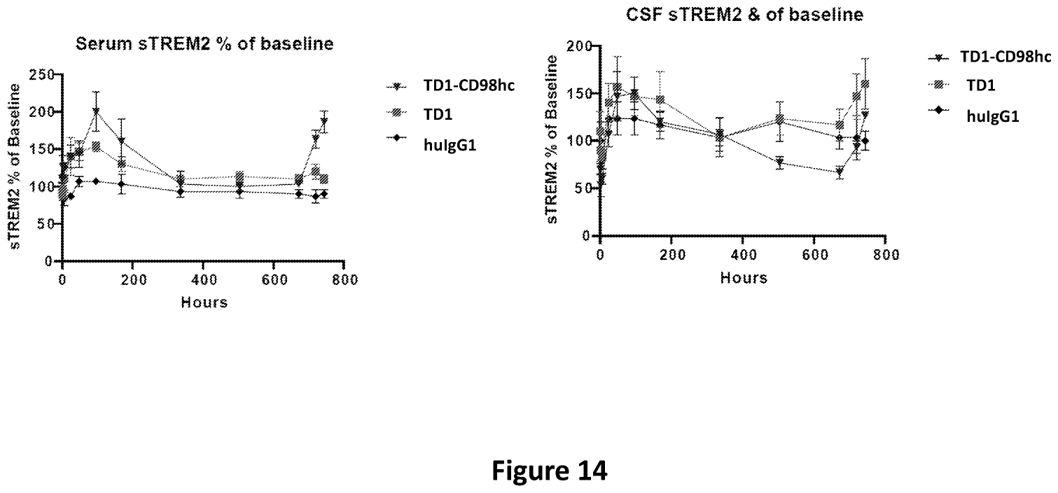

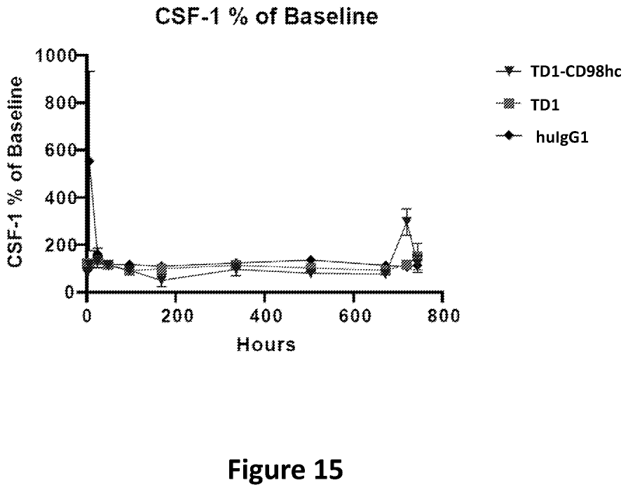

FIG. 14 illustrates that serum levels of soluble TREM2 (sTREM2) were increased in NHPs treated with TD1-CD98hc as compared to TD1 alone or isotype control following administration of the first and the second dose. FIG. 14 further illustrates that soluble TREM2 in the CSF of NHPs treated with CD98hc was not elevated compared to TD1 alone, suggesting that TD1-98hc is being cleared from CSF at a higher rate than TD1 alone. FIG. 15 illustrates CSF-1 levels in CSF from NHPs treated with TD1-CD98hc or TD-1 alone. No consistent increase in CSF-1 levels was seen for any group.

1. An antigen-binding domain that specifically binds to human CD98 heavy chain (CD98hc), wherein the antigen-binding domain comprises heavy chain variable region (VH) complementarity determining region (CDR) 1, VH CDR2, VH CDR3 and light chain variable region (VL) CDR1, CDR2, and CDR3 sequences comprising the amino acid sequences of:
(i) SEQ ID NOs: 413, 414, 112, and 176-178, respectively;
(ii) SEQ ID NOs: 50-52 and 116-118, respectively;
(iii) SEQ ID NOs: 53-55 and 119-121, respectively;
(iv) SEQ ID NOs: 56-58 and 122-124, respectively;
(v) SEQ ID NOs: 59-61 and 125-127, respectively;
(vi) SEQ ID NOs: 62-64 and 128-130, respectively;
(vii) SEQ ID NOs: 65-67 and 131-133, respectively;
(viii) SEQ ID NOs: 68-70 and 134-136, respectively;
(ix) SEQ ID NOs: 71-73 and 137-139, respectively;
(x) SEQ ID NOs: 74-76 and 140-142, respectively;
(xi) SEQ ID NOs: 77-79 and 143-145, respectively;
(xii) SEQ ID NOs: 80-82 and 146-148, respectively;
(xiii) SEQ ID NOs: 83-85 and 149-151, respectively;
(xiv) SEQ ID NOs: 86-88 and 152-154, respectively;
(xv) SEQ ID NOs: 89-91 and 155-157, respectively;
(xvi) SEQ ID NOs: 92-94 and 158-160, respectively;
(xvii) SEQ ID NOs: 95-97 and 161-163, respectively;
(xviii) SEQ ID NOs: 98-100 and 164-166, respectively;
(xix) SEQ ID NOs: 101-103 and 167-169, respectively;
(xx) SEQ ID NOs: 104-106 and 170-172, respectively;
(xxi) SEQ ID NOs: 107-109 and 173-175, respectively;
(xxii) SEQ ID NOs: 110-112 and 176-178, respectively;
(xxiii) SEQ ID NOs: 113-115 and 179-181, respectively;
(xxiv) SEQ ID NOs: 226-228 and 273-275, respectively;
(xxv) SEQ ID NOs: 229-231 and 276-278, respectively;
(xxvi) SEQ ID NOs: 232-234 and 279-281, respectively;
(xxvii) SEQ ID NOs: 235-237 and 282-284, respectively;
(xxviii) SEQ ID NOs: 238-240 and 285-287, respectively;
(xxix) SEQ ID NOs: 241-243 and 288-290, respectively;
(xxx) SEQ ID NOs: 244-246, 273, 274, and 291, respectively;
(xxxi) SEQ ID NOs: 247-249, 292, 274, and 293, respectively;
(xxxii) SEQ ID NOs: 250-252, 294, 274, and 291, respectively;
(xxxiii) SEQ ID NOs: 253-255 and 295-297, respectively;
(xxxiv) SEQ ID NOs: 256-258, 298, 274, and 299, respectively;
(xxxv) SEQ ID NOs: 259-261 and 300-302, respectively;
(xxxvi) SEQ ID NOs: 262-264, 303, 274, and 304, respectively;
(xxxvii) SEQ ID NOs: 265-267, 305, 306, and 291, respectively;
(xxxviii) SEQ ID NOs: 268-270 and 307-309, respectively;
(xxxix) SEQ ID NOs: 265, 271, 272, 310, 274, and 299, respectively;
(xl) SEQ ID NOs: 110, 414, 112, and 176-178, respectively;
(xli) SEQ ID NOs: 110, 414, 112, and 176-178, respectively;
(xlii) SEQ ID NOs: 413, 415, 112, and 176-178, respectively;
(xliii) SEQ ID NOs: 110, 415, 112, and 176-178, respectively;
(xliv) SEQ ID NOs: 110, 416, 112, and 176-178, respectively;
(xlv) SEQ ID NOs: 413, 416, 112, and 176-178, respectively;
(xlvi) SEQ ID NOs: 413, 415, 112, 417, 418, and 178, respectively;
(xlvii) SEQ ID NOs: 419, 422, 112, and 176-178, respectively;
(xlviii) SEQ ID NOs: 419, 423, 112, and 176-178, respectively;
(xlix) SEQ ID NOs: 419, 424, 112, and 176-178, respectively;
(l) SEQ ID NOs: 419, 425, 112, and 176-178, respectively;
(li) SEQ ID NOs: 419, 426, 112, and 176-178, respectively;
(lii) SEQ ID NOs: 419, 422, 112, 427, 177, and 178, respectively;
(liii) SEQ ID NOs: 419, 422, 112, 428, 177, and 178, respectively;
(liv) SEQ ID NOs: 419, 422, 112, 429, 177, and 178, respectively;
(lv) SEQ ID NOs: 419, 422, 112, 430, 177, and 178, respectively;
(lvi) SEQ ID NOs: 419, 422, 112, 431, 177, and 178, respectively;
(lvii) SEQ ID NOs: 419, 422, 112, 432, 177, and 178, respectively;
(lviii) SEQ ID NOs: 419, 422, 112, 433, 177, and 178, respectively;
(lix) SEQ ID NOs: 420, 422, 112, and 176-178, respectively;
(lx) SEQ ID NOs: 421, 422, 112, and 176-178, respectively; or
(lxi) SEQ ID NOs: 421, 426, 112, 434, 177, and 178, respectively.

2-4. (canceled)

5. The antigen-binding domain of claim 1, wherein the antigen-binding domain comprises a VH and VL comprising the amino acid sequences of:
(i) SEQ ID NOs: 363 and 364, respectively;
(ii) SEQ ID NOs: 6 and 7, respectively;
(iii) SEQ ID NOs: 8 and 9, respectively;
(iv) SEQ ID NOs: 10 and 11, respectively;
(v) SEQ ID NOs: 12 and 13, respectively;
(vi) SEQ ID NOs: 14 and 15, respectively;
(vii) SEQ ID NOs: 16 and 17, respectively;
(viii) SEQ ID NOs: 18 and 19, respectively;
(ix) SEQ ID NOs: 20 and 21, respectively;
(x) SEQ ID NOs: 22 and 23, respectively;
(xi) SEQ ID NOs: 24 and 25, respectively;
(xii) SEQ ID NOs: 26 and 27, respectively;
(xiii) SEQ ID NOs: 28 and 29, respectively;
(xiv) SEQ ID NOs: 30 and 31, respectively;
(xv) SEQ ID NOs: 32 and 33, respectively;
(xvi) SEQ ID NOs: 34 and 35, respectively;
(xvii) SEQ ID NOs: 36 and 37, respectively;
(xviii) SEQ ID NOs: 38 and 39, respectively;
(xix) SEQ ID NOs: 40 and 41, respectively;
(xx) SEQ ID NOs: 42 and 43, respectively;
(xxi) SEQ ID NOs: 44 and 45, respectively;
(xxii) SEQ ID NOs: 46 and 47, respectively;
(xxiii) SEQ ID NOs: 48 and 49, respectively;
(xxiv) SEQ ID NOs: 194 and 195, respectively;
(xxv) SEQ ID NOs: 196 and 197, respectively;
(xxvi) SEQ ID NOs: 198 and 199, respectively;
(xxvii) SEQ ID NOs: 200 and 201, respectively;
(xxviii) SEQ ID NOs: 202 and 203, respectively;
(xxix) SEQ ID NOs: 204 and 205, respectively;
(xxx) SEQ ID NOs: 206 and 207, respectively;
(xxxi) SEQ ID NOs: 208 and 209, respectively;
(xxxii) SEQ ID NOs: 210 and 211, respectively;
(xxxiii) SEQ ID NOs: 212 and 213, respectively;
(xxxiv) SEQ ID NOs: 214 and 215, respectively;
(xxxv) SEQ ID NOs: 216 and 217, respectively;
(xxxvi) SEQ ID NOs: 218 and 219, respectively;
(xxxvii) SEQ ID NOs: 220 and 221, respectively;
(xxxviii) SEQ ID NOs: 222 and 223, respectively;
(xxxix) SEQ ID NOs: 224 and 225, respectively;
(xl) SEQ ID NOs: 355 and 356, respectively;
(xli) SEQ ID NOs: 357 and 358, respectively;
(xlii) SEQ ID NOs: 359 and 360, respectively;
(xliii) SEQ ID NOs: 361 and 362, respectively;
(xliv) SEQ ID NOs: 365 and 366, respectively;
(xlv) SEQ ID NOs: 367 and 368, respectively;
(xlvi) SEQ ID NOs: 369 and 370, respectively;
(xlvii) SEQ ID NOs: 371 and 372, respectively;
(xlviii) SEQ ID NOs: 373 and 374, respectively;
(xlix) SEQ ID NOs: 375 and 376, respectively;
(l) SEQ ID NOs: 377 and 378, respectively;
(li) SEQ ID NOs: 379 and 380, respectively;
(lii) SEQ ID NOs: 381 and 382, respectively;
(liii) SEQ ID NOs: 383 and 384, respectively;
(liv) SEQ ID NOs: 385 and 386, respectively;
(lv) SEQ ID NOs: 387 and 388, respectively;
(lvi) SEQ ID NOs: 389 and 390, respectively;
(lvii) SEQ ID NOs: 391 and 392, respectively;
(lviii) SEQ ID NOs: 393 and 394, respectively;
(lix) SEQ ID NOs: 395 and 396, respectively;
(lx) SEQ ID NOs: 397 and 398, respectively;
(lxi) SEQ ID NOs: 399 and 400, respectively;
(lxii) SEQ ID NOs: 401 and 402, respectively; or
(lxiii) SEQ ID NOs: 403 and 404, respectively.

6. The antigen-binding domain of claim 1, wherein the antigen-binding domain is capable of crossing the blood brain barrier (BBB).

7-30. (canceled)

31. An antigen-binding domain that specifically binds to human CD98hc, wherein the antigen-binding domain is a VHH comprising (i) the VH CDR1, VH CDR2, and VH CDR3 of the antigen-binding domain of claim 1 or (ii) the VH of the antigen-binding domain of claim 1, optionally wherein the VHH is capable of crossing the blood brain barrier (BBB).

32. A fusion protein comprising the antigen-binding domain of claim 1 and a heterologous protein or peptide.

33. (canceled)

34. An antibody comprising the antigen-binding domain of claim 1.

35-36. (canceled)

37. A multi-specific protein comprising a first antigen-binding domain that is the antigen-binding domain of claim 1 linked to (a) a second antigen-binding domain, optionally wherein the second antigen-binding domain specifically binds to a CNS antigen or (b) an antibody or antigen-binding fragment thereof, optionally wherein the antibody or antigen-binding fragment thereof specifically binds to a CNS antigen.

38-69. (canceled)

70. The fusion protein of claim 32, wherein the fusion protein is linked to an imaging agent.

71. A composition comprising a first polynucleotide, a second polynucleotide, and a third polynucleotide, wherein the first, second, and third polynucleotides encode the multi-specific protein of claim 37, wherein the first polynucleotide encodes a first heavy chain, the second polynucleotide encodes a second heavy chain and the antigen-binding domain that specifically binds to human CD98hc, and the third polynucleotide encodes a light chain.

72. A composition comprising a first polynucleotide, a second polynucleotide, and a third polynucleotide, wherein the first, second, and third polynucleotides encode the multi-specific protein of claim 37, wherein the first polynucleotide encodes a first heavy chain and a first antigen-binding domain that specifically binds to human CD98hc, the second polynucleotide encodes a second heavy chain and a second antigen-binding domain that specifically binds to human CD98, and the third polynucleotide encodes a light chain, optionally wherein the first and second antigen-binding domains that bind to human CD98hc comprise the same amino acid sequence.

73-75. (canceled)

76. A composition comprising a first polynucleotide and a second polynucleotide, wherein the first and second polynucleotides encode the multi-specific protein of claim 37, wherein the first polynucleotide encodes a heavy chain and the antigen-binding domain that bind to human CD98hc, and wherein the second polynucleotide encodes a light chain.

77. A host cell comprising the composition of claim 71.

78. An isolated polynucleotide comprising a nucleic acid molecule encoding the heavy chain of the antigen-binding domain of claim 1 or a nucleic acid molecule encoding the light chain variable region of the antigen-binding domain of claim 1.

79. (canceled)

80. An isolated vector comprising the polynucleotide of claim 78.

81. (canceled)

82. A host cell comprising the polynucleotide of claim 78.

83. (canceled)

84. A method of producing a multi-specific protein comprising culturing the host cell of claim 77 so that the multi-specific protein is produced, optionally wherein the method further comprises isolating the multi-specific protein from the culture.

85. (canceled)

86. A pharmaceutical composition comprising (i) the antigen-binding domain of claim 1 and (ii) a pharmaceutically acceptable carrier.

87-88. (canceled)

89. A method of treating a neurological disease or disorder in a subject comprising administering the fusion protein of claim 32 to the subject.

90-100. (canceled)

101. A method of treating a lysosomal storage disease in a subject, comprising administering the fusion protein of claim 32 to the subject.

102. (canceled)

103. A method of transporting a fusion protein, across the BBB of a subject, comprising administering to the subject the fusion protein of claim 32.

104-107. (canceled)

108. A method of increasing the concentration of a CNS binding antigen in the CSF of a subject, comprising administering the multi-specific protein of claim 37, wherein the concentration of the CNS binding antigen is increased as compared to administering the CNS binding antigen alone to the subject.

109. A method of imaging a CNS antigen within a subject, comprising administering to the subject the fusion protein of claim 70 and locating the imaging agent within the subject.

110. A method of detecting a CNS antigen in vitro, comprising contacting an in vitro sample with the fusion protein of claim 70 and locating the imaging agent within the sample.

111-112. (canceled)


SEQUENCE LISTING (435 sequences)

SEQ ID NO: 1 - protein, 451 aa, synthetic construct
  source 1..451: note = huCD98hc ECD Avi-His
RAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAAGGGLNDIFEAQKIEWHEHHHHHHHHH

SEQ ID NO: 2 - protein, 451 aa, synthetic construct
  source 1..451: note = Cyno CD98hc ECD Avi-His
RAPRCRELPAQKWWHTGALYRIGDLQAFQGHGSGNLAGLKGRLDYLSSLKVKGLVLGPLHKNQKDDVAQTDLLQIDPNFGSKEDFDNLLQSAKKKSIRVILDLTPNYRGENLWFSTQVDSVATKVKDALEFWLQAGVDGFQVRDIENLKDASSFLAEWENITKGFSEDRLLIAGTNSSDLQQIVSLLESNKDLLLTSSYLSDSSFTGEHTKSLVTQYLNATGNRWCSWSLSQAGLLTSFLPAQLLRLYQLMLFTLPGTPVFSYGDEIGLKAAALPGQPVEAPVMLWDESSFPDIPGAVSANMTVKGQSEDPGSLLSLFRQLSDQRSKERSLLHGDFHTFSSGPGLFSYIRHWDQNERFLVVLNFGDVGLSAGLQASDLPASASLPTKADLVLSTQPGREEGSPLELERLKLEPHEGLLLRFPYVAGGGLNDIFEAQKIEWHEHHHHHHHHH

SEQ ID NO: 3 - protein, 453 aa, synthetic construct
  source 1..453: note = muCD98hc ECD Avi-His
APRCRELPVQRWWHKGALYRIGDLQAFVGRDAGGIAGLKSHLEYLSTLKVKGLVLGPIHKNQKDEINETDLKQINPTLGSQEDFKDLLQSAKKKSIHIILDLTPNYQGQNAWFLPAQADIVATKMKEALSSWLQDGVDGFQFRDVGKLMNAPLYLAEWQNITKNLSEDRLLIAGTESSDLQQIVNILESTSDLLLTSSYLSNSTFTGERTESLVTRFLNATGSQWCSWSVSQAGLLADFIPDHLLRLYQLLLFTLPGTPVFSYGDELGLQGALPGQPAKAPLMPWNESSIFHIPRPVSLNMTVKGQNEDPGSLLTQFRRLSDLRGKERSLLHGDFHALSSSPDLFSYIRHWDQNERYLVVLNFRDSGRSARLGASNLPAGISLPASAKLLLSTDSARQSREEDTSLKLENLSLNPYEGLLLQFPFVAGGGLNDIFEAQKIEWHEHHHHHHHHH

SEQ ID NO: 4 - protein, 630 aa, synthetic construct
  source 1..630: note = Full length human CD98hc
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 5 - protein, 526 aa, synthetic construct
  source 1..526: note = Full length mouse CD98hc
MSQDTEVDMKDVELNELEPEKQPMNAADGAAAGEKNGLVKIKVAEDETEAGVKFTGLSKEELLKVAGSPGWVRTRWALLLLFWLGWLGMLAGAVVIIVRAPRCRELPVQRWWHKGALYRIGDLQAFVGRDAGGIAGLKSHLEYLSTLKVKGLVLGPIHKNQKDEINETDLKQINPTLGSQEDFKDLLQSAKKKSIHIILDLTPNYQGQNAWFLPAQADIVATKMKEALSSWLQDGVDGFQFRDVGKLMNAPLYLAEWQNITKNLSEDRLLIAGTESSDLQQIVNILESTSDLLLTSSYLSNSTFTGERTESLVTRFLNATGSQWCSWSVSQAGLLADFIPDHLLRLYQLLLFTLPGTPVFSYGDELGLQGALPGQPAKAPLMPWNESSIFHIPRPVSLNMTVKGQNEDPGSLLTQFRRLSDLRGKERSLLHGDFHALSSSPDLFSYIRHWDQNERYLVVLNFRDSGRSARLGASNLPAGISLPASAKLLLSTDSARQSREEDTSLKLENLSLNPYEGLLLQFPFVA

SEQ ID NO: 6 - protein, 120 aa, synthetic construct
  source 1..120: note = CD98HC.03.002 VH
DVQLVESGGGLVQPGGSRKLSCAASGFTFSTFGMHWVRQAPEKGLEWVAYISGGSTNIYYADTVKGRFTISRDNPKNTLFLQMTSLRSEDTAMYYCARETTLLATNFDYWGQGTTLTVSS

SEQ ID NO: 7 - protein, 107 aa, synthetic construct
  source 1..107: note = CD98HC.03.002 VL
DIQMTQTTSSLSASLGDRVTISCRASQDISNYLNWYQQKPDGTVKLLIYYTSRLHSGVPSRFSGSGSGTDYSLTISNLEQEDIATYFCQQGSTLPRTFGGGTKLEIK

SEQ ID NO: 8 - protein, 121 aa, synthetic construct
  source 1..121: note = CD98HC.03.004 VH
DVQLQESGPGLVKPSQSLSLTCTVTGYSITNNYAWNWIRRFPGNKLEWMAYISYSGYTSYNPSLKSRISITRDTSKNQFFLQLNSVTTEDTATYYCTGYFYYYGGGSFDYWGQGTTLTVSS

SEQ ID NO: 9 - protein, 109 aa, synthetic construct
  source 1..109: note = CD98HC.03.004 VL
QAVVTQESALTTSPGETVTLTCRSSTGAVTTSNFANWVQEKPDHLFTGLIRGSNNRAPGVPARFSGSLIGDKAALTITGARTEDEAIYFCALWYSDHWVFGGGTKLTVL

SEQ ID NO: 10 - protein, 120 aa, synthetic construct
  source 1..120: note = CD98HC.03.010 VH
EVQLQQSGTELVKPGASVKLSCTASGLDIKDTYLHWVKQRPEQGLEWIGKIDPANGNTKYNPKFQGKATITADTSSNTAYLQLSSLTSEDTAVHYCTTPYYSGSSYGDYWGQGTTLTVSS

SEQ ID NO: 11 - protein, 108 aa, synthetic construct
  source 1..108: note = CD98HC.03.010 VL
QIVLTQSPAIMSASPGEKVTLTCSASSSVSSSYLYWYQQKPGSSPKLWIYSTSNLASGVPARFSGSGSGTSYSLTISSMEAEDAASYFCHQWSRYPLTFGAGTKLELK

SEQ ID NO: 12 - protein, 119 aa, synthetic construct
  source 1..119: note = CD98HC.03.031 VH
QVQLQQSGAELVRPGVSVKISCKASGYTFTDYAMHWVKQSHAKSLEWIGVISTYYGDTTYNQKFKDKATMTVDKSSSTAYLELARLTSEDSAIYYCARVWLLSYAMDNWGQGTSVTVSS

SEQ ID NO: 13 - protein, 107 aa, synthetic construct
  source 1..107: note = CD98HC.03.031 VL
DIVMTQSQRFMSTSVGDGVRITCKASQNVRTAVAWYQQKPGQSPKALINLAPNRHTGVPDRFTARGSGTDFTLTISNVQSEDLADYFCLQHWNYPLTFGAGTKLELK

SEQ ID NO: 14 - protein, 120 aa, synthetic construct
  source 1..120: note = CD98HC.04.003 VH
QVQLQQSGAELAKPGASLKMSCKASGYTFTDYYIHWVKQRPGQDLEWIGYINPNSAYTDSNQKFKDKATLTADKSSNTAYMQLSSLTSEDSAVYYCARKGHFYAPSFAYWGQGTLVTVSA

SEQ ID NO: 15 - protein, 107 aa, synthetic construct
  source 1..107: note = CD98HC.04.003 VL
DIQMTQTTSSLSASLGDRVTISCRASQDISNYLNWYQQKPDGTVKLLIYYTSRLHSGVPSRFSGSGSGADYSLTISNLQQEDIATYFCQQGDTLPYTFGGGTKLEIE

SEQ ID NO: 16 - protein, 122 aa, synthetic construct
  source 1..122: note = CD98HC.04.006 VH
EVNLVESGGDLVQPGGSLRLSCATSGFTFTDYFMSWVRQPPGKALEWLGFIRNKAYGYTTEYSASVKGRFTISRDNSQSIFYLQMNTLRLEDSATYYCARGPFISTAPFAYWGQGTLVTVSA

SEQ ID NO: 17 - protein, 113 aa, synthetic construct
  source 1..113: note = CD98HC.04.006 VL
DIVMSQSPSSLAVSVGETVTMSCKSSQSLLYSSNQKNYLAWYQQKPGQSPKLLIYWGSTRESGVPDRFTGSGSGTDFTLTISSVKAEDLAVYYCQHYYGYPWTFGGGTKLEIK

SEQ ID NO: 18 - protein, 118 aa, synthetic construct
  source 1..118: note = CD98HC.04.009 VH
QVQLKESGPGLVAPSQSLSITCTVSGFSLTSYGVHWVRQPPGKGLEWLGVIWAGGSTYYNSALMSRLSISKDNAKSQVLLKMSSLQTDDTAMYYCARDNYGGVYLDVWGAGTTVTVSS

SEQ ID NO: 19 - protein, 108 aa, synthetic construct
  source 1..108: note = CD98HC.04.009 VL
ENVLTQSPASMSASPGEKVTMTCRASSSVSSSYLHWYQQKSGASPKLWIYSTSNLASGVPARFSGSGSGTSYSLTISSVEAEDAATYYCQQYSGYPLTFGSGTKLEIK

SEQ ID NO: 20 - protein, 121 aa, synthetic construct
  source 1..121: note = CD98HC.04.010 VH
QVQLQQSGAELMKPGASVKISCKATGYTFSTYWIEWVKQRPGHGLEWIGEILPGSGTTTYNEKFKGKATFTADTSSNTAYMQLSSLTSEDSAVYYCARRTVYYRYEGIDYWGQGTTLTVSS

SEQ ID NO: 21 - protein, 106 aa, synthetic construct
  source 1..106: note = CD98HC.04.010 VL
QIVLTQSPAIMSASPGEKVTITCSASSSVSYMFWFQQRPGTSPKLWIYTTSNLASGVPARFSGSGSGTSYSLTISRMEAEDAATYYCQQKNNYPFTFGSGTKLEIK

SEQ ID NO: 22 - protein, 120 aa, synthetic construct
  source 1..120: note = CD98HC.04.011 VH
DVQLQESGPDLVKPSQSLSLTCSVTVYSFTSVYSWHWIRQFPGNKLEWMGYIHDSGTTNYNPSLKSRISVTRDTSKNQFFLQLNSVTTEDTATYYCARGRPYGYDGFAYWGQGTLVTVSA

SEQ ID NO: 23 - protein, 107 aa, synthetic construct
  source 1..107: note = CD98HC.04.011 VL
DIQMTQSSSYLSVSLGDRVTITCKASDHINNWLAWYQQKPGNAPRLLITGAVNLETGVPSRFSGSGSGKDYTLSITSLQTEDVATYYCQQYWSIPRTFGGGTNLEIK

SEQ ID NO: 24 - protein, 122 aa, synthetic construct
  source 1..122: note = CD98HC.04.012 VH
QVQLQQSGADLVRPGDSVKISCKGSGYTFTDNTLHWVKQSHAKSLEWIGVISPYYGDASYNQKFKGKATMTVDKSSSTAYMELARLTSEDSAIYYCARVGGRLRLGYYFDHWGQGTTLTVSS

SEQ ID NO: 25 - protein, 112 aa, synthetic construct
  source 1..112: note = CD98HC.04.012 VL
NIMMTQSPSSLAVSAGEKVTMNCKSSQSVLSSSNQKNYLAWYQQKPGQSPKLLIYWASTRESGVPARFTGSGSGTDFSLTITSVQAEDLAVYYCLQYLSSYTFGGGTKLEIK

SEQ ID NO: 26 - protein, 119 aa, synthetic construct
  source 1..119: note = CD98HC.04.013 VH
EVQLVESGGDLGMPGGSLKLSCAASGFALSGYDMSWVRQTPDKRLEWVAYLSSGGGSTYYPYTVKGRFTISRDNAQNTLYLQMSSLKSEDTAIYYCARHSEVRPWFGYWGQGTLVTVSA

SEQ ID NO: 27 - protein, 113 aa, synthetic construct
  source 1..113: note = CD98HC.04.013 VL
DIVMSQSPSSLAVSVGEKVTLTCKSSQSLLYSGNQKNYLAWYQQNPGQSPKLLIYWASTRESGVPDRFTGSGSGTDFTLTISSVKAEDLAVYYCQQYYTYPPTFGAGTKLGLK

SEQ ID NO: 28 - protein, 118 aa, synthetic construct
  source 1..118: note = CD98HC.04.018 VH
QIQLVQSGPELKKPGETVKISCKASGYTFRNYGMNWVKQAPGKGLKWMGWINTYTGEPTYAEDFKGRFAFSLETSASTAYLQINKLKNEDEATYFCARGEAHWYFDVWGAGTTVTVSS

SEQ ID NO: 29 - protein, 108 aa, synthetic construct
  source 1..108: note = CD98HC.04.018 VL
QIVLTQSPAIMSASLGERVTMTCTASSSVSSSYLYWYQQKPGSSPKLWIYSIFNLASGVPARFSGSGSGTSYSLTISSMEAEDAATYYCHQYHRSPPTFGGGTKLEIK

SEQ ID NO: 30 - protein, 115 aa, synthetic construct
  source 1..115: note = CD98HC.04.022 VH
EVQLQQSGAELVKPGASVKLSCTASGFNIKDTFMHWMKQRPEQGLEWIGRIDPANANTRYDPKFQGKATITTDTSSNTAYLQLSSLTSEDTAVYYCARDVYYDVWGAGTTVTVSS

SEQ ID NO: 31 - protein, 107 aa, synthetic construct
  source 1..107: note = CD98HC.04.022 VL
DILMTQSPSSMSVSLGDTVTITCHASQGISSNIGWLQQKPGRSFKGLIYHGSNLEDGVPSRFTGSGSGTDYSLTISGLESEDFADYYCVQYAQFPRTFGGGTKLEIQ

SEQ ID NO: 32 - protein, 118 aa, synthetic construct
  source 1..118: note = CD98HC.04.045 VH
QIQLVQSGPELKKPGETVKISCKASGYSITNFGMNWVKQAPGKGLKWMGWVNTYTGEPTYADDFKGRFAFSLETSASTASLQINNLKNEDTATYFCTRSVITYTMDYWGQGTSVTVSS

SEQ ID NO: 33 - protein, 107 aa, synthetic construct
  source 1..107: note = CD98HC.04.045 VL
DIQLTQSPSSLSASLGERVSLTCRASQEISGYLSWLQQKPDGTIKRLIYAASTLDSGVPKRFSGSRSGSDYSLTISSLESEDFADYYCLQYASYPRTFGGGTKVEIK

SEQ ID NO: 34 - protein, 118 aa, synthetic construct
  source 1..118: note = CD98HC.04.046 VH
QIQLVQSGPVLKKPGETVKISCKASGYTFTNFGMNWVKQAPGKGLKWMGWIDTYTREPTYVDDFKGRFAFSLETSVTTAYLQINDLKNEDSATYFCARGESHWYFDVWGAGTTVTVSS

SEQ ID NO: 35 - protein, 108 aa, synthetic construct
  source 1..108: note = CD98HC.04.046 VL
QIVLTQSPAIMSASLGERVTMTCIVSSSVSSSYLYWYQQKPGSSPKLWIYSTSILASGVPGRFSGSGSGTSYSLTISSMEAEDAATYYCHQYHRSPPTFGGGTKLEIK

SEQ ID NO: 36 - protein, 119 aa, synthetic construct
  source 1..119: note = CD98HC.04.047 VH
EVQLQESGPSLVKPSQTLSLTCSVTGDSITSGYWNWIRKFPGNKLEYMGYISYSGSTYYNPSLKGRVSITRDTSKNQYYLQLNSVTTEDTATYYCQRHVIYDYTWFAYWGQGTLVTVSA

SEQ ID NO: 37 - protein, 106 aa, synthetic construct
  source 1..106: note = CD98HC.04.047 VL
QIVLTQSPAIMSASPGEKVTISCSASSSVGYMYWLQQKPGSSPKPWIYRTSILASGVPARFSGSGSGTSYSLTISSMEAEDAATYYCQQYQSYPLTFGAGTKLELK

SEQ ID NO: 38 - protein, 116 aa, synthetic construct
  source 1..116: note = CD98HC.04.054 VH
QVQLQQSAAELARPGASVKMSCETSGYTFTRYTMNWVKQRPGQGLDWIGYINPSSGYAEYNQKFRGKTTLTADKSSSTAYIQLNSLTSEDSAVYYCVRYDGNYVYWGQGTTLTVSS

SEQ ID NO: 39 - protein, 107 aa, synthetic construct
  source 1..107: note = CD98HC.04.054 VL
DIVMTQSQKFMSTSFGDRVDITCKASQNVGSAVAWYQQKPGQSPKLLIYSASNRYTGVPDRFTGSGSGTDFTLTINNMQSADLADYFCQQYNFYPLTFGAGTKLDLK

SEQ ID NO: 40 - protein, 122 aa, synthetic construct
  source 1..122: note = CD98HC.04.058 VH
QVQLQQSGAELVRPGVSVKISCKASGYTFTDHTMHWVKQSHARSLEWIGVISTYFGDASYNPKFKGKATMTVDKSSNTAYMELARLTSEDSAIYYCARVGGRLRLGYYFDYWGQGTTLTVSS

SEQ ID NO: 41 - protein, 112 aa, synthetic construct
  source 1..112: note = CD98HC.04.058 VL
NIMMTQSPSSLAVSAGEKVTMNCKSSQTVLYSSNQKNYLAWYQQKPGQSPKLLIYWASTRESGVPDRFTGSGSGTDFTLTISSVQVEDLAVYYCHQYLSSYTFGGGTKLEIK

SEQ ID NO: 42 - protein, 119 aa, synthetic construct
  source 1..119: note = CD98HC.04.062 VH
EVQLVESGGDLVKPGGSLKLSCTASRFAFSTYDMSWVRQTPEKRLEWVAYISSGGGSTYFPDTVKGRFTISRDNAKNTLYLQMSSLKSEDTAMYYCARQDGHYPWFPYWGQGTLVTVSA

SEQ ID NO: 43 - protein, 113 aa, synthetic construct
  source 1..113: note = CD98HC.04.062 VL
DIVMSQSPSSLAVSVGEKVTMSCKSSQSLLYSGNQKTYLAWYQQKPGQSPKPLIYWASTRESGIPDRFTGSGSGTDFTLTISSVKAEDLAVYYCQQYYSYPPTFGAGTKLELK

SEQ ID NO: 44 - protein, 117 aa, synthetic construct
  source 1..117: note = CD98HC.04.063 VH
EVKLEESGGGLVQPGGSMILSCVASGFTFSNSWMNWVRQSPEKGLEWVAEIRLRSNNYATHYAESVKGRFTISRDDSKSTVYLQMNNLRAEDTGIYYCSRGDPGAYWGQGTLVTVSA

SEQ ID NO: 45 - protein, 107 aa, synthetic construct
  source 1..107: note = CD98HC.04.063 VL
DIQMTQSSSYLSVSLGNRVTITCKASDHIKNWLAWFQQKPGNAPRLLISGATILETGVPSRFSGSGSGKDYTLSITSLQTEDVATYYCQQYWSTPYTFGGGTKLEIK

SEQ ID NO: 46 - protein, 119 aa, synthetic construct
  source 1..119: note = CD98HC.04.064 VH
QVQLQQPGAELVRPGASVKLSCKASGYTFTSYWMNWVRQRPGQGLEWIGLIDPSDSETHYNQMFKDRATLTVDKSSSTAYMQLSSLTSADPAVYYCARASYGKGYFDYWGQGTTLTVSS

SEQ ID NO: 47 - protein, 113 aa, synthetic construct
  source 1..113: note = CD98HC.04.064 VL
DIVMTQSPSSLAMSVGQKVTMSCKSSQSLLNNINQKNYLAWYQQKPGQSPKLLVYFASTRESGVPDRFIGSGSGTDFTLTINTVQAEDLADYFCQQHYSSPFTFGGGTKLEIK

SEQ ID NO: 48 - protein, 119 aa, synthetic construct
  source 1..119: note = CD98HC.04.065 VH
EVQLQESGPSLVKPSQTLSLTCSVTGDSITSGYWNWIRKFPGNKLEYMGYISYSGRTYYNPSLKSRVSITRDTSKNQYYLQLNSVTTEDTATYYCQRHVIYDYTWFAYWGQGTLVTVSA

SEQ ID NO: 49 - protein, 106 aa, synthetic construct
  source 1..106: note = CD98HC.04.065 VL
QIVLTQSPAIMSASPGEKVTISCSASSSVHYMYWFQQKPGSSPKPWIYRTSILASGVPDRFSGSGSGTSYSLTISSMEAEDAATYYCQQYQSYPLTFGAGTKLELK

SEQ ID NO: 50 - protein, 5 aa, synthetic construct
  source 1..5: note = CD98HC.03.002 CDR-H1
TFGMH

SEQ ID NO: 51 - protein, 17 aa, synthetic construct
  source 1..17: note = CD98HC.03.002 CDR-H2
YISGGSTNIYYADTVKG

SEQ ID NO: 52 - protein, 11 aa, synthetic construct
  source 1..11: note = CD98HC.03.002 CDR-H3
ETTLLATNFDY

SEQ ID NO: 53 - protein, 6 aa, synthetic construct
  source 1..6: note = CD98HC.03.004 CDR-H1
NNYAWN

SEQ ID NO: 54 - protein, 16 aa, synthetic construct
  source 1..16: note = CD98HC.03.004 CDR-H2
YISYSGYTSYNPSLKS

SEQ ID NO: 55 - protein, 12 aa, synthetic construct
  source 1..12: note = CD98HC.03.004 CDR-H3
YFYYYGGGSFDY

SEQ ID NO: 56 - protein, 5 aa, synthetic construct
  source 1..5: note = CD98HC.03.010 CDR-H1
DTYLH

SEQ ID NO: 57 - protein, 17 aa, synthetic construct
  source 1..17: note = CD98HC.03.010 CDR-H2
KIDPANGNTKYNPKFQG

SEQ ID NO: 58 - protein, 11 aa, synthetic construct
  source 1..11: note = CD98HC.03.010 CDR-H3
PYYSGSSYGDY

SEQ ID NO: 59 - protein, 5 aa, synthetic construct
  source 1..5: note = CD98HC.03.031 CDR-H1
DYAMH

SEQ ID NO: 60 - protein, 17 aa, synthetic construct
  source 1..17: note = CD98HC.03.031 CDR-H2
VISTYYGDTTYNQKFKD

SEQ ID NO: 61 - protein, 10 aa, synthetic construct
  source 1..10: note = CD98HC.03.031 CDR-H3
VWLLSYAMDN

SEQ ID NO: 62 - protein, 5 aa, synthetic construct
  source 1..5: note = CD98HC.04.003 CDR-H1
DYYIH

SEQ ID NO: 63 - protein, 17 aa, synthetic construct
  source 1..17: note = CD98HC.04.003 CDR-H2
YINPNSAYTDSNQKFKD

SEQ ID NO: 64 - protein, 11 aa, synthetic construct
  source 1..11: note = CD98HC.04.003 CDR-H3
KGHFYAPSFAY

SEQ ID NO: 65 - protein, 5 aa, synthetic construct
  source 1..5: note = CD98HC.04.006 CDR-H1
DYFMS

SEQ ID NO: 66 - protein, 19 aa, synthetic construct
  source 1..19: note = CD98HC.04.006 CDR-H2
FIRNKAYGYTTEYSASVKG

SEQ ID NO: 67 - protein, 11 aa, synthetic construct
  source 1..11: note = CD98HC.04.006 CDR-H3
GPFISTAPFAY

SEQ ID NO: 68 - protein, 5 aa, synthetic construct
  source 1..5: note = CD98HC.04.009 CDR-H1
SYGVH

SEQ ID NO: 69 - protein, 16 aa, synthetic construct
  source 1..16: note = CD98HC.04.009 CDR-H2
VIWAGGSTYYNSALMS

SEQ ID NO: 70 - protein, 10 aa, synthetic construct
  source 1..10: note = CD98HC.04.009 CDR-H3
DNYGGVYLDV

SEQ ID NO: 71 - protein, 5 aa, synthetic construct
  source 1..5: note = CD98HC.04.010 CDR-H1
TYWIE

SEQ ID NO: 72 - protein, 17 aa, synthetic construct
  source 1..17: note = CD98HC.04.010 CDR-H2
EILPGSGTTTYNEKFKG

SEQ ID NO: 73 - protein, 12 aa, synthetic construct
  source 1..12: note = CD98HC.04.010 CDR-H3
RTVYYRYEGIDY

SEQ ID NO: 74 - protein, 6 aa, synthetic construct
  source 1..6: note = CD98HC.04.011 CDR-H1
SVYSWH

SEQ ID NO: 75 - protein, 16 aa, synthetic construct
  source 1..16: note = CD98HC.04.011 CDR-H2
YIHDSGTTNYNPSLKS

SEQ ID NO: 76 - protein, 11 aa, synthetic construct
  source 1..11: note = CD98HC.04.011 CDR-H3
GRPYGYDGFAY

SEQ ID NO: 77 - protein, 5 aa, synthetic construct
  source 1..5: note = CD98HC.04.012 CDR-H1
DNTLH

SEQ ID NO: 78 - protein, 17 aa, synthetic construct
  source 1..17: note = CD98HC.04.012 CDR-H2
VISPYYGDASYNQKFKG

SEQ ID NO: 79 - protein, 13 aa, synthetic construct
  source 1..13: note = CD98HC.04.012 CDR-H3
VGGRLRLGYYFDH

SEQ ID NO: 80 - protein, 5 aa, synthetic construct
  source 1..5: note = CD98HC.04.013 CDR-H1
GYDMS

SEQ ID NO: 81 - protein, 17 aa, synthetic construct
  source 1..17: note = CD98HC.04.013 CDR-H2
YLSSGGGSTYYPYTVKG

SEQ ID NO: 82 - protein, 10 aa, synthetic construct
  source 1..10: note = CD98HC.04.013 CDR-H3
HSEVRPWFGY

SEQ ID NO: 83 - protein, 5 aa, synthetic construct
  source 1..5: note = CD98HC.04.018 CDR-H1
NYGMN

SEQ ID NO: 84 - protein, 17 aa, synthetic construct
  source 1..17: note = CD98HC.04.018 CDR-H2
WINTYTGEPTYAEDFKG

SEQ ID NO: 85 - protein, 9 aa, synthetic construct
  source 1..9: note = CD98HC.04.018 CDR-H3
GEAHWYFDV

SEQ ID NO: 86 - protein, 5 aa, synthetic construct
  source 1..5: note = CD98HC.04.022 CDR-H1
DTFMH

SEQ ID NO: 87 - protein, 17 aa, synthetic construct
  source 1..17: note = CD98HC.04.022 CDR-H2
RIDPANANTRYDPKFQG

SEQ ID NO: 88 - protein, 6 aa, synthetic construct
  source 1..6: note = CD98HC.04.022 CDR-H3
DVYYDV

SEQ ID NO: 89 - protein, 5 aa, synthetic construct
  source 1..5: note = CD98HC.04.045 CDR-H1
NFGMN

SEQ ID NO: 90 - protein, 17 aa, synthetic construct
  source 1..17: note = CD98HC.04.045 CDR-H2
WVNTYTGEPTYADDFKG

SEQ ID NO: 91 - protein, 9 aa, synthetic construct
  source 1..9: note = CD98HC.04.045 CDR-H3
SVITYTMDY

SEQ ID NO: 92 - protein, 5 aa, synthetic construct
  source 1..5: note = CD98HC.04.046 CDR-H1
NFGMN

SEQ ID NO: 93 - protein, 17 aa, synthetic construct
  source 1..17: note = CD98HC.04.046 CDR-H2
WIDTYTREPTYVDDFKG

SEQ ID NO: 94 - protein, 9 aa, synthetic construct
  source 1..9: note = CD98HC.04.046 CDR-H3
GESHWYFDV

SEQ ID NO: 95 - protein, 5 aa, synthetic construct
  source 1..5: note = CD98HC.04.047 CDR-H1
SGYWN

SEQ ID NO: 96 - protein, 16 aa, synthetic construct
  source 1..16: note = CD98HC.04.047 CDR-H2
YISYSGSTYYNPSLKG

SEQ ID NO: 97 - protein, 11 aa, synthetic construct
  source 1..11: note = CD98HC.04.047 CDR-H3
HVIYDYTWFAY

SEQ ID NO: 98 - protein, 5 aa, synthetic construct
  source 1..5: note = CD98HC.04.054 CDR-H1
RYTMN

SEQ ID NO: 99 - protein, 17 aa, synthetic construct
  source 1..17: note = CD98HC.04.054 CDR-H2
YINPSSGYAEYNQKFRG

SEQ ID NO: 100 - protein, 7 aa, synthetic construct
  source 1..7: note = CD98HC.04.054 CDR-H3
YDGNYVY

SEQ ID NO: 101 - protein, 5 aa, synthetic construct
  source 1..5: note = CD98HC.04.058 CDR-H1
DHTMH

SEQ ID NO: 102 - protein, 17 aa, synthetic construct
  source 1..17: note = CD98HC.04.058 CDR-H2
VISTYFGDASYNPKFKG

SEQ ID NO: 103 - protein, 13 aa, synthetic construct
  source 1..13: note = CD98HC.04.058 CDR-H3
VGGRLRLGYYFDY

SEQ ID NO: 104 - protein, 5 aa, synthetic construct
  source 1..5: note = CD98HC.04.062 CDR-H1
TYDMS

SEQ ID NO: 105 - protein, 17 aa, synthetic construct
  source 1..17: note = CD98HC.04.062 CDR-H2
YISSGGGSTYFPDTVKG

SEQ ID NO: 106 - protein, 10 aa, synthetic construct
  source 1..10: note = CD98HC.04.062 CDR-H3
QDGHYPWFPY

SEQ ID NO: 107 - protein, 5 aa, synthetic construct
  source 1..5: note = CD98HC.04.063 CDR-H1
NSWMN

SEQ ID NO: 108 - protein, 19 aa, synthetic construct
  source 1..19: note = CD98HC.04.063 CDR-H2
EIRLRSNNYATHYAESVKG

SEQ ID NO: 109 - protein, 6 aa, synthetic construct
  source 1..6: note = CD98HC.04.063 CDR-H3
GDPGAY

SEQ ID NO: 110 - protein, 5 aa, synthetic construct
  source 1..5: note = CD98HC.04.064 CDR-H1
SYWMN

SEQ ID NO: 111 - protein, 17 aa, synthetic construct
  source 1..17: note = CD98HC.04.064 CDR-H2
LIDPSDSETHYNQMFKD

SEQ ID NO: 112 - protein, 10 aa, synthetic construct
  source 1..10: note = CD98HC.04.064 CDR-H3
ASYGKGYFDY

SEQ ID NO: 113 - protein, 5 aa, synthetic construct
  source 1..5: note = CD98HC.04.065 CDR-H1
SGYWN

SEQ ID NO: 114 - protein, 16 aa, synthetic construct
  source 1..16: note = CD98HC.04.065 CDR-H2
YISYSGRTYYNPSLKS

SEQ ID NO: 115 - protein, 11 aa, synthetic construct
  source 1..11: note = CD98HC.04.065 CDR-H3
HVIYDYTWFAY

SEQ ID NO: 116 - protein, 11 aa, synthetic construct
  source 1..11: note = CD98HC.03.002 CDR-L1
RASQDISNYLN

SEQ ID NO: 117 - protein, 7 aa, synthetic construct
  source 1..7: note = CD98HC.03.002 CDR-L2
YTSRLHS

SEQ ID NO: 118 - protein, 9 aa, synthetic construct
  source 1..9: note = CD98HC.03.002 CDR-L3
QQGSTLPRT

SEQ ID NO: 119 - protein, 14 aa, synthetic construct
  source 1..14: note = CD98HC.03.004 CDR-L1
RSSTGAVTTSNFAN

SEQ ID NO: 120 - protein, 7 aa, synthetic construct
  source 1..7: note = CD98HC.03.004 CDR-L2
GSNNRAP

SEQ ID NO: 121 - protein, 9 aa, synthetic construct
  source 1..9: note = CD98HC.03.004 CDR-L3
ALWYSDHWV

SEQ ID NO: 122 - protein, 12 aa, synthetic construct
  source 1..12: note = CD98HC.03.010 CDR-L1
SASSSVSSSYLY

SEQ ID NO: 123 - protein, 7 aa, synthetic construct
  source 1..7: note = CD98HC.03.010 CDR-L2
STSNLAS

SEQ ID NO: 124 - protein, 9 aa, synthetic construct
  source 1..9: note = CD98HC.03.010 CDR-L3
HQWSRYPLT

SEQ ID NO: 125 - protein, 5 aa, synthetic construct
  source 1..5: note = CD98HC.03.031 CDR-L1
DYAMH

SEQ ID NO: 126 - protein, 17 aa, synthetic construct
  source 1..17: note = CD98HC.03.031 CDR-L2
VISTYYGDTTYNQKFKD

SEQ ID NO: 127 - protein, 10 aa, synthetic construct
  source 1..10: note = CD98HC.03.031 CDR-L3
VWLLSYAMDN

SEQ ID NO: 128 - protein, 5 aa, synthetic construct
  source 1..5: note = CD98HC.04.003 CDR-L1
DYYIH

SEQ ID NO: 129 - protein, 17 aa, synthetic construct
  source 1..17: note = CD98HC.04.003 CDR-L2
YINPNSAYTDSNQKFKD

SEQ ID NO: 130 - protein, 11 aa, synthetic construct
  source 1..11: note = CD98HC.04.003 CDR-L3
KGHFYAPSFAY

SEQ ID NO: 131 - protein, 5 aa, synthetic construct
  source 1..5: note = CD98HC.04.006 CDR-L1
DYFMS

SEQ ID NO: 132 - protein, 19 aa, synthetic construct
  source 1..19: note = CD98HC.04.006 CDR-L2
FIRNKAYGYTTEYSASVKG

SEQ ID NO: 133 - protein, 11 aa, synthetic construct
  source 1..11: note = CD98HC.04.006 CDR-L3
GPFISTAPFAY

SEQ ID NO: 134 - protein, 12 aa, synthetic construct
  source 1..12: note = CD98HC.04.009 CDR-L1
RASSSVSSSYLH

SEQ ID NO: 135 - protein, 7 aa, synthetic construct
  source 1..7: note = CD98HC.04.009 CDR-L2
STSNLAS

SEQ ID NO: 136 - protein, 9 aa, synthetic construct
  source 1..9: note = CD98HC.04.009 CDR-L3
QQYSGYPLT

SEQ ID NO: 137 - protein, 10 aa, synthetic construct
  source 1..10: note = CD98HC.04.010 CDR-L1
SASSSVSYMF

SEQ ID NO: 138 - protein, 7 aa, synthetic construct
  source 1..7: note = CD98HC.04.010 CDR-L2
TTSNLAS

SEQ ID NO: 139 - protein, 9 aa, synthetic construct
  source 1..9: note = CD98HC.04.010 CDR-L3
QQKNNYPFT

SEQ ID NO: 140 - protein, 11 aa, synthetic construct
  source 1..11: note = CD98HC.04.011 CDR-L1
KASDHINNWLA

SEQ ID NO: 141 - protein, 7 aa, synthetic construct
  source 1..7: note = CD98HC.04.011 CDR-L2
GAVNLET

SEQ ID NO: 142 - protein, 9 aa, synthetic construct
  source 1..9: note = CD98HC.04.011 CDR-L3
QQYWSIPRT

SEQ ID NO: 143 - protein, 17 aa, synthetic construct
  source 1..17: note = CD98HC.04.012 CDR-L1
KSSQSVLSSSNQKNYLA

SEQ ID NO: 144 - protein, 7 aa, synthetic construct
  source 1..7: note = CD98HC.04.012 CDR-L2
WASTRES

SEQ ID NO: 145 - protein, 8 aa, synthetic construct
  source 1..8: note = CD98HC.04.012 CDR-L3
LQYLSSYT

SEQ ID NO: 146 - protein, 17 aa, synthetic construct
  source 1..17: note = CD98HC.04.013 CDR-L1
KSSQSLLYSGNQKNYLA

SEQ ID NO: 147 - protein, 7 aa, synthetic construct
  source 1..7: note = CD98HC.04.013 CDR-L2
WASTRES

SEQ ID NO: 148 - protein, 9 aa, synthetic construct
  source 1..9: note = CD98HC.04.013 CDR-L3
QQYYTYPPT

SEQ ID NO: 149 - protein, 12 aa, synthetic construct
  source 1..12: note = CD98HC.04.018 CDR-L1
TASSSVSSSYLY

SEQ ID NO: 150 - protein, 7 aa, synthetic construct
  source 1..7: note = CD98HC.04.018 CDR-L2
SIFNLAS

SEQ ID NO: 151 - protein, 9 aa, synthetic construct
  source 1..9: note = CD98HC.04.018 CDR-L3
HQYHRSPPT

SEQ ID NO: 152 - protein, 11 aa, synthetic construct
  source 1..11: note = CD98HC.04.022 CDR-L1
HASQGISSNIG

SEQ ID NO: 153 - protein, 7 aa, synthetic construct
  source 1..7: note = CD98HC.04.022 CDR-L2
HGSNLED

SEQ ID NO: 154 - protein, 9 aa, synthetic construct
  source 1..9: note = CD98HC.04.022 CDR-L3
VQYAQFPRT

SEQ ID NO: 155 - protein, 11 aa, synthetic construct
  source 1..11: note = CD98HC.04.045 CDR-L1
RASQEISGYLS

SEQ ID NO: 156 - protein, 7 aa, synthetic construct
  source 1..7: note = CD98HC.04.045 CDR-L2
AASTLDS

SEQ ID NO: 157 - protein, 9 aa, synthetic construct
  source 1..9: note = CD98HC.04.045 CDR-L3
LQYASYPRT

SEQ ID NO: 158 - protein, 12 aa, synthetic construct
  source 1..12: note = CD98HC.04.046 CDR-L1
IVSSSVSSSYLY

SEQ ID NO: 159 - protein, 7 aa, synthetic construct
  source 1..7: note = CD98HC.04.046 CDR-L2
STSILAS

SEQ ID NO: 160 - protein, 9 aa, synthetic construct
  source 1..9: note = CD98HC.04.046 CDR-L3
HQYHRSPPT

SEQ ID NO: 161 - protein, 10 aa, synthetic construct
  source 1..10: note = CD98HC.04.047 CDR-L1
SASSSVGYMY

SEQ ID NO: 162 - protein, 7 aa, synthetic construct
  source 1..7: note = CD98HC.04.047 CDR-L2
RTSILAS

SEQ ID NO: 163 - protein, 9 aa, synthetic construct
  source 1..9: note = CD98HC.04.047 CDR-L3
QQYQSYPLT

SEQ ID NO: 164 - protein, 11 aa, synthetic construct
  source 1..11: note = CD98HC.04.054 CDR-L1
KASQNVGSAVA

SEQ ID NO: 165 - protein, 7 aa, synthetic construct
  source 1..7: note = CD98HC.04.054 CDR-L2
SASNRYT

SEQ ID NO: 166 - protein, 9 aa, synthetic construct
  source 1..9: note = CD98HC.04.054 CDR-L3
QQYNFYPLT

SEQ ID NO: 167 - protein, 17 aa, synthetic construct
  source 1..17: note = CD98HC.04.058 CDR-L1
KSSQTVLYSSNQKNYLA

SEQ ID NO: 168 - protein, 7 aa, synthetic construct
  source 1..7: note = CD98HC.04.058 CDR-L2
WASTRES

SEQ ID NO: 169 - protein, 8 aa, synthetic construct
  source 1..8: note = CD98HC.04.058 CDR-L3
HQYLSSYT

SEQ ID NO: 170 - protein, 17 aa, synthetic construct
  source 1..17: note = CD98HC.04.062 CDR-L1
KSSQSLLYSGNQKTYLA

SEQ ID NO: 171 - protein, 7 aa, synthetic construct
  source 1..7: note = CD98HC.04.062 CDR-L2
WASTRES

SEQ ID NO: 172 - protein, 9 aa, synthetic construct
  source 1..9: note = CD98HC.04.062 CDR-L3
QQYYSYPPT

SEQ ID NO: 173 - protein, 11 aa, synthetic construct
  source 1..11: note = CD98HC.04.063 CDR-L1
KASDHIKNWLA

SEQ ID NO: 174 - protein, 7 aa, synthetic construct
  source 1..7: note = CD98HC.04.063 CDR-L2
GATILET

SEQ ID NO: 175 - protein, 9 aa, synthetic construct
  source 1..9: note = CD98HC.04.063 CDR-L3
QQYWSTPYT

SEQ ID NO: 176 - protein, 17 aa, synthetic construct
  source 1..17: note = CD98HC.04.064 CDR-L1
KSSQSLLNNINQKNYLA

SEQ ID NO: 177 - protein, 7 aa, synthetic construct
  source 1..7: note = CD98HC.04.064 CDR-L2
FASTRES

SEQ ID NO: 178 - protein, 9 aa, synthetic construct
  source 1..9: note = CD98HC.04.064 CDR-L3
QQHYSSPFT

SEQ ID NO: 179 - protein, 10 aa, synthetic construct
  source 1..10: note = CD98HC.04.065 CDR-L1
SASSSVHYMY

SEQ ID NO: 180 - protein, 7 aa, synthetic construct
  source 1..7: note = CD98HC.04.065 CDR-L2
RTSILAS

SEQ ID NO: 181 - protein, 9 aa, synthetic construct
  source 1..9: note = CD98HC.04.065 CDR-L3
QQYQSYPLT

SEQ ID NO: 182 - protein, 20 aa, synthetic construct
GGSEGKSSGSGSESKSTGGS

SEQ ID NO: 183 - protein, 5 aa, synthetic construct
  REGION 1..5: note = The entire sequence of amino acids 1-5 is repeated 3 times
GGGGS

SEQ ID NO: 184 - protein, 486 aa, synthetic construct
  source 1..486: note = Isotype control heavy chain wt hIgG1 holescFv (T366S_L368A_Y407V)-(G4S)3 linker-VH-20AA linker
  SITE 465: note = X is the amino acid sequence of any one of SEQ ID NOs:6, 8, 10, 12, 14, 16, 18, 20, 22, 24, 26, 28, 30, 32, 34, 36, 38, 40, 42, 44, 46, 48, 194, 196, 198, 200, 202, 204, 206, 208, 210, 212, 214, 216, 218, 220, 222, 224, 355, 357, 359, 361, 363, 365, 367, 369, 371, 373, 375, 377, 379, 381, 383, 385, 387, 389, 401, and 403
  SITE 486: note = X is the amino acid sequence of any one of SEQ ID NOs:SEQ ID NO:7, 9, 11, 13, 15, 17, 19, 21, 23, 25, 27, 29, 31, 33, 35, 37, 39, 41, 43, 45, 47, 49, 195, 197, 199, 201, 203, 205, 207, 209, 211, 213, 215, 217, 219, 221, 223, 225, 356, 358, 360, 362, 364, 366, 368, 370, 372, 374, 376, 378, 380, 382, 384, 386, 388, 400, 402, and 404
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSXGGSEGKSSGSGSESKSTGGSX

SEQ ID NO: 185 - protein, 486 aa, synthetic construct
  source 1..486: note = Isotype control heavy chain wt hIgG1 hole scFv (T366S_L368A_Y407V)-(G4S)3 linker-VH-20AA linker-VL with mutations (H435R, Y436F)
  SITE 465: note = X is the amino acid sequence of any one of SEQ ID NOs:6, 8, 10, 12, 14, 16, 18, 20, 22, 24, 26, 28, 30, 32, 34, 36, 38, 40, 42, 44, 46, 48, 194, 196, 198, 200, 202, 204, 206, 208, 210, 212, 214, 216, 218, 220, 222, and 224, 355, 357, 359, 361, 363, 365, 367, 369, 371, 373, 375, 377, 379, 381, 383, 385, 387, 389, 401, and 403
  SITE 486: note = X is the amino acid sequence of any one of SEQ ID NOs:SEQ ID NO:7, 9, 11, 13, 15, 17, 19, 21, 23, 25, 27, 29, 31, 33, 35, 37, 39, 41, 43, 45, 47, 49, 195, 197, 199, 201, 203, 205, 207, 209, 211, 213, 215, 217, 219, 221, 223, 225, 356, 358, 360, 362, 364, 366, 368, 370, 372, 374, 376, 378, 380, 382, 384, 386, 388, 400, 402, and 404
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGKGGGGSGGGGSGGGGSXGGSEGKSSGSGSESKSTGGSX

SEQ ID NO: 186 - protein, 449 aa, synthetic construct
  source 1..449: note = Isotype control heavy chain hIgG1 wt knob
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 187 - protein, 214 aa, synthetic construct
  source 1..214: note = Isotype control light chain
DIQMTQSPSSLSASVGDRVTITCRASQSISSYLNWYQQKPGKAPKLLIYAASSLQSGVPSRFSGSGSGTDFTLTISSLQPEDFATYYCQQADLPAFAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 188 - protein, 486 aa, synthetic construct
  source 1..486: note = Isotype control heavy chain hIgG1 LALAPS hole scFv (T366S_L368A_Y407V) (L234A_L235A_P331S)-(G4S)3 linker-VH-20AA linker-VL
  SITE 465: note = X is the amino acid sequence of any one of SEQ ID NOs:6, 8, 10, 12, 14, 16, 18, 20, 22, 24, 26, 28, 30, 32, 34, 36, 38, 40, 42, 44, 46, 48, 194, 196, 198, 200, 202, 204, 206, 208, 210, 212, 214, 216, 218, 220, 222, and 224, 355, 357, 359, 361, 363, 365, 367, 369, 371, 373, 375, 377, 379, 381, 383, 385, 387, 389, 401, and 403
  SITE 486: note = X is the amino acid sequence of any one of SEQ ID NOs:SEQ ID NO:7, 9, 11, 13, 15, 17, 19, 21, 23, 25, 27, 29, 31, 33, 35, 37, 39, 41, 43, 45, 47, 49, 195, 197, 199, 201, 203, 205, 207, 209, 211, 213, 215, 217, 219, 221, 223, 225, 356, 358, 360, 362, 364, 366, 368, 370, 372, 374, 376, 378, 380, 382, 384, 386, 388, 400, 402, and 404
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSXGGSEGKSSGSGSESKSTGGSX

SEQ ID NO: 189 - protein, 486 aa, synthetic construct
  source 1..486: note = Isotype control heavy chain hIgG1 LALAPS hole scFv (T366S_L368A_Y407V) (L234A_L235A_P331S)-(G4S)3 linker-VH-20AA linker-VL with mutations (H435R, Y436F)
  SITE 465: note = X is the amino acid sequence of any one of SEQ ID NOs:6, 8, 10, 12, 14, 16, 18, 20, 22, 24, 26, 28, 30, 32, 34, 36, 38, 40, 42, 44, 46, 48, 194, 196, 198, 200, 202, 204, 206, 208, 210, 212, 214, 216, 218, 220, 222, and 224, 355, 357, 359, 361, 363, 365, 367, 369, 371, 373, 375, 377, 379, 381, 383, 385, 387, 389, 401, and 403
  SITE 486: note = X is the amino acid sequence of any one of SEQ ID NOs:SEQ ID NO:7, 9, 11, 13, 15, 17, 19, 21, 23, 25, 27, 29, 31, 33, 35, 37, 39, 41, 43, 45, 47, 49, 195, 197, 199, 201, 203, 205, 207, 209, 211, 213, 215, 217, 219, 221, 223, 225, 356, 358, 360, 362, 364, 366, 368, 370, 372, 374, 376, 378, 380, 382, 384, 386, 388, 400, 402, and 404
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGKGGGGSGGGGSGGGGSXGGSEGKSSGSGSESKSTGGSX

SEQ ID NO: 190 - protein, 449 aa, synthetic construct
  source 1..449: note = Isotype control heavy chain hIgG1 LALAPS knob
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 191 - protein, 486 aa, synthetic construct
  source 1..486: note = Isotype control heavy chain hIgG1 NSLF hole scFv (T366S_L368A_Y407V) (N325S_L328F)-(G4S)3 linker-VH-20AA linker-VL
  SITE 465: note = X is the amino acid sequence of any one of SEQ ID NOs:6, 8, 10, 12, 14, 16, 18, 20, 22, 24, 26, 28, 30, 32, 34, 36, 38, 40, 42, 44, 46, 48, 194, 196, 198, 200, 202, 204, 206, 208, 210, 212, 214, 216, 218, 220, 222, and 224, 355, 357, 359, 361, 363, 365, 367, 369, 371, 373, 375, 377, 379, 381, 383, 385, 387, 389, 401, and 403
  SITE 486: note = X is the amino acid sequence of any one of SEQ ID NOs:SEQ ID NO:7, 9, 11, 13, 15, 17, 19, 21, 23, 25, 27, 29, 31, 33, 35, 37, 39, 41, 43, 45, 47, 49, 195, 197, 199, 201, 203, 205, 207, 209, 211, 213, 215, 217, 219, 221, 223, 225, 356, 358, 360, 362, 364, 366, 368, 370, 372, 374, 376, 378, 380, 382, 384, 386, 388, 400, 402, and 404
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSXGGSEGKSSGSGSESKSTGGSX

SEQ ID NO: 192 - protein, 486 aa, synthetic construct
  source 1..486: note = Isotype control heavy chain hIgG1 NSLF hole scFv (T366S_L368A_Y407V) (N325S_L328F)-(G4S)3 linker-VH-20AA linker-VL mutations (H435R, Y436F)
  SITE 465: note = X is the amino acid sequence of any one of SEQ ID NOs:6, 8, 10, 12, 14, 16, 18, 20, 22, 24, 26, 28, 30, 32, 34, 36, 38, 40, 42, 44, 46, 48, 194, 196, 198, 200, 202, 204, 206, 208, 210, 212, 214, 216, 218, 220, 222, and 224, 355, 357, 359, 361, 363, 365, 367, 369, 371, 373, 375, 377, 379, 381, 383, 385, 387, 389, 401, and 403
  SITE 486: note = X is the amino acid sequence of any one of SEQ ID NOs:SEQ ID NO:7, 9, 11, 13, 15, 17, 19, 21, 23, 25, 27, 29, 31, 33, 35, 37, 39, 41, 43, 45, 47, 49, 195, 197, 199, 201, 203, 205, 207, 209, 211, 213, 215, 217, 219, 221, 223, 225, 356, 358, 360, 362, 364, 366, 368, 370, 372, 374, 376, 378, 380, 382, 384, 386, 388, 400, 402, and 404
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGKGGGGSGGGGSGGGGSXGGSEGKSSGSGSESKSTGGSX

SEQ ID NO: 193 - protein, 449 aa, synthetic construct
  source 1..449: note = Isotype control heavy chain hIgG1 NSLF knob (T366W) (N325S_L328F)
EVRLLESGGGLVQPGGSLRLSCAASGFTFSNYAMGWVRQAPGKGLEWVSAISGSGGSTYYADSVKGRFTTSRDDSKNALYLQMNSLRAEDTAVYYCARGGPGWYAADVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 194 - protein, 119 aa, synthetic construct
  source 1..119: note = CD98hc.05.006 Heavy Chain Variable Region
EVKLVESGGNLVQPGGSLKLSCAASGFTFSGYSMSWVRQTPDKRLEWVAYIGNGGDSTYYPYTVKGRFTISRDNAKNTLYLQMNSLKSEDSAMYYCARQYVNYPFFDYWGQGTTLTVSS

SEQ ID NO: 195 - protein, 113 aa, synthetic construct
  source 1..113: note = CD98hc.05.006 Light Chain Variable Region
DIVMSQSPSSLTVSVGAKIILGCKSSQSLLYSGNQKNYLAWYQQKPGQSPKLLIYWASTRESGVPDRFTGSGSGTDFTLTISSVKAEDVAVYYCQQYYTYPPAFGAGTKLEIK

SEQ ID NO: 196 - protein, 119 aa, synthetic construct
  source 1..119: note = CD98hc.05.007 Heavy Chain Variable Region
DVKLVESGGDLVKPGGSLKLSCAASGFTFSSYSMSWVRQTPEKRLEWVATITSGGSYTYFPDSVKGRFTISRDNAKNTLYLQVSNLKSEDTAMYYCTRGPSFGNYLDYWGQGTTLTVSS

SEQ ID NO: 197 - protein, 107 aa, synthetic construct
  source 1..107: note = CD98hc.05.007 Light Chain Variable Region
DILMTQSPSSMSASLGDTVSITCHASQGISSNIGWLQQKPGKSFKGLIYRGTNLEDGVPSRFSGSGSGADYSLTISSLESEDFADYYCVQYAHFPYTFGGGTKLEIK

SEQ ID NO: 198 - protein, 123 aa, synthetic construct
  source 1..123: note = CD98hc.05.046 Heavy Chain Variable Region
EVQLVESGGGLVKPGGSLKLSCAASGFTFSDYYMYWVRQTPEKRLEWVATISDGGSYTYYPDSVKGRFTISRDNAKNNLYLQMSSLKSEDTAMYYCARDRAYYYGSSYGFGYWGQGTLVTVSS

SEQ ID NO: 199 - protein, 112 aa, synthetic construct
  source 1..112: note = CD98hc.05.046 Light Chain Variable Region
DVVMTQTPLSLPVSLGNQASISCRSSQSLVHSNGNTFFHWYLQKPGQSPKLLIYKVSNRFSGVPDRFSGSGSGTDFTLKISRVEAEDLGLYFCSQTTHVPPTFGGGTKLEIK

SEQ ID NO: 200 - protein, 120 aa, synthetic construct
  source 1..120: note = CD98hc.05.052 Heavy Chain Variable Region
DMQLQESGPGLVKPSQSLSLTCTVTGYSITSDYAWNWIRQFPGNKLEWMGFISYSGTTSYTPSLKRRISITRDTSKNQFFLRLNSVTPEDTATYYCAKSAFYGNVWFAYWGQGTLVTVSS

SEQ ID NO: 201 - protein, 106 aa, synthetic construct
  source 1..106: note = CD98hc.05.052 Light Chain Variable Region
QFVLTQSPAIMSASPGEKVTISCSASSSVSYMFWYQQKPGSSPKPWISHTSNLASGVPARFSGSGSGTSYSLTISNMEAEDAATYYCQQFHDYPWTFGGGTKLEIK

SEQ ID NO: 202 - protein, 116 aa, synthetic construct
  source 1..116: note = CD98hc.05.059 Heavy Chain Variable Region
EIQLQQSGPELVKPGASVKVSCKASGYSFNMFWVKQSHGKSLEWIGHIDPFNGDTSYSPNFEDRASLTVDRSSNTAYMHLNSLTSEDSAVYYCARSVGAHWYFDVWGAGTTVTVSS

SEQ ID NO: 203 - protein, 108 aa, synthetic construct
  source 1..108: note = CD98hc.05.059 Light Chain Variable Region
QIVLTQSPAIMSASLGERVTMTCTASSSVTSSYLYWYQQKPGSSPKLWIYSTSNLASGVPPRFSGSGSGTSYSLTISSMEAEDAATYHCQQYHRSPATFGAGTKLEIK

SEQ ID NO: 204 - protein, 119 aa, synthetic construct
  source 1..119: note = CD98hc.05.081 Heavy Chain Variable Region
EVQLQQSRPELVKPGASVKMSCKASGYTFTSYVMHWVKQKPGQGLEWIGYISPYNGDTKYNEKFKGKATLTSDISSTTAYMELSSLTSEDSGVYYCARPGSSPSWFAYWGQGTLVTVSS

SEQ ID NO: 205 - protein, 109 aa, synthetic construct
  source 1..109: note = CD98hc.05.081 Light Chain Variable Region
DIVMTQFPATLSVTPGDRVSLSCRASQSISNYLHWYQQKSHESPRLLIKYASQSISGIPSRFSGSGSGSDFTLSINSVEPEDVGVYYCQNGHSFPYMYTFGGGTKLEIK

SEQ ID NO: 206 - protein, 119 aa, synthetic construct
  source 1..119: note = CD98hc.05.126 Heavy Chain Variable Region
EVKLVESGGNLVQPGGSLKLSCAASGFTFSGYPMSWVRRTPDKRLEWVAYIGSGGDSTYYPYAVKGRFTISRDNAKNTLYLQMNSLKSEDSAMYYCARQYDNYPFFDYWGQGTTLTVSS

SEQ ID NO: 207 - protein, 113 aa, synthetic construct
  source 1..113: note = CD98hc.05.126 Light Chain Variable Region
DFVMSQSPSSLTVSVGAKVIMSCKSSQSLLYSGNQKNYLAWYQQKPGQSPKLLIYWASTRESGVPDRFTGSGSGTDFTLTISSVKAEDLAVYYCQQYYTYPPTFGAGTKLEIK

SEQ ID NO: 208 - protein, 118 aa, synthetic construct
  source 1..118: note = CD98hc.05.144 Heavy Chain Variable Region
EVQLVESGGALVKPGGSLKLSCAASGFAFSSFDMSWVRQTPERRVEWVAYISGGGGSTYHPDTVKGRFAISRDNAKNTLYLHMNSLKSEDTAMYYCVRQGSRPWFAYWGQGTLVTVSS

SEQ ID NO: 209 - protein, 113 aa, synthetic construct
  source 1..113: note = CD98hc.05.144 Light Chain Variable Region
DFVMSQSPSSLTVSVGEKVTMTCKSSQSLLYSYTQKNYLAWYQQKPGQSPNLLIYWASTRESGVPDRFAGSGSGTDFTLTISSVEAEDLAVYYCQQYSNYPPTFGGGTKLEIK

SEQ ID NO: 210 - protein, 119 aa, synthetic construct
  source 1..119: note = CD98hc.05.153 Heavy Chain Variable Region
EVQLVESGGGLVKPGGSLKLSCAASGFDFSNCDMSWVRQTPEKRLEWVAYISNSGDSTYFPHTVKGRFTISRDNAKNALYLQMSSLKSEDTAMYYCARQGGHYPWFVYWGQGTLVTVSS

SEQ ID NO: 211 - protein, 113 aa, synthetic construct
  source 1..113: note = CD98hc.05.153 Light Chain Variable Region
DIVMSQSPSFLDVSVGEKVAMTCKSIQSLLYSGNQKNYLAWYQQKPGQSPKVLIYWASTRESGVPDRFTGSGSGTDFTLTISSVKAEDLAVYYCQQYYTYPPTFGGGTKLEIK

SEQ ID NO: 212 - protein, 126 aa, synthetic construct
  source 1..126: note = CD98hc.05.166 Heavy Chain Variable Region
EVQLLQSGAELVKPGASVKLSCTASGFNILDTYIHWVMQRPEQGLEWIGRIDPANGHVKYDPKFQGKATITADTSSNTAYLQLSSLTSEDTAVYYCASPFTTVVATNIYYYGLGYWGHGTSVTVSS

SEQ ID NO: 213 - protein, 107 aa, synthetic construct
  source 1..107: note = CD98hc.05.166 Light Chain Variable Region
DIVMTQSHKFMSTSLGDRVSITCKASQGVFTAVAWYQQKPGQSPKLLIYSASYRYTGVPDRFTGSGSGTDFTFTISSVQAEDLAVYYCQQHYSTPWTFGGGTKLEIK

SEQ ID NO: 214 - protein, 118 aa, synthetic construct
  source 1..118: note = CD98hc.05.168 Heavy Chain Variable Region
EVQLVESGGGLVKPGGSLKLSCAASGFAFSTYDMSWVRQTPEKRLEWVAYISSGGDTTYYPDTVKGRFTISRDNAKNTLYLQMSSLKSEDSAIYYCARQDYRPYFAYWGQGTLVTVSS

SEQ ID NO: 215 - protein, 113 aa, synthetic construct
  source 1..113: note = CD98hc.05.168 Light Chain Variable Region
DIVMSQSPSSLAVSVGEKVTVSCKSSQNLLYSSNQKNYLAWYQQKPGQSPKLLIYWASTRESGVPDRFTGSGSGTDFTLTISSVKAEDLAVYYCQQYSYYPPTFGGGTKLEIK

SEQ ID NO: 216 - protein, 120 aa, synthetic construct
  source 1..120: note = CD98hc.05.188 Heavy Chain Variable Region
QVQLQQPGAELVRPGASVKLSCKASGYTFTSYWMSWVKQRPGQGLEWIGFITPSDSEALYSQMFKDKATLTVDKSSSTAHLQLSSLTSEDSAVYYCARWSYGNFAAMDYWGQGTSVTVSS

SEQ ID NO: 217 - protein, 113 aa, synthetic construct
  source 1..113: note = CD98hc.05.188 Light Chain Variable Region
DIVMTQSPSSLAMSVGQKVTMSCKSSQSLLNSNNQKNYLAWYQQKPGQSPKLLVYFSSTRESGVPDRFIGSGSGTDFTLTITSVQAEDLADYFCQQHYSTPLTFGAGTKLEIK

SEQ ID NO: 218 - protein, 119 aa, synthetic construct
  source 1..119: note = CD98hc.05.193 Heavy Chain Variable Region
EVQLVESGGGLVQPGGSLKVSCTTSGFTFSDFDMSWIRQTPEKRLEWVAYISSGGGSTYFPDTVKGRFTISRDNAKSTLYLQMSSLKSEDTAMYYCARHVGVRPWFDYWGQGTLVTVSS

SEQ ID NO: 219 - protein, 113 aa, synthetic construct
  source 1..113: note = CD98hc.05.193 Light Chain Variable Region
DIVMSQSPSSLAVSVGEKVTMTCKSSQSLLYSSNQKNYLAWYQQKPGQSPKLLIYWASTRESGVPDRFTGSGSGTDFTLTISSVKAEDLALYYCQQYHYYPPTFGAGTKLEIK

SEQ ID NO: 220 - protein, 120 aa, synthetic construct
  source 1..120: note = CD98hc.05.207 Heavy Chain Variable Region
EVQLVESGGGLVKPGGSLKISCAASGFAFSSYDMSWVRQTPEKRLEWVAYISSAGGSTYFPGTVKGRFTISRDNAKNTLYLQMRSLKSEDTAMYYCARHENGHYPFFDFWGQGTTLTVSS

SEQ ID NO: 221 - protein, 113 aa, synthetic construct
  source 1..113: note = CD98hc.05.207 Light Chain Variable Region
DIVMSQSPSSLAVSVGEKVTLSCKSSQSLFYSGNQKTYLAWYQQKPGQSPKLLIYWTSTRESGVPDRFTGSGSGTDFTLTISSVKAEDLAVYFCQQYYTYPPTFGAGTKLEIK

SEQ ID NO: 222 - protein, 124 aa, synthetic construct
  source 1..124: note = CD98hc.05.208 Heavy Chain Variable Region
QVQLQQSGPELVRPGVSVKISCKSSGYAFTDYAVHWVRQSRAMSLEWIGIISTYSGNIDYNQQFKGKATMTVDKSSSTVYMELARLTSEDSAIYFCARGGRHYYGSYYYAFDYWGQGTSVTVSS

SEQ ID NO: 223 - protein, 113 aa, synthetic construct
  source 1..113: note = CD98hc.05.208 Light Chain Variable Region
DIVMTQSPSSLAISVGQKVTMSCKSSQSLLNRNNQKNYLAWYQQKPGQSPKLLLYFTSTRESGVPDRFIGSGSGTDFTLIISSVQAEDLADYFCQQHYSTPPTFGGGTKLEIK

SEQ ID NO: 224 - protein, 119 aa, synthetic construct
  source 1..119: note = CD98hc.05.226 Heavy Chain Variable Region
EVQLVESGGGLGKPGGSLKLSCAASGFAFSSYDMSWVRQTPEKRLDWVAYISSGGGGTYYPDTVKGRFTISRDNAKNTLYLQMSSLKSEDTAIYYCARHVGVRPWFGYWGQGTLVTVSS

SEQ ID NO: 225 - protein, 113 aa, synthetic construct
  source 1..113: note = CD98hc.05.226 Light Chain Variable Region
DIVMSQSPSSLAVSVGEKITMTCKSSQSLLYSGSQKNYLAWYQQKPGQSPKLLIYWASTRESGVPDRFTGSGSGTDFTLTISSVKAEDLAVYYCQQYSYYPPTFGAGTKLEIK

SEQ ID NO: 226 - protein, 5 aa, synthetic construct
  source 1..5: note = CD98hc.05.006 CDR-H1
GYSMS

SEQ ID NO: 227 - protein, 17 aa, synthetic construct
  source 1..17: note = CD98hc.05.006 CDR-H2
YIGNGGDSTYYPYTVKG

SEQ ID NO: 228 - protein, 10 aa, synthetic construct
  source 1..10: note = CD98hc.05.006 CDR-H3
QYVNYPFFDY

SEQ ID NO: 229 - protein, 5 aa, synthetic construct
  source 1..5: note = CD98hc.05.007 CDR-H1
SYSMS

SEQ ID NO: 230 - protein, 17 aa, synthetic construct
  source 1..17: note = CD98hc.05.007 CDR-H2
TITSGGSYTYFPDSVKG

SEQ ID NO: 231 - protein, 10 aa, synthetic construct
  source 1..10: note = CD98hc.05.007 CDR-H3
GPSFGNYLDY

SEQ ID NO: 232 - protein, 5 aa, synthetic construct
  source 1..5: note = CD98hc.05.046 CDR-H1
DYYMY

SEQ ID NO: 233 - protein, 17 aa, synthetic construct
  source 1..17: note = CD98hc.05.046 CDR-H2
TISDGGSYTYYPDSVKG

SEQ ID NO: 234 - protein, 14 aa, synthetic construct
  source 1..14: note = CD98hc.05.046 CDR-H3
DRAYYYGSSYGFGY

SEQ ID NO: 235 - protein, 6 aa, synthetic construct
  source 1..6: note = CD98hc.05.052 CDR-H1
SDYAWN

SEQ ID NO: 236 - protein, 16 aa, synthetic construct
  source 1..16: note = CD98hc.05.052 CDR-H2
FISYSGTTSYTPSLKR

SEQ ID NO: 237 - protein, 11 aa, synthetic construct
  source 1..11: note = CD98hc.05.052 CDR-H3
SAFYGNVWFAY

SEQ ID NO: 238 - protein, 5 aa, synthetic construct
  source 1..5: note = CD98hc.05.059 CDR-H1
SFNMF

SEQ ID NO: 239 - protein, 17 aa, synthetic construct
  source 1..17: note = CD98hc.05.059 CDR-H2
HIDPFNGDTSYSPNFED

SEQ ID NO: 240 - protein, 10 aa, synthetic construct
  source 1..10: note = CD98hc.05.059 CDR-H3
SVGAHWYFDV

SEQ ID NO: 241 - protein, 5 aa, synthetic construct
  source 1..5: note = CD98hc.05.081 CDR-H1
SYVMH

SEQ ID NO: 242 - protein, 17 aa, synthetic construct
  source 1..17: note = CD98hc.05.081 CDR-H2
YISPYNGDTKYNEKFKG

SEQ ID NO: 243 - protein, 10 aa, synthetic construct
  source 1..10: note = CD98hc.05.081 CDR-H3
PGSSPSWFAY

SEQ ID NO: 244 - protein, 5 aa, synthetic construct
  source 1..5: note = CD98hc.05.126 CDR-H1
GYPMS

SEQ ID NO: 245 - protein, 17 aa, synthetic construct
  source 1..17: note = CD98hc.05.126 CDR-H2
YIGSGGDSTYYPYAVKG

SEQ ID NO: 246 - protein, 10 aa, synthetic construct
  source 1..10: note = CD98hc.05.126 CDR-H3
QYDNYPFFDY

SEQ ID NO: 247 - protein, 5 aa, synthetic construct
  source 1..5: note = CD98hc.05.144 CDR-H1
SFDMS

SEQ ID NO: 248 - protein, 17 aa, synthetic construct
  source 1..17: note = CD98hc.05.144 CDR-H2
YISGGGGSTYHPDTVKG

SEQ ID NO: 249 - protein, 9 aa, synthetic construct
  source 1..9: note = CD98hc.05.144 CDR-H3
QGSRPWFAY

SEQ ID NO: 250 - protein, 5 aa, synthetic construct
  source 1..5: note = CD98hc.05.153 CDR-H1
NCDMS

SEQ ID NO: 251 - protein, 17 aa, synthetic construct
  source 1..17: note = CD98hc.05.153 CDR-H2
YISNSGDSTYFPHTVKG

SEQ ID NO: 252 - protein, 10 aa, synthetic construct
  source 1..10: note = CD98hc.05.153 CDR-H3
QGGHYPWFVY

SEQ ID NO: 253 - protein, 5 aa, synthetic construct
  source 1..5: note = CD98hc.05.166 CDR-H1
DTYIH

SEQ ID NO: 254 - protein, 17 aa, synthetic construct
  source 1..17: note = CD98hc.05.166 CDR-H2
RIDPANGHVKYDPKFQG

SEQ ID NO: 255 - protein, 17 aa, synthetic construct
  source 1..17: note = CD98hc.05.166 CDR-H3
PFTTVVATNIYYYGLGY

SEQ ID NO: 256 - protein, 5 aa, synthetic construct
  source 1..5: note = CD98hc.05.168 CDR-H1
TYDMS

SEQ ID NO: 257 - protein, 17 aa, synthetic construct
  source 1..17: note = CD98hc.05.168 CDR-H2
YISSGGDTTYYPDTVKG

SEQ ID NO: 258 - protein, 9 aa, synthetic construct
  source 1..9: note = CD98hc.05.168 CDR-H3
QDYRPYFAY

SEQ ID NO: 259 - protein, 5 aa, synthetic construct
  source 1..5: note = CD98hc.05.188 CDR-H1
SYWMS

SEQ ID NO: 260 - protein, 17 aa, synthetic construct
  source 1..17: note = CD98hc.05.188 CDR-H2
FITPSDSEALYSQMFKD

SEQ ID NO: 261 - protein, 11 aa, synthetic construct
  source 1..11: note = CD98hc.05.188 CDR-H3
WSYGNFAAMDY

SEQ ID NO: 262 - protein, 5 aa, synthetic construct
  source 1..5: note = CD98hc.05.193 CDR-H1
DFDMS

SEQ ID NO: 263 - protein, 17 aa, synthetic construct
  source 1..17: note = CD98hc.05.193 CDR-H2
YISSGGGSTYFPDTVKG

SEQ ID NO: 264 - protein, 10 aa, synthetic construct
  source 1..10: note = CD98hc.05.193 CDR-H3
HVGVRPWFDY

SEQ ID NO: 265 - protein, 5 aa, synthetic construct
  source 1..5: note = CD98hc.05.226 CDR-H1
SYDMS

SEQ ID NO: 266 - protein, 17 aa, synthetic construct
  source 1..17: note = CD98hc.05.207 CDR-H2
YISSAGGSTYFPGTVKG

SEQ ID NO: 267 - protein, 11 aa, synthetic construct
  source 1..11: note = CD98hc.05.207 CDR-H3
HENGHYPFFDF

SEQ ID NO: 268 - protein, 5 aa, synthetic construct
  source 1..5: note = CD98hc.05.208 CDR-H1
DYAVH

SEQ ID NO: 269 - protein, 17 aa, synthetic construct
  source 1..17: note = CD98hc.05.208 CDR-H2
IISTYSGNIDYNQQFKG

SEQ ID NO: 270 - protein, 15 aa, synthetic construct
  source 1..15: note = CD98hc.05.208 CDR-H3
GGRHYYGSYYYAFDY

SEQ ID NO: 271 - protein, 17 aa, synthetic construct
  source 1..17: note = CD98hc.05.226 CDR-H2
YISSGGGGTYYPDTVKG

SEQ ID NO: 272 - protein, 10 aa, synthetic construct
  source 1..10: note = CD98hc.05.226 CDR-H3
HVGVRPWFGY

SEQ ID NO: 273 - protein, 17 aa, synthetic construct
  source 1..17: note = CD98hc.05.126 CDR-L1
KSSQSLLYSGNQKNYLA

SEQ ID NO: 274 - protein, 7 aa, synthetic construct
  source 1..7: note = CD98hc.05.226 CDR-L2
WASTRES

SEQ ID NO: 275 - protein, 9 aa, synthetic construct
  source 1..9: note = CD98hc.05.006 CDR-L3
QQYYTYPPA

SEQ ID NO: 276 - protein, 11 aa, synthetic construct
  source 1..11: note = CD98hc.05.007 CDR-L1
HASQGISSNIG

SEQ ID NO: 277 - protein, 7 aa, synthetic construct
  source 1..7: note = CD98hc.05.007 CDR-L2
RGTNLED

SEQ ID NO: 278 - protein, 9 aa, synthetic construct
  source 1..9: note = CD98hc.05.007 CDR-L3
VQYAHFPYT

SEQ ID NO: 279 - protein, 16 aa, synthetic construct
  source 1..16: note = CD98hc.05.046 CDR-L1
RSSQSLVHSNGNTFFH

SEQ ID NO: 280 - protein, 7 aa, synthetic construct
  source 1..7: note = CD98hc.05.046 CDR-L2
KVSNRFS

SEQ ID NO: 281 - protein, 9 aa, synthetic construct
  source 1..9: note = CD98hc.05.046 CDR-L3
SQTTHVPPT

SEQ ID NO: 282 - protein, 10 aa, synthetic construct
  source 1..10: note = CD98hc.05.052 CDR-L1
SASSSVSYMF

SEQ ID NO: 283 - protein, 7 aa, synthetic construct
  source 1..7: note = CD98hc.05.052 CDR-L2
HTSNLAS

SEQ ID NO: 284 - protein, 9 aa, synthetic construct
  source 1..9: note = CD98hc.05.052 CDR-L3
QQFHDYPWT

SEQ ID NO: 285 - protein, 12 aa, synthetic construct
  source 1..12: note = CD98hc.05.059 CDR-L1
TASSSVTSSYLY

SEQ ID NO: 286 - protein, 7 aa, synthetic construct
  source 1..7: note = CD98hc.05.059 CDR-L2
STSNLAS

SEQ ID NO: 287 - protein, 9 aa, synthetic construct
  source 1..9: note = CD98hc.05.059 CDR-L3
QQYHRSPAT

SEQ ID NO: 288 - protein, 11 aa, synthetic construct
  source 1..11: note = CD98hc.05.081 CDR-L1
RASQSISNYLH

SEQ ID NO: 289 - protein, 7 aa, synthetic construct
  source 1..7: note = CD98hc.05.081 CDR-L2
YASQSIS

SEQ ID NO: 290 - protein, 11 aa, synthetic construct
  source 1..11: note = CD98hc.05.081 CDR-L3
QNGHSFPYMYT

SEQ ID NO: 291 - protein, 9 aa, synthetic construct
  source 1..9: note = CD98hc.05.207 CDR-L3
QQYYTYPPT

SEQ ID NO: 292 - protein, 17 aa, synthetic construct
  source 1..17: note = CD98hc.05.144 CDR-L1
KSSQSLLYSYTQKNYLA

SEQ ID NO: 293 - protein, 9 aa, synthetic construct
  source 1..9: note = CD98hc.05.144 CDR-L3
QQYSNYPPT

SEQ ID NO: 294 - protein, 17 aa, synthetic construct
  source 1..17: note = CD98hc.05.153 CDR-L1
KSIQSLLYSGNQKNYLA

SEQ ID NO: 295 - protein, 11 aa, synthetic construct
  source 1..11: note = CD98hc.05.166 CDR-L1
KASQGVFTAVA

SEQ ID NO: 296 - protein, 7 aa, synthetic construct
  source 1..7: note = CD98hc.05.166 CDR-L2
SASYRYT

SEQ ID NO: 297 - protein, 9 aa, synthetic construct
  source 1..9: note = CD98hc.05.166 CDR-L3
QQHYSTPWT

SEQ ID NO: 298 - protein, 17 aa, synthetic construct
  source 1..17: note = CD98hc.05.168 CDR-L1
KSSQNLLYSSNQKNYLA

SEQ ID NO: 299 - protein, 9 aa, synthetic construct
  source 1..9: note = CD98hc.05.226 CDR-L3
QQYSYYPPT

SEQ ID NO: 300 - protein, 17 aa, synthetic construct
  source 1..17: note = CD98hc.05.188 CDR-L1
KSSQSLLNSNNQKNYLA

SEQ ID NO: 301 - protein, 7 aa, synthetic construct
  source 1..7: note = CD98hc.05.188 CDR-L2
FSSTRES

SEQ ID NO: 302 - protein, 9 aa, synthetic construct
  source 1..9: note = CD98hc.05.188 CDR-L3
QQHYSTPLT

SEQ ID NO: 303 - protein, 17 aa, synthetic construct
  source 1..17: note = CD98hc.05.193 CDR-L1
KSSQSLLYSSNQKNYLA

SEQ ID NO: 304 - protein, 9 aa, synthetic construct
  source 1..9: note = CD98hc.05.193 CDR-L3
QQYHYYPPT

SEQ ID NO: 305 - protein, 17 aa, synthetic construct
  source 1..17: note = CD98hc.05.207 CDR-L1
KSSQSLFYSGNQKTYLA

SEQ ID NO: 306 - protein, 7 aa, synthetic construct
  source 1..7: note = CD98hc.05.207 CDR-L2
WTSTRES

SEQ ID NO: 307 - protein, 17 aa, synthetic construct
  source 1..17: note = CD98hc.05.208 CDR-L1
KSSQSLLNRNNQKNYLA

SEQ ID NO: 308 - protein, 7 aa, synthetic construct
  source 1..7: note = CD98hc.05.208 CDR-L2
FTSTRES

SEQ ID NO: 309 - protein, 9 aa, synthetic construct
  source 1..9: note = CD98hc.05.208 CDR-L3
QQHYSTPPT

SEQ ID NO: 310 - protein, 17 aa, synthetic construct
  source 1..17: note = CD98hc.05.226 CDR-L1
KSSQSLLYSGSQKNYLA

SEQ ID NO: 311 - protein, 252 aa, synthetic construct
  source 1..252: note = mouse CD98hc.04.064
QVQLQQPGAELVRPGASVKLSCKASGYTFTSYWMNWVRQRPGQGLEWIGLIDPSDSETHYNQMFKDRATLTVDKSSSTAYMQLSSLTSADPAVYYCARASYGKGYFDYWGQGTTLTVSSGGSEGKSSGSGSESKSTGGSDIVMTQSPSSLAMSVGQKVTMSCKSSQSLLNNINQKNYLAWYQQKPGQSPKLLVYFASTRESGVPDRFIGSGSGTDFTLTINTVQAEDLADYFCQQHYSSPFTFGGGTKLEIK

SEQ ID NO: 312 - protein, 252 aa, synthetic construct
  source 1..252: note = CD98hc.04.064.1a
QVQLVQSGAEVKKPGASVKVSCKASGYTFTSYWMNWVRQAPGQGLEWMGIIDPSDSETHYAQKFQGRVTMTVDKSTSTVYMELSSLRSEDTAVYYCARASYGKGYFDYWGQGTLVTVSSGGSEGKSSGSGSESKSTGGSDIVMTQSPDSLAVSLGERATINCKSSQSLLNNINQKNYLAWYQQKPGQPPKLLIYFASTRESGVPDRFSGSGSGTDFTLTISSLQAEDVAVYYCQQHYSSPFTFGGGTKVEIK

SEQ ID NO: 313 - protein, 252 aa, synthetic construct
  source 1..252: note = CD98hc.04.064.1b
QVQLVQSGAEVKKPGASVKVSCKASGYTFTSYWMNWVRQAPGQGLEWMGIIDPSDSETHYAQKFQGRVTMTVDKSTSTVYMELSSLRSEDTAVYYCARASYGKGYFDYWGQGTLVTVSSGGSEGKSSGSGSESKSTGGSDIVMTQSPDSLAVSLGERATINCKSSQSLLNNINQKNYLAWYQQKPGQPPKLLIYFASTRESGVPDRFSGSGSGTDFTLTISSLQAEDVAVYFCQQHYSSPFTFGGGTKVEIK

SEQ ID NO: 314 - protein, 252 aa, synthetic construct
  source 1..252: note = CD98hc.04.064.1c
QVQLVQSGAEVKKPGASVKVSCKASGYTFTSYWMHWVRQAPGQGLEWMGIIDPSDSETSYAQKFQGRVTMTVDKSTSTVYMELSSLRSEDTAVYYCARASYGKGYFDYWGQGTLVTVSSGGSEGKSSGSGSESKSTGGSDIVMTQSPDSLAVSLGERATINCKSSQSLLNNINQKNYLAWYQQKPGQPPKLLIYFASTRESGVPDRFSGSGSGTDFTLTISSLQAEDVAVYYCQQHYSSPFTFGGGTKVEIK

SEQ ID NO: 315 - protein, 252 aa, synthetic construct
  source 1..252: note = CD98hc.04.064.1d
QVQLVQSGAEVKKPGASVKVSCKASGYTFTSYWMNWVRQAPGQGLEWMGIIDPSDSETSYAQKFQGRVTMTVDKSTSTVYMELSSLRSEDTAVYYCARASYGKGYFDYWGQGTLVTVSSGGSEGKSSGSGSESKSTGGSDIVMTQSPDSLAVSLGERATINCKSSQSLLNNINQKNYLAWYQQKPGQPPKLLIYFASTRESGVPDRFSGSGSGTDFTLTISSLQAEDVAVYYCQQHYSSPFTFGGGTKVEIK

SEQ ID NO: 316 - protein, 252 aa, synthetic construct
  source 1..252: note = CD98hc.04.064.1e
QVQLVQSGAEVKKPGASVKVSCKASGYTFTSYWMHWVRQAPGQGLEWMGIIDPSDSETHYAQKFQGRVTMTVDKSTSTVYMELSSLRSEDTAVYYCARASYGKGYFDYWGQGTLVTVSSGGSEGKSSGSGSESKSTGGSDIVMTQSPDSLAVSLGERATINCKSSQSLLNNINQKNYLAWYQQKPGQPPKLLIYFASTRESGVPDRFSGSGSGTDFTLTISSLQAEDVAVYYCQQHYSSPFTFGGGTKVEIK

SEQ ID NO: 317 - protein, 252 aa, synthetic construct
  source 1..252: note = CD98hc.04.064.1f
QVQLVQSGAEVKKPGASVKVSCKASGYTFTSYWMNWVRQAPGQGLEWMGIIDPSDSETHYAQKFQGRVTMTRDTSTSTVYMELSSLRSEDTAVYYCARASYGKGYFDYWGQGTLVTVSSGGSEGKSSGSGSESKSTGGSDIVMTQSPDSLAVSLGERATINCKSSQSLLNNINQKNYLAWYQQKPGQPPKLLIYFASTRESGVPDRFSGSGSGTDFTLTISSLQAEDVAVYYCQQHYSSPFTFGGGTKVEIK

SEQ ID NO: 318 - protein, 252 aa, synthetic construct
  source 1..252: note = CD98hc.04.064.4a
QVQLVQSGAEVKKPGASVKVSCKASGYTFTSYWMNWVRQAPGQGLEWMGIIDPSDSETNYAQKFQGRVTSTVDKSISTAYMELSRLRSDDTVVYYCARASYGKGYFDYWGQGTLVTVSSGGSEGKSSGSGSESKSTGGSDIVMTQSPDSLAVSLGERATINCKSSQSLLNNINQKNYLAWYQQKPGQPPKLLIYFASTRESGVPDRFSGSGSGTDFTLTISSLQAEDVAVYYCQQHYSSPFTFGGGTKVEIK

SEQ ID NO: 319 - protein, 252 aa, synthetic construct
  source 1..252: note = CD98hc.04.064.4b
QVQLVQSGAEVKKPGASVKVSCKASGYTFTSYWMNWVRQAPGQGLEWMGIIDPSDSETNYAQKFQGRVTSTVDKSISTAYMELSRLRSDDTVVYYCARASYGKGYFDYWGQGTLVTVSSGGSEGKSSGSGSESKSTGGSDIVMTQSPDSLAVSLGERATINCKSSQSLLNNINQKNYLAWYQQKPGQPPKLLIYFASTRESGVPDRFSGSGSGTDFTLTISSLQAEDVAVYFCQQHYSSPFTFGGGTKVEIK

SEQ ID NO: 320 - protein, 252 aa, synthetic construct
  source 1..252: note = CD98hc.04.064.4c
QVQLVQSGAEVKKPGASVKVSCKASGYTFTSYWMHWVRQAPGQGLEWMGIIDPSDSETNYAQKFQGRVTSTVDKSISTAYMELSRLRSDDTVVYYCARASYGKGYFDYWGQGTLVTVSSGGSEGKSSGSGSESKSTGGSDIVMTQSPDSLAVSLGERATINCKSSQSLLNNINQKNYLAWYQQKPGQPPKLLIYFASTRESGVPDRFSGSGSGTDFTLTISSLQAEDVAVYYCQQHYSSPFTFGGGTKVEIK

SEQ ID NO: 321 - protein, 252 aa, synthetic construct
  source 1..252: note = CD98hc.04.064.4d
QVQLVQSGAEVKKPGASVKVSCKASGYTFTSYWMHWVRQAPGQGLEWMGIIDPSDSETSYAQKFQGRVTMTRDTSTSTVYMELSSLRSEDTAVYYCARASYGKGYFDYWGQGTLVTVSSGGSEGKSSGSGSESKSTGGSDIQMTQSPSSLSASVGDRVTITCRASQSLLNNINQKNYLAWYQQKPGKAPKLLIYFASSLQSGVPSRFSGSGSGTDFTLTISSLQPEDFATYYCQQHYSSPFTFGGGTKVEIK

SEQ ID NO: 322 - protein, 252 aa, synthetic construct
  source 1..252: note = CD98hc.04.064.4a.1
QVQLVQSGAEVKKPGASVKVSCKASGYTFTSYYMNWVRQAPGQGLEWMGIIDPSESETNYAQKFQGRVTSTVDKSISTAYMELSRLRSDDTVVYYCARASYGKGYFDYWGQGTLVTVSSGGSEGKSSGSGSESKSTGGSDIVMTQSPDSLAVSLGERATINCKSSQSLLNNINQKNYLAWYQQKPGQPPKLLIYFASTRESGVPDRFSGSGSGTDFTLTISSLQAEDVAVYYCQQHYSSPFTFGGGTKVEIK

SEQ ID NO: 323 - protein, 252 aa, synthetic construct
  source 1..252: note = CD98hc.04.064.4a.2
QVQLVQSGAEVKKPGASVKVSCKASGYTFTSYYMNWVRQAPGQGLEWMGIIDPSQSETNYAQKFQGRVTSTVDKSISTAYMELSRLRSDDTVVYYCARASYGKGYFDYWGQGTLVTVSSGGSEGKSSGSGSESKSTGGSDIVMTQSPDSLAVSLGERATINCKSSQSLLNNINQKNYLAWYQQKPGQPPKLLIYFASTRESGVPDRFSGSGSGTDFTLTISSLQAEDVAVYYCQQHYSSPFTFGGGTKVEIK

SEQ ID NO: 324 - protein, 252 aa, synthetic construct
  source 1..252: note = CD98hc.04.064.4a.3
QVQLVQSGAEVKKPGASVKVSCKASGYTFTSYYMNWVRQAPGQGLEWMGIIDPSSSETNYAQKFQGRVTSTVDKSISTAYMELSRLRSDDTVVYYCARASYGKGYFDYWGQGTLVTVSSGGSEGKSSGSGSESKSTGGSDIVMTQSPDSLAVSLGERATINCKSSQSLLNNINQKNYLAWYQQKPGQPPKLLIYFASTRESGVPDRFSGSGSGTDFTLTISSLQAEDVAVYYCQQHYSSPFTFGGGTKVEIK

SEQ ID NO: 325 - protein, 252 aa, synthetic construct
  source 1..252: note = CD98hc.04.064.4a.4
QVQLVQSGAEVKKPGASVKVSCKASGYTFTSYYMNWVRQAPGQGLEWMGIIDPSGSETNYAQKFQGRVTSTVDKSISTAYMELSRLRSDDTVVYYCARASYGKGYFDYWGQGTLVTVSSGGSEGKSSGSGSESKSTGGSDIVMTQSPDSLAVSLGERATINCKSSQSLLNNINQKNYLAWYQQKPGQPPKLLIYFASTRESGVPDRFSGSGSGTDFTLTISSLQAEDVAVYYCQQHYSSPFTFGGGTKVEIK

SEQ ID NO: 326 - protein, 252 aa, synthetic construct
  source 1..252: note = CD98hc.04.064.4a.5
QVQLVQSGAEVKKPGASVKVSCKASGYTFTSYYMNWVRQAPGQGLEWMGIIDPSASETNYAQKFQGRVTSTVDKSISTAYMELSRLRSDDTVVYYCARASYGKGYFDYWGQGTLVTVSSGGSEGKSSGSGSESKSTGGSDIVMTQSPDSLAVSLGERATINCKSSQSLLNNINQKNYLAWYQQKPGQPPKLLIYFASTRESGVPDRFSGSGSGTDFTLTISSLQAEDVAVYYCQQHYSSPFTFGGGTKVEIK

SEQ ID NO: 327 - protein, 252 aa, synthetic construct
  source 1..252: note = CD98hc.04.064.4a.6
QVQLVQSGAEVKKPGASVKVSCKASGYTFTSYYMNWVRQAPGQGLEWMGIIDPSESETNYAQKFQGRVTSTVDKSISTAYMELSRLRSDDTVVYYCARASYGKGYFDYWGQGTLVTVSSGGSEGKSSGSGSESKSTGGSDIVMTQSPDSLAVSLGERATINCKSSQSLLENINQKNYLAWYQQKPGQPPKLLIYFASTRESGVPDRFSGSGSGTDFTLTISSLQAEDVAVYYCQQHYSSPFTFGGGTKVEIK

SEQ ID NO: 328 - protein, 252 aa, synthetic construct
  source 1..252: note = CD98hc.04.064.4a.7
QVQLVQSGAEVKKPGASVKVSCKASGYTFTSYYMNWVRQAPGQGLEWMGIIDPSESETNYAQKFQGRVTSTVDKSISTAYMELSRLRSDDTVVYYCARASYGKGYFDYWGQGTLVTVSSGGSEGKSSGSGSESKSTGGSDIVMTQSPDSLAVSLGERATINCKSSQSLLQNINQKNYLAWYQQKPGQPPKLLIYFASTRESGVPDRFSGSGSGTDFTLTISSLQAEDVAVYYCQQHYSSPFTFGGGTKVEIK

SEQ ID NO: 329 - protein, 252 aa, synthetic construct
  source 1..252: note = CD98hc.04.064.4a.8
QVQLVQSGAEVKKPGASVKVSCKASGYTFTSYYMNWVRQAPGQGLEWMGIIDPSESETNYAQKFQGRVTSTVDKSISTAYMELSRLRSDDTVVYYCARASYGKGYFDYWGQGTLVTVSSGGSEGKSSGSGSESKSTGGSDIVMTQSPDSLAVSLGERATINCKSSQSLLSNINQKNYLAWYQQKPGQPPKLLIYFASTRESGVPDRFSGSGSGTDFTLTISSLQAEDVAVYYCQQHYSSPFTFGGGTKVEIK

SEQ ID NO: 330 - protein, 252 aa, synthetic construct
  source 1..252: note = CD98hc.04.064.4a.9
QVQLVQSGAEVKKPGASVKVSCKASGYTFTSYYMNWVRQAPGQGLEWMGIIDPSESETNYAQKFQGRVTSTVDKSISTAYMELSRLRSDDTVVYYCARASYGKGYFDYWGQGTLVTVSSGGSEGKSSGSGSESKSTGGSDIVMTQSPDSLAVSLGERATINCKSSQSLLGNINQKNYLAWYQQKPGQPPKLLIYFASTRESGVPDRFSGSGSGTDFTLTISSLQAEDVAVYYCQQHYSSPFTFGGGTKVEIK

SEQ ID NO: 331 - protein, 252 aa, synthetic construct
  source 1..252: note = CD98hc.04.064.4a.10
QVQLVQSGAEVKKPGASVKVSCKASGYTFTSYYMNWVRQAPGQGLEWMGIIDPSESETNYAQKFQGRVTSTVDKSISTAYMELSRLRSDDTVVYYCARASYGKGYFDYWGQGTLVTVSSGGSEGKSSGSGSESKSTGGSDIVMTQSPDSLAVSLGERATINCKSSQSLLANINQKNYLAWYQQKPGQPPKLLIYFASTRESGVPDRFSGSGSGTDFTLTISSLQAEDVAVYYCQQHYSSPFTFGGGTKVEIK

SEQ ID NO: 332 - protein, 252 aa, synthetic construct
  source 1..252: note = CD98hc.04.064.4a.11
QVQLVQSGAEVKKPGASVKVSCKASGYTFTSYYMNWVRQAPGQGLEWMGIIDPSESETNYAQKFQGRVTSTVDKSISTAYMELSRLRSDDTVVYYCARASYGKGYFDYWGQGTLVTVSSGGSEGKSSGSGSESKSTGGSDIVMTQSPDSLAVSLGERATINCKSSQSLLHNINQKNYLAWYQQKPGQPPKLLIYFASTRESGVPDRFSGSGSGTDFTLTISSLQAEDVAVYYCQQHYSSPFTFGGGTKVEIK

SEQ ID NO: 333 - protein, 252 aa, synthetic construct
  source 1..252: note = CD98hc.04.064.4a.12
QVQLVQSGAEVKKPGASVKVSCKASGYTFTSYYMNWVRQAPGQGLEWMGIIDPSESETNYAQKFQGRVTSTVDKSISTAYMELSRLRSDDTVVYYCARASYGKGYFDYWGQGTLVTVSSGGSEGKSSGSGSESKSTGGSDIVMTQSPDSLAVSLGERATINCKSSQSLLYNINQKNYLAWYQQKPGQPPKLLIYFASTRESGVPDRFSGSGSGTDFTLTISSLQAEDVAVYYCQQHYSSPFTFGGGTKVEIK

SEQ ID NO: 334 - protein, 252 aa, synthetic construct
  source 1..252: note = CD98hc.04.064.4a.13
QVQLVQSGAEVKKPGASVKVSCKASGYTFTSYAMNWVRQAPGQGLEWMGIIDPSESETNYAQKFQGRVTSTVDKSISTAYMELSRLRSDDTVVYYCARASYGKGYFDYWGQGTLVTVSSGGSEGKSSGSGSESKSTGGSDIVMTQSPDSLAVSLGERATINCKSSQSLLNNINQKNYLAWYQQKPGQPPKLLIYFASTRESGVPDRFSGSGSGTDFTLTISSLQAEDVAVYYCQQHYSSPFTFGGGTKVEIK

SEQ ID NO: 335 - protein, 252 aa, synthetic construct
  source 1..252: note = CD98hc.04.064.4a.15
QVQLVQSGAEVKKPGASVKVSCKASGYTFTSYFMNWVRQAPGQGLEWMGIIDPSESETNYAQKFQGRVTSTVDKSISTAYMELSRLRSDDTVVYYCARASYGKGYFDYWGQGTLVTVSSGGSEGKSSGSGSESKSTGGSDIVMTQSPDSLAVSLGERATINCKSSQSLLNNINQKNYLAWYQQKPGQPPKLLIYFASTRESGVPDRFSGSGSGTDFTLTISSLQAEDVAVYYCQQHYSSPFTFGGGTKVEIK

SEQ ID NO: 336 - protein, 252 aa, synthetic construct
  source 1..252: note = CD98hc.04.064.4a.16
QVQLVQSGAEVKKPGASVKVSCKASGYTFTSYFMNWVRQAPGQGLEWMGIIDPSASETNYAQKFQGRVTSTVDKSISTAYMELSRLRSDDTVVYYCARASYGKGYFDYWGQGTLVTVSSGGSEGKSSGSGSESKSTGGSDIVMTQSPDSLAVSLGERATINCKSSQSLLGNINQKNYLAWYQQKPGQPPKLLIYFASTRESGVPDRFSGSGSGTDFTLTISSLQAEDVAVYYCQQHYSSPFTFGGGTKVEIK

SEQ ID NO: 337 - protein, 20 aa, synthetic construct
GGGGSGGGGSGGGGSGGGGS

SEQ ID NO: 338 - protein, 5 aa, synthetic construct
  REGION 1..5: note = The entire sequence of amino acids 1-5 is repeated 3 times
GGSGG

SEQ ID NO: 339 - protein, 119 aa, synthetic construct
  source 1..119: note = BACE1 VH
EVQLVESGGGLVQPGGSLRLSCAASGFTFSGYAIHWVRQAPGKGLEWVGWISPAGGSTDYADSVKGRFTISADTSKNTAYLQMNSLRAEDTAVYYCARGPFSPWVMDYWGQGTLVTVSS

SEQ ID NO: 340 - protein, 108 aa, synthetic construct
  source 1..108: note = BACE1 VL
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKR

SEQ ID NO: 341 - protein, 126 aa, synthetic construct
  source 1..126: note = BACE1 VH
EVQLVESGGGLVQPGGSLRLSCAASGFNFYYSSIHWVRQAPGKGLEWVASISPYSGYTSYADSVKGRFTISADTSKNTAYLQMNSLRAEDTAVYYCARQPTHYYYYAKGYKAMDYWGQGTLVTVSS

SEQ ID NO: 342 - protein, 108 aa, synthetic construct
  source 1..108: note = BACE1 VL
DIQMTQSPSSLSASVGDRVTITCRASQSVSSAVAWYQQKPGKAPKLLIYSASSLYSGVPSRFSGSGSGTDFTLTISSLQPEDFATYYCQQYSYSPFTFGQGTKVEIKR

SEQ ID NO: 343 - protein, 116 aa, synthetic construct
  source 1..116: note = Alpha-synuclein VH
EVQLLESGGGLVQPGGSLRLSCAASGFTFSRFTMTWVRQAPGKGLEWVSAISGSGGGTSYADSVKGRLTVSRDNSKNTLYLQMNSLRAEDTAVYYCAKNWAPFDYWGQGTLVTVSS

SEQ ID NO: 344 - protein, 108 aa, synthetic construct
  source 1..108: note = Alpha-synuclein VL
EIVLTQSPGTLSLSPGERATLSCRASQSVSRSYLAWYQQKPGQAPRLLIYGASSRATGIPDRFSGSGSGTDFTLTVSRLEPEDFAVYYCQQYGSSPWTFGQGTKVEIK

SEQ ID NO: 345 - protein, 116 aa, synthetic construct
  source 1..116: note = Alpha-synuclein VH
EVQLLESGGGLVQTGGSLRLSCAASGFTFSSYAMTWVRQAPGKGLEWVSAIRSNGDRTDYADSVKGRFTISRDNSQNTLYLQMNSLRAEDTAVYYCAKNWAPFDSWGQGTLVTVSS

SEQ ID NO: 346 - protein, 108 aa, synthetic construct
  source 1..108: note = Alpha-synuclein VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 347 - protein, 115 aa, synthetic construct
  source 1..115: note = Abeta VH
QVQLVQSGAEVKKPGSSVKVSCKASGYDFTRYYINWVRQAPGQGLEWMGWINPGSGNTKYNEKFKGRVTITADESTSTAYMELSSLRSEDTAVYYCAREGITVYWGQGTTVTVSS

SEQ ID NO: 348 - protein, 112 aa, synthetic construct
  source 1..112: note = Abeta VL
DIVMTQTPLSLSVTPGQPASISCKSSQSLLYSRGKTYLNWLLQKPGQSPQLLIYAVSKLDSGVPDRFSGSGSGTDFTLKISRVEAEDVGVYYCVQGTHYPFTFGQGTKLEIK

SEQ ID NO: 349 - protein, 121 aa, synthetic construct
  source 1..121: note = Tau VH
EVQLVESGGGLVQPGGSLKLSCAASGFNFNISAIHWVRQASGKGLEWVGRIRSKSHNYATLYAASLKGRFTLSRDDSRNTAYLQMSSLQTEDMAVYYCTVLSANYDTFDYWGQGTLVTVSS

SEQ ID NO: 350 - protein, 107 aa, synthetic construct
  source 1..107: note = Tau VL
SYELTQPPSVSVSPGQTARISCFGDTLPKQYTYWYQQKPGQAPVLVIYKDTERPSGIPERFSGSSSGTTVTLTISGVQAEDEADYYCLSADNSATWVFGGGTKVTVL

SEQ ID NO: 351 - protein, 4 aa, synthetic construct
GGGG

SEQ ID NO: 352 - protein, 6 aa, synthetic construct
GGGXGG

SEQ ID NO: 353 - protein, 4 aa, synthetic construct
GGGS

SEQ ID NO: 354 - protein, 5 aa, synthetic construct
GGSGG

SEQ ID NO: 355 - protein, 119 aa, synthetic construct
  source 1..119: note = CD98hc.04.064.1a VH
QVQLVQSGAEVKKPGASVKVSCKASGYTFTSYWMNWVRQAPGQGLEWMGIIDPSDSETHYAQKFQGRVTMTVDKSTSTVYMELSSLRSEDTAVYYCARASYGKGYFDYWGQGTLVTVSS

SEQ ID NO: 356 - protein, 113 aa, synthetic construct
  source 1..113: note = CD98hc.04.064.1a VL
DIVMTQSPDSLAVSLGERATINCKSSQSLLNNINQKNYLAWYQQKPGQPPKLLIYFASTRESGVPDRFSGSGSGTDFTLTISSLQAEDVAVYYCQQHYSSPFTFGGGTKVEIK

SEQ ID NO: 357 - protein, 119 aa, synthetic construct; identical to SEQ ID NO: 355
  source 1..119: note = CD98hc.04.064.1b VH

SEQ ID NO: 358 - protein, 113 aa, synthetic construct
  source 1..113: note = CD98hc.04.064.1b VL
DIVMTQSPDSLAVSLGERATINCKSSQSLLNNINQKNYLAWYQQKPGQPPKLLIYFASTRESGVPDRFSGSGSGTDFTLTISSLQAEDVAVYFCQQHYSSPFTFGGGTKVEIK

SEQ ID NO: 359 - protein, 119 aa, synthetic construct
  source 1..119: note = CD98hc.04.064.1c VH
QVQLVQSGAEVKKPGASVKVSCKASGYTFTSYWMHWVRQAPGQGLEWMGIIDPSDSETSYAQKFQGRVTMTVDKSTSTVYMELSSLRSEDTAVYYCARASYGKGYFDYWGQGTLVTVSS

SEQ ID NO: 360 - protein, 113 aa, synthetic construct; identical to SEQ ID NO: 356
  source 1..113: note = CD98hc.04.064.1c VL

SEQ ID NO: 361 - protein, 119 aa, synthetic construct
  source 1..119: note = CD98hc.04.064.1d VH
QVQLVQSGAEVKKPGASVKVSCKASGYTFTSYWMNWVRQAPGQGLEWMGIIDPSDSETSYAQKFQGRVTMTVDKSTSTVYMELSSLRSEDTAVYYCARASYGKGYFDYWGQGTLVTVSS

SEQ ID NO: 362 - protein, 113 aa, synthetic construct; identical to SEQ ID NO: 356
  source 1..113: note = CD98hc.04.064.1d VL

SEQ ID NO: 363 - protein, 119 aa, synthetic construct
  source 1..119: note = CD98hc.04.064.1e VH
QVQLVQSGAEVKKPGASVKVSCKASGYTFTSYWMHWVRQAPGQGLEWMGIIDPSDSETHYAQKFQGRVTMTVDKSTSTVYMELSSLRSEDTAVYYCARASYGKGYFDYWGQGTLVTVSS

SEQ ID NO: 364 - protein, 113 aa, synthetic construct; identical to SEQ ID NO: 356
  source 1..113: note = CD98hc.04.064.1e VL

SEQ ID NO: 365 - protein, 119 aa, synthetic construct
  source 1..119: note = CD98hc.04.064.1f VH
QVQLVQSGAEVKKPGASVKVSCKASGYTFTSYWMNWVRQAPGQGLEWMGIIDPSDSETHYAQKFQGRVTMTRDTSTSTVYMELSSLRSEDTAVYYCARASYGKGYFDYWGQGTLVTVSS

SEQ ID NO: 366 - protein, 113 aa, synthetic construct; identical to SEQ ID NO: 356
  source 1..113: note = CD98hc.04.064.1f VL

SEQ ID NO: 367 - protein, 119 aa, synthetic construct
  source 1..119: note = CD98hc.04.064.4a VH
QVQLVQSGAEVKKPGASVKVSCKASGYTFTSYWMNWVRQAPGQGLEWMGIIDPSDSETNYAQKFQGRVTSTVDKSISTAYMELSRLRSDDTVVYYCARASYGKGYFDYWGQGTLVTVSS

SEQ ID NO: 368 - protein, 113 aa, synthetic construct; identical to SEQ ID NO: 356
  source 1..113: note = CD98hc.04.064.4a VL

SEQ ID NO: 369 - protein, 119 aa, synthetic construct; identical to SEQ ID NO: 367
  source 1..119: note = CD98hc.04.064.4b VH

SEQ ID NO: 370 - protein, 113 aa, synthetic construct; identical to SEQ ID NO: 358
  source 1..113: note = CD98hc.04.064.4b VL

SEQ ID NO: 371 - protein, 119 aa, synthetic construct
  source 1..119: note = CD98hc.04.064.4c VH
QVQLVQSGAEVKKPGASVKVSCKASGYTFTSYWMHWVRQAPGQGLEWMGIIDPSDSETNYAQKFQGRVTSTVDKSISTAYMELSRLRSDDTVVYYCARASYGKGYFDYWGQGTLVTVSS

SEQ ID NO: 372 - protein, 113 aa, synthetic construct; identical to SEQ ID NO: 356
  source 1..113: note = CD98hc.04.064.4c VL

SEQ ID NO: 373 - protein, 119 aa, synthetic construct
  source 1..119: note = CD98hc.04.064.4d VH
QVQLVQSGAEVKKPGASVKVSCKASGYTFTSYWMHWVRQAPGQGLEWMGIIDPSDSETSYAQKFQGRVTMTRDTSTSTVYMELSSLRSEDTAVYYCARASYGKGYFDYWGQGTLVTVSS

SEQ ID NO: 374 - protein, 113 aa, synthetic construct
  source 1..113: note = CD98hc.04.064.4d VL
DIQMTQSPSSLSASVGDRVTITCRASQSLLNNINQKNYLAWYQQKPGKAPKLLIYFASSLQSGVPSRFSGSGSGTDFTLTISSLQPEDFATYYCQQHYSSPFTFGGGTKVEIK

SEQ ID NO: 375 - protein, 119 aa, synthetic construct
  source 1..119: note = CD98hc.04.064.4a.1 VH
QVQLVQSGAEVKKPGASVKVSCKASGYTFTSYYMNWVRQAPGQGLEWMGIIDPSESETNYAQKFQGRVTSTVDKSISTAYMELSRLRSDDTVVYYCARASYGKGYFDYWGQGTLVTVSS

SEQ ID NO: 376 - protein, 113 aa, synthetic construct; identical to SEQ ID NO: 356
  source 1..113: note = CD98hc.04.064.4a.1 VL

SEQ ID NO: 377 - protein, 119 aa, synthetic construct
  source 1..119: note = CD98hc.04.064.4a.2 VH
QVQLVQSGAEVKKPGASVKVSCKASGYTFTSYYMNWVRQAPGQGLEWMGIIDPSQSETNYAQKFQGRVTSTVDKSISTAYMELSRLRSDDTVVYYCARASYGKGYFDYWGQGTLVTVSS

SEQ ID NO: 378 - protein, 113 aa, synthetic construct; identical to SEQ ID NO: 356
  source 1..113: note = CD98hc.04.064.4a.2 VL

SEQ ID NO: 379 - protein, 119 aa, synthetic construct
  source 1..119: note = CD98hc.04.064.4a.3 VH
QVQLVQSGAEVKKPGASVKVSCKASGYTFTSYYMNWVRQAPGQGLEWMGIIDPSSSETNYAQKFQGRVTSTVDKSISTAYMELSRLRSDDTVVYYCARASYGKGYFDYWGQGTLVTVSS

SEQ ID NO: 380 - protein, 113 aa, synthetic construct; identical to SEQ ID NO: 356
  source 1..113: note = CD98hc.04.064.4a.3 VL

SEQ ID NO: 381 - protein, 119 aa, synthetic construct
  source 1..119: note = CD98hc.04.064.4a.4 VH
QVQLVQSGAEVKKPGASVKVSCKASGYTFTSYYMNWVRQAPGQGLEWMGIIDPSGSETNYAQKFQGRVTSTVDKSISTAYMELSRLRSDDTVVYYCARASYGKGYFDYWGQGTLVTVSS

SEQ ID NO: 382 - protein, 113 aa, synthetic construct; identical to SEQ ID NO: 356
  source 1..113: note = CD98hc.04.064.4a.4 VL

SEQ ID NO: 383 - protein, 119 aa, synthetic construct
  source 1..119: note = CD98hc.04.064.4a.5 VH
QVQLVQSGAEVKKPGASVKVSCKASGYTFTSYYMNWVRQAPGQGLEWMGIIDPSASETNYAQKFQGRVTSTVDKSISTAYMELSRLRSDDTVVYYCARASYGKGYFDYWGQGTLVTVSS

SEQ ID NO: 384 - protein, 113 aa, synthetic construct; identical to SEQ ID NO: 356
  source 1..113: note = CD98hc.04.064.4a.5 VL

SEQ ID NO: 385 - protein, 119 aa, synthetic construct; identical to SEQ ID NO: 375
  source 1..119: note = CD98hc.04.064.4a.6 VH

SEQ ID NO: 386 - protein, 113 aa, synthetic construct
  source 1..113: note = CD98hc.04.064.4a.6 VL
DIVMTQSPDSLAVSLGERATINCKSSQSLLENINQKNYLAWYQQKPGQPPKLLIYFASTRESGVPDRFSGSGSGTDFTLTISSLQAEDVAVYYCQQHYSSPFTFGGGTKVEIK

SEQ ID NO: 387 - protein, 119 aa, synthetic construct; identical to SEQ ID NO: 375
  source 1..119: note = CD98hc.04.064.4a.7 VH

SEQ ID NO: 388 - protein, 113 aa, synthetic construct
  source 1..113: note = CD98hc.04.064.4a.7 VL
DIVMTQSPDSLAVSLGERATINCKSSQSLLQNINQKNYLAWYQQKPGQPPKLLIYFASTRESGVPDRFSGSGSGTDFTLTISSLQAEDVAVYYCQQHYSSPFTFGGGTKVEIK

SEQ ID NO: 389 - protein, 119 aa, synthetic construct; identical to SEQ ID NO: 375
  source 1..119: note = CD98hc.04.064.4a.8 VH

SEQ ID NO: 390 - protein, 113 aa, synthetic construct
  source 1..113: note = CD98hc.04.064.4a.8 VL
DIVMTQSPDSLAVSLGERATINCKSSQSLLSNINQKNYLAWYQQKPGQPPKLLIYFASTRESGVPDRFSGSGSGTDFTLTISSLQAEDVAVYYCQQHYSSPFTFGGGTKVEIK

SEQ ID NO: 391 - protein, 119 aa, synthetic construct; identical to SEQ ID NO: 375
  source 1..119: note = CD98hc.04.064.4a.9 VH

SEQ ID NO: 392 - protein, 113 aa, synthetic construct
  source 1..113: note = CD98hc.04.064.4a.9 VL
DIVMTQSPDSLAVSLGERATINCKSSQSLLGNINQKNYLAWYQQKPGQPPKLLIYFASTRESGVPDRFSGSGSGTDFTLTISSLQAEDVAVYYCQQHYSSPFTFGGGTKVEIK

SEQ ID NO: 393 - protein, 119 aa, synthetic construct; identical to SEQ ID NO: 375
  source 1..119: note = CD98hc.04.064.4a.10 VH

SEQ ID NO: 394 - protein, 113 aa, synthetic construct
  source 1..113: note = CD98hc.04.064.4a.10 VL
DIVMTQSPDSLAVSLGERATINCKSSQSLLANINQKNYLAWYQQKPGQPPKLLIYFASTRESGVPDRFSGSGSGTDFTLTISSLQAEDVAVYYCQQHYSSPFTFGGGTKVEIK

SEQ ID NO: 395 - protein, 119 aa, synthetic construct; identical to SEQ ID NO: 375
  source 1..119: note = CD98hc.04.064.4a.11 VH

SEQ ID NO: 396 - protein, 113 aa, synthetic construct
  source 1..113: note = CD98hc.04.064.4a.11 VL
DIVMTQSPDSLAVSLGERATINCKSSQSLLHNINQKNYLAWYQQKPGQPPKLLIYFASTRESGVPDRFSGSGSGTDFTLTISSLQAEDVAVYYCQQHYSSPFTFGGGTKVEIK

SEQ ID NO: 397 - protein, 119 aa, synthetic construct; identical to SEQ ID NO: 375
  source 1..119: note = CD98hc.04.064.4a.12 VH

SEQ ID NO: 398 - protein, 113 aa, synthetic construct
  source 1..113: note = CD98hc.04.064.4a.12 VL
DIVMTQSPDSLAVSLGERATINCKSSQSLLYNINQKNYLAWYQQKPGQPPKLLIYFASTRESGVPDRFSGSGSGTDFTLTISSLQAEDVAVYYCQQHYSSPFTFGGGTKVEIK

SEQ ID NO: 399 - protein, 119 aa, synthetic construct
  source 1..119: note = CD98hc.04.064.4a.13 VH
QVQLVQSGAEVKKPGASVKVSCKASGYTFTSYAMNWVRQAPGQGLEWMGIIDPSESETNYAQKFQGRVTSTVDKSISTAYMELSRLRSDDTVVYYCARASYGKGYFDYWGQGTLVTVSS

SEQ ID NO: 400 - protein, 113 aa, synthetic construct; identical to SEQ ID NO: 356
  source 1..113: note = CD98hc.04.064.4a.13 VL

SEQ ID NO: 401 - protein, 119 aa, synthetic construct
  source 1..119: note = CD98hc.04.064.4a.15 VH
QVQLVQSGAEVKKPGASVKVSCKASGYTFTSYFMNWVRQAPGQGLEWMGIIDPSESETNYAQKFQGRVTSTVDKSISTAYMELSRLRSDDTVVYYCARASYGKGYFDYWGQGTLVTVSS

SEQ ID NO: 402 - protein, 113 aa, synthetic construct; identical to SEQ ID NO: 356
  source 1..113: note = CD98hc.04.064.4a.15 VL

SEQ ID NO: 403 - protein, 119 aa, synthetic construct
  source 1..119: note = CD98hc.04.064.4a.16 VH
QVQLVQSGAEVKKPGASVKVSCKASGYTFTSYFMNWVRQAPGQGLEWMGIIDPSASETNYAQKFQGRVTSTVDKSISTAYMELSRLRSDDTVVYYCARASYGKGYFDYWGQGTLVTVSS

SEQ ID NO: 404 - protein, 113 aa, synthetic construct; identical to SEQ ID NO: 392
  source 1..113: note = CD98hc.04.064.4a.16 VL

SEQ ID NO: 405 - protein, 112 aa, synthetic construct
  source 1..112: note = Variable light chain region
DVVMTQSPLSLPVTLGQPASISCKSSRSLLYSAGKTYLSWFQQRPGQSPRRLIYLVSKLDSGVPDRFSGSGSGTDFTLKISRVEAEDVGVYYCWQGIDFHQTFGGGTKVEIK

SEQ ID NO: 406 - protein, 107 aa, synthetic construct [sequence omitted]

SEQ ID NO: 407 - protein, 114 aa, synthetic construct
  source 1..114: note = Variable heavy chain region
QVQLVQSGSELKKPGASVKVSCKASGYAFTSYGLSWVRQAPGQGLEWMGWINTYSGVPTYAQGFTGRFVFSLDTSVSTAYLQISSLKAEDTAVYYCARTMADYWGQGTLVTVSS

SEQ ID NO: 408 - protein, 330 aa, synthetic construct
  source 1..330: note = CH+Fc #1 (knob, T366W)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 409 - protein, 330 aa, synthetic construct
  source 1..330: note = CH+Fc #2 (hole, T366S, L368A, Y407V)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 410 - protein, 267 aa, synthetic construct
  source 1..267: note = Linker-targeting domain
GGGGSGGGGSGGGGSQVQLVQSGAEVKKPGASVKVSCKASGYTFTSYWMHWVRQAPGQGLEWMGIIDPSDSETHYAQKFQGRVTMTVDKSTSTVYMELSSLRSEDTAVYYCARASYGKGYFDYWGQGTLVTVSSGGSEGKSSGSGSESKSTGGSDIVMTQSPDSLAVSLGERATINCKSSQSLLNNINQKNYLAWYQQKPGQPPKLLIYFASTRESGVPDRFSGSGSGTDFTLTISSLQAEDVAVYYCQQHYSSPFTFGGGTKVEIK

SEQ ID NO: 411 - protein, 444 aa, synthetic construct
  source 1..444: note = TD1 heavy chain hIgG1 NSLF (N325S, L328F)
QVQLVQSGSELKKPGASVKVSCKASGYAFTSYGLSWVRQAPGQGLEWMGWINTYSGVPTYAQGFTGRFVFSLDTSVSTAYLQISSLKAEDTAVYYCARTMADYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSSKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 412 - protein, 219 aa, synthetic construct
  source 1..219: note = TD1 light chain
DVVMTQSPLSLPVTLGQPASISCKSSRSLLYSAGKTYLSWFQQRPGQSPRRLIYLVSKLDSGVPDRFSGSGSGTDFTLKISRVEAEDVGVYYCWQGIDFHQTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 413 - protein, 5 aa, synthetic construct
  source 1..5: note = CD98hc.04.064.1c CDR-H1
SYWMH

SEQ ID NO: 414 - protein, 17 aa, synthetic construct
  source 1..17: note = CD98hc.04.064.1b CDR-H2
IIDPSDSETHYAQKFQG

SEQ ID NO: 415 - protein, 17 aa, synthetic construct
  source 1..17: note = CD98hc.04.064.1d CDR-H2
IIDPSDSETSYAQKFQG

SEQ ID NO: 416 - protein, 17 aa, synthetic construct
  source 1..17: note = CD98hc.04.064.4a CDR-H2
IIDPSDSETNYAQKFQG

SEQ ID NO: 417 - protein, 17 aa, synthetic construct
  source 1..17: note = CD98hc.04.064.4d CDR-H1
RASQSLLNNINQKNYLA

SEQ ID NO: 418 - protein, 7 aa, synthetic construct
  source 1..7: note = CD98hc.04.064.4d CDR-H2
FASSLQS

SEQ ID NO: 419 - protein, 5 aa, synthetic construct
  source 1..5: note = CD98hc.04.064.4a.1 CDR-H1
SYYMN

SEQ ID NO: 420 - protein, 5 aa, synthetic construct
  source 1..5: note = CD98hc.04.064.4a.13 CDR-H1
SYAMN

SEQ ID NO: 421 - protein, 5 aa, synthetic construct
  source 1..5: note = CD98hc.04.064.4a.15 CDR-H1
SYFMN

SEQ ID NO: 422 - protein, 17 aa, synthetic construct
  source 1..17: note = CD98hc.04.064.4a.15 CDR-H2
IIDPSESETNYAQKFQG

SEQ ID NO: 423 - protein, 17 aa, synthetic construct
  source 1..17: note = CD98hc.04.064.4a.2 CDR-H2
IIDPSQSETNYAQKFQG

SEQ ID NO: 424 - protein, 17 aa, synthetic construct
  source 1..17: note = CD98hc.04.064.4a.3 CDR-H2
IIDPSSSETNYAQKFQG

SEQ ID NO: 425 - protein, 17 aa, synthetic construct
  source 1..17: note = CD98hc.04.064.4a.4 CDR-H2
IIDPSGSETNYAQKFQG

SEQ ID NO: 426 - protein, 17 aa, synthetic construct
  source 1..17: note = CD98hc.04.064.4a.5 CDR-H2
IIDPSASETNYAQKFQG

SEQ ID NO: 427 - protein, 17 aa, synthetic construct
  source 1..17: note = CD98hc.04.064.4a.6 CDR-H1
KSSQSLLENINQKNYLA

SEQ ID NO: 428 - protein, 17 aa, synthetic construct
  source 1..17: note = CD98hc.04.064.4a.7 CDR-H1
KSSQSLLQNINQKNYLA

SEQ ID NO: 429 - protein, 17 aa, synthetic construct
  source 1..17: note = CD98hc.04.064.4a.8 CDR-H1
KSSQSLLSNINQKNYLA

SEQ ID NO: 430 - protein, 17 aa, synthetic construct
  source 1..17: note = CD98hc.04.064.4a.9 CDR-H1
KSSQSLLGNINQKNYLA

SEQ ID NO: 431 - protein, 17 aa, synthetic construct
  source 1..17: note = CD98hc.04.064.4a.10 CDR-H1
KSSQSLLANINQKNYLA

SEQ ID NO: 432 - protein, 17 aa, synthetic construct
  source 1..17: note = CD98hc.04.064.4a.11 CDR-H1
KSSQSLLHNINQKNYLA

SEQ ID NO: 433 - protein, 17 aa, synthetic construct
  source 1..17: note = CD98hc.04.064.4a.12 CDR-H1
KSSQSLLYNINQKNYLA

SEQ ID NO: 434 - protein, 18 aa, synthetic construct
  source 1..18: note = CD98hc.04.064.4a.16 CDR-H1
KSSQSLLGNNINQKNYLA

SEQ ID NO: 435 - protein, 630 aa, synthetic construct; identical to SEQ ID NO: 4
  source 1..630: note = human CD98Hc